SEQUENCE LISTING (411 sequences)

SEQ ID NO: 1 - DNA, 1846 bp, Thielavia terrestris
  source 1..1846: mol_type = genomic DNA
aattgaaggagggagtggcggagtggccaccaagtcaggcggctgtcaactaaccaaggatgggaacagttcggctcgccttgcccgagggcagcgttccctgatggggacgaaccatgggactggggtcagctgctgtataaaagttcaaatcgatgatctctcagatggcgctgctggggtgttctgcgcttttccatcctcgcaacctggtatcccactagtccagcgttcggcaccatgaagtcgttcaccattgccgccttggcagccctatgggcccaggaggccgccgcccacgcgaccttccaggacctctggattgatggagtcgactacggctcgcaatgtgtccgcctcccggcgtccaactcccccgtcaccaatgttgcgtccgacgatatccgatgcaatgtcggcacctcgaggcccaccgtcaagtgcccggtcaaggccggctccacggtcacgatcgagatgcaccaggttcgcacgcctctctgcgtaggccccccagctactatatggcactaacacgacctccagcaacctggcgaccggtcttgcgccaacgaggctatcggcggcgaccactacggccccgtaatggtgtacatgtccaaggtcgatgacgcggtgacagccgacggttcatcgggctggttcaaggtgttccaggacagctgggccaagaacccgtcgggttcgacgggcgacgacgactactggggcaccaaggacctcaactcgtgctgcggcaagatgaacgtcaagatccccgaagacatcgagccgggcgactacctgctccgcgccgaggttatcgcgctgcacgtggccgccagctcgggcggcgcgcagttctacatgtcctgctaccagctgaccgtgacgggctccggcagcgccaccccctcgaccgtgaatttcccgggcgcctactcggccagcgacccgggcatcctgatcaacatccacgcgcccatgtcgacctacgtcgtcccgggcccgaccgtgtacgcgggcggctcgaccaagtcggctggcagctcctgctccggctgcgaggcgacctgcacggttggttccggccccagcgcgacactgacgcagcccacctccaccgcgaccgcgacctccgcccctggcggcggcggctccggctgcacggcggccaagtaccagcagtgcggcggcaccggctacactgggtgcaccacctgcgctgtaagttccctcgtgatatgcagcggaacaccgtctggactgttttgctaactcgcgtcgtagtccgggtctacctgcagcgccgtctcgcctccgtactactcgcagtgcctctaagccgggagcgcttgctcagcgggctgctgtgaaggagctccatgtccccatgccgccatggccggagtaccgggctgagcgcccaattcttgtatatagttgagttttcccaatcatgaatacatatgcatctgcatggactgttgcgtcgtcagtctacatcctttgctccactgaactgtgagaccccatgtcatccggaccattcgatcggtgctcgctctaccatctcggttgatgggtctgggcttgagagtcactggcacgtcctcggcggtaatgaaatgtggaggaaagtgtgagctgtctgacgcactcggcgctgatgagacgttgagcgcggcccacactggtgttctgtaagccagcacacaaaagaatactccaggatggcccatagcggcaaatatacagtatcagggatgcaaaaagtgcaaaagtaaggggctcaatcggggatcgaacccgagacctcgcacatgacttatttcaagtcaggggt

SEQ ID NO: 2 - protein, 326 aa, Thielavia terrestris
MKSFTIAALAALWAQEAAAHATFQDLWIDGVDYGSQCVRLPASNSPVTNVASDDIRCNVGTSRPTVKCPVKAGSTVTIEMHQQPGDRSCANEAIGGDHYGPVMVYMSKVDDAVTADGSSGWFKVFQDSWAKNPSGSTGDDDYWGTKDLNSCCGKMNVKIPEDIEPGDYLLRAEVIALHVAASSGGAQFYMSCYQLTVTGSGSATPSTVNFPGAYSASDPGILINIHAPMSTYVVPGPTVYAGGSTKSAGSSCSGCEATCTVGSGPSATLTQPTSTATATSAPGGGGSGCTAAKYQQCGGTGYTGCTTCASGSTCSAVSPPYYSQCL

SEQ ID NO: 3 - DNA, 880 bp, Thielavia terrestris
  source 1..880: mol_type = genomic DNA
accccgggatcactgcccctaggaaccagcacacctcggtccaatcatgcggttcgacgccctctccgccctcgctcttgcgccgcttgtggctggccacggcgccgtgaccagctacatcatcggcggcaaaacctatcccggctacgagggcttctcgcctgcctcgagcccgccgacgatccagtaccagtggcccgactacaacccgaccctgagcgtgaccgacccgaagatgcgctgcaacggcggcacctcggcagagctcagcgcgcccgtccaggccggcgagaacgtgacggccgtctggaagcagtggacccaccagcaaggccccgtcatggtctggatgttcaagtgccccggcgacttctcgtcgtgccacggcgacggcaagggctggttcaagatcgaccagctgggcctgtggggcaacaacctcaactcgaacaactggggcaccgcgatcgtctacaagaccctccagtggagcaacccgatccccaagaacctcgcgccgggcaactacctcatccgccacgagctgctcgccctgcaccaggccaacacgccgcagttctacgccgagtgcgcccagctggtcgtctccggcagcggctccgccctgcccccgtccgactacctctacagcatccccgtctacgcgccccagaacgaccccggcatcaccgtgagtgggcttccgttccgcggcgagctctgtggaaatcttgctgacgatgggctaggttgacatctacaacggcgggcttacctcctacaccccgcccggcggccccgtctggtctggcttcgagttttaggcgcattgagtcgggggctacgaggggaaggcatctgttcgcatgagcgtgggtac

SEQ ID NO: 4 - protein, 239 aa, Thielavia terrestris
MRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEF

SEQ ID NO: 5 - DNA, 1000 bp, Thielavia terrestris
  source 1..1000: mol_type = genomic DNA
ctcctgttcctgggccaccgcttgttgcctgcactattggtagagttggtctattgctagagttggccatgcttctcacatcagtcctcggctcggctgccctgcttgctagcggcgctgcggcacacggcgccgtgaccagctacatcatcgccggcaagaattacccggggtgggtagctgattattgagggcgcattcaaggttcataccggtgtgcatggctgacaaccggctggcagataccaaggcttttctcctgcgaactcgccgaacgtcatccaatggcaatggcatgactacaaccccgtcttgtcgtgcagcgactcgaagcttcgctgcaacggcggcacgtcggccaccctgaacgccacggccgcaccgggcgacaccatcaccgccatctgggcgcagtggacgcacagccagggccccatcctggtgtggatgtacaagtgcccgggctccttcagctcctgtgacggctccggcgctggctggttcaagatcgacgaggccggcttccacggcgacggcgtcaaggtcttcctcgacaccgagaacccgtccggctgggacatcgccaagctcgtcggcggcaacaagcagtggagcagcaaggtccccgagggcctcgcccccggcaactacctcgtccgccacgagttgatcgccctgcaccaggccaacaacccgcagttctacccggagtgcgcccaggtcgtcatcaccggctccggcaccgcgcagccggatgcctcatacaaggcggctatccccggctactgcaaccagaatgacccgaacatcaaggtgagatccaggcgtaatgcagtctactgctggaaagaaagtggtccaagctaaaccgcgctccaggtgcccatcaacgaccactccatccctcagacctacaagattcccggccctcccgtcttcaagggcaccgccagcaagaaggcccgggacttcaccgcctgaagttgttgaatcgatggag

SEQ ID NO: 6 - protein, 258 aa, Thielavia terrestris
MLLTSVLGSAALLASGAAAHGAVTSYIIAGKNYPGYQGFSPANSPNVIQWQWHDYNPVLSCSDSKLRCNGGTSATLNATAAPGDTITAIWAQWTHSQGPILVWMYKCPGSFSSCDGSGAGWFKIDEAGFHGDGVKVFLDTENPSGWDIAKLVGGNKQWSSKVPEGLAPGNYLVRHELIALHQANNPQFYPECAQVVITGSGTAQPDASYKAAIPGYCNQNDPNIKVPINDHSIPQTYKIPGPPVFKGTASKKARDFTA

SEQ ID NO: 7 - DNA, 681 bp, Thielavia terrestris
  source 1..681: mol_type = genomic DNA
atgctcgcaaacggtgccatcgtcttcctggccgccgccctcggcgtcagtggccactacacctggccacgggttaacgacggcgccgactggcaacaggtccgtaaggcggacaactggcaggacaacggctacgtcggggatgtcacgtcgccacagatccgctgtttccaggcgaccccgtccccggccccatccgtcctcaacaccacggccggctcgaccgtgacctactgggccaaccccgacgtctaccaccccgggcctgtgcagttttacatggcccgcgtgcccgatggcgaggacatcaactcgtggaacggcgacggcgccgtgtggttcaaggtgtacgaggaccatcctacctttggcgctcagctcacatggcccagcacgggcaagagctcgttcgcggttcccatccccccgtgcatcaagtccggctactacctcctccgggcggagcaaatcggcctgcacgtcgcccagagcgtaggcggagcgcagttctacatctcatgcgcccagctcagcgtcaccggcggcggcagcaccgagccgccgaacaaggtggccttccccggcgcttacagtgcgacggacccgggcattctgatcaacatctactaccctgttcccacgtcctaccagaaccccggcccggccgtcttcagctgctga

SEQ ID NO: 8 - protein, 226 aa, Thielavia terrestris
MLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSC

SEQ ID NO: 9 - DNA, 960 bp, Thielavia terrestris
  source 1..960: mol_type = genomic DNA
atgaagggacttttcagtgccgccgccctctccctggccgtcggccaggcttcggcccattacatcttccagcaactctccatcaacgggaaccagtttccggtgtaccaatatattcgcaagaacaccaattataacagtcccgttaccgatctcacgtccgacgatcttcggtgcaatgtcggcgcccagggtgctgggacagacaccgtcacggtgaaggccggcgaccagttcaccttcacccttgacacccctgtttaccaccaggggcccatctccatctacatgtccaaggccccgggcgcggcgtcagactacgatggcagcggcggctggttcaagatcaaggactggggcccgactttcaacgccgacggcacggccacctgggacatggccggctcatacacctacaacatcccgacctgcattcccgacggcgactatctgctccgcatccagtcgctggccatccacaacccctggccggcgggcatcccgcagttctacatctcctgcgcccagatcaccgtgaccggcggcggcaacggcaaccctggcccgacggccctcatccccggcgccttcaaggacaccgacccgggctacacggtgaacatctacacgaacttccacaactacacggttcccggcccggaggtcttcagctgcaacggcggcggctcgaacccgcccccgccggtgagtagcagcacgcccgcgaccacgacgctggtcacgtcgacgcgcaccacgtcctccacgtcctccgcctcgacgccggcctcgaccggcggctgcaccgtcgccaagtggggccagtgcggcggcaacgggtacaccggctgcacgacctgcgcggccgggtccacctgcagcaagcagaacgactactactcgcagtgcttgtaagggaggccgcaaagcatgaggtgtttgaagaggaggagaggggtc

SEQ ID NO: 10 - protein, 304 aa, Thielavia terrestris
MKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCL

SEQ ID NO: 11 - DNA, 954 bp, Thielavia terrestris
  source 1..954: mol_type = genomic DNA
atgaagggcctcagcctcctcgccgctgcgtcggcagcgactgctcataccatcttcgtgcagctcgagtcagggggaacgacctatccggtatcctacggcatccgggaccctagctacgacggtcccatcaccgacgtcacctccgactcactggcttgcaatggtcccccgaaccccacgacgccgtccccgtacatcatcaacgtcaccgccggcaccacggtcgcggcgatctggaggcacaccctcacatccggccccgacgatgtcatggacgccagccacaaggggccgaccctggcctacctcaagaaggtcgatgatgccttgaccgacacgggtatcggcggcggctggttcaagatccaggaggccggttacgacaatggcaattgggctaccagcacggtgatcaccaacggtggcttccaatatattgacatccccgcctgcattcccaacggccagtatctgctccgcgccgagatgatcgcgctccacgccgccagcacgcagggtggtgcccagctctacatggagtgcgcgcagatcaacgtggtgggcggctccggcagcgccagcccgcagacgtacagcatcccgggcatctaccaggcaaccgacccgggcctgctgatcaacatctactccatgacgccgtccagccagtacaccattccgggtccgcccctgttcacctgcagcggcagcggcaacaacggcggcggcagcaacccgtcgggcgggcagaccacgacggcgaagcccacgacgacgacggcggcgacgaccacctcctccgccgctcctaccagcagccaggggggcagcagcggttgcaccgttccccagtggcagcagtgcggtggcatctcgttcaccggctgcaccacctgcgcggcgggctacacctgcaagtatctgaacgactattactcgcaatgccagtaa

SEQ ID NO: 12 - protein, 317 aa, Thielavia terrestris
MKGLSLLAAASAATAHTIFVQLESGGTTYPVSYGIRDPSYDGPITDVTSDSLACNGPPNPTTPSPYIINVTAGTTVAAIWRHTLTSGPDDVMDASHKGPTLAYLKKVDDALTDTGIGGGWFKIQEAGYDNGNWATSTVITNGGFQYIDIPACIPNGQYLLRAEMIALHAASTQGGAQLYMECAQINVVGGSGSASPQTYSIPGIYQATDPGLLINIYSMTPSSQYTIPGPPLFTCSGSGNNGGGSNPSGGQTTTAKPTTTTAATTTSSAAPTSSQGGSSGCTVPQWQQCGGISFTGCTTCAAGYTCKYLNDYYSQCQ

SEQ ID NO: 13 - DNA, 799 bp, Thermoascus aurantiacus
  source 1..799: mol_type = genomic DNA
atgtccttttccaagataattgctactgccggcgttcttgcctctgcttctctagtggctggccatggcttcgttcagaacatcgtgattgatggtaaaaagtatgtcattgcaagacgcacataagcggcaacagctgacaatcgacagttatggcgggtatctagtgaaccagtatccatacatgtccaatcctccagaggtcatcgcctggtctactacggcaactgatcttggatttgtggacggtactggataccaaaccccagatatcatctgccataggggcgccaagcctggagccctgactgctccagtctctccaggaggaactgttgagcttcaatggactccatggcctgattctcaccatggcccagttatcaactaccttgctccgtgcaatggtgattgttccactgtggataagacccaattagaattcttcaaaattgccgagagcggtctcatcaatgatgacaatcctcctgggatctgggcttcagacaatctgatagcagccaacaacagctggactgtcaccattccaaccacaattgcacctggaaactatgttctgaggcatgagattattgctcttcactcagctcagaaccaggatggtgcccagaactatccccagtgcatcaatctgcaggtcactggaggtggttctgataaccctgctggaactcttggaacggcactctaccacgataccgatcctggaattctgatcaacatctatcagaaactttccagctatatcatccctggtcctcctctgtatactggttaa

SEQ ID NO: 14 - protein, 249 aa, Thermoascus aurantiacus
MSFSKIIATAGVLASASLVAGHGFVQNIVIDGKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 15 - DNA, 1172 bp, Trichoderma reesei
  source 1..1172: mol_type = genomic DNA
ggatctaagccccatcgatatgaagtcctgcgccattcttgcagcccttggctgtcttgccgggagcgttctcggccatggacaagtccaaaacttcacgatcaatggacaatacaatcagggtttcattctcgattactactatcagaagcagaatactggtcacttccccaacgttgctggctggtacgccgaggacctagacctgggcttcatctcccctgaccaatacaccacgcccgacattgtctgtcacaagaacgcggccccaggtgccatttctgccactgcagcggccggcagcaacatcgtcttccaatggggccctggcgtctggcctcacccctacggtcccatcgttacctacgtggctgagtgcagcggatcgtgcacgaccgtgaacaagaacaacctgcgctgggtcaagattcaggaggccggcatcaactataacacccaagtctgggcgcagcaggatctgatcaaccagggcaacaagtggactgtgaagatcccgtcgagcctcaggcccggaaactatgtcttccgccatgaacttcttgctgcccatggtgcctctagtgcgaacggcatgcagaactatcctcagtgcgtgaacatcgccgtcacaggctcgggcacgaaagcgctccctgccggaactcctgcaactcagctctacaagcccactgaccctggcatcttgttcaacccttacacaacaatcacgagctacaccatccctggcccagccctgtggcaaggctagatccaggggtacggtgttggcgttcgtgaagtcggagctgttgacaaggatatctgatgatgaacggagaggactgatgggcgtgactgagtgtatatatttttgatgaccaaattgtatacgaaatccgaacgcatggtgatcattgtttatccctgtagtatattgtctccaggctgctaagagcccaccgggtgtattacggcaacaaagtcaggaatttgggtggcaatgaacgcaggtctccatgaatgtatatgtgaagaggcatcggctggcatgggcattaccagatataggccctgtgaaacatatagtacttgaacgtgctactggaacggatcataagcaagtcatcaacatgtgaaaaaacactacatgtaaaaaaaaaaaaaaaaaaaaa

SEQ ID NO: 16 - protein, 249 aa, Trichoderma reesei
MKSCAILAALGCLAGSVLGHGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 17 - DNA, 924 bp, Myceliophthora thermophila
  source 1..924: mol_type = genomic DNA
atgaagttcacctcgtccctcgctgtcctggccgctgccggcgcccaggctcactgttagtcgaccctcgaacccaacacccccctccccccttttctcctccatctcctcggcctcacttagtagccgctgacaacgactagataccttccctagggccggcactggtggctcgctctctggcgagtgggaggtggtccgcatgaccgagaaccattactcgcacggcccggtcaccgatgtcaccagccccgagatgacctgctatcagtccggcgtgcagggtgcgccccagaccgtccaggtcaaggcgggctcccaattcaccttcagcgtggatccctcgatcggccaccccggccctctccagttctacatggctaaggtgccgtcgggccagacggccgccacctttgacggcacgggagccgtgtggttcaagatctaccaagacggcccgaacggcctcggcaccgacagcattacctggcccagcgccggttcgtgacttcctccccactcgcttttttttttttattttttatttttttttctttcggaactcaagaatctttctctctctctcccgtctttggccttgaacaacactaaaactcttccttactgtattaattaggcaaaaccgaggtctcggtcaccatccccagctgcatcgatgatggcgagtacctgctccgggtcgagcacatcgcgctccacagcgccagcagcgtgggcggcgctcagttctacattgcctgcgcccagctctccgtcaccggcggctccggcaccctcaacacgggctcgctcgtctccctgcccggcgcctacaaggccaccgacccgggcatcctcttccagctctactggcccatcccgaccgagtacatcaaccccggcccggcccccgtctcttgctaa

SEQ ID NO: 18 - protein, 232 aa, Myceliophthora thermophila
MKFTSSLAVLAAAGAQAHYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIDDGEYLLRVEHIALHSASSVGGAQFYIACAQLSVTGGSGTLNTGSLVSLPGAYKATDPGILFQLYWPIPTEYINPGPAPVSC

SEQ ID NO: 19 - DNA, 854 bp, Myceliophthora thermophila
  source 1..854: mol_type = genomic DNA
atgaaggccctctctctccttgcggctgcctcggcagtctctgcgcataccatcttcgtccagctcgaagcagacggcacgaggtacccggtctcgtacgggatccgggacccaagctacgacggccccatcaccgacgtcacatccaacgacgttgcttgcaacggcgggccgaacccgacgaccccctccagcgacgtcatcaccgtcaccgcgggcaccacggtcaaggccatctggaggcacaccctccaatccggcccggacgatgtcatggacgccagccacaagggcccgaccctggcctacctcaagaaggtcggcgatgccaccaaggactcgggcgtcggcggtggctggttcaagattcaggaggacggctacaacaacggccagtggggcaccagcaccgttatctccaacggcggcgagcactacatgtgagccattcctccgagagaagaccaagactcttgacgatctcgctgacccgtgcaacaagtgacatcccggcctgcatccccgagggtcagtacctcctccgcgccgagatgatcgccctccacgcggccgggtcccccggcggtgcccagctctacgtaagcctctgcccttccccccttcctcttgatcgaatcggactgcccaccccccttttcgactccgactaacaccgttgccagatggaatgtgcccagatcaacatcgtcggcggctccggctcggtgcccagctcgaccgtcagcttccccggcgcgtacagccccaacgacccgggtctcctcatcaacatctattccatgtcgccctcgagctcgtacaccatcccgggcccgcccgtcttcaagtgctag

SEQ ID NO: 20 - protein, 235 aa, Myceliophthora thermophila
MKALSLLAAASAVSAHTIFVQLEADGTRYPVSYGIRDPSYDGPITDVTSNDVACNGGPNPTTPSSDVITVTAGTTVKAIWRHTLQSGPDDVMDASHKGPTLAYLKKVGDATKDSGVGGGWFKIQEDGYNNGQWGTSTVISNGGEHYIDIPACIPEGQYLLRAEMIALHAAGSPGGAQLYMECAQINIVGGSGSVPSSTVSFPGAYSPNDPGLLINIYSMSPSSSYTIPGPPVFKC

SEQ ID NO: 21 - DNA, 1242 bp, Myceliophthora thermophila
  source 1..1242: mol_type = genomic DNA
atgaagtccttcgccctcaccactctggccgccctggccggcaacgccgccgctcacgcgaccttccaggccctctgggtcgacggcgtcgactacggcgcgcagtgtgcccgtctgcccgcgtccaactccccggtcaccgacgtgacctccaacgcgatccgctgcaacgccaacccgtcgcccgctcggggcaagtgcccggtcaaggccggctcgaccgttacggtcgagatgcatcaggtacgttggatgaatgaaaggggaaaggaagcagaggcagaaggggaaggcgaagggaaagaaaaagaaaaagaaatggaaaagaaaaagaaatggaaaagaaaaagaaaaatgaaaaagaaagtggaaaccgtcagactaactggggctcctcccccccacccctcctttgatatcagcaacccggtgaccggtcgtgcagcagcgaggcgatcggcggggcgcactacggccccgtcatggtgtacatgtccaaggtgtcggacgcggcgtcggcggacgggtcgtcgggctggttcaaggtgttcgaggacggctgggccaagaacccgtccggcgggtcgggcgacgacgactactggggcaccaaggacctgaactcgtgctgcgggaagatgaacgtcaagatccccgccgacctgccctcgggcgactacctgctccgggccgaggccctcgcgctgcacacggcgggcagcgccggcggcgcccagttctacatgacgtgctaccagctcaccgtgacgggctccggcagcgccagcccgcccaccgtctccttcccgggcgcctacaaggccaccgacccgggcatcctcgtcaacatccacgccccgctgtccggctacaccgtgcccggcccggccgtctactccggcggctccaccaagaaggccggcagcgcctgcaccggctgcgagtccacctgcgccgtcggctccggccccaccgccaccgtctcccagtcgcccggttccaccgccacctccgcccccggcggcggcggcggctgcaccgtccagaagtaccagcagtgcggcggcgagggctacaccggctgcaccaactgcgcggtacgtttttcaaccccgtttttttttttccttccctaccttatttggttacctaattaattactttccggctgctgactttttgctttagtccggctctacctgcagcgccgtctcgccgccctactactcgcagtgcgtctaa

SEQ ID NO: 22 - protein, 323 aa, Myceliophthora thermophila
MKSFALTTLAALAGNAAAHATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGEGYTGCTNCASGSTCSAVSPPYYSQCV

SEQ ID NO: 23 - DNA, 1253 bp, Myceliophthora thermophila
  source 1..1253: mol_type = genomic DNA
atgaagccttttagcctcgtcgccctggcgaccgccgtgagcggccatgccatcttccagcgggtgtcggtcaacgggcaggaccagggccagctcaagggggtgcgggcgccgtcgagcaactccccgatccagaacgtcaacgatgccaacatggcctgcaacgccaacattgtgtaccacgacagcaccatcatcaaggtgcccgcgggagcccgcgtcggcgcgtggtggcagcacgtcatcggcgggccgcagggcgccaacgacccggacaacccgatcgcggcctcccacaagggtatgatgatcgatgatgcctctctcttcccccgttcttgatggacaggcgatggctcccaggaacacgcgtgactgaccaccgaatccaggccccatccaggtctacctggccaaggtggacaacgcggcgacggcgtcgccgtcgggcctcaggtggttcaaggtggccgagcgcggcctgaacaacggcgtgtgggccgtcgatgagctcatcgccaacaacggctggcactacttcgacctgccgtcgtgcgtggcccccggccagtacctgatgcgcgtcgagctgctcgccctgcacagcgcctcaagccccggcggcgcccagttctacatgggctgcgcacagatcgaaggtgcgtcgatctttgttctccttccgtgtcctctctgatcctttctctcttctttttctttcttttactccctttccttccatcttcggagaagcaacgaagggggaaagggatagaagagaggaatgagagacgacgaaagagaggattggggaaagacaagacagggaaaaaaagacaagaaaaaaaaaaaaaaaaaaaaacagagtgagctaacaagaacaatcagtcactggctccggcaccaactcgggctccgactttgtctcgttccccggcgcctactcggccaacgatccgggcatcttgctaagcatctacgacagctcgggcaagcccaccaacggcgggcgctcgtacccgatccccggcccgcgccccatctcctgctccggcagcggcgacggcggcaacaacggcggcggcggcgacgacaacaacaataacaacggtggtggcaacaacggcggcggcggcggcggcagcgtccccctgtacgggcagtgcggcggcatcggctacacgggcccgaccacctgtgcccagggaacttgcaaggtgtcgaacgaatactacagccagtgcctcccctag

SEQ ID NO: 24 - protein, 310 aa, Myceliophthora thermophila
MKPFSLVALATAVSGHAIFQRVSVNGQDQGQLKGVRAPSSNSPIQNVNDANMACNANIVYHDSTIIKVPAGARVGAWWQHVIGGPQGANDPDNPIAASHKGPIQVYLAKVDNAATASPSGLRWFKVAERGLNNGVWAVDELIANNGWHYFDLPSCVAPGQYLMRVELLALHSASSPGGAQFYMGCAQIEVTGSGTNSGSDFVSFPGAYSANDPGILLSIYDSSGKPTNGGRSYPIPGPRPISCSGSGDGGNNGGGGDDNNNNNGGGNNGGGGGGSVPLYGQCGGIGYTGPTTCAQGTCKVSNEYYSQCLP

SEQ ID NO: 25 - DNA, 814 bp, Myceliophthora thermophila
  source 1..814: mol_type = genomic DNA
atgaagctctccctcttctccgtcctggccactgccctcaccgtcgaggggcatgccatcttccagaaggtctccgtcaacggagcggaccagggctccctcaccggcctccgcgctcccaacaacaacaaccccgtgcaggatgtcaacagccaggacatgatctgcggccagtcgggatcgacgtcgaacactatcatcgaggtcaaggccggcgataggatcggtgcctggtatcagcatgtcatcggcggtgcccagttccccaacgacccagacaacccgattgccaagtcgcacaagggccccgtcatggcctacctcgccaaggttgacaatgccgcaaccgccagcaagacgggcctgaagtggtatgtattcccgcggcccgagggacatcgggttgggcaagtcgagactgacggagctcgcttctccgtataggttcaagatttgggaggatacctttaatcccagcaccaagacctggggtgtcgacaacctcatcaataacaacggctgggtgtacttcaacctcccgcagtgcatcgccgacggcaactacctcctccgcgtcgaggtcctcgctctgcactcggcctactctcagggccaggctcagttctaccagtcctgcgcccagatcaacgtatccggcggcggctccttcacaccgccgtcgactgtcagcttcccgggtgcctacagcgccagcgaccccggtatcctgatcaacatctacggcgccaccggccagcccgacaacaacggccagccgtacactgcccctgggcccgcgcccatctcctgctga

SEQ ID NO: 26 - protein, 246 aa, Myceliophthora thermophila
MKLSLFSVLATALTVEGHAIFQKVSVNGADQGSLTGLRAPNNNNPVQDVNSQDMICGQSGSTSNTIIEVKAGDRIGAWYQHVIGGAQFPNDPDNPIAKSHKGPVMAYLAKVDNAATASKTGLKWFKIWEDTFNPSTKTWGVDNLINNNGWVYFNLPQCIADGNYLLRVEVLALHSAYSQGQAQFYQSCAQINVSGGGSFTPPSTVSFPGAYSASDPGILINIYGATGQPDNNGQPYTAPGPAPISC

SEQ ID NO: 27 - DNA, 1115 bp, Thermoascus aurantiacus
  source 1..1115: mol_type = genomic DNA
atgtcgttctcgaagattgctgcgatcaccggggccattacctatgcgtctctggccgccgctcacggttatgttacaggaatcgtagccgatggcacctagtatgtaacgctcatgccaagatccgcattgctgtactaacaattagcagctacgggggctatatcgtgacccaatacccctacatgtcgacaccgccggatgtcatcgcctggtctaccaaagcaactgatcttggtttcgtggatcccagtagctatgcttcgtctgatattatctgccacaagggtgctgagcctggtgccctgagcgccaaggtggctgctggagggaccgtcgagctgcagtggacggattggcctgagagtcacaagggcccggtcattgactacctcgccgcctgtaacggggactgctcgactgtcgacaagaccaaactagagttcttcaagattgatgagagtggcctaattgacggcagcagcgccccaggcacatgggcctctgacaacttgattgccaataacaacagctggaccgtcaccatcccgagcacgattgctcccggcaactatgtcctgagacatgaaatcattgccctccactccgccggaaatacaaatggtgctcagaactacccccagtgtatcaaccttgaggtcacaggcagtggcaccgacacccctgccggcaccctcggaacggagctttataaggcaacggaccctggcattctggtcaacatctaccagaccctgaccagctacgatattcccggccctgctctgtacaccggtggtagctctggtagctctggttcctccaacaccgccaaggccaccacttcgacggcttctagctctatcgtgaccccgacgcctgttaacaacccaaccgttactcagactgccgttgttgatgtcacccagactgtttcccagaatgctgccgtcgccaccacgactccggcctccactgcagttgctacagctgtcccaacgggaaccacctttagctttgattcgatgacctcggatgaattcgtcagcctgatgcgtgcgaccgtgaattggctgctttctaacaagaagcatgcccgggatctttcttactaa

SEQ ID NO: 28 - protein, 354 aa, Thermoascus aurantiacus
MSFSKIAAITGAITYASLAAAHGYVTGIVADGTYYGGYIVTQYPYMSTPPDVIAWSTKATDLGFVDPSSYASSDIICHKGAEPGALSAKVAAGGTVELQWTDWPESHKGPVIDYLAACNGDCSTVDKTKLEFFKIDESGLIDGSSAPGTWASDNLIANNNSWTVTIPSTIAPGNYVLRHEIIALHSAGNTNGAQNYPQCINLEVTGSGTDTPAGTLGTELYKATDPGILVNIYQTLTSYDIPGPALYTGGSSGSSGSSNTAKATTSTASSSIVTPTPVNNPTVTQTAVVDVTQTVSQNAAVATTTPASTAVATAVPTGTTFSFDSMTSDEFVSLMRATVNWLLSNKKHARDLSY

SEQ ID NO: 29 - DNA, 862 bp, Aspergillus fumigatus
  source 1..862: mol_type = genomic DNA
atgactttgtccaagatcacttccattgctggccttctggcctcagcgtctctcgtggctggccacggctttgtttctggcattgttgctgatgggaaatagtatgtgcttgaaccacacaaatgacagctgcaacagctaacttctattccagttacggagggtaccttgttaaccaatacccctacatgagcaaccctcccgacaccattgcctggtccaccaccgccaccgacctcggctttgtggacggcaccggctaccagtctccggatattatctgccacagagacgcaaagaatggcaagttgaccgcaaccgttgcagccggttcacagatcgaattccagtggacgacgtggccagagtctcaccatggaccggtacgacgccgaagagaagagaacatattgtgaccagataggctaacatagcatagttgattacttacctcgctccatgcaacggcgactgtgccaccgtggacaagaccaccctgaagtttgtcaagatcgccgctcaaggcttgatcgacggctccaacccacctggtgtttgggctgatgatgaaatgatcgccaacaacaacacggccacagtgaccattcctgcctcctatgcccccggaaactacgtccttcgccacgagatcatcgcccttcactctgcgggtaacctgaacggcgcgcagaactacccccagtgtttcaacatccaaatcaccggtggcggcagtgctcagggatctggcaccgctggcacgtccctgtacaagaatactgatcctggcatcaagtttgacatctactcggatctgagcggtggataccctattcctggtcctgcactgttcaacgcttaa

SEQ ID NO: 30 - protein, 250 aa, Aspergillus fumigatus [sequence omitted]

SEQ ID NO: 31 - DNA, 1021 bp, Penicillium pinophilum
  source 1..1021: mol_type = genomic DNA
atgccttctactaaagtcgctgccctttctgctgttctagctttggcctccacggttgctggccatggttttgtgcaaaacatcgttatcgacggtaaatcgtaagcagtgatgcatccattattaaactagacatgcttacaaaaaaatcagttactctggataccttgtgaatcagttcccctacgagtccaacccaccagctgttattgggtgggcaacaactgcaaccgacctgggattcgtcgctcccagtgagtacaccaatgcagacattatctgccacaagaacgccacacctggcgcgctttctgctccagttgctgcagggggcactgtcgagctccagtggactacatggcccgatagtcatcacggtcctgtcatcagctacctcgccaactgcaatggcaattgttctaccgtggataagactaagctagactttgtcaagattgaccaaggtggtttgatcgacgatactacccccccgggtacatgggcttccgacaaacttatcgctgccaacaacagctggactgtaactatcccctccaccatcgcgcctggaaactacgttttgcgccacgaaatcattgctcttcactccgctggaaacgcagacggtgcccaaaactaccctcaatgcatcaacttggagatcaccggcagcggaaccgccgctccctctggtaccgctggcgaaaagctctacacctctactgaccccggtatcttggtcaatatctaccaatccttgtcgacctacgttattcccggaccaactctgtggagcggtgctgccaatggcgctgttgccactggttctgctactgcggttgctacgactgccactgcttctgcgaccgctactcctaccacacttgttacctctgtcgctccagcttcatctacctttgccactgctgttgtgaccactgtcgctcctgcagtaactgatgtcgtgactgtcaccgatgtagttaccgtgaccaccgtcatcaccactactgtcctttga

SEQ ID NO: 32 - protein, 322 aa, Penicillium pinophilum
MPSTKVAALSAVLALASTVAGHGFVQNIVIDGKSYSGYLVNQFPYESNPPAVIGWATTATDLGFVAPSEYTNADIICHKNATPGALSAPVAAGGTVELQWTTWPDSHHGPVISYLANCNGNCSTVDKTKLDFVKIDQGGLIDDTTPPGTWASDKLIAANNSWTVTIPSTIAPGNYVLRHEIIALHSAGNADGAQNYPQCINLEITGSGTAAPSGTAGEKLYTSTDPGILVNIYQSLSTYVIPGPTLWSGAANGAVATGSATAVATTATASATATPTTLVTSVAPASSTFATAVVTTVAPAVTDVVTVTDVVTVTTVITTTVL

SEQ ID NO: 33 - DNA, 1486 bp, Thermoascus sp.
  source 1..1486: mol_type = genomic DNA
atgttgtcgttcgcttctgccaagtcagctgtgctgacgacccttctacttcttggatccgctcaggctcacactttgatgaccaccctgtttgtggatggcgtcaatcagggagatggtgtctgtattcgcatgaacaacaacggtagtactgccaacacctatatccagcctgtcacgagcaaggatattgcctgcggtaagtacagtaccggtccagatatcatactctatttcaatccgacaacagtcagagctggagagcaatgctaaacatccccaggcattcaaggcgaaattggcgccgctcgagtctgtccagccaaggcttcatccaccctcacgttccaattccgagagcagccatccaacccgaattccgctcctctcgatccctcgcacaaaggccccgctgcggtgtacctgaaaaaggtagactccgccatcgcgagcaacaacgccgctggagacggctggttcaagatctgggagtccgtctacgacgagtccacgggcaaatggggtacgaccaagatgatcgagaacaacgggcacatctctgtcaaggtccccgacgatatcgagggtgggtattatctcgcgcgtacggagcttctggcgctgcacgcggcgaacgaaggggatccgcagttctacgttggctgcgcgcagctgttcatcgattcagcggggacagcgaaaccgcctactgtctctattggagaggggacctacgatctgagcatgcctgccatgacgtacaatatctaccagactccgttggctctaccatacccgatgtatgggcctcctgtctacacacctggctctggctcgggttctggctctggttccgggtcagcttctgcaacgagatcttctgctattcctactgccaccgctgttacggactgttcttccgaagaggacagggaagactcagtcatggcaaccggtgttcccgttgcaagaagcacactcagaacctgggttgacagactgtcatggcatggtaaggcccgtgagaacgtgaaaccagccgccaggagaagcgcccttgtccagaccgagggtctgaagccggaaggctgcatcttcgtcaacggcaactggtgcggtttcgaggtccccgattacaacgatgcggaaagctgctgggctgtacgttcccgtctaattacttaaaacgaaataaaagctaacagtacttttctttttctaatcccaggcctccgacaactgctggaaacagtccgactcgtgctggaaccagacccagcccaccggctacaacaactgccagatctggcaagaccagaaatgcaagcccatccaggactcgtgtagccaatccaacccgactggaccgccgaacaagggcaaggatataactccaacgtggccgcccctggagggctcgatgaagaccttcaccaagcgcactgtcagttaccgtgattggattatgaaaaggaaaggagcataa

SEQ ID NO: 34 - protein, 444 aa, Thermoascus sp.
MLSFASAKSAVLTTLLLLGSAQAHTLMTTLFVDGVNQGDGVCIRMNNNGSTANTYIQPVTSKDIACGIQGEIGAARVCPAKASSTLTFQFREQPSNPNSAPLDPSHKGPAAVYLKKVDSAIASNNAAGDGWFKIWESVYDESTGKWGTTKMIENNGHISVKVPDDIEGGYYLARTELLALHAANEGDPQFYVGCAQLFIDSAGTAKPPTVSIGEGTYDLSMPAMTYNIYQTPLALPYPMYGPPVYTPGSGSGSGSGSGSASATRSSAIPTATAVTDCSSEEDREDSVMATGVPVARSTLRTWVDRLSWHGKARENVKPAARRSALVQTEGLKPEGCIFVNGNWCGFEVPDYNDAESCWAASDNCWKQSDSCWNQTQPTGYNNCQIWQDQKCKPIQDSCSQSNPTGPPNKGKDITPTWPPLEGSMKTFTKRTVSYRDWIMKRKGA

SEQ ID NO: 35 - DNA, 835 bp, Penicillium sp.
  source 1..835: mol_type = genomic DNA
atgctgtcttcgacgactcgcaccctcgcctttacaggccttgcgggccttctgtccgctcccctggtcaaggcccatggctttgtccagggcattgtcatcggtgaccaattgtaagtccctctcttgcagttctgtcgattaactgctggactgcttgcttgactccctgctgactcccaacagctacagcgggtacatcgtcaactcgttcccctacgaatccaacccaccccccgtcatcggctgggccacgaccgccaccgacctgggcttcgtcgacggcacaggataccaaggcccggacatcatctgccaccggaatgcgacgcccgcgccgctgacagcccccgtggccgccggcggcaccgtcgagctgcagtggacgccgtggccggacagccaccacggacccgtcatcacctacctggcgccgtgcaacggcaactgctcgaccgtcgacaagacgacgctggagttcttcaagatcgaccagcagggcctgatcgacgacacgagcccgccgggcacctgggcgtcggacaacctcatcgccaacaacaatagctggaccgtcaccattcccaacagcgtcgcccccggcaactacgtcctgcgccacgagatcatcgccctgcactcggccaacaacaaggacggcgcccagaactacccccagtgcatcaacatcgaggtcacgggcggcggctccgacgcgcctgagggtactctgggcgaggatctctaccatgacaccgacccgggcattctggtcgacatttacgagcccattgcgacgtataccattccggggccgcctgagccgacgttctag

SEQ ID NO: 36 - protein, 253 aa, Penicillium sp.
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 37 - DNA, 878 bp, Thielavia terrestris
  source 1..878: mol_type = genomic DNA
atgaagttctcactggtgtctctgctggcttacggcctctcggtcgaggcgcactccatcttccaggttcgtctcgcacatcacgctcaactcggctcgtggcgtaagggcaaggattaacacggccggcagagagtctcggtcaacggccaagaccaaggcctgctcaccggcctccgcgctccaagcaacaacaacccagtgcaagatgtcaacagccagaacatgatttgcggccagtcgggctccaagtcgcagaccgttatcaacgtcaaggccggcgacaggatcggctcgctctggcagcatgtcatcggcggcgcccagttttcgggtgacccggacaacccgatcgcccactcgcacaagggccccgtgatggcgtaccttgctaaggtcgacaatgccgcgtccgcgagccaaacgggtctgaagtggtaagtagcgggcgacgctcaggggacggggatcgggggcctgctccatccgagactaacaccgtggacaggttcaagatctggcaggacgggttcgataccagcagcaagacatggggcgtcgacaacctgatcaagaacaacggctgggtgtacttccacctgccgcagtgcctcgctccgggccagtatctcctgcgcgtcgaggttctggcgctgcactcggcgtaccagcagggccaggcccagttctaccagtcctgcgcccagatcaacgtctccggctccgggtccttcagcccgtcccagacggtcagcatcccgggcgtctacagcgccaccgacccgagcatcctcatcaacatctacggcagcacggggcagcccgacaacggcggcaaggcttacaacccccctggacccgccccgatctcctgctga

SEQ ID NO: 38 - protein, 246 aa, Thielavia terrestris
MKFSLVSLLAYGLSVEAHSIFQRVSVNGQDQGLLTGLRAPSNNNPVQDVNSQNMICGQSGSKSQTVINVKAGDRIGSLWQHVIGGAQFSGDPDNPIAHSHKGPVMAYLAKVDNAASASQTGLKWFKIWQDGFDTSSKTWGVDNLIKNNGWVYFHLPQCLAPGQYLLRVEVLALHSAYQQGQAQFYQSCAQINVSGSGSFSPSQTVSIPGVYSATDPSILINIYGSTGQPDNGGKAYNPPGPAPISC

SEQ ID NO: 39 - DNA, 1253 bp, Thielavia terrestris
  source 1..1253: mol_type = genomic DNA
atgaggacgacattcgccgccgcgttggcagccttcgctgcgcaggaagtggcaggccatgccatcttccaacagctctgggtggacggcaccgactatatacgtgctccccttttccttttgtgtttgcccatcctcgattgataacccgaggccatccaatgctgactcttacagcacggctcctcctgcgtccgcatgccgctgtcgaactcgcccgtcacgaacgtcggcagcagggacatgatctgcaacgccggcacgcgccccgtcagcgggaagtgccccgtcaaggccggcggcaccgtgacggttgagatgcaccaggtgggctgatttcctgagcgtcctattcctcccggaagcccctttcccatcctttgccctggctaacccctccgcccctcccagcaacccggggatcggtcgtgtaacaacgaagccatcggcggcgcccactggggaccggtgcaggtgtacctcagcaaggtggaggacgcgagcacggcggacgggtcgacgggctggttcaagatcttcgcggacacgtggtccaagaaggcgggcagctcggtgggggacgacgacaactggggcacgcgcgacctcaacgcgtgctgcggcaagatgcaggtcaagatcccggcggacatcccgtcgggcgactacctgctgcgggcggaggcgctggcgctgcacacggcgggccaggtgggcggcgcgcagttctacatgagctgctaccagatcaccgtgtcgggcggcggcagcgccagcccggccaccgtcaagttccccggcgcctacagcgccaacgacccgggcatccacatcaacatccacgcggccgtgtccaactacgtcgcgcccggcccggccgtctattccggcggcacgaccaaggtggccgggtccgggtgccaaggctgcgagaacacgtgcaaggtcggctcgtcgcccacggcgacggcgccgtcgggcaagagcggcgcgggttccgacggcggcgctgggaccgacggcgggtcttcgtcttcgagccccgacacgggcagcgcgtgcagcgtgcaggcctacgggcagtgcggcgggaacgggtactcgggttgcacccagtgcgcggtaagttcggggtcgtctgtcttttgtaggaacatccgagaggcttggctgacgaggcgttgttgtagcccggctatacttgcaaggcggtctctccgccgtactattcgcagtgcgccccttcttcttag

SEQ ID NO: 40 - protein, 334 aa, Thielavia terrestris
MRTTFAAALAAFAAQEVAGHAIFQQLWHGSSCVRMPLSNSPVTNVGSRDMICNAGTRPVSGKCPVKAGGTVTVEMHQQPGDRSCNNEAIGGAHWGPVQVYLSKVEDASTADGSTGWFKIFADTWSKKAGSSVGDDDNWGTRDLNACCGKMQVKIPADIPSGDYLLRAEALALHTAGQVGGAQFYMSCYQITVSGGGSASPATVKFPGAYSANDPGIHINIHAAVSNYVAPGPAVYSGGTTKVAGSGCQGCENTCKVGSSPTATAPSGKSGAGSDGGAGTDGGSSSSSPDTGSACSVQAYGQCGGNGYSGCTQCAPGYTCKAVSPPYYSQCAPSS

SEQ ID NO: 41 - DNA, 798 bp, Thielavia terrestris
  source 1..798: mol_type = genomic DNA
atgaagctgagcgttgccatcgccgtgctggcgtcggctcttgccgaggctcactgtgagtgcatcgtctcactccagctactgcgaagcttgctgacgatggtccctagacaccttccccagcatcggaaacaccgctgactggcagtatgtgcggattacaacgaactaccagagcaacgggccggtgacggacgtcacctcggatcaaattcggtgctacgaacggaacccaggcacgggagcgcagggcatatacaacgtcaccgccggccagaccatcaactacaacgcgaaggcgtccatctcccacccggggcccatgtccttctacattgctaaggttcccgccggccaaaccgctgcgacctgggacggtaagggggctgtgtggaccaagatctaccaggacatgcccaagttcggcagcagcctgacctggcccaccatgggtaagaattctcaccctggaaatgaacgcacatttgcacagatctaacatggcctacaggcgccaagtctgtccccgtcaccatccctcgttgcctccagaacggcgattaccttctgcgagccgagcacatcgctctacacagcgcgagcagcgtcggtggcgcccagttctacctctcgtgcgcccagcttactgtcagcggcggcagtggcacctggaaccccaagaaccgggtctccttccccggcgcttacaaggcaacagacccgggcatcttgatcaacatctactaccccgtgccgaccagctactcgccgcccggcccgccggctgagacgtgctaa

SEQ ID NO: 42 - protein, 227 aa, Thielavia terrestris
MKLSVAIAVLASALAEAHYTFPSIGNTADWQYVRITTNYQSNGPVTDVTSDQIRCYERNPGTGAQGIYNVTAGQTINYNAKASISHPGPMSFYIAKVPAGQTAATWDGKGAVWTKIYQDMPKFGSSLTWPTMGAKSVPVTIPRCLQNGDYLLRAEHIALHSASSVGGAQFYLSCAQLTVSGGSGTWNPKNRVSFPGAYKATDPGILINIYYPVPTSYSPPGPPAETC

SEQ ID NO: 43 - DNA, 977 bp, Thielavia terrestris
  source 1..977: mol_type = genomic DNA
atgaagctgtcatcccagctcgccgccctcacgctggccgcggcctccgtgtcaggccactacatcttcgagcagattgcccatggcggcaccaagttcccaccttacgagtacatccgaagaaacacgaactataacagccctgtcaccagtctctcgtcgaacgacctgcgatgcaacgtaggcggcgagacggctggcaacacgaccgtcctcgacgtgaaggcgggcgactccttcaccttctactcggacgtggccgtgtaccaccaggggcccatctcactgtgcgtgccccgggccaactttgatcagtcccaagcggactgtccgctcgcctggataaccacaattgactgacagcccgcacagctacatgtccaaggctcccggctccgtcgtggactacgacggctccggcgactggttcaagatccacgactggggcccgaccttcagcaacggccaggcctcgtggccgctgcggggtgcgtcccttccctttccctcccccttcctcccccttcctccccccctttccccccttttctgtctggtcgcacgccctgctgacgtccccgtagacaactaccagtacaacatcccgacgtgcatcccgaacggcgagtacctgctgcgcatccagtcgctggcgatccacaacccgggcgccacgccgcagttctacatcagctgcgcgcaggtccgggtctcgggcggcggcagcgcctccccctccccaacggccaagatccccggcgcgttcaaggcgaccgatcccgggtataccgcgaatgtgagtgccctatgttccttgcgctccttgttccttgctccttgctcggcgtgcttgaacgctacgggctgtggagggagggatggatggatgaataggatgctgactgatggtgggacaccagatttacaataacttccactcgtatacggtgccgggtccggcggtctttcagtgctag

SEQ ID NO: 44 - protein, 223 aa, Thielavia terrestris
MKLSSQLAALTLAAASVSGHYIFEQIAHGGTKFPPYEYIRRNTNYNSPVTSLSSNDLRCNVGGETAGNTTVLDVKAGDSFTFYSDVAVYHQGPISLYMSKAPGSVVDYDGSGDWFKIHDWGPTFSNGQASWPLRDNYQYNIPTCIPNGEYLLRIQSLAIHNPGATPQFYISCAQVRVSGGGSASPSPTAKIPGAFKATDPGYTANIYNNFHSYTVPGPAVFQC

SEQ ID NO: 45 - DNA, 1107 bp, Thielavia terrestris
  source 1..1107: mol_type = genomic DNA
atgccttctttcgcctccaagactctcctttccaccctggcgggtgccgcatccgtggccgcccacgggcacgtgtcgaacatcgtcatcaacggggtctcgtaccagggttacgatccgacctccttcccttacatgcagaacccgcccatcgtggtcggctggactgccgccgacacggacaacggctttgttgccccggatgccttcgccagtggcgatatcatctgccacaagaacgccaccaacgccaagggccacgccgtggtcgccgcgggagacaagatcttcatccagtggaacacatggcccgagtcccaccacggccccgtcatcgactacctcgcgagctgcggcagcgcgtcctgcgagaccgtcgacaagaccaagctcgagttcttcaagatcgacgaggtcggcctggtcgacggcagctcggcgcccggtgtgtggggctccgaccagctcatcgccaacaacaactcgtggctcgtcgagatcccgcccaccatcgcgccgggcaactacgtcctgcgccacgagatcatcgcgctgcacagcgccgaaaacgccgacggcgcccagaactacccgcagtgcttcaacctgcagatcaccggcaccggcaccgccaccccctccggcgtccccggcacctcgctctacaccccgaccgacccgggcatcctcgtcaacatctacagcgccccgatcacctacaccgtcccggggccggccctcatctccggcgccgtcagcatcgcccagtcctcctccgccatcaccgcctccggcaccgccctgaccggctctgccaccgcacccgccgccgccgctgctaccacaacttccaccaccaacgccgcggctgctgctacctctgctgctgctgctgctggtacttccacaaccaccaccagcgccgcggccgtggtccagacctcctcctcctcctcctccgccccgtcctctgccgccgccgccgccaccaccaccgcggctgccagcgcccgcccgaccggctgctcctctggccgctccaggaagcagccgcgccgccacgcgcgggatatggtggttgcgcgaggggctgaggaggcaaactga

SEQ ID NO: 46 - protein, 368 aa, Thielavia terrestris
MPSFASKTLLSTLAGAASVAAHGHVSNIVINGVSYQGYDPTSFPYMQNPPIVVGWTAADTDNGFVAPDAFASGDIICHKNATNAKGHAVVAAGDKIFIQWNTWPESHHGPVIDYLASCGSASCETVDKTKLEFFKIDEVGLVDGSSAPGVWGSDQLIANNNSWLVEIPPTIAPGNYVLRHEIIALHSAENADGAQNYPQCFNLQITGTGTATPSGVPGTSLYTPTDPGILVNIYSAPITYTVPGPALISGAVSIAQSSSAITASGTALTGSATAPAAAAATTTSTTNAAAAATSAAAAAGTSTTTTSAAAVVQTSSSSSSAPSSAAAAATTTAAASARPTGCSSGRSRKQPRRHARDMVVARGAEEAN

SEQ ID NO: 47 - DNA, 993 bp, Thielavia terrestris
  source 1..993: mol_type = genomic DNA
atgccgcccgcactccctcaactcctaaccacggtcctgaccgccctcaccctcggttccaccgccctcgcccactcacacctcgcgtacattatcgttaacggcaagctctaccagggcttcgacccgcgcccgcaccaggccaactacccttcccgggtcgggtggtccaccggcgccgtcgacgacggcttcgtcacgccggccaactactccaccccggacatcatttgccacatcgccggcaccagcccggccggccacgcgcccgtgcgcccgggcgaccgcatccacgtccagtggaacggctggccggtcggccacatcggtcccgtgctgtcgtacctcgcccgctgcgagtcggacacgggctgcacgggccagaacaagaccgcgctgcggtggaccaagatcgacgactccagcccgaccatgcagaacgtcgccggcgcgggcacccagggcgagggcacccccggcaagcgctgggccaccgacgtgctgatcgccgccaacaacagctggcaggtcgccgtgccggcggggctgccgaccggcgcgtacgtgctgcgcaacgagatcatcgcgctgcactacgcggcgaggaagaacggggcgcagaactatccgctctgcatgaacctgtgggtggacgccagtggtgataatagtagtgtggctgcaacgacggcggcggtgacggcggggggtctgcagatggatgcgtatgacgcgcgcgggttctacaaggagaacgatccgggcgtgctggtcaatgtcacggccgcgctgtcgtcgtatgtcgtgcccgggccgacggtggcggcgggcgccacgccggtgccgtacgcgcagcagagcccgagcgtgtcgacggcggcgggcacgcccgtcgtcgttacaaggactagcgagacggcgccgtacacgggcgccatgacgccgacggttgcggcgaggatgaaggggagggggtatgatcggcggggttag

SEQ ID NO: 48 - protein, 330 aa, Thielavia terrestris
MPPALPQLLTTVLTALTLGSTALAHSHLAYIIVNGKLYQGFDPRPHQANYPSRVGWSTGAVDDGFVTPANYSTPDIICHIAGTSPAGHAPVRPGDRIHVQWNGWPVGHIGPVLSYLARCESDTGCTGQNKTALRWTKIDDSSPTMQNVAGAGTQGEGTPGKRWATDVLIAANNSWQVAVPAGLPTGAYVLRNEIIALHYAARKNGAQNYPLCMNLWVDASGDNSSVAATTAAVTAGGLQMDAYDARGFYKENDPGVLVNVTAALSSYVVPGPTVAAGATPVPYAQQSPSVSTAAGTPVVVTRTSETAPYTGAMTPTVAARMKGRGYDRRG

SEQ ID NO: 49 - DNA, 1221 bp, Thielavia terrestris
  source 1..1221: mol_type = genomic DNA
atgaagacattcaccgccctcctggccgcagccggcctcgtcgccggccatggatatgtcgacaacgccaccattggcggccagttttatcaggtactctaccgcttcacccaaggtccgctggccacaactctataggtgtcataaattaacaagccaccgtcccgcagttctatcaggtgtgctcgctaccgaccatgtggtcccgtctcagcaagccactcacacgcccatgatcccctagccttacgtcgacccgtatttagcaaccttggcacgtagtatttattgtcccaaatattgagctgaactgcacctccctagaatcccgcggtgctaacattctttcagcccgacagggtctctcgatccatcccgggcaacggcccggtcacggacgtcactctcatcgacctgcagtgcaacgccaattccaccccggccaagctccacgccactgccgctgccggctcggacgtgattctccgctggacgctctggcctgagtcgcacgttggccccgtcatcacctacatggcccgctgccccgacacgggctgccaggactggatgccgggcacttcgtaggagcccatcttgcaccatatccatttcaaccggccacacgcactgacccatatgtctgtctacccctgcagtgcggtctggttcaagatcaaggagggcggccgcgacggcacttccaacacctgggccgacgtacgtgtaccccgtcccagagagccaaagcccccccttcaacaaagcaaacatctcaatagcccgagcctacgcactaacccctctccttccccctcgaaaacacagaccccgctgatgacggcgcccacctcgtacacgtacacgatcccctcctgcctgaagaagggctactacctggtccgccacgagatcatcgcgctgcacgccgcctacacctaccccggcgcgcagttctacccgggctgccaccagctcaacgtcacgggcggcgggtccaccgtaccgtcgagcggcctggtggcctttcccggggcgtacaagggcagtgaccccgggattacgtacgatgcgtataaaggtgggttggctggttggcccaggtcttggtgatgggggaatgtggtgatgaggtttattatttgggatcccgtggctaacgtaaccctgggtgtagcgcaaacgtaccagattcctgggccggcggtctttacttgctga

SEQ ID NO: 50 - protein, 236 aa, Thielavia terrestris
MKTFTALLAAAGLVAGHGYVDNATIGGQFYQNPAVLTFFQPDRVSRSIPGNGPVTDVTLIDLQCNANSTPAKLHATAAAGSDVILRWTLWPESHVGPVITYMARCPDTGCQDWMPGTSAVWFKIKEGGRDGTSNTWADTPLMTAPTSYTYTIPSCLKKGYYLVRHEIIALHAAYTYPGAQFYPGCHQLNVTGGGSTVPSSGLVAFPGAYKGSDPGITYDAYKAQTYQIPGPAVFTC

SEQ ID NO: 51 - DNA, 933 bp, Thielavia terrestris
  source 1..933: mol_type = genomic DNA
atggccttgctgctcttggcaggcttggccattctggccgggccggctcatgcccacggcggcctcgccaactacacagtgggcaacacctggtatagggggtgcgtaaggggggcaccgacaacgcctgcttagtaactccaccatttcgagcgggctaacaccgggcgcagctacgaccccttcacgccggcggccgaccagatcggccagccgtggatgatccaacgcgcgtgggactcgatcgacccgatcttcagcgtcaacgacaaggcgctcgcctgcaacaccccggccacggcgccgacctcttacattcccatccgcgcgggcgagaacatcacggccgtgtactggtactggctgcacccggtgggccccatgacggcgtggctggcgcggtgcgacggcgactgccgcgacgccgacgtcaacgaggcgcgctggttcaagatctgggaggccggcctgctcagcgggccgaacctggccgagggcatgtggtaccagaaggcgttccagaactgggacggcagcccggacctgtggcccgtcacgatcccggccgggctgaagagcggcctgtacatgatccggcacgagatcttgtcgatccacgtcgaggataaaccgcagttttatcccgagtgtgcgcatctgaatgtgaccgggggtggggacctgctgccgcctgatgagtttttggtgaagttcccgggcgcttacaaagaagatagtgagtgaaacgcgaagcttcggtagccattgggttgcgctgatggaggttagacccgtcgatcaagatcaatatctactcggaccagtacgccaatacaacggtgagtgtaacaggtcgagcaaaaccaaacagatgccgatgactgatgatctcagaattacacaattcccggagggccgatatgggatgggtga

SEQ ID NO: 52 - protein, 250 aa, Thielavia terrestris
MALLLLAGLAILAGPAHAHGGLANYTVGNTWYRGYDPFTPAADQIGQPWMIQRAWDSIDPIFSVNDKALACNTPATAPTSYIPIRAGENITAVYWYWLHPVGPMTAWLARCDGDCRDADVNEARWFKIWEAGLLSGPNLAEGMWYQKAFQNWDGSPDLWPVTIPAGLKSGLYMIRHEILSIHVEDKPQFYPECAHLNVTGGGDLLPPDEFLVKFPGAYKEDNPSIKINIYSDQYANTTNYTIPGGPIWDG

SEQ ID NO: 53 - DNA, 1584 bp, Thielavia terrestris
  source 1..1584: mol_type = genomic DNA
atgatgccgtcccttgttcgcttctcaatgggtctggcgaccgccttcgcctcgctgtccacagcacataccgtcttcaccacgcttttcatcaacggcgtcgaccaaggggacgggacctgcatccgcatggccaagaagggcagcgtttgcacccatcccattgctggtggcctcgacagcccagacatggcttgtggtatgccctctgcgtttcccctgcgagagctttcctcgagctaacccaatgccgcgttgcccaggccgagacggacaacaagccgtggcattcacctgcccagccccggcgggctccaagttgagcttcgagttccgcatgtgggccgacgcctctcagcccggctctatcgacccatcccacctcggctcgacggcaatctacctcaaacaagtctccaacatcagctccgactcggctgccggccctggctggttcaagatctacgccgagggctacgacacagccgccaagaagtgggccacagagaagctcatcgacaacggcggcctgctgagcatcgagcttccgcccactctgccggcgggatactacctcgcccgcagcgagatcgtcaccatccagaacgtcaccaacgaccacgtcgacccgcagttctacgttggctgcgcacagctcttcgtccaggggcctccgaccacccccaccgtcccgccagacagactcgtctccatcccgggccacgtccatgcctccgacccggggctgaccttcaacatctggcgcgacgacccctccaagacggcctacaccgtcgtcggcccggcccccttctcccccaccgccgcccccacccccacctccaccaacaccaacgggcagcaacaacaacaacagcaacaggcgataaagcagacggacggcgtgatccccgccgactgccagctcaagaacgccaactggtgcggcgccgaggtgcccgcgtacgccgacgaggccggctgctgggcgtcgtcggccgactgcttcgcccagctggacgcctgctacacgtcggcgccgcccacgggcagccgcggctgccggctgtgggaggactggtgcaccggcattcagcagggctgccgcgcggggcggtggcgggggccgccgccctttcatggggagggggcagcagcggaggtgtgaacggttcggggacgggtggcggtggtggtggtggtggtggtggcactggctcttcttcggcttctgccccgacggagacggcctctgctggccgggggggcgcaagaatagctgccgtggccggctgcggaggcgggacaggagacatggttgaagaggttttcctcttttattgggacgcttgcagcggctggcgacggagccgtggtggtggttcgattcttgcgaggcttatccttcatgtccttcttccacttttgagaccgaggcgagcccctcgagtccatttacttctcttccacctgtacctcaacttctgttatccaggaaccagtggtttctataatcgcctgagcattaaactaggcatatggccaagcaaaatgtcgcctgatgtagcgcattacgtgaaataa

SEQ ID NO: 54 - protein, 478 aa, Thielavia terrestris
MMPSLVRFSMGLATAFASLSTAHTVFTTLFINGVDQGDGTCIRMAKKGSVCTHPIAGGLDSPDMACGRDGQQAVAFTCPAPAGSKLSFEFRMWADASQPGSIDPSHLGSTAIYLKQVSNISSDSAAGPGWFKIYAEGYDTAAKKWATEKLIDNGGLLSIELPPTLPAGYYLARSEIVTIQNVTNDHVDPQFYVGCAQLFVQGPPTTPTVPPDRLVSIPGHVHASDPGLTFNIWRDDPSKTAYTVVGPAPFSPTAAPTPTSTNTNGQQQQQQQQAIKQTDGVIPADCQLKNANWCGAEVPAYADEAGCWASSADCFAQLDACYTSAPPTGSRGCRLWEDWCTGIQQGCRAGRWRGPPPFHGEGAAAETASAGRGGARIAAVAGCGGGTGDMVEEVFLFYWDACSGWRRSRGGGSILARLILHVLLPLLRPRRAPRVHLLLFHLYLNFCYPGTSGFYNRLSIKLGIWPSKMSPDVAHYVK

SEQ ID NO: 55 - DNA, 868 bp, Thielavia terrestris
  source 1..868: mol_type = genomic DNA
atgcagctcctcgtgggcttgctgcttgcagccgtggctgctcgagcacattgtatttctacccctttccgcgtgcctcccagcctcaaggcaagaagacgcacgcagcagctaacggaccctatcagacacatttcccagactcgtggtaaatgggcagcccgaggacaaggactggtcggttacgcgcatgaccaagaacgcgcagagcaagcagggagtccaggacccgaccagtcccgacattcgctgctacacgtcgcagacggcgcctaacgtggctacggtccctgccggagccaccgtccattacatatcgactcagcagatcaaccacccgggcccgacgcagtactacctcgccaaggtaccggcggggtcgtcggccaagacgtgggacgggtcaggggccgtctggttcaagatctcgaccaccatgccttacttggacaacaacaagcagcttgtctggccgaatcagagtaggaacaattcccgctccaatcttcgatttggccttgagctacggccgattgcatgggagagaccgttgactgacggggcaacccaaccttcatcagacacgtacacgacggtcaacacgaccatccccgccgatacgcccagtggggaatacctcctccgggtcgagcagatcgcgctgcacctggcctcgcagcccaacggggctcagttctacctggcctgctcgcagatccagattacgggcggcggcaacggcacgcccggcccgctagtcgcgttgccgggggcgtacaagagcaacgacccgggcattttggtcaacatctactctatgcagcccggcgattacaagccgcccgggccgccggtgtggagtggctga

SEQ ID NO: 56 - protein, 230 aa, Thielavia terrestris
MQLLVGLLLAAVAARAHYTFPRLVVNGQPEDKDWSVTRMTKNAQSKQGVQDPTSPDIRCYTSQTAPNVATVPAGATVHYISTQQINHPGPTQYYLAKVPAGSSAKTWDGSGAVWFKISTTMPYLDNNKQLVWPNQNTYTTVNTTIPADTPSGEYLLRVEQIALHLASQPNGAQFYLACSQIQITGGGNGTPGPLVALPGAYKSNDPGILVNIYSMQPGDYKPPGPPVWSG

SEQ ID NO: 57 - DNA, 1068 bp, Thielavia terrestris
  source 1..1068: mol_type = genomic DNA
atgaagctgtacctggcggcctttctaggcgccgtcgccaccccgggagcgttcgctcatcgtaggttccccgtctatctccctaggggtagcaccacgactaatttctcgtcgtccccctgtagaaatccacgggattctacttgtcaacggcaccgaaacgccggaatggaaatacgtccggtaatatctaccttgctctccttcttccacaaccagcctaacacatcatcagtgacgtggcctgggagggcgcctacgaaccggaaaaataccccaacaccgagttctttaagacgcccccgcagacggacatcaacaacccgaacatcacctgcggcaggaacgcgttcgactcggccagcaagactgagacggccgacatactggccggctcagaggtcggcttccgcgtctcgtgggacggcaacggcaagtacggcgtgttctggcatcccgggccggggcagatctacctctctcgtgctccgaacgacgacctggaggactaccgcggcgacggagactggttcaagatcgcaaccggcgccgccgtctccaataccgagtggctgctgtggaacaagcatgacgtgagccccaacattcctcgcccaatcgatccccaacctggtcaccatggcggcgtccgggatgcaaagagactaactccagaggaacctacctagttcaacttcaccatccccaagacgacgccgccgggcaagtacctgatgcgcatcgagcagttcatgccctccacggtcgaatacagccagtggtacgtcaactgcgcccacgtcaacatcatcggccccggcggaggcacgccgacgggctttgccaggtttcccggcacctacactgttgacgatcccggtaagccggacctaccggacacagaggcctcgggatagcttgctaaccttgtttgctctctctctttttctctcccgactaggcatcaaggtgccgttgaaccagatcgtcaacagcggagagttgccgcaggaccaactgaggctgctcgagtacaagcccccgggcccagcgctgtggactggttga

SEQ ID NO: 58 - protein, 257 aa, Thielavia terrestris
MKLYLAAFLGAVATPGAFAHQIHGILLVNGTETPEWKYVRDVAWEGAYEPEKYPNTEFFKTPPQTDINNPNITCGRNAFDSASKTETADILAGSEVGFRVSWDGNGKYGVFWHPGPGQIYLSRAPNDDLEDYRGDGDWFKIATGAAVSNTEWLLWNKHDFNFTIPKTTPPGKYLMRIEQFMPSTVEYSQWYVNCAHVNIIGPGGGTPTGFARFPGTYTVDDPGIKVPLNQIVNSGELPQDQLRLLEYKPPGPALWTG

SEQ ID NO: 59 - DNA, 871 bp, Thermoascus crustaceus
  source 1..871: mol_type = genomic DNA
atggccttttcccagataatggctattaccggcgtttttcttgcctctgcttccctggtggctggccatggctttgttcagaatatcgtgattgatggtaaaaggtacctaactacctaccttactatctgatgtcatttacaagaaagggcacagacacaagcggcaaaaaaaagaaagaaagaaagaaagaaagaaagctgacaaaaattcaacaagttatggcgggtacatcgtgaaccaatatccatacatgtcagatcctccggaggtcgtcggctggtctaccaccgcaaccgacctcggattcgtggacggtaccggataccaaggacctgatatcatctgccacaggggcgccaagcctgcagccctgactgcccaagtggccgccggaggaaccgtcaagctggaatggactccatggcctgattctcaccacggcccggtgatcaactaccttgctccttgcaacggtgactgttccaccgtggacaagacccaattgaaattcttcaagatcgcccaggccggtctcatcgatgacaacagtcctcctggtatctgggcctcagacaatctgatagcggccaacaacagctggactgtcaccatcccaaccacaactgcacctggaaactatgttctaaggcatgagatcattgctctccactcagctgggaacaaggatggtgcgcagaactatccccagtgcatcaacctgaaggtcactggaaatggttctggcaatcctcctgctggtgctcttggaacggcactctacaaggatacagatccgggaattctgatcaatatctaccagaaactttccagctatgttattcctggtcctgctttgtacactggttag

SEQ ID NO: 60 - protein, 251 aa, Thermoascus crustaceus
MAFSQIMAITGVFLASASLVAGHGFVQNIVIDGKSYGGYIVNQYPYMSDPPEVVGWSTTATDLGFVDGTGYQGPDIICHRGAKPAALTAQVAAGGTVKLEWTPWPDSHHGPVINYLAPCNGDCSTVDKTQLKFFKIAQAGLIDDNSPPGIWASDNLIAANNSWTVTIPTTTAPGNYVLRHEIIALHSAGNKDGAQNYPQCINLKVTGNGSGNPPAGALGTALYKDTDPGILINIYQKLSSYVIPGPALYTG

SEQ ID NO: 61 - DNA, 1102 bp, Thermoascus crustaceus
  source 1..1102: mol_type = genomic DNA
atgtcattctcgaagatacttgctatcgctggggccattacctacgcatcttcagctgccgctcatggttatgtccagggaattgttgtcgatggcagctagtatgtcactctggatggaaccttcagcacgtactgtactaacaatcagcagctacgggggatatatggtgacccaatatccctacaccgctcaacctccggaactcatcgcctggtccactaaagcaaccgatcttgggtttgtggacggcagtggctatacttctcctgatatcatctgccataagggtgctgagcctggtgcccagagcgccaaagtggcagctggagggaccgttgagctgcagtggacggcatggcccgagtctcacaagggcccagttattgactacctcgccgcctgcgacggggactgctcatctgttgataagactgcactaaagttctttaagattgacgagagtggtctgattgacggcaacggtgctggaacatgggcctctgatacgttgatcaaaaataacaacagctggactgtcaccatcccaagcacaattgcttccggaaactacgtactaagacacgaaataattgcgctccattctgccggaaacaaagatggtgctcagaactatccccagtgtatcaacctcgaggtcactggtagtggcaccgaaaaccctgctggcactctcggaacagcgctttacacagacactgatcctggccttctggtcaacatctaccagggtctgtccaactattcaatccctggtcctgctctgtatagcggcaacagtgataacgctggttccctcaaccctaccaccacgccgtcaattcagaatgctgctgctgctccctccacttccacagcatctgttgtcactgattcttcgtcagccacccagactgctagtgtcgccgccacgactccagcctccacttcggctgttacagcctcaccagctcccgatactggaagcgacgtaaccaaatatctggattcgatgagctcggatgaggtcctcaccctggtgcgcgggaccctgtcttggctggtttctaacaagaaacatgcgcgggatctttctcactga

SEQ ID NO: 62 - protein, 349 aa, Thermoascus crustaceus
MSFSKILAIAGAITYASSAAAHGYVQGIVVDGSYYGGYMVTQYPYTAQPPELIAWSTKATDLGFVDGSGYTSPDIICHKGAEPGAQSAKVAAGGTVELQWTAWPESHKGPVIDYLAACDGDCSSVDKTALKFFKIDESGLIDGNGAGTWASDTLIKNNNSWTVTIPSTIASGNYVLRHEIIALHSAGNKDGAQNYPQCINLEVTGSGTENPAGTLGTALYTDTDPGLLVNIYQGLSNYSIPGPALYSGNSDNAGSLNPTTTPSIQNAAAAPSTSTASVVTDSSSATQTASVAATTPASTSAVTASPAPDTGSDVTKYLDSMSSDEVLTLVRGTLSWLVSNKKHARDLSH

SEQ ID NO: 63 - DNA, 1493 bp, Thermoascus crustaceus
  source 1..1493: mol_type = genomic DNA
atgttgtcattcattcccaccaagtcagctgcgctgacgactcttctacttcttggaacagctcatgctcacactttgatgaccaccatgtttgtggacggcgtcaaccagggagatggtgtctgcattcgcatgaacaatgacggcggaactgccaatacctatatccagcctatcacgagcaaggatatcgcctgcggtaagtacccagatgtcatcatactctgccataacatccgtcatatctactagaatcggagcaatgttaagtatttccaggcatccaaggcgaaatcggcgcctcccgagtctgcccagtcaaggcatcttccaccctaaccttccaattccgcgagcaacccaacaacccaaactcctcccctctcgatccatcgcacaaaggccccgccgcggtgtacctgaaaaaggtcgactccgccatcgcgagcaacaacgccgccggagacagctggttcaagatctgggagtccgtctacgacgagtccacgggcaaatggggcacgaccaagatgatcgagaacaacgggcacatctccgtcaaggtgcccgatgatatcgagggtggttactatcttgcccggacggagctgctggcgctacattctgcggatcagggggatccgcagttctatgttggctgtgcgcagctgtttatcgattcggatgggacggcgaaaccgcccactgtttctattggagaggggacgtacgatctgagcatgcctgccatgacgtataatatctgggagacaccgttggctctgccgtatccgatgtatgggcctcctgtctatacgcctggctctggttctggatcagtccgtgcgacgagctcttctgctgtccctactgcaaccgaatcctcttttgtagaggaaagagcaaaccccgtcacggcaaacagtgtttattctgcaaggggcaaattcaaaacctggattgataaactgtcatggcgcgggaaggtccgtgagaacgtcagacaagccgcgggaagaagaagcactctcgtccagactgtgggtctaaagccaaaaggctgcatcttcgtcaatggaaactggtgcggcttcgaggttcccgactacaacgatgcggagagctgctgggctgtatgttcccctccttagcctcttacatccctaagtactacatttgaaaacaacaaaaagaaatgtatatactaactacgtacgctctactctaggcctccgacaactgctggaaacagtccgacgcctgctggaacaagacccaacccacgggctacaataactgccagatctggcaggacaagaaatgcaaggtcatccaggattcctgtagcggacccaacccgcatggaccaccgaataagggcaaggatttgactccggagtggccgccactgaagggctcgatggatacgttctccaagcgtactatcggttaccgcgattggattgttagaaggagaggtgcatgagggtgta

SEQ ID NO: 64 - protein, 436 aa, Thermoascus crustaceus
MLSFIPTKSAALTTLLLLGTAHAHTLMTTMFVDGVNQGDGVCIRMNNDGGTANTYIQPITSKDIACGIQGEIGASRVCPVKASSTLTFQFREQPNNPNSSPLDPSHKGPAAVYLKKVDSAIASNNAAGDSWFKIWESVYDESTGKWGTTKMIENNGHISVKVPDDIEGGYYLARTELLALHSADQGDPQFYVGCAQLFIDSDGTAKPPTVSIGEGTYDLSMPAMTYNIWETPLALPYPMYGPPVYTPGSGSGSVRATSSSAVPTATESSFVEERANPVTANSVYSARGKFKTWIDKLSWRGKVRENVRQAAGRRSTLVQTVGLKPKGCIFVNGNWCGFEVPDYNDAESCWAASDNCWKQSDACWNKTQPTGYNNCQIWQDKKCKVIQDSCSGPNPHGPPNKGKDLTPEWPPLKGSMDTFSKRTIGYRDWIVRRRGA

SEQ ID NO: 65 - DNA, 1035 bp, Aspergillus aculeatus
  source 1..1035: mol_type = genomic DNA
atgaagtatattcctctcgttattgcagttgctgccggcctggcacgtccggctactgcccactacatcttcagcaagctcgtgctgaacggagaggcatctgcggactggcaatacatccgcgagactactcgcagcatagtctatgagccgaccaagtacacctctaccttcgataacctaacacccagcgatagcgacttccgctgtaatctcggttccttcagcaatgctgcgaagaccgaggtcgctgaggttgcggcaggcgataccatcgcaatgaagctattctacgacaccagtattgcgcatcctggcccgggacaagtttatatgtccaaggcaccgaccggcaatgttcaggaataccaaggagacggggattggttcaaaatctgggaaaagaccctttgcaacacggatggtgatctgactacagaggcctggtgcacctggggcatgtcacagtttgaatttcaaatcccagctgcgaccccggcaggagagtacctagtgcgcgccgagcatataggcctgcatggcgctcaagcgaacgaggccgaattcttctacagctgtgcgcagatcaaggttacaggctcgggaactggatctcccagtctcacgtatcaaattcctggtctctataacgacactatgaccctgttcaatggcctcaatctttggactgattcagccgagaaggtgcagctggatttcctggagacgccaattggggacgacgtgtggagcggagcaggctcggggagcccatctgctgccacctcttcgaccagcggtgcaactcttgcagctcagggtacaactacctctgccgcgcatgctcaggcccagaccaccattaccaccagcaccagcaccatcacgtctctcgaatcagccagctcaaccgatctcgttgcgcagtatggtcagtgcggaggccttaactggtccggtccaaccgagtgtgagacaccttatacctgtgtgcagcagaacccttactaccatcaatgcgtgaattcgtgctga

SEQ ID NO: 66 - protein, 344 aa, Aspergillus aculeatus
MKYIPLVIAVAAGLARPATAHYIFSKLVLNGEASADWQYIRETTRSIVYEPTKYTSTFDNLTPSDSDFRCNLGSFSNAAKTEVAEVAAGDTIAMKLFYDTSIAHPGPGQVYMSKAPTGNVQEYQGDGDWFKIWEKTLCNTDGDLTTEAWCTWGMSQFEFQIPAATPAGEYLVRAEHIGLHGAQANEAEFFYSCAQIKVTGSGTGSPSLTYQIPGLYNDTMTLFNGLNLWTDSAEKVQLDFLETPIGDDVWSGAGSGSPSAATSSTSGATLAAQGTTTSAAHAQAQTTITTSTSTITSLESASSTDLVAQYGQCGGLNWSGPTECETPYTCVQQNPYYHQCVNSC

SEQ ID NO: 67 - DNA, 1203 bp, Aspergillus aculeatus
  source 1..1203: mol_type = genomic DNA
atgtctgttgctaagtttgctggtgttatcctcggttcggccgctctcgtcgctggccacggttacgtgtcgggtgctgttgtcgacggaacctactatggcggctacattgtcacttcctacccctattccagcgatcccccggagaccattggatggtctaccgaggcgaccgacttgggtttcgtcgatggtagcgagtatgctgatgccgacatcatttgccacaagagtgccaagcccggtgccatctctgctgaggtcaaggccggtggtactgttgagctccagtggactacctggcccgacagccaccacggccctgtcctgacctaccttgccaactgcaatggtgactgcagcagcgtcaccaagaccgacctcgagtttttcaagattgacgagagcggtctcatcaacgacgacgacgtccccggtacctgggccagtgataacttgatcgccaacaacaacagctggactgtgaccatcccctctgacattgcggctggcaactacgtcctccgtcacgaaatcattgcccttcactctgctggtaacaaggatggtgctcagaactaccctcagtgcctcaacttgaaggtcactggcggcggtgatctcgctccttctggcactgctggtgagagcctgtacaaggacaccgatgctggtatcctcgtcaacatctaccagtctctttcctcctacgatattcccggacctgctatgtacaacgctacctccagctcctccagctcctccagctccagctccagctccagctccagctccagctccggctcttccagctccgccgccgcctccagcagctccagcagctccagcactactgccgccgccgccgccgctaccagcgctgcttcttccgtcacctctgctgctggctccgtcgttactcagactgctaccgctgttgagactgatactgccactgcctaccagacctccactgaggttgcgcaagtcaccgtcaccggtagcgctccccagcagacctacgttgccactcccagcagctccagctctgcctccagcagctccagtgcttccgtatccaccagcaccagcctcaccagctacttcgagtccctgagcgctgatcagttcctcagcgttctcaagcagactttcacctggttggtcagcgagaagaagcacgcccgtgacctctccgcctaa

SEQ ID NO: 68 - protein, 400 aa, Aspergillus aculeatus
MSVAKFAGVILGSAALVAGHGYVSGAVVDGTYYGGYIVTSYPYSSDPPETIGWSTEATDLGFVDGSEYADADIICHKSAKPGAISAEVKAGGTVELQWTTWPDSHHGPVLTYLANCNGDCSSVTKTDLEFFKIDESGLINDDDVPGTWASDNLIANNNSWTVTIPSDIAAGNYVLRHEIIALHSAGNKDGAQNYPQCLNLKVTGGGDLAPSGTAGESLYKDTDAGILVNIYQSLSSYDIPGPAMYNATSSSSSSSSSSSSSSSSSSSGSSSSAAASSSSSSSSTTAAAAAATSAASSVTSAAGSVVTQTATAVETDTATAYQTSTEVAQVTVTGSAPQQTYVATPSSSSSASSSSSASVSTSTSLTSYFESLSADQFLSVLKQTFTWLVSEKKHARDLSA

SEQ ID NO: 69 - DNA, 1170 bp, Aspergillus aculeatus
  source 1..1170: mol_type = genomic DNA
atgaagtcctctactttcggtatgctcgctctggcagcagcagccaagatggtcgatgcccacaccaccgtcttcgccgtctggatcaacggcgaggaccagggtctgggcaacagtgccagtggctacatccggtctccccccagcaacagccccgtcaaggacgtgacctcgaccgacatcacctgcaacgtcaacggcgaccaggcggcggctaagaccctctccgtcaagggcggcgacgtcgtcaccttcgagtggcaccacgacagccgggacgcctccgacgacatcatcgcctcctcccacaagggccccgtcatggtctacatggccccgaccaccgccggcagcagcggcaagaactgggtcaagatcgccgaggacggatactccgacggcacctgggccgtcgacaccctgatcgccaacagcggcaagcacaacatcaccgtccccgacgtccccgccggcgactacctcttccgcccggagatcatcgccctccacgaggccgagaacgagggcggcgcccagttctacatggagtgtgtccagttcaaggtcacctccgacggtgccaacactctgcccgacggtgtcagcctgcccggcgcctactccgccactgaccccggtatcctcttcaacatgtacggctccttcgacagctatcccatccccggtccctccgtctgggatggcactagctctggctcttcctcttcttcctcttcttcctcttccagctcttccgccgccgctgccgttgttgccacctcctcttcctcttcctctgcttccatcgaggccgtgaccaccaagggtgccgtcgccgccgtctccaccgccgccgccgtggctcctaccaccaccaccgctgcccccaccaccttcgccacggccgtcgcctccaccaagaaggccactgcctgccgcaacaagaccaagtcctcctccgctgccaccaccgccgccgccgtcgccgagaccacctcttccaccgctgccgccaccgctgctgcttcctctgcctcttccgcctccggcaccgccggcaagtacgagcgctgcggtggccagggctggaccggtgccaccacctgcgttgatggctggacctgcaagcagtggaacccttactactaccagtgcgttgagtctgcctag

SEQ ID NO: 70 - protein, 389 aa, Aspergillus aculeatus
MKSSTFGMLALAAAAKMVDAHTTVFAVWINGEDQGLGNSASGYIRSPPSNSPVKDVTSTDITCNVNGDQAAAKTLSVKGGDVVTFEWHHDSRDASDDIIASSHKGPVMVYMAPTTAGSSGKNWVKIAEDGYSDGTWAVDTLIANSGKHNITVPDVPAGDYLFRPEIIALHEAENEGGAQFYMECVQFKVTSDGANTLPDGVSLPGAYSATDPGILFNMYGSFDSYPIPGPSVWDGTSSGSSSSSSSSSSSSSAAAAVVATSSSSSSASIEAVTTKGAVAAVSTAAAVAPTTTTAAPTTFATAVASTKKATACRNKTKSSSAATTAAAVAETTSSTAAATAAASSASSASGTAGKYERCGGQGWTGATTCVDGWTCKQWNPYYYQCVESA

SEQ ID NO: 71 - DNA, 1221 bp, Aspergillus aculeatus
  source 1..1221: mol_type = genomic DNA
atgcgtcaggctcagtctttgtccctcttgacagctcttctgtctgccacgcgtgtggctggacacggtcacgtcactaacgttgtcgtcaacggtgtttactacgagggcttcgatatcaacagcttcccctacgagtccgatccccctaaggtggcggcttggaccactcctaacactggcaacggtttcatttcccccagcgactacggtaccgatgacattatttgccaccagaatgccaccaacgcccaggcccacattgttgttgcggctggtgacaagatcaacatccagtggaccgcgtggcccgattcccaccacggtcctgtccttgactacctcgctcgctgcgacggtgagtgtgagacggttgataagaccactcttgagtttttcaagatcgacggcgtcggtctcatcagtgacaccgaagtgcccggtacctggggagatgaccagctgatcgccaacaacaacagctggttggtcgagatccccccgaccattgctcctggcaactatgttcttcgccacgagcttatcgctctccacagcgccggcactgaagatggtgctcagaactacccccagtgtttcaacctccaggtcactggctccggtactgacgagcccgctggtaccctcggcaccaagctctacactgaggatgaggctggtatcgttgtgaacatctacacctctctgtcttcctatgccgtccccggccccacccagtacagcggcgccgtctctgtcagccaatccacttcggccattacctccaccggaactgctgttgtcggtagcggcagcgctgttgccacctctgccgccgcggctaccaccagcgctgctgcttcttctgccgctgctgctaccaccgctgctgccgttaccagcgccaatgccaacactcagattgcccagcccagcagcagctcttcttactcccagatcgccgtgcaggtgccctcctcctggaccacccttgtgaccgtcactcctcccgccgccgccgccaccacccctgctgccgtccctgagcctcagaccccctctgccagctctggagccaccactaccagcagcagcagcggcgccgcccagtctctctacggccagtgcggtggtatcaactggaccggagctacctcttgcgttgagggcgctacttgctaccagtacaacccttactactaccagtgcatctctgcctaa

SEQ ID NO: 72 - protein, 406 aa, Aspergillus aculeatus
MRQAQSLSLLTALLSATRVAGHGHVTNVVVNGVYYEGFDINSFPYESDPPKVAAWTTPNTGNGFISPSDYGTDDIICHQNATNAQAHIVVAAGDKINIQWTAWPDSHHGPVLDYLARCDGECETVDKTTLEFFKIDGVGLISDTEVPGTWGDDQLIANNNSWLVEIPPTIAPGNYVLRHELIALHSAGTEDGAQNYPQCFNLQVTGSGTDEPAGTLGTKLYTEDEAGIVVNIYTSLSSYAVPGPTQYSGAVSVSQSTSAITSTGTAVVGSGSAVATSAAAATTSAAASSAAAATTAAAVTSANANTQIAQPSSSSSYSQIAVQVPSSWTTLVTVTPPAAAATTPAAVPEPQTPSASSGATTTSSSSGAAQSLYGQCGGINWTGATSCVEGATCYQYNPYYYQCISA

SEQ ID NO: 73 - DNA, 1284 bp, Aspergillus aculeatus
  source 1..1284: mol_type = genomic DNA
atgtctctttccaagattgccactcttctgctgggctcggtctcgctggtcgctggtcatgggtatgtctcgagcatcgaggtggacggtaccacctatggagggtacttggtcgacacttattactacgaatccgacccgcccgagttaatcgcctggtccacaaatgccacggatgatggctatgtatcgccctccgactacgagagcgtgaacatcatctgccacaaggggtctgcgcccggcgcgttgtcggcccctgtcgcgcccggaggctgggtgcagatgacctggaacacctggcccaccgaccatcacggccctgtcatcacgtatatggccaattgccacggttcttgcgcagatgtggacaagaccaccctcgagttcttcaagatcgatgctggcggcttgatcgatgacacggacgtgcctggaacttgggcgaccgatgagctcattgaagatagctatagtcgcaacatcactatccccagcgatattgcccccgggtactatgttttgcgacacgagatcattgctctgcacagcgccgagaacctggacggagcccagaactacccccagtgcatcaatctggaagtcaccggcagcgagacagcaaccccgagtggcaccttgggcactgctctgtacaaggagaccgaccccggcatctatgttgacatctggaacacgttgagcacgtatactattcccggccccgcgctgtacactgctggtagcactgcgaccgcagccgctgctgccgataccaccactacttctgctggcaccaccgctgaggccaccaccgctgccgccgccgtgagtaccaccgcggacgctgttccgaccgagtcttcagctccttccgagaccagcgcgactaccgcgaaccctgctcggcccactgccggcagcgacatccgcttccagcccggtcaggtcaaggctggtgcttcagtcaacaactcggctactgagacttcctctggtgagtctgccacgacgaccacaacatcagtggccactgcggcttcgagcgcggattcgtcgacgacttctggggttttgagtggcgcctgcagccaggagggctactggtactgcaacgggggcactgcgttccagcgctgtgtcaacggggaatgggatgcgtcccagagtgtggctgcgggcacggtctgcaccgccggtatctcggagaccatcaccatttcagccgccgccacgcgccgggatgccatgcgtcgtcatctggcgcgtcccaagcgtcactga

SEQ ID NO: 74 - protein, 427 aa, Aspergillus aculeatus
MSLSKIATLLLGSVSLVAGHGYVSSIEVDGTTYGGYLVDTYYYESDPPELIAWSTNATDDGYVSPSDYESVNIICHKGSAPGALSAPVAPGGWVQMTWNTWPTDHHGPVITYMANCHGSCADVDKTTLEFFKIDAGGLIDDTDVPGTWATDELIEDSYSRNITIPSDIAPGYYVLRHEIIALHSAENLDGAQNYPQCINLEVTGSETATPSGTLGTALYKETDPGIYVDIWNTLSTYTIPGPALYTAGSTATAAAAADTTTTSAGTTAEATTAAAAVSTTADAVPTESSAPSETSATTANPARPTAGSDIRFQPGQVKAGASVNNSATETSSGESATTTTTSVATAASSADSSTTSGVLSGACSQEGYWYCNGGTAFQRCVNGEWDASQSVAAGTVCTAGISETITISAAATRRDAMRRHLARPKRH

SEQ ID NO: 75 - DNA, 804 bp, Aspergillus aculeatus
  source 1..804: mol_type = genomic DNA
atgcttgtcaaactcatctcttttctttcagctgctaccagcgtagctgctcatggtcatgtgtcaaacattgtgatcaacggggtgtcctaccgcggatgggacatcaattcggacccttacaattccaaccctccggtggtggttgcatggcaaacacccaacacagctaatggcttcatctcccctgatgcatacgacacagatgatgttatttgccatctgagcgctacgaatgccagaggccacgcagtcgtcgctgctggcgacaagatcagcctccagtggacgacctggcctgacagtcaccatggccctgtcatcagctacctagccaactgcggctccagctgcgagacagtcgataagaccaccctcgagttcttcaagatcgatggtgttggcttggtggatgagagcaatccccctggtatctggggagacgatgagctcattgccaacaacaactcttggctggtagagattccagctagtatcgcgccaggatactatgtgctgcgtcacgagttgatcgctctgcatggagcagggagtgagaatggagcccagaattacatgcaatgtttcaaccttcaggttactgggactggcacggtccagccttccggggtcctgggcacggagctgtacaaacccacagacgctggaattcttgtcaatatctaccagtcgctctccacctatgttgttcctggcccgaccctgatcccccaggccgtttccctcgttcagtcgagctccaccattaccgcctcgggcacggcagtgacaaccacggcttga

SEQ ID NO: 76 - protein, 267 aa, Aspergillus aculeatus
MLVKLISFLSAATSVAAHGHVSNIVINGVSYRGWDINSDPYNSNPPVVVAWQTPNTANGFISPDAYDTDDVICHLSATNARGHAVVAAGDKISLQWTTWPDSHHGPVISYLANCGSSCETVDKTTLEFFKIDGVGLVDESNPPGIWGDDELIANNNSWLVEIPASIAPGYYVLRHELIALHGAGSENGAQNYMQCFNLQVTGTGTVQPSGVLGTELYKPTDAGILVNIYQSLSTYVVPGPTLIPQAVSLVQSSSTITASGTAVTTTA

SEQ ID NO: 77 - DNA, 822 bp, Aspergillus aculeatus
  source 1..822: mol_type = genomic DNA
atgaagtatcttgcgatcttcgcggcagcagcagctggactggcccgcccgacagcagcgcactacatcttcagcaagctgattctggacggcgaagtctctgaggactggcagtatattcgtaaaaccacccgggagacatgctatttgccgaccaagttcaccgacaccttcgacaacttgactccgaacgaccaggatttccggtgcaatctcggctcgttcagcaacgccgccaagaccgaagtggccgaggtggaagcgggctccacgattggcatgcagcttttcgctggtagccacatgcgtcacccgggacctgcgcaagtcttcatgtctaaggccccgtccggcaacgtacagagctacgagggtgacggctcctggttcaagatctgggagcgtacactctgcgacaaaagtggcgatctgactggagatgcgtggtgtacatacggccagaccgagatcgagtttcaaatccccgaggcgaccccgacgggcgaatacctggtccgagcggagcacatcggtcttcaccgcgcacagagtaatcaagccgagttctactacagctgcgcccaggtcaaggtcacgggcaatggtaccggggtgccgagccagacatatcagatccctggcatgtacaatgaccgctcggagcttttcaacgggctgaacttgtggtcctactcggtggagaacgtcgaggcagccatgaagaattctatcgtgggtgatgaaatttggaatggaagttctgttccctctgagtcccatgtcccgaagtataagaagagtcatgcttgtcgtgtttattga

SEQ ID NO: 78 - protein, 273 aa, Aspergillus aculeatus
MKYLAIFAAAAAGLARPTAAHYIFSKLILDGEVSEDWQYIRKTTRETCYLPTKFTDTFDNLTPNDQDFRCNLGSFSNAAKTEVAEVEAGSTIGMQLFAGSHMRHPGPAQVFMSKAPSGNVQSYEGDGSWFKIWERTLCDKSGDLTGDAWCTYGQTEIEFQIPEATPTGEYLVRAEHIGLHRAQSNQAEFYYSCAQVKVTGNGTGVPSQTYQIPGMYNDRSELFNGLNLWSYSVENVEAAMKNSIVGDEIWNGSSVPSESHVPKYKKSHACRVY

SEQ ID NO: 79 - DNA, 969 bp, Aurantiporus alborubescens
  source 1..969: mol_type = genomic DNA
atgcgaaccatcgccacgtttgttacgcttgtagcctcagttctccctgcggtcctcgcacacggaggtgtcctctcctattcsaacggggggaattggtactggggatggaagccttacaattcacctgacgggcagaccaccatccaacgcccgtgggcaacatacaatccgatcactgatgcgacggatcctaccattgcttgcaacaacgacgggacatctggagctctgcagttgactgcgacagtcgcggcgggatctgccatcacggcgtattggaaccaggtgtggccgcatgataaagggccgatgacgacatacctcgcacaatgccccggcagtacctgcacaggagtcaacgcgaagactctgaaatggttcaagatcgatcacgccgggttgctttctggtactgtctacagtggctcgtgggcatcaggcaagatgattgcacagaactcgacctggacaactaccattccagcgacggtgccttcagggaactatctgatacgtttcgagactattgccctgcactctttgccagcgcaattttaccctgagtgcgcacaaattcaaatcacgggcggaggttcccgtgctccaaccgctgcagagcttgttagcttccctggcgcgtacagcaacaatgatcctggtgtcaacattgacatctactccaatgccgcgcagagtgcaaccacatacgtaataccaggacctccattgtacggcggtgcttccggatctggtccatcttccgcgcctccatcaagtaccccaggtagttcgtccacttcccacggtcccacgtccgtcagcacgtccagcagtgctgcaccatcgacgacaggaaccgtgacgcagtacggtcagtgcggtggcattggttgggctggagctaccggctgtatctcaccattcaagtgcacggtcatcaacgattattactaccagtgcctctga

SEQ ID NO: 80 - protein, 322 aa, Aurantiporus alborubescens
MRTIATFVTLVASVLPAVLAHGGVLSYSNGGNWYWGWKPYNSPDGQTTIQRPWATYNPITDATDPTIACNNDGTSGALQLTATVAAGSAITAYWNQVWPHDKGPMTTYLAQCPGSTCTGVNAKTLKWFKIDHAGLLSGTVYSGSWASGKMIAQNSTWTTTIPATVPSGNYLIRFETIALHSLPAQFYPECAQIQITGGGSRAPTAAELVSFPGAYSNNDPGVNIDIYSNAAQSATTYVIPGPPLYGGASGSGPSSAPPSSTPGSSSTSHGPTSVSTSSSAAPSTTGTVTQYGQCGGIGWAGATGCISPFKCTVINDYYYQCL

SEQ ID NO: 81 - DNA, 705 bp, Aurantiporus alborubescens
  source 1..705: mol_type = genomic DNA
atgaaggctatcttggctattttctcggcccttgctccacttgccgctgcgcattataccttccctgattttattgtcaacggaacaacaactgccgattgggtctacatccgagagaccgcgaaccactactcgaatggtcctgtaaccaacgtgaacgatccagaattccgatgctacgagctggacctgcaaaacacggcagcgagtaccctcaccgccacggtctctgcaggctccagcgtcggctttaaagctaacagcgccctttaccatcctggttatctcgatgtgtatatgtccaaagcgaccccagctgctaattcacccagtgctggaacggaccaaagctggttcaaggtctatgaatccgctccggtcttcgcgaatggggccctaagcttcccttcggagaacatccaatctttcacgttcacaatcccgaagtcccttcccagtggccaatatctcatccgtgtggaacacatcgctctccactccgccagtagctacggaggtgcacaattctacatcagctgcgctcaagtcaatgtcgtcaacggcgggaacggaaacccaggaccgttagtcaagattcccggcgtttacactgggaacgagcctggcatcctcatcaacatctacagcttcccaccgggtttcagtggctaccaatccccgggacctgctgtgtggcgtggttga

SEQ ID NO: 82 - protein, 234 aa, Aurantiporus alborubescens
MKAILAIFSALAPLAAAHYTFPDFIVNGTTTADWVYIRETANHYSNGPVTNVNDPEFRCYELDLQNTAASTLTATVSAGSSVGFKANSALYHPGYLDVYMSKATPAANSPSAGTDQSWFKVYESAPVFANGALSFPSENIQSFTFTIPKSLPSGQYLIRVEHIALHSASSYGGAQFYISCAQVNVVNGGNGNPGPLVKIPGVYTGNEPGILINIYSFPPGFSGYQSPGPAVWRG

SEQ ID NO: 83 - DNA, 702 bp, Trichophaea saccata
  source 1..702: mol_type = genomic DNA
atgacgcccctgaaactccgcccccttctcctcctggtgctttccacgaccctcagcctcgtgcacgcgcactatcgcttctacgaactgatcgccaacggggccacccacgcttccttcgaatacatccgccaatgggtgcccatctacagcaactctcccgtaaccgacgtcaccagcgtcaacctccgctgcaacgtcaacgccactcccgccgccgaggtgatcaccgttgctgccggtagcaccgtcggcttcgtagcagacacaacagtaacgcaccccggtgcgttcaccgcgtacatggcgaaagcgcccgaagacatcacggaatgggatggcaacggggactggttcaagatctgggagaagggtccaacgagtataaccagtagcgggataacctgggacgtcacggatacccaatggaccttcaccatcccttccgcgacaccaaacggtcaatacctactccgcttcgagcacatagcgctccacgccgccagcaccgtggggggtgctcaattctacatgtcgtgcgcgcagatacaagtaacgaacggcggcaacgggagtcccgggcccaccatcaagttcccgggcggatacagcgccacagaccccggtatcctgatcaatatctattatcccatccccactagttacactattcctggtccaccggtttggaccggtaagtaa

SEQ ID NO: 84 - protein, 233 aa, Trichophaea saccata
MTPLKLRPLLLLVLSTTLSLVHAHYRFYELIANGATHASFEYIRQWVPIYSNSPVTDVTSVNLRCNVNATPAAEVITVAAGSTVGFVADTTVTHPGAFTAYMAKAPEDITEWDGNGDWFKIWEKGPTSITSSGITWDVTDTQWTFTIPSATPNGQYLLRFEHIALHAASTVGGAQFYMSCAQIQVTNGGNGSPGPTIKFPGGYSATDPGILINIYYPIPTSYTIPGPPVWTGK

SEQ ID NO: 85 - DNA, 714 bp, Trichophaea saccata
  source 1..714: mol_type = genomic DNA
atgaaatgccttctctccctccttctcgccgcgacagcggtctccgctcacacgatcttccaagaaatcggcataaacggggtgatgcaagctcgctacgactacatgcggctgccgtcctacgacggtcccattacggacgtaacgagcacctacatggcgtgcaacggtggtcccaatccattggtccaaatctcgaacgacgtcgctttcgtaaaagccggcgacagcatcacgctgcaatgggcgcaaacgttgacgacagatttcaacacggggctgatcatcgatccatcgcacttgggtcctgtgatggtctacatggccaaagtaccctccgccaccggtccgatccccaacagcggctggttcaaaatctacgaagacggctacgacccgacaacaaagacatgggcggtaaccaagctcatcaacaacaagggaaaagtgaccgtcaccatcccatcgtgtctaccggcaggggactacttgctgcgcggtgaaatcattgccttgcacgcggctagtacctatccaggcgcacagttttacatggagtgtgcgcagttgcggcttaccagtggcggcactaagatgcctaccacgtataacattccggggatctattcgcccactgatccgggtgttacgttcaatctttacaatggattcacgagttataccattcctggcccaaggccgtttacatgctag

SEQ ID NO: 86 - protein, 237 aa, Trichophaea saccata
MKCLLSLLLAATAVSAHTIFQEIGINGVMQARYDYMRLPSYDGPITDVTSTYMACNGGPNPLVQISNDVAFVKAGDSITLQWAQTLTTDFNTGLIIDPSHLGPVMVYMAKVPSATGPIPNSGWFKIYEDGYDPTTKTWAVTKLINNKGKVTVTIPSCLPAGDYLLRGEIIALHAASTYPGAQFYMECAQLRLTSGGTKMPTTYNIPGIYSPTDPGVTFNLYNGFTSYTIPGPRPFTC

SEQ ID NO: 87 - DNA, 1455 bp, Penicillium thomii
  source 1..1455: mol_type = genomic DNA
atgtctctgtctaagatttctggattgatcctcggatctgctgccttggtggctggccacggttacgtgagcggaatcgtcgttgacgatacctactatggtggataccttgtcacccagtacccttatgagagtgacgccccagagctcattgcctggtcggagcaagagaccgatctgggttacatcgatggctctgagtatgccaactccaacatcatctgtcacaaggaggccaaacctggtgctttggaagcacccgttaaggctggtggctccgtcgagctccagtggaccacttggcctaccagccaccacggtcctgtcattacctacatggccaactgtaacggcgactgtgacgacgttgacaagactactttgcagttcttcaagattgaccagggtggtttgatcagcgataccaccgagcccggtacctgggcaactgacaacctcatcgccaacaacaatagccgtactgtcaccgtccccagcgacattgccgatggaaactacgtcctccgtcacgagatcattgccctccactccgccggggagaccaacggtgcccagaactacccccaatgtatcaacttgaaggtcactggcggcggtagcgctactccttctggtaccctgggtaccgccctgtacaagaacaccgaccccggtatcctgatcaacatctacacttccctcagcacctacgatatccccggcccaaccctgtacactgccggcgccgccgctgctaccgctgcctccacggctgcctcttccaccgccgctgccgttactactgccgacgccgtcactaccgccgctgccgtcaccagcagctctgcatccgtggaagttgtgcccacaactactcccagctcatcaatcgtcagtgccttcccaacctggagcccctcttctaccccacccttctccaactcttccaacggatggcgtccgtcattcagccgcggacctggtggcccccgcttcacatctgctcctgctcctcagttctccgctcctagcggcgctcagcagaagcagtctgccactgctacccccatcgtggctacccctgtcgtgatcaccatgaccgagaccagcacctcctgggtcaccgaaatggttactcttactgacaagtctgttgtgcagaccaccagcgctgtcccagtcgtcgtcgccgccaccactacccttaccgagggaagcgagcctgctcagacagcctcccccagcgttgtctccggctcctctagctccggctctagctcctcatctaccaccaccacctcaaagacctcaactggatccgactacgtctccagcgactggatgtcttacctcagctccttgagcgctgctgaggtcctccagatgctgcgccagaccttccgttggatggtcagcaacgacaaggtgcacgctcgtgatattaccatcaactag

SEQ ID NO: 88 - protein, 484 aa, Penicillium thomii
MSLSKISGLILGSAALVAGHGYVSGIVVDDTYYGGYLVTQYPYESDAPELIAWSEQETDLGYIDGSEYANSNIICHKEAKPGALEAPVKAGGSVELQWTTWPTSHHGPVITYMANCNGDCDDVDKTTLQFFKIDQGGLISDTTEPGTWATDNLIANNNSRTVTVPSDIADGNYVLRHEIIALHSAGETNGAQNYPQCINLKVTGGGSATPSGTLGTALYKNTDPGILINIYTSLSTYDIPGPTLYTAGAAAATAASTAASSTAAAVTTADAVTTAAAVTSSSASVEVVPTTTPSSSIVSAFPTWSPSSTPPFSNSSNGWRPSFSRGPGGPRFTSAPAPQFSAPSGAQQKQSATATPIVATPVVITMTETSTSWVTEMVTLTDKSVVQTTSAVPVVVAATTTLTEGSEPAQTASPSVVSGSSSSGSSSSSTTTTSKTSTGSDYVSSDWMSYLSSLSAAEVLQMLRQTFRWMVSNDKVHARDITIN

SEQ ID NO: 89 - DNA, 1021 bp, Talaromyces stipitatus
  source 1..1021: mol_type = genomic DNA
atgccttccactaaagttgctgctctatctgccgtcctggctttggcctccacggttgctggccatggctttgtgcaaaatattgtcattgacggtaaatcgtaagtgacttgcttttgtactatagagctagataaatacttatactaaataattcagctacactggctacctcgtgaaccagtatccttaccagtccaacccaccagctgttattgggtggtcaaccactgcaaccgacttgggatttgtcgatggatctggatacaccaacccggatatcatctgccacaaaaacgccaaacccggtcagctttctgctccggttgccgcaggaggcaaggttgagctcgaatggacaacatggcccgagagccatcacggccctgtcatcagctatctcgccaattgcaatggcgattgtactaccgtggataagacgaagctcgaatttgtcaaaatcgatcagcggggtctgatcgacgacagcaatcctcccggtacatgggccgccgaccagctcatcgccgccaacaacagctggactgtaactattcccgagagcatcgcgcctggaaactacgtccttcgccacgaaatcatcgctcttcactccgccaacaacgcaaccggagctcaaaactaccctcaatgcatcaacttgcaaatcactggcagcgggacggccaacccatctggtacccctggcgagaaactctataccccaactgacccaggtatcttggtcaacatctaccagtcattgtcgtcttatgttattcccggtccgactttgtggagtggtgctgcagcgcacgttgttgccactgcagccggttctgctactggggttgcttctgccaccgctactccgaccactcttgtgactgccgtttcatcgcctaccggtgctccttcagtggtgactcctgaggctccttcagtaacctcgttcgccccagtggtgactgttactgatgtcgttactgtgactaccgtcatcactactactatctcttag

SEQ ID NO: 90 - protein, 320 aa, Talaromyces stipitatus
MPSTKVAALSAVLALASTVAGHGFVQNIVIDGKSYTGYLVNQYPYQSNPPAVIGWSTTATDLGFVDGSGYTNPDIICHKNAKPGQLSAPVAAGGKVELEWTTWPESHHGPVISYLANCNGDCTTVDKTKLEFVKIDQRGLIDDSNPPGTWAADQLIAANNSWTVTIPESIAPGNYVLRHEIIALHSANNATGAQNYPQCINLQITGSGTANPSGTPGEKLYTPTDPGILVNIYQSLSSYVIPGPTLWSGAAAHVVATAAGSATGVASATATPTTLVTAVSSPTGAPSVVTPEAPSVTSFAPVVTVTDVVTVTTVITTTIS

SEQ ID NO: 91 - DNA, 872 bp, Thermomyces lanuginosus
  source 1..872: mol_type = genomic DNA
atgaagggctccagcgctgcgtcggtgcttcttaccttcctcgcgggcatctcccgtacctctgcgcacgggtatgtctccaacctcgttatcaacggcgtctactatcggggctggctccccggcgaagacccctacaaccctgaccccccgattggcgttggctgggagacgcccaacctgggcaacggcttcgtgacgccgtcggaagcgtcgaccgacgccgtcatctgccacaaggaagccacaccagcccgcggtcatgtctccgtgaaggccggtgacaagatctacatccaatggcagccgaatccatggccggattcccaccacggtgcgtcaaacttctgcccgaaagctgttcacactcactaacaacacttttaggccccgtcctggactatctggccccttgcaacgggccctgtgagtccgtcgacaagaccagcctgcgcttcttcaagatcgacggagtgggtcttatcgacggctcttctcctccgggctactgggccgacgacgaactcattgcgaacggcaacgggtggctggttcagatccccgaggacatcaagccgggtaactacgtcctgcgacacgagatcatcgccttgcacagcgccgggaacccggacggcgcccagctgtacccgcagtgcttcaaccttgagattacgggatccggcaccgtcgagccggagggcgtgccagccaccgagttctactcgcccgatgacccgggcatcctggtcaacatctacgagcccctgtccacgtatgaggtgccgggtccctcgctcatcccgcaggcggttcagatcgagcagtcttcgtctgcgattacggcgacgggcacgccgacgccggcatga

SEQ ID NO: 92 - protein, 272 aa, Thermomyces lanuginosus
MKGSSAASVLLTFLAGISRTSAHGYVSNLVINGVYYRGWLPGEDPYNPDPPIGVGWETPNLGNGFVTPSEASTDAVICHKEATPARGHVSVKAGDKIYIQWQPNPWPDSHHGPVLDYLAPCNGPCESVDKTSLRFFKIDGVGLIDGSSPPGYWADDELIANGNGWLVQIPEDIKPGNYVLRHEIIALHSAGNPDGAQLYPQCFNLEITGSGTVEPEGVPATEFYSPDDPGILVNIYEPLSTYEVPGPSLIPQAVQIEQSSSAITATGTPTPA

SEQ ID NO: 93 - DNA, 1039 bp, Thermomyces lanuginosus
  source 1..1039: mol_type = genomic DNA
atggcattctctacggttacagtttttgttacgttcctggccttcatctccatagcttctgctcatggcttcgtgacaaaaatcaccgtactcggagataataataaggagtacgtctcagtctcgctaggttgctaacacaggagagatcgctgaccattgcagctaccccggctttgacccgagcactcccaaggaggttcctccgggtctcgatgtcgcttggtctactagtgccagtgatcagggatacatgagcagttcaaatgcctcgtatcacagtaaggactttatctgccacagaaacgccaaacctgctccagacgcagctcaagttcatgcgggcgacaaggtgcagcttcactggactcaatggcctggacctgaggatcaccagggtcctatccttgattacctcgcgagctgcaacggaccctgctcaaacgtggagaaggcgagccttaagtggacgaagattgacgaggcagggcgctttcccaacggaacgtgggcaacggacctgctcaggaatgggggaaacacgtggaatgtgacgattccatcggatcttgctcctggagaatatgtcctccgcaacgagatcattgcacttcactcggcgagaaatatgggtggagctcagcactacatgcaatgtgtcaatctgaacgtcactggcaccggccatagagagctacagggcgtctccgccgcagaattttacaatcctacggatcctggaattttgattaacgtctggcaaactcaaagcctttcctcctaccatattcccggacctacactgttagccgccgataccggcaacgacggtggccattctgcatcatctaccttggcgactgtgacaagcagacgtctttccactccgagcgacgccatgcccgggaatggttcatacggtgcaatttcgccgcccctcaaacctgctaaaggattccatcctgtttgtaacgcccgattcagacatggcagcactttcactttgactaccctggtcgcaccaccagccaggacctaa

SEQ ID NO: 94 - protein, 327 aa, Thermomyces lanuginosus
MAFSTVTVFVTFLAFISIASAHGFVTKITVLGDNNKDYPGFDPSTPKEVPPGLDVAWSTSASDQGYMSSSNASYHSKDFICHRNAKPAPDAAQVHAGDKVQLHWTQWPGPEDHQGPILDYLASCNGPCSNVEKASLKWTKIDEAGRFPNGTWATDLLRNGGNTWNVTIPSDLAPGEYVLRNEIIALHSARNMGGAQHYMQCVNLNVTGTGHRELQGVSAAEFYNPTDPGILINVWQTQSLSSYHIPGPTLLAADTGNDGGHSASSTLATVTSRRLSTPSDAMPGNGSYGAISPPLKPAKGFHPVCNARFRHGSTFTLTTLVAPPART

SEQ ID NO: 95 - DNA, 881 bp, Thermomyces lanuginosus
  source 1..881: mol_type = genomic DNA
atgaaaggctccaccactgcgtctttgcttcttccgctcctggcgagcgttactcgcacctctgcgcacgggtttgtctccaacctcgtcatcaatggcgtcttctatcggggctggctcccgaccgaggacccctacaaggctgaccccccgattggcgtcggctgggagacgcctaacctgggcaacggcttcgtgctgcccgaagaagcgtcgaccgatgccatcgtctgccacaaagaggccgagccggcccgcggctatgccagcgtcgctgccggtgacaagatctacattcagtggcagccgaacccatggccggagtctcatcacggtacgtcaaactgcccattgttgcaattcagaatcatctactaacaactcttcaaggccccgtcattgactacctggccccttgcaacggtgactgctcgactgtcaacaagaccagtttggagttcttcaagatcgacggcgtgggcctcatcgacggctcctccccgccgggtaagtgggctgacgacgagctcattgccaacggcaacggctggctggtccagatccccgaggacatcaagccgggcaactacgtcctgcgccatgagatcatcgccttgcacgaggcgttcaaccagaacggcgctcagatctacccgcagtgcttcaacctccagattaccggctccggcactgtcgagcccgagggcacgccggctaccgagctgtattcgcccaccgatccgggcattctggttgacatctacaaccccttgagcacgtacgtcgtgcccggcccgacgctcatcccgcaggcggttgagattgagcagtcttcgtcggctgtcacggcgactggtacgccgacgccggcggcggcgtaa

SEQ ID NO: 96 - protein, 274 aa, Thermomyces lanuginosus
MKGSTTASLLLPLLASVTRTSAHGFVSNLVINGVFYRGWLPTEDPYKADPPIGVGWETPNLGNGFVLPEEASTDAIVCHKEAEPARGYASVAAGDKIYIQWQPNPWPESHHGPVIDYLAPCNGDCSTVNKTSLEFFKIDGVGLIDGSSPPGKWADDELIANGNGWLVQIPEDIKPGNYVLRHEIIALHEAFNQNGAQIYPQCFNLQITGSGTVEPEGTPATELYSPTDPGILVDIYNPLSTYVVPGPTLIPQAVEIEQSSSAVTATGTPTPAAA

SEQ ID NO: 97 - DNA, 821 bp, Humicola insolens
  source 1..821: mol_type = genomic DNA
atgaagctcagcgttgtcctcacaggcctggcggcagccctcgccgaggctcattgtcagtccatacgacagcgaaacccctggatgatcacgagactaaccagtcctaccagacaccttccccagcgtcggcaacaccgccgactggcaggtcgtgcgccagacgaccaacttccagagcaacggccccgtgacggacgtcaactcggaccagatccggtgctacgagcgcttccccggccagggggcgcccggcatctacaacgtcaccgccggccagaccatctcgtacaacgccaaggcctctatctcccacccgggccccatggccttctacatcgccaaggtccctgccggctacaccgccgccaactgggatggcaggggcgccgtgtggtccaagatctaccaggacatgccgcgcattgcggggagtctgacctggcctaccaatggtacgaaatcctcttctatccttcatacttgctattcctccaactgcctggcagctcacactaacttccacacacccaggcgcccgttccgtctcggtaaccatcccccgctgcctgcaagacggccactacctgttgcgcgccgagcacatcggcctgcacagcgcgagcggcgtgggcggcgcgcagttctacatctcgtgtgcccagctctacgtcagcggcggcaccggcacttggaacccgcgcaacaaggtcgcgttccccggcgcctacagcccgacgcacccgggcatcatgatcaacatctactggccggtgccgacgagctacacgccgccggggccgccggttgagacgtgctga

SEQ ID NO: 98 - protein, 227 aa, Humicola insolens
MKLSVVLTGLAAALAEAHYTFPSVGNTADWQVVRQTTNFQSNGPVTDVNSDQIRCYERFPGQGAPGIYNVTAGQTISYNAKASISHPGPMAFYIAKVPAGYTAANWDGRGAVWSKIYQDMPRIAGSLTWPTNGARSVSVTIPRCLQDGHYLLRAEHIGLHSASGVGGAQFYISCAQLYVSGGTGTWNPRNKVAFPGAYSPTHPGIMINIYWPVPTSYTPPGPPVETC

SEQ ID NO: 99 - DNA, 1120 bp, Humicola insolens
  source 1..1120: mol_type = genomic DNA
atgcgccccttcctcgccgccctcgccgcggccaccacggtccacgcccacggctgggtcgacaacgccaccatcgacggcgtcttctaccagctctaccacccgtacatggacccgtacatgggcgagttcgccccgcctcgcatctcgcgcaagctggtgtggaacggctacgtgaacgacgtgacgtccatcgacctgcaatgcggcggacacacggccgaagggcaaatcggcacggaacccgcgccgctgcacgcccccgccacggccgggtcgacggtcaacctccgctggacgctgtggccggactcgcacatggggcccatcatgacgtacatggcgcggtgtccggacgagggttgtgataagtggttgccgggggaggagtaagtgtttcctggcgggaatggctgtgtatttgagaaggagatattatgagtgaaactgggagaggcgagaagaagagatgctgacgcgggttttgctctcctcagaccagtctggttcaaaatccacgaagccggccggtacaccaccgacaagtcttaccccgacgacatctgggaagttgtaagtgccctgcctacctatccatccctaattccctccctcccctctccacctcctccttccgcgcccccctccccccccttatttgctaaccaaccccctcccttacagacccgcctcatgtaccccgccaacgaaggctacaactacaccatccccgcctgcctcgcatccggccactacctggtccggcacgagatcatcgccttacactcggcctgggccaaaggcgaagcgcagttctatccctcgtgccaccagctgaccgtcacctccatcggcggtaacgtgcgcgaagcgccggccgagtaccgcgtcagtttccccggcgcgtacaaggacgatgatccgggtattttcatcaacgtttggaaccgtaagttctttttttgttccccttcctcccaacctacctaggtgtcgtaatgtggtccgtaagggtttgtttgttgttgagggatatagctgacaatggatgtgtgataacacagctggcccctacaccattcccggaccgccggtctggacgtgccccgagtctgagtaa

SEQ ID NO: 100 - protein, 257 aa, Humicola insolens
MRPFLAALAAATTVHAHGWVDNATIDGVFYQLYHPYMDPYMGEFAPPRISRKLVWNGYVNDVTSIDLQCGGHTAEGQIGTEPAPLHAPATAGSTVNLRWTLWPDSHMGPIMTYMARCPDEGCDKWLPVWFKIHEAGRYTTDKSYPDDIWEVTRLMYPANEGYNYTIPACLASGHYLVRHEIIALHSAWAKGEAQFYPSCHQLTVTSIGGNVREAPAEYRVSFPGAYKDDDPGIFINVWNPGPYTIPGPPVWTCPESE

SEQ ID NO: 101 - DNA, 878 bp, Humicola insolens
  source 1..878: mol_type = genomic DNA
atgagactctccctgacaaccctcctggcctctgccctgtccgtccagggtcacgccatcttccaggtgcgttcctttcaccacccacatcatcatgatgaacctcaaagttgctaacccccgctgggcagagagttaccgtcaacggccaggaccaaggctcgttgactggtctccgggccccgaataacaacaaccccgtgcagaacgtcaacagccaggacatcatctgtggcgctcccgggtcgcggtcacagtccgtcatcaacgtcaatgccggcgaccgcatcggtgcctggtaccagcatgtcatcggcggcgcccagttccccggcgacccggacaacccgatcgccaggtcccacaagggccccatctccgtctatctggccaaggtggacaacgctgccacggcgaaccaccagggtctgcaatggtaaacatacctcgggtcaagtcagaacctctgtgatcgccgagacgactaacccctctttcccataaacaggttcaagatctggcacgacggcttcaacccctccacccggcaatgggccgtcgacaccatgatcaacaacaacggctgggtctatttcaacctcccgcagtgcatcgctcccggccactatctcatgcgcgtcgagctgctcgctctccactcggccacctaccaaggccaggcgcagttctacatctcgtgcgcccagatcaacgtccagtcgggcggcaactttactccctggcagacggttagcttccccggcgcctaccaggccaaccaccccggcattcaggtcaacatttacggcgccatgggccagccggataacggcggcaggccctaccagattccgggcccggagccgattcagtgctga

SEQ ID NO: 102 - protein, 246 aa, Humicola insolens
MRLSLTTLLASALSVQGHAIFQRVTVNGQDQGSLTGLRAPNNNNPVQNVNSQDIICGAPGSRSQSVINVNAGDRIGAWYQHVIGGAQFPGDPDNPIARSHKGPISVYLAKVDNAATANHQGLQWFKIWHDGFNPSTRQWAVDTMINNNGWVYFNLPQCIAPGHYLMRVELLALHSATYQGQAQFYISCAQINVQSGGNFTPWQTVSFPGAYQANHPGIQVNIYGAMGQPDNGGRPYQIPGPEPIQC

SEQ ID NO: 103 - DNA, 1067 bp, Humicola insolens
  source 1..1067: mol_type = genomic DNA
atgggaccgacctgggcagtgattctggggctgattgctccttctgtgctcagtcacagttgcgtctcccaacagacctctcgacttttatcaagctggtactgactcataacccaactcacctagatatccatgggatcctcctggtcaatggcacagagacaccagagtggaaatacgtcctgtatgtttcctcatatcctagccccattgtacgagttgttgacgtgatacagcgatgttgcgccggcggttccaatttcaaacccagactctctcccccctggataccaaggctataaggttgatcccatcatcggatccgggaaccccaacatcacttgtggccggctagcatttgactcggcacccaagacgcaaatcgccgatgtgctagccggctccgaggtaggattccgagtctcggctgatggcttgggaaatcgggatctggagaagggctacatcccgacgttctggcacccaggtccggcccaggcatacttgtcacgtgccccgaacgacgacctgtacagctacaaaggcgacggggactggttcaagattgcctacgctggcccggtggacgacctgacgtggtccctttggccgggagtttcagatgtatgttcatcctccatagtcctgtttttgccctctccaggaccaaattattaatatcgagtcgcagttcaacttcaccattccgttgtcgacaccccctggcaagtatttgctccgaatcgagaacttcatgccaacggcctcgacaggatatcttcagttctacgtcaattgtgcatttgtcaacatcattggaccaggaggtgggaccccgaccgagttcattcgaattcccggggattacaccgacgaggatccaggtgagtttgtgttatgagacatgttcaactcgcaccgacgaatgcttgtttcctgacagagatttgtaaaaactaggctttctcgttcccccggagcaaagctccttggatggcagagtcccaagggaccagttgaaactgatgagctacacgccaccaggtcctgcggtgtggacggggtga

SEQ ID NO: 104 - protein, 265 aa, Humicola insolens
MGPTWAVILGLIAPSVLNIHGILLVNGTETPEWKYVLDVAPAVPISNPDSLPPGYQGYKVDPIIGSGNPNITCGRLAFDSAPKTQIADVLAGSEVGFRVSADGLGNRDLEKGYIPTFWHPGPAQAYLSRAPNDDLYSYKGDGDWFKIAYAGPVDDLTWSLWPGVSDFNFTIPLSTPPGKYLLRIENFMPTASTGYLQFYVNCAFVNIIGPGGGTPTEFIRIPGDYTDEDPGFLVPPEQSSLDGRVPRDQLKLMSYTPPGPAVWTG

SEQ ID NO: 105 - DNA, 1035 bp, Humicola insolens
  source 1..1035: mol_type = genomic DNA
atgaaggccctcaccctcctcgccgccgcgaccgcggcctcggcgcacaccatcttcgtgcagctcgaggccgacggcacgcgctaccccgtctcgcacggcgtgcgcaccccgcagtacgacggccccatcaccgacgtctcgtccaacgacctggcctgcaacggcgggcccaacccgaccatgaagacggacaagatcatcaccgtgacggcgggcagcaccgtcaaggccatctggcggcacacgctgcagtcgggccccaacgacgtcatggaccccagccacaagggcccgacgctggcgtacctgaagaaggtggacaacgcgctgacggattcgggcgtgggcggcggctggttcaagatccaggaggacgggcacagcaatgggaattggggcacgctcaaggtaatcaacaaccagggcattcactatatcgatatccccgactgcatcgacagcgggcagtatttgttgcgggccgagatgatcgctctgcacgctgccgggtcgccgggcggtgcgcagctttatgtgagtttcttccttcttttcttcttctctccctttgtgataagaataaagatccacaccacagtcaaaccaaagcatcctaacctcggcatctactcacagatggaatgcgcccaaatcgaaatcgtcggcggcaagggcaccgtcaagccccagacctactccatcccgggcatctacaagtccaacgacccgggcatcctcatcaacatctactccatgtcgccctcgagccagtacatcatccccggcccgcccctcttcacctgcaacggcggcggcggcagcaacaacggcggcggcaacaacggcggcagcaacccccccgtccagcagccccccgccaccaccctcaccaccgccatcgcccagcccacgcccatctgctccgtccagcagtggggtcagtgcggcggccagggctatagcggctgcaccacctgcgcgtcgccgtataggtgtaacgagatcaacgcgtggtattcgcagtgcttgtaa

SEQ ID NO: 106 - protein, 310 aa, Humicola insolens
MKALTLLAAATAASAHTIFVQLEADGTRYPVSHGVRTPQYDGPITDVSSNDLACNGGPNPTMKTDKIITVTAGSTVKAIWRHTLQSGPNDVMDPSHKGPTLAYLKKVDNALTDSGVGGGWFKIQEDGHSNGNWGTLKVINNQGIHYIDIPDCIDSGQYLLRAEMIALHAAGSPGGAQLYMECAQIEIVGGKGTVKPQTYSIPGIYKSNDPGILINIYSMSPSSQYIIPGPPLFTCNGGGGSNNGGGNNGGSNPPVQQPPATTLTTAIAQPTPICSVQQWGQCGGQGYSGCTTCASPYRCNEINAWYSQCL

SEQ ID NO: 107 - DNA, 1065 bp, Humicola insolens
  source 1..1065: mol_type = genomic DNA
atggctcccaagacctcgacgttccttgcctccctcacgggcgccgccctcgtggctgcccacggccatgtcagccacatcattgtcaatggcgtccagtaccggaactacgaccccaccaccgacttctacagcggcaaccctccgaccgtgatcggctggtcggccctcaaccaggacaacggcttcatcgagcccaacaacttcggcacccccgacatcatctgccataagtcggccaagcccggcggcggccacgtcacggtgagggccggtgacaagatcagcatcgtctggacccccgagtggcccgagtcgcacgtcggccccgtcatcgactaccttgccgcgtgcaacggcgactgcgagacggtcgacaagacctccctccgcttcttcaagatcgacggcgccggctacgacgccgcggccggccgctgggccgccgacgctctgcgcgccaacggcaactcgtggcttgtgcagatccccgccgacctcaaggccggcaactacgtgcttcggcacgagatcatcgccctgcacggcgccgccaaccccaacggcgcccaggcctacccgcagtgcatcaacatccgcgtcaccggcggcggcaacaaccagccctcgggcgtccccggcacccagctctacaaggcctcggacccgggcatcctcttcaacccctgggtcgccaaccctcagtaccccgtcccgggcccggccctcatccccggcgccgtgagctccatccctcagagccgctcgaccgccaccgccacgggcaccgccacccgccccggcgccgacacggacccgacgggcgtccctcccgtcgtcaccaccacttctgccccggctcaggtgaccaccaccaccagcagccgcaccacctccctccctcagatcaccaccaccttcgcgaccagcaccaccccgccgcccccggccgctacccagagcaagtggggccagtgcggcggcaacggctggaccggcccgaccgtctgcgcgccgggctcgagctgcaacaagctcaacgactggtactcgcagtgcatctaa

SEQ ID NO: 108 - protein, 354 aa, Humicola insolens
MAPKTSTFLASLTGAALVAAHGHVSHIIVNGVQYRNYDPTTDFYSGNPPTVIGWSALNQDNGFIEPNNFGTPDIICHKSAKPGGGHVTVRAGDKISIVWTPEWPESHVGPVIDYLAACNGDCETVDKTSLRFFKIDGAGYDAAAGRWAADALRANGNSWLVQIPADLKAGNYVLRHEIIALHGAANPNGAQAYPQCINIRVTGGGNNQPSGVPGTQLYKASDPGILFNPWVANPQYPVPGPALIPGAVSSIPQSRSTATATGTATRPGADTDPTGVPPVVTTTSAPAQVTTTTSSRTTSLPQITTTFATSTTPPPPAATQSKWGQCGGNGWTGPTVCAPGSSCNKLNDWYSQCI

SEQ ID NO: 109 - DNA, 804 bp, Humicola insolens
  source 1..804: mol_type = genomic DNA
atgtatcttttacctatcgccgcggccgccctagcgttcaccaccaccgcatacgcccacgcccaagtctacggcttgcgtgtcaacgaccaacaccaaggcgatgggcgcaacaaatacatccgctcgcccagcagcaattcccccatccggtgggaccacgtaacccacccattcctcatctgcaacatccgcgacgacaaccaacccccgggtcccgcgcctgactttgtccgcgccttcgccggcgaccgcgtggcgttccaatggtaccacgcccgccccaacgacccgacggattacgtcctcgacagctcccacctcggcgtcctcgttacctggatcgcgccgtacacggacgggcccgggaccggccccatttggaccaagatccaccaggacgggtggaacggcacgcactgggccacgagccggctcatcagcaacggcgggttcgtcgagttccggctgcccggctcgctaaagcccgggaagtacctggtgcggcaggagattatcgctctgcaccaggccgacatgcccggtccgaaccgcgggcctgagttctaccccagctgcgcgcaattggaggtttttgggtctggtgaggcggcgccgccgcaggggtatgatatcaacaaggggtatgcggagagcggggataagttgtggttcaacatttacatcaacaagaatgatgagttcaaaatgcctggaccggaggtttgggatggtgggtgtcggtttggagagcgatgggcaaccgaggaaccaggcaagcccaaggtgaaccaacacggataa

SEQ ID NO: 110 - protein, 267 aa, Humicola insolens
MYLLPIAAAALAFTTTAYAHAQVYGLRVNDQHQGDGRNKYIRSPSSNSPIRWDHVTHPFLICNIRDDNQPPGPAPDFVRAFAGDRVAFQWYHARPNDPTDYVLDSSHLGVLVTWIAPYTDGPGTGPIWTKIHQDGWNGTHWATSRLISNGGFVEFRLPGSLKPGKYLVRQEIIALHQADMPGPNRGPEFYPSCAQLEVFGSGEAAPPQGYDINKGYAESGDKLWFNIYINKNDEFKMPGPEVWDGGCRFGERWATEEPGKPKVNQHG

SEQ ID NO: 111 - DNA, 843 bp, Humicola insolens
  source 1..843: mol_type = genomic DNA
atgaagctcctcgctcctctgatgctggctggcgccgccagcgcccgtgagtaacccctggctggatctcatgctggtgccagtgttccatgactgacaaccaccctcagacaccatcttcacctccctcgaggttgatggccgcaactacggcacgggcaacggcgtccgcgtcccctcctacaacggccccgtcgaggatgtcacgtccaactcgatcgcctgcaacggcccgccgaacccgaccagcccgaccgacacggtcatcaccgtccaggctggccagaacgtgactgccatctggcggtacatgctcaacacccagggcacctcgcccaacgacatcatggacagcagccacaagggtcctactctcgcctacctcaagaaggtcaacgatgcccggactgactcgggcgtcggcgatggctggttcaagatccagcacgacggcttcgacggcaccacctggggcaccgagcgcgtcatcttcggccagggccgtcacaccatcaagatccccgagtgcatcgagcccggccagtacctgctgcgtgctgagatgatcgccctccacggcgcccagaactacccgggtgctcagttctacatggagtgcgcccagctcaacattgtcggtggcaccggcaccaagaaacccagcaccgtcagcttccctggcgcttacaaggtatgtccgagtttggtaccgagataactggagatgagaaaagtgatgctaacaaaccatgacagggcaccgaccccggcgtcaagctcagcatctggtggccgcccgtcaccaactacgtcattcccggccccgatgtcttcaagtgctaa

SEQ ID NO: 112 - protein, 237 aa, Humicola insolens
MKLLAPLMLAGAASAHTIFTSLEVDGRNYGTGNGVRVPSYNGPVEDVTSNSIACNGPPNPTSPTDTVITVQAGQNVTAIWRYMLNTQGTSPNDIMDSSHKGPTLAYLKKVNDARTDSGVGDGWFKIQHDGFDGTTWGTERVIFGQGRHTIKIPECIEPGQYLLRAEMIALHGAQNYPGAQFYMECAQLNIVGGTGTKKPSTVSFPGAYKGTDPGVKLSIWWPPVTNYVIPGPDVFKC

SEQ ID NO: 113 - DNA, 705 bp, Humicola insolens
  source 1..705: mol_type = genomic DNA
atgaagctcctctcaaccctcgccgccattgcggccaccttggccacggcggatgcgcactacatcttcaacatcctgtacgtcaacggccagcgcatgggcggcgagtacacctacgtgcggcgcaactccaactcgtacttccccgtgttccccgacatcctcaactccaacgacatgcgttgcaacgtgggtgccagaccgggcaacacccaaaccgccaccgtcagggccggcgacaggatcggcttcaaggtcttcaacaacgaggtcatcgagcaccctggtcccggcttcatctacatgtccaaagccccgggcagcgtcaacaactatgacggcagcggggactggttcaaggtttacgagaccggtctctgccgcggtggtggcaacgtcgacaccaactggtgctcgtactacaaggaccggctcgagtttaccatcccgcccaagactcctcccggcgagtatctggtgcgtatcgagcatatcggtctgcacgagggccacgtcaacagggcgcagttctacatcacctgcgcgcagctcaagattgaggggcccggcggcggcaacccgaacccactcgtgaagatcccgggcatctacagggccaacgaccccggcatcgcctacaacaagtggaccaacaacccggcgccgtacatcatgccgggtcccaaggtgtgggatggcaactaa

SEQ ID NO: 114 - protein, 234 aa, Humicola insolens
MKLLSTLAAIAATLATADAHYIFNILYVNGQRMGGEYTYVRRNSNSYFPVFPDILNSNDMRCNVGARPGNTQTATVRAGDRIGFKVFNNEVIEHPGPGFIYMSKAPGSVNNYDGSGDWFKVYETGLCRGGGNVDTNWCSYYKDRLEFTIPPKTPPGEYLVRIEHIGLHEGHVNRAQFYITCAQLKIEGPGGGNPNPLVKIPGIYRANDPGIAYNKWTNNPAPYIMPGPKVWDGN

SEQ ID NO: 115 - DNA, 753 bp, Humicola insolens
  source 1..753: mol_type = genomic DNA
atgctgggaagcgctcttctgctcctgggcactgccctgggcgccaccgcccactacacgttccctaggatcaacagcggcggcgactggcagtatgtccgccgggccgacaactggcaggacaacggcttcgttggcaacgtcaactcgcctcagatccggtgcttccagagcaggcaccaggccgccccggccaccctcaacgtcaccgccggctccacggtgacctactacgccaatcccaacgtctatcaccccggcccgatggccttctacatggcccgcgtccccgatggccaggatatcaactcgtggaccggcgagggtgccgtgtggttcaagatctaccacgagcagcctaccggcctgggccagcagctgaggtggtctagcgatggtacgtgaatggtgatcctgtggcatctcaacctcttccagacttctgacccgagcccccgcggccctacaggcaagaactcgttccaggttcagatcccccgctgcatccgctctggctactacctgctccgtgctgagcacatcggcttgcacagcgccggcagccctggtggcgctcagttctacatctcttgcgcccagctcgccgtcaacggcggtggcagcaccgagccccccaacaaggtgtccttccctggtgcctacagcccgtccgaccccggcattcagatcaacatctactggcctgttccgacctcgtacaagaaccccggccccccggtcttccagtgctaa

SEQ ID NO: 116 - protein, 226 aa, Humicola insolens
MLGSALLLLGTALGATAHYTFPRINSGGDWQYVRRADNWQDNGFVGNVNSPQIRCFQSRHQAAPATLNVTAGSTVTYYANPNVYHPGPMAFYMARVPDGQDINSWTGEGAVWFKIYHEQPTGLGQQLRWSSDGKNSFQVQIPRCIRSGYYLLRAEHIGLHSAGSPGGAQFYISCAQLAVNGGGSTEPPNKVSFPGAYSPSDPGIQINIYWPVPTSYKNPGPPVFQC

SEQ ID NO: 117 - DNA, 854 bp, Humicola insolens
  source 1..854: mol_type = genomic DNA
atgaagctgcttcctgggttgcttctggcagccacggctgcccaagcccattgtacgtttccgatccccaagaccatcttcgagaattttcgagccagatctttctgagagagttgctgacaattcctgctagacacattccccaggctcgttgtcaacgggcagcctgaggagagggactggtcggtcactcggatgacaaagaaccaccagagcaagtcgggaattgaaaacccaactagccccgacatccgttgctacagctcgcagactgcccctaacgtggcgattgtgccggccgggtctaccatccactacatctcgacccaacaaatcaaccatcctggcccgactcagtactatctcgccaaggtcccagctggtcagtcagccaagacctgggatggctctggcaacgtgtggttcaagatcgccacgagcatgccggagtacgatcaaaacaggcagctggtttggcccggtcatagtaaggactcactctcgtccgatcatctcttttgagtgagtcttgggcatacccactgactacgtctgctatgacagatacctatcagaccatcaacgccaccatcccggccaacacgccgagcggagagtacctcctgcgtgtcgagcaaattgccctccacatggccagccagccgaacaaggcccagttctacatctcgtgctctcagattcagattaccaatggcggaaacggcactccgggccctctagttgcattcccgggggcatacaggagcaacgaccctggcatcctggtcaatctctacagcggcatgcagccttcgcagtaccagccccctggaccggccgtgtggcgtggctga

SEQ ID NO: 118 - protein, 231 aa, Humicola insolens
MKLLPGLLLAATAAQAHYTFPRLVVNGQPEERDWSVTRMTKNHQSKSGIENPTSPDIRCYSSQTAPNVAIVPAGSTIHYISTQQINHPGPTQYYLAKVPAGQSAKTWDGSGNVWFKIATSMPEYDQNRQLVWPGHNTYQTINATIPANTPSGEYLLRVEQIALHMASQPNKAQFYISCSQIQITNGGNGTPGPLVAFPGAYRSNDPGILVNLYSGMQPSQYQPPGPAVWRG

SEQ ID NO: 119 - DNA, 863 bp, Humicola insolens
  source 1..863: mol_type = genomic DNA
atgctcctgaactcggtcatcggctcggccgtcctcctggccaccggcgccgccgcccacggtgccgtgaccagctacgtcattgccgggaagaactaccctgggtaggtaacctcgtggaagcgaatgcaggcagttcattcactaacacatacctccgttagctacaacggctacgccccgtccaccacccccaacacgatccagtggcaatggtcgacctacgaccccatctactccgccaccgaccccaagctccgctgcaacggcggccgctcggccacgcagtccgccccggctgctccgggcgacaacatcaccgccatctggcagcagtggacgcatagccagggccccatcctcgtctggatgtacaagtgtcccggcgccttcagctcgtgcgacggctcgggccagggctggttcaagattgacgaggccggcttcaatggcgacggcaagaccgtgttcctcgacaccgagcgcccctccggctgggagatcgccaagctggttggcggcaacaagggctggaccagcaccatccccaagaacctggccccgggcaactacctggtccgccacgagttgattgcccttcaccaggccaacgccccgcagtggtaccctgagtgcgcgcaggtcgtgatcaccggctcgggcactaaggagccgcctgcgtcgtacaaggctgccattcccggctactgcaaccagaacgatcccaacattcgggtatgtgaggcctatttggagttcggctaaggcatgatactaactctaccccccaggttcctatcaacgaccactccatcccccagacctacaagatccctggccctccggtctggcgcggcgagtaa

SEQ ID NO: 120 - protein, 248 aa, Humicola insolens
MLLNSVIGSAVLLATGAAAHGAVTSYVIAGKNYPGYNGYAPSTTPNTIQWQWSTYDPIYSATDPKLRCNGGRSATQSAPAAPGDNITAIWQQWTHSQGPILVWMYKCPGAFSSCDGSGQGWFKIDEAGFNGDGKTVFLDTERPSGWEIAKLVGGNKGWTSTIPKNLAPGNYLVRHELIALHQANAPQWYPECAQVVITGSGTKEPPASYKAAIPGYCNQNDPNIRVPINDHSIPQTYKIPGPPVWRGE

SEQ ID NO: 121 - DNA, 883 bp, Humicola insolens
  source 1..883: mol_type = genomic DNA
atgaagctcaccacctccatcgccctgctggctgcggccggcgcgcaggctcactgtacgtgctccctcatctcatccatctcctcagaccatgttttacctattggttactaacaagctctcacgcagacaccttcccccgcaccaaggtcgacggcgtcacctcgggcgagtgggagacgatccgcatcaccgagaaccactggtcgcacggccccgtgacggacgtgacctcgcaggccatgacgtgctacgagaagacgcccggccagggcgcgcccaagacggttaacgtgaaggccggcggcaccgtcaccttcaccgtcgacacggacgtgggccacccgggcccgctgcacttctacttggccaaggtgcccgcgggcaagacggccgcgacgtttgacggcaagggcgccgtgtggttcaagatttaccaggacggccccggcgggttggggaccagctcgttgacttggcctagctttggtgagctttcttttctttattttcttcaatcctcccataattacctcccgacgaggaaataaatataccttacctgatattaacccatccccccccacctcctccaggcaagaaggaagtctctgtccaaatccccccctgcgtgcaggacggcgagtacctgctgcgcgtcgagcacattgcgctgcacagcgccgcgagcgtcggcggcgcgcagctctacatttcgtgcgcgcaaatcaacgtcaccggcggcaccggcacgctcaacccgggccagctcgtctcgttcccgggcgcctacaagcccaccgacccgggcatcctgttccagctctactggccgccgccgacccagtacatcaaccccggtccggcgccggtgaagtgctga

SEQ ID NO: 122 - protein, 233 aa, Humicola insolens
MKLTTSIALLAAAGAQAHYTFPRTKVDGVTSGEWETIRITENHWSHGPVTDVTSQAMTCYEKTPGQGAPKTVNVKAGGTVTFTVDTDVGHPGPLHFYLAKVPAGKTAATFDGKGAVWFKIYQDGPGGLGTSSLTWPSFGKKEVSVQIPPCVQDGEYLLRVEHIALHSAASVGGAQLYISCAQINVTGGTGTLNPGQLVSFPGAYKPTDPGILFQLYWPPPTQYINPGPAPVKC

SEQ ID NO: 123 - DNA, 928 bp, Humicola insolens
  source 1..928: mol_type = genomic DNA
atgaagactctcgcatccgccctcattgccgcgggccttctggcccagtacgccgctgcccatgccattttccagtttgccagcagcggtggcactgactttgggacgtcctgtgttaggatgccggtgagtgaacgggtgcccctgaacatgtgttgctcacgaaacaaggttatgttgactctatacagcccaacaactctcccgtcacgagcgtcaccagcagtgacatggcttgcaatgttggcggatctcgcggtgtatctggcatttgcgaggtgaacggtaagagttctcctcagccttttctctgtcaagcactaaacagcactcgctaaccatttcaatctcagccggctccgacttcaccgtcgagatgcacgcgcagcccaacgaccggtcgtgcgccagcgaagccattggcggcaaccacttcgggcccgtcatggtgtacatggccaaggtggacgacgcgacgcgggcggacggtgcgtcggcgtcttggttcaaggtggacgagttcggctacgacgccggctccaagacatggggaaccgacatgctcaacaagaactgcggcaagcggacgttccgcatcccgagcaaaatcccgtctggggactatctggtgcgtgcggaggctattgctttgcacaccgcgggccagccgtcgggtgcgcagttttatatgagctgctatgtgagttcttccatgcttccccttgtggtgtcactgtatagaagatgctaatatctcccacagcaagttcgcatcaagggcagcaacaacggtcagcttccggctggtgttcggattcctggcgcctacagcgcgacggacccgggcatcctcgtcgatatctggggcaatggtttcagccagtacactattcctggccctcgtgtcattgatgggagctttttctga

SEQ ID NO: 124 - protein, 243 aa, Humicola insolens
MKTLASALIAAGLLAQYAAAHAIFQFASSGGTDFGTSCVRMPPNNSPVTSVTSSDMACNVGGSRGVSGICEVNAGSDFTVEMHAQPNDRSCASEAIGGNHFGPVMVYMAKVDDATRADGASASWFKVDEFGYDAGSKTWGTDMLNKNCGKRTFRIPSKIPSGDYLVRAEAIALHTAGQPSGAQFYMSCYQVRIKGSNNGQLPAGVRIPGAYSATDPGILVDIWGNGFSQYTIPGPRVIDGSFF

SEQ ID NO: 125 - DNA, 1092 bp, Humicola insolens
  source 1..1092: mol_type = genomic DNA
atgcctcgcttcaccaagtccattgtctcggccctggccggcgcttccctggtcgcagcccacggccatgtcacccacatcgtcatcaacggcgtgctgtacccgaacttcgaccctacatcccacccttacctgcagaacccgccgaccgttgtgggctggaccgccgccaacaccgacaacggcttcgttgctcccgaccagttcgcctcgggcgatatcatctgccacaaccaggccaccaacgcgggcggccacgccgtggtcgcggccggcgacaagatttggatccagtgggaccagtggcctgagagccaccacggccccgtcctcgactacctcgcctcctgcggcagctcgggctgcgagtcggtcaacaagctcgacctcgagttcttcaagatcggcgaaaagggcctgatcgacggctcctccgcgccgggccggtgggcgtcggacgagctgatcgccaacaacgccggctggctggtccagatccccgccgacattgcgcccggccactacgtcctgcgccacgaaatcatcgccctccacgccgccggccagcccaacggcgcccagaactacccgcagtgcttcaacctcctcgtcacgggctccggcaccgcgcggccgcagggcgtcaagggaacagcgctgtacacccccaacgacaagggcatcttggcgggcatctacaatgcccccgtctcgtacgagattcccggccccgcgctctactccggcgccgccaggaacttgcagcagagctcgtcccaggccacgtcgactgccacggctttgactggggacgcggtgcctgttccgacccaagcccccgtcactaccacttcctcttcttcggccgatgccgccaccgccacctccaccaccgtccagccgccccagcaaaccaccctcacgaccgccatcgccacgtcgaccgctgctgctgccccgacgaccaccgccggcagcggaaacggtggcaaccggcccttcccaacccgctgccctggcctggctgggctcgggtttgacaagcgccgtcgccagctccgcgctgaggagggtgtgcaggtggttgcttga

SEQ ID NO: 126 - protein, 363 aa, Humicola insolens
MPRFTKSIVSALAGASLVAAHGHVTHIVINGVLYPNFDPTSHPYLQNPPTVVGWTAANTDNGFVAPDQFASGDIICHNQATNAGGHAVVAAGDKIWIQWDQWPESHHGPVLDYLASCGSSGCESVNKLDLEFFKIGEKGLIDGSSAPGRWASDELIANNAGWLVQIPADIAPGHYVLRHEIIALHAAGQPNGAQNYPQCFNLLVTGSGTARPQGVKGTALYTPNDKGILAGIYNAPVSYEIPGPALYSGAARNLQQSSSQATSTATALTGDAVPVPTQAPVTTTSSSSADAATATSTTVQPPQQTTLTTAIATSTAAAAPTTTAGSGNGGNRPFPTRCPGLAGLGFDKRRRQLRAEEGVQVVA

SEQ ID NO: 127 - DNA, 1086 bp, Humicola insolens
  source 1..1086: mol_type = genomic DNA
atgaagggacttctcagcatcgccgccctttccctggcggttggtgaggcttcggcccactacatcttccagcagctctcgacgggtggcaccaagcaccccatgtggaagtacatccgccagcacaccaactacaactctcccgtcatcgacctcgactccaacgacctccgctgcaatgtcggtgcccggggtgctggaactgagaccgttacggtcgctgctggctcgagcctgaccttccacctcgacacccccgtctaccaccagggccctgtgtcggtgtaagtagaagttctcagacgaaccaccaatgtcggcagataatttctaactccgatgtccagctatatgtccaaggctcccggctccgtgtcggactatgacggcagcggcggctggttcaagattcaagactggggcccgaccttcaccggcagcggcgccacctggaagctggatgactcctacaccttcaacatcccctcgtgcattcccgacggcgagtacctcgtccgcatccagtccctgggtatccacaacccctggccggcgggtattccgcagttctatatctcgtgcgctcaggtgcgcgtcaccggcggtggcaacgcgaacccgagcccgcaggtgtcgatcccaggtgccttcaaggagaccgacccgggctacactgccaacgtgagtttccatccatgctacatatcccttttacgctctcgatcccatgactaacccccccctgaaaagatctacaacaacttccgcagctacaccgtccccggcccgtccgtcttcacctgcagcggcaacagcggcggcggctccaaccccagcaaccctaaccccccgaccccgacgaccttcaccacccaggtgaccaccccgaccccggcgtctccgccctcttgcaccgtcgcgaagtggtacgtctgaaaaaaaatctcctccaggccggacatgagaaaactaacatgaacgaaaaacaggggccagtgcggtggccagggctacagcggctgcaccaactgcgaggccggctcgacctgcaggcagcagaacgcttactattctcagtgcatctaa

SEQ ID NO: 128 - protein, 296 aa, Humicola insolens
MKGLLSIAALSLAVGEASAHYIFQQLSTGGTKHPMWKYIRQHTNYNSPVIDLDSNDLRCNVGARGAGTETVTVAAGSSLTFHLDTPVYHQGPVSVYMSKAPGSVSDYDGSGGWFKIQDWGPTFTGSGATWKLDDSYTFNIPSCIPDGEYLVRIQSLGIHNPWPAGIPQFYISCAQVRVTGGGNANPSPQVSIPGAFKETDPGYTANIYNNFRSYTVPGPSVFTCSGNSGGGSNPSNPNPPTPTTFTTQVTTPTPASPPSCTVAKWGQCGGQGYSGCTNCEAGSTCRQQNAYYSQCI

SEQ ID NO: 129 - DNA, 1032 bp, Humicola insolens
  source 1..1032: mol_type = genomic DNA
atgaggcccttctcactcgtcgctctggcgacggccgtcagcggccacgccatcttccagcgcgtgtcggttaacggcgtcgaccaaggccagctcaagggcgtccgcgctccctcgagcaactaccccatcgagaacgtcaaccaccccgactttgcctgcaacaccaacatccagcaccgcgacggcaccgtcatcaagatccccgccggcgccaccgtcggcgcctggtggcagcacgagatcggcgggccctcgttcccgggtgacccggataacccgatcgctgcttcgcacaagggtgagttcccatagatagatctcttctctcccgaccccttgtatcctctcataactaaccacctcaaccccccaggccctatccaagtctacctcgccaaggtcgacaacgccgcgaccgcctcccccaacggcctgcggtggttcaagattgccgagaagggcctgtcgggcggcgtctgggccgtcgacgagatgatccgcaacaacggctggcactacttcaccatgccgcagtgcatcgcgcccggccactacctgatgcgcgtcgagctgttggcgctgcactcggccagcttccccggcggcgcccagttctacatggagtgcgcccagatcgaggtcaccggctcgggcaacttctcgccctccgagacggtcagcttccccggcgcctacccggccaaccacccgggtatcgtcgtcagcatctacgacgcccagggtaacgccaacaacggcgggcgcgagtaccagatccccgggccgcggccgatcacctgctccggcggtggaagcaacaatggtggcgggaacaacaatggtggtggaaacaacaacggcggcggcaacaacaacggcggtgggaacaacaacggtggtggtaacaccggtggcggctcggcgccgctctggggccagtgcggcggcaatgggtataccggcccgacgacttgtgccgagggtacttgcaagaagcagaatgactggtactcgcagtgtacgccttag

SEQ ID NO: 130 - protein, 318 aa, Humicola insolens
MRPFSLVALATAVSGHAIFQRVSVNGVDQGQLKGVRAPSSNYPIENVNHPDFACNTNIQHRDGTVIKIPAGATVGAWWQHEIGGPSFPGDPDNPIAASHKGPIQVYLAKVDNAATASPNGLRWFKIAEKGLSGGVWAVDEMIRNNGWHYFTMPQCIAPGHYLMRVELLALHSASFPGGAQFYMECAQIEVTGSGNFSPSETVSFPGAYPANHPGIVVSIYDAQGNANNGGREYQIPGPRPITCSGGGSNNGGGNNNGGGNNNGGGNNNGGGNNNGGGNTGGGSAPLWGQCGGNGYTGPTTCAEGTCKKQNDWYSQCTP

SEQ ID NO: 131 - DNA, 1113 bp, Humicola insolens
  source 1..1113: mol_type = genomic DNA
atggtgttgcggtctctctctatcctggccttcgtagccagaggcgtcttcgcccacggtggcctctccaactacacggtcggcgacacgtggtatagcgggtgcgtccatgaacaactcctatatcttccccccctccacattgcgaccgctgcacatctcactcgtccataaacaacaacatcaatcggtagacactgtccaaaagctaaccaccgtacctcctgaacacagctacgaccccttcacccccgccgccgcccaactctcccaaccctggctgatccaacgccaatggaccagcatcgacccgctcttctccccgacctctccctacctcgcctgcaacttccccggcaccgcgccaccatcttacatccctctccgcgccggcgacatcctcaccgcggtttactggttctggctgcaccccgtggggccgatgagcgtttggctggcgcggtgcgcaggggactgccgcgacgaggacgtgacgcgggcgcgctggttcaagatctggcatgcggggtttctggaggggccgaatttggagctcgggatgtggtatcagaagaagttccagcggtgggatggcgggccggcgctctggcgggtgaggataccgagggggttgaagaaggggttgtacatggtcaggcatgagattttgtcgattcatgtgggtggacggccccagttttatcccgagtgtgcgcacttgaatgtgacggagggtggtgaggtggtagtgccgggggagtggacgagaaggttccctggggcgtatgacgatgatggtgagtgccttgctagacgggaaggctctatggatggggcggatgagacgaaaggctggtgtgagactgtcagcactgacggcctgcagacaagtcagtcttcatcgatatctaccggccggaacatgaaaacaggacggtacgtgggacaagcaagcctcggatttttcagattttcgactctgacaacgaacaggactatgagatccctggaggcccgatttgggaaaggtacgtacaatcgcatcatcttgactctgtattcaggggctaacataaacacagcttgggggagatggagttatggcctgaatga

SEQ ID NO: 132 - protein, 259 aa, Humicola insolens
MVLRSLSILAFVARGVFAHGGLSNYTVGDTWYSGYDPFTPAAAQLSQPWLIQRQWTSIDPLFSPTSPYLACNFPGTAPPSYIPLRAGDILTAVYWFWLHPVGPMSVWLARCAGDCRDEDVTRARWFKIWHAGFLEGPNLELGMWYQKKFQRWDGGPALWRVRIPRGLKKGLYMVRHEILSIHVGGRPQFYPECAHLNVTEGGEVVVPGEWTRRFPGAYDDDDKSVFIDIYRPEHENRTDYEIPGGPIWESLGEMELWPE

SEQ ID NO: 133 - DNA, 1103 bp, Humicola insolens
  source 1..1103: mol_type = genomic DNA
atgaggaccgtcttcgccgccgcactggcagcactcgctgcccgggaagtcgccggccatgccacgttccagcaactctgggttgacggaaccgattatataagtgccccccttttctcggttccatttgatatcatgatgctgacacccccagcacggcagcacctgcgtccgcctccccgccagcaacagccccctgaccgacgtcaccagcagcgacttcgcctgcaacatcggcggccggcgcggcgtgggcggcaaatgccccgtcaaagccggcggcgtggtcacgatcgagatgcatcagcagcccaacgaccggaactgccgcagcgaggccatcggcggcatgcactggggtccggtgcaggtctacctcagcaaggtccccgacgcgtcgaccgccgagccgacgcaggtgggctggttcaagatcttctccaacgcgtgggccaagaagcccggcggcaactcgggcgacgacgactactggggcacgcgcgagctcaacggctgctgcgggcgcatggacgtgccgatccccaccgacctggaagacggcgactacctgctgcgcgccgaggcgctggcgctgcacgccatgccgggccagttctacatgtcgtgctaccagatcaccatcacgggcggcacgggcaccgcgaagccggcgactgtccgcttccccggagcgtacaccaacaacgacgccggcatccgcgccaacatccacgccccgctgagcacctacatcgcgcccggcccggaggtgtactccggcggtaccacccgggcgcccggtgagggctgcccgggatgtgctacgacctgccaggttggctcgtcgcccagcgcgcaggctccaggccatggcacggccgtgggcggcggagctggtggcccgtctgcttgcaccgtccaggcgtatggccagtgcggtggccagggatacacgggttgcaccgagtgcgcggtaagttgggacttccttgtcattaaaatcgcaaatggaacggatgggctaacatttgcgggtgcaggatggtttcgtttgccgcgacgtctcggctccgtggtactctcagtgccagcctgctttctaa

SEQ ID NO: 134 - protein, 325 aa, Humicola insolens
MRTVFAAALAALAAREVAGHATFQQLWVDGTDYGSTCVRLPASNSPLTDVTSSDFACNIGGRRGVGGKCPVKAGGVVTIEMHQQPNDRNCRSEAIGGMHWGPVQVYLSKVPDASTAEPTQVGWFKIFSNAWAKKPGGNSGDDDYWGTRELNGCCGRMDVPIPTDLEDGDYLLRAEALALHAMPGQFYMSCYQITITGGTGTAKPATVRFPGAYTNNDAGIRANIHAPLSTYIAPGPEVYSGGTTRAPGEGCPGCATTCQVGSSPSAQAPGHGTAVGGGAGGPSACTVQAYGQCGGQGYTGCTECADGFVCRDVSAPWYSQCQPAF

SEQ ID NO: 135 - DNA, 1039 bp, Humicola insolens
  source 1..1039: mol_type = genomic DNA
atgaggctcccccaagtggcttccgttctggccctcgcggcccaggtccacggtcacggctacatctaccgtgtcaccgccgacaacattgtgtaagcgccctcagattccggacctcttcctacctggtggctaaccttctctcaactcttcagctacccgggatacgacatctatgtcgatcccctcctccaaccgcccccgtaccgcattgcctacggtggtggccagacgggtcccgtctatgatatcaacagcaaggatatcgcctgccagcgcgtccacagccccgctccgggtctgattgcccaggctcgcgcgggcagcaacatcaccttctggtggtcgcggtggctgtacagccacaagggtcccatctcggcatggatggctccgtatgagggcgacattgccaatgtggacgtcaaccagctcgagttcttcaagattggcgaggagttccacgatgagaccggcaagtgggcgacggagaagctggtggacgaccccgagggcaagtggaccgtcaagatccccgccgatatcaagcccggtctctatgtcgtgcggaacgaggtaagtttcatccgtcccaaaaaaggggtcccatcccatgcatggtgcatgcccagtctaatcatcatctcccggatagatcatcgccctccacttcgccgtccgcatgcctcccttctttgccgccttcacccccctcggaccgcagttctacatgacctgcttcgccttcaacatcaccggcgacggcacggccactccccagggctacaagttccctggcgcctacagcaaggacgatccggccctgtggtgggatctggaggagaacaagaacccgtaccccggcgccggccccaagccccacgtctcggcctacgatgtcgacctcgtccccaacgagttgtacatcgtcagcccgacgaacaacgcgacggctgatgagctctactgggaggcccagaggcaggcgcttgctgcccaggcggcgacgacggagtactttgactcgattggtggctaa

SEQ ID NO: 136 - protein, 298 aa, Humicola insolens
MRLPQVASVLALAAQVHGHGYIYRVTADNIVYPGYDIYVDPLLQPPPYRIAYGGGQTGPVYDINSKDIACQRVHSPAPGLIAQARAGSNITFWWSRWLYSHKGPISAWMAPYEGDIANVDVNQLEFFKIGEEFHDETGKWATEKLVDDPEGKWTVKIPADIKPGLYVVRNEIIALHFAVRMPPFFAAFTPLGPQFYMTCFAFNITGDGTATPQGYKFPGAYSKDDPALWWDLEENKNPYPGAGPKPHVSAYDVDLVPNELYIVSPTNNATADELYWEAQRQALAAQAATTEYFDSIGG

SEQ ID NO: 137 - DNA, 1025 bp, Humicola insolens
  source 1..1025: mol_type = genomic DNA
atgcacgtccagtctctccttgccggagcgctcgctctggctccgtcggcgtctgctcacttcctcttcccgcacctgatgctgaacggtgtccgcacgggagcctacgagtatgtccgggagcacgacttcggcttcatgccgcacaacaacgactggatcaactcgcccgatttccgttgcaacgaggggtcctggcgtcatcgccgcgagcccaagaccgccgtagtcactgccggcgttgacgtcgtgggcttcaacctgcacctggactttgacctgtaccatccgggccccgtgacggtaagcacatctgagtcagaacatacctccctgtgacgtagactaatgagtctcttaccgcagatctatctctcccgcgcccccggcgacgtgcgtgactacgacggatctggtgactggttcaaggtgtaccagctgggcacccgccaacccttcaacggcactgacgagggctgggccacttggaagatgaagaactggcagttccgcctgcccgctgagatcccggcgggcgagtacctgatgcgcatcgagcagatgagcgtgcaccctccttaccgccagaaggagtggtacgtgcagtgcgcccacctaaagatcaacagcaactacaacggccccgcgcccggcccgaccatcaagattcccggagggtacaagatcagcgatcctgcgattcaatatgaccagtgggcgcagccgccgccgacgtacgcgcccatgccgggaccgccgctgtggcccaacaacaatcctcagcagggcaacccgaatcagggcggaaataacggcggtggcaaccagggcggcggcaatggtggctgcaccgttccgaagtggtatgtagagttcttcactattatcatgagatgcagcgttggacttgtgcttacacctagaacaggggccaatgcggtggtcagggttacagcgggtgcaggaactgcgagtctggctcgacatgccgtgcccagaacgactggtactcgcagtgcctgtaa

SEQ ID NO: 138 - protein, 298 aa, Humicola insolens
MHVQSLLAGALALAPSASAHFLFPHLMLNGVRTGAYEYVREHDFGFMPHNNDWINSPDFRCNEGSWRHRREPKTAVVTAGVDVVGFNLHLDFDLYHPGPVTIYLSRAPGDVRDYDGSGDWFKVYQLGTRQPFNGTDEGWATWKMKNWQFRLPAEIPAGEYLMRIEQMSVHPPYRQKEWYVQCAHLKINSNYNGPAPGPTIKIPGGYKISDPAIQYDQWAQPPPTYAPMPGPPLWPNNNPQQGNPNQGGNNGGGNQGGGNGGCTVPKWGQCGGQGYSGCRNCESGSTCRAQNDWYSQCL

SEQ ID NO: 139 - DNA, 1035 bp, Humicola insolens
  source 1..1035: mol_type = genomic DNA
atgccaccaccactactggccaccgtcctctccttgctagccctcacccgcggcgccctttcccattcccacctagcccacgtcatcatcaacggccagctctaccacggcttcgacccacgtccaaaccaaaacaaccatccagcccgtgtcggctggtccacgaccgccacagatgacggcttcgtcaccccgggcaattactcccatcccgacatcatctgccaccgcggcggcgtcagcccgcgcgcccacgctcccgtcaccgccggcggcaaggtccaggtccaatggaacggctggccgatcggacacgtcgggccgatcctgacctacatcgcgccgtgcggcggactgccgggcgccgaagaagggtgtacgggcgtggacaaaaccgacctgcggtggaccaagatcgacgactcgatgccgccgttccggtttaccgacgccaccaagccagtctctggcagagcgcagttcccgataggccaggtctgggcgacggatgcgctggtcgaggcgaataatagctggtcggtggtcattcccaggaatatcccgccggggccgtacgttttgaggcaggagattgtggccctgcattacgcggcgaagttgaacggggcgcagaactatccgttgtgtctgaacctctgggtggaaaaggggcagcaggatcagggagagcccttcaaattcgatgcttacgacgcgagggagttttacagcgaggaccatccgggtgtgttgattgatgttatgacgatggttgggccgagagccgtgtaccggatacctggaccgaccgtggccagtggtgccacgagaattccgcactcattgcagacgagcgccgagacgtgggtggaagggacgccggtggccgtgacgagggcgacggaaacggttcagatggagataactacgacacctgcaggtcagggagctggtgtgaggacagctacccctgccatgccaacaccaacagtgacgaagaggtggaagggaagatttgagatgggtaggccatga

SEQ ID NO: 140 - protein, 344 aa, Humicola insolens
MPPPLLATVLSLLALTRGALSHSHLAHVIINGQLYHGFDPRPNQNNHPARVGWSTTATDDGFVTPGNYSHPDIICHRGGVSPRAHAPVTAGGKVQVQWNGWPIGHVGPILTYIAPCGGLPGAEEGCTGVDKTDLRWTKIDDSMPPFRFTDATKPVSGRAQFPIGQVWATDALVEANNSWSVVIPRNIPPGPYVLRQEIVALHYAAKLNGAQNYPLCLNLWVEKGQQDQGEPFKFDAYDAREFYSEDHPGVLIDVMTMVGPRAVYRIPGPTVASGATRIPHSLQTSAETWVEGTPVAVTRATETVQMEITTTPAGQGAGVRTATPAMPTPTVTKRWKGRFEMGRP

SEQ ID NO: 141 - DNA, 1057 bp, Humicola insolens
  source 1..1057: mol_type = genomic DNA
atgaagtccctgacctacgccgcgctggccgccctctgggcccagcagaccgctgctcatgccaccttccagcaactctgggtcgacggcgtcgactacggcagtcagtgcgcccgcctgccgccgtccaactcccccatcgccagcgtcacctcgaccgccatgcgctgcaacaacggtccccgcgctgccgccaagtgccccgtcaaggctggcggcaccgtcaccatcgagatgcaccaggttggtttccttgaagtgttcccctaccacatatacagaccgtagctaacacacccatccttagcaacccggtgaccggtcctgcaaccaggacgccattggcggtgcccaccacggccccgtgatggtgtacatgtccaaggtctctgatgccttcaccgccgacggctcgtcaggctggttcaagatcttccaggacggctgggccaagaaccccaacggccgcgttggcgacgacgacttctggggcaccaaggacctcaacacctgctgcggcaagatgaacgtcaagatccccgccgacatcgcccccggcgactacctgctccgcgccgaggccatcgcgctgcacgccgccggccccagcggtggcgcccagccctacgtcacctgctaccagctcaccgtcacgggcggcggcaacgccaacccgcccaccgtcaacttccccggcgcctacagcgagcgtgaccctggcatcgccgtcagcatccacggcgctctgtccaactacgtcgtccccggtcctccggtctactcgggcggcagcgagaagcgcgctggcagcccctgcgagggctgcgaggccacctgcaaggtcggctcgagccccagccagactcttgctccttccaacccggccccgacctctcccgccaacggcggcggcaacaacggtggtggcaacactggcggcggctgcaccgtgcccaagtggcagcagtgcggcggccagggctactcgggctgcaccgtctgcgagtctggctcgacttgccgcgctcagaaccagtggtactctcagtgcgtgtaa

SEQ ID NO: 142 - protein, 330 aa, Humicola insolens
MKSLTYAALAALWAQQTAAHATFQQLWVDGVDYGSQCARLPPSNSPIASVTSTAMRCNNGPRAAAKCPVKAGGTVTIEMHQQPGDRSCNQDAIGGAHHGPVMVYMSKVSDAFTADGSSGWFKIFQDGWAKNPNGRVGDDDFWGTKDLNTCCGKMNVKIPADIAPGDYLLRAEAIALHAAGPSGGAQPYVTCYQLTVTGGGNANPPTVNFPGAYSERDPGIAVSIHGALSNYVVPGPPVYSGGSEKRAGSPCEGCEATCKVGSSPSQTLAPSNPAPTSPANGGGNNGGGNTGGGCTVPKWQQCGGQGYSGCTVCESGSTCRAQNQWYSQCV

SEQ ID NO: 143 - DNA, 772 bp, Humicola insolens
  source 1..772: mol_type = genomic DNA
atgaagctcctcctccccgccctcctggctctggccgccgagtccgtctcggcgcactacatcttccaacaactcaccgtcgccggcaccaagtaccccgtgtggaagtacatccggcgcaacagcaatccggcgtggcttcaaaacggccctgtgaccgacctcgcctcgaccgacctgcgctgcaacgtgggcgggcaggtcagcaacggcaccgagactctcacggtccgcgcgggcgaccagttcacgttccacctcgacacggcggtgtaccaccagggcccgacctcgctgtacatgtcgcgcgctccgggcaaggtggaggactatgatggcagcgggccgtggtttaagatttatgattgggggccgacagggaataattgggtcatgaggggtatggtttcccctattaattattattattgtttacttggggcatcatctggtggtggtgctggtgacgatgataagagtgatggagaaggacctggctgacgacctaaaaacccgatcagattcgtacacgtacaacatcccccgctgcatccccgacggcgagtatctcctgcgcatccagcagctgggtctgcacaatccgggcgccgcgccgcagttctacatcagctgcgcccagatcaaggtcaccggcggcggcactaccaacccgacccccacggctctgattccgggagcgttcagggctacggatccgggatacactgtcaacgtaagtcaaactttgagcaactccatatcaacctcgtga

SEQ ID NO: 144 - protein, 216 aa, Humicola insolens
MKLLLPALLALAAESVSAHYIFQQLTVAGTKYPVWKYIRRNSNPAWLQNGPVTDLASTDLRCNVGGQVSNGTETLTVRAGDQFTFHLDTAVYHQGPTSLYMSRAPGKVEDYDGSGPWFKIYDWGPTGNNWVMRDSYTYNIPRCIPDGEYLLRIQQLGLHNPGAAPQFYISCAQIKVTGGGTTNPTPTALIPGAFRATDPGYTVNVSQTLSNSISTS

SEQ ID NO: 145 - DNA, 1536 bp, Humicola insolens
  source 1..1536: mol_type = genomic DNA
atgcgttctgtttcccttcttgcggccgctttcgcgccgctggctacggcacacacggtctttacagctcttttcatcaacaatgtccaccagggcgacggcacttgcgtccgtatggctaagcagggcaacctcgccacccatcccgtcagtctgaacagcaatgagatggcctgcggtgggtaggccccgttcctcgagcagctgatctcgaactaacatgttgattcttgaactccaggtcgcgatggccaacaaccagtggcatttacttgcccagcacctgcgggagccaagctgaccttattgtttcgtatgtgggcagatggctctcagccaggttccatcgacaagtctcacgttggtcccatgtccatctacctcaagaaagtctcagatatgaacaccgactcggccgcagggcccgggtggttcaagatctggagtgagggctacgacgctgcgacgaagaaatgggccacggagaaactcatcgccaacaacggtttgctcagcgtcaacctacctcccggcctccctgcaggctactacctcgcccgccacgaaatcgtcactctccaaaacgtcaccaacaacaaggccgatccgcagttctacgtcggctgtgcgcagctgttcgtccaagggttgggcaccgccgcctccgtgcctgctgacaaaaccgtttccatccccggccatctgaaccccaacgacccggcgctggtattcaacccctatacccaaaacgctgcgacatacccaagcttcggcccaccgctcttcttcccaaatgctgcttcggcgggatcaaacaaggcccagtcaacactcaagcaaacctccggcgtcatcccctccgactgcctcatcaaaaacgccaactggtgcggccgtgaagttccagactataccaacgaggcgggatgctggacggcggcggggaactgttgggagcaggctgatcaatgctacaagacagccccgccatcgggccataagggatgcaagacctgggaggagcagaagtgcaacgtcatccagaactcctgtgaagcgaagaggttttcgggcccgccaaacaggggggtcaagtttgctgatatggatgtgaatcagcttgttccgggggcgatccctgaagcagtgaacgccggtcagaatggggaggcggttgttgttgacggcacaacgagctctgcagatgagaaggcgagtgtggatttgacaacatcgtctctaccgacgccgacgcctgcggctgaagaaaacgggaaggaggatgaaagactggctcttgatccgaccctgacggaggacgagtcgtttttctcagttgagccaacgtctgagcccactggtgttcaggttgaggtgcctttgacaactgtggtcctccttccaacgctcacctcatctttgaatccattgccaaccccgacctcaatttcccagccggctcacccgggaagaccatgcacaggtcgccgtcgtaggccgaggccagggtttccgaaacacccgcgcgatttttaa

SEQ ID NO: 146 - protein, 490 aa, Humicola insolens
MRSVSLLAAAFAPLATAHTVFTALFINNVHQGDGTCVRMAKQGNLATHPVSLNSNEMACGRDGQQPVAFTCPAPAGAKLTLLFRMWADGSQPGSIDKSHVGPMSIYLKKVSDMNTDSAAGPGWFKIWSEGYDAATKKWATEKLIANNGLLSVNLPPGLPAGYYLARHEIVTLQNVTNNKADPQFYVGCAQLFVQGLGTAASVPADKTVSIPGHLNPNDPALVFNPYTQNAATYPSFGPPLFFPNAASAGSNKAQSTLKQTSGVIPSDCLIKNANWCGREVPDYTNEAGCWTAAGNCWEQADQCYKTAPPSGHKGCKTWEEQKCNVIQNSCEAKRFSGPPNRGVKFADMDVNQLVPGAIPEAVNAGQNGEAVVVDGTTSSADEKASVDLTTSSLPTPTPAAEENGKEDERLALDPTLTEDESFFSVEPTSEPTGVQVEVPLTTVVLLPTLTSSLNPLPTPTSISQPAHPGRPCTGRRRRPRPGFPKHPRDF

SEQ ID NO: 147 - DNA, 921 bp, Humicola insolens
  source 1..921: mol_type = genomic DNA
atgttcttccgcaacgccgccactcttgctctggcctacgccaccaccggcgtctcggcccacgcgctcatgtacggcgtctgggtcaacggcgtcgaccaaggcgacggccgcaacgtctacatccgcacgccccccaacaacagcccggtcaaagacctcgccagcccggacatcgtctgcaacgtcaacggcgggcgcgccgttccggacttcgtccaggcctcggcgggggacaccctcaccttcgagtggctgcacaacacccgcggcgacgacatcatcgaccgctcccacctcggccccatcatcacctacatcgccccttttaccacgggcaacccgacggggcccgtctggaccaaaatcgccgaacagggcttcaacccttccacccgccgctgggccgtcgacgatctgatcgacaacggcggcaagaccgacttcgtcctgcccgcgtccctcgcgccgggcaggtacatcatccggcaggagatcatcgcgcaccacgagtccgaaaccacgttcgaatccaacccggcgcggggtgcccagttctacccgtcgtgcgtgcagatccaagtctcttctggctcgggcaccgccgtgccggatcagaactttgacttcaacacgggctacacgtacgccgaccccggcatccacttcaacatctacacctcgttcaacagctactccatccccggcccggaggtttggacgggcgctagcaccggcggcggcaacggcaacggcaacggcaacggcaatgccacgcctacgcagcctactcccactcccactgtcactcccactcccatcgagaccgcccagccggttaccacgacgaccacctcgacccggccgttccctacccgctgccctggccgccgcctcaagcgtgaggagcccaaggcttga

SEQ ID NO: 148 - protein, 306 aa, Humicola insolens
MFFRNAATLALAYATTGVSAHALMYGVWVNGVDQGDGRNVYIRTPPNNSPVKDLASPDIVCNVNGGRAVPDFVQASAGDTLTFEWLHNTRGDDIIDRSHLGPIITYIAPFTTGNPTGPVWTKIAEQGFNPSTRRWAVDDLIDNGGKTDFVLPASLAPGRYIIRQEIIAHHESETTFESNPARGAQFYPSCVQIQVSSGSGTAVPDQNFDFNTGYTYADPGIHFNIYTSFNSYSIPGPEVWTGASTGGGNGNGNGNGNATPTQPTPTPTVTPTPIETAQPVTTTTTSTRPFPTRCPGRRLKREEPKA

SEQ ID NO: 149 - DNA, 1092 bp, Humicola insolens
  source 1..1092: mol_type = genomic DNA
atggctcatccatgggcacgttgcgtctatacagccatctggctcgctgcctccgcttctggacgtaggtacaagactccggcagtgccatttatgaacccacaacgtggactggtcccgtgctaacacatcacagactcgcgcgtttggagtgtctcggtcaatggacgctaccagggaccgggtgttgatgactacctgcgcgcaccgccaagtgactctccggtggtggacctggactcaccaaccctcaactgcaatgtcaatggaaacaagcctgttccagggtttgttgaggtgtctgcgggagattctctggaatggaagtggtactacatcaacccgtacaacccaagcgacatgatcatcgcggcagaacaccgcggaccgatcatcacctacatcacgaattacaccgatggccagcctcaaggagctgtctggaccaagattgatcacgaaggctacgatcctgtgacagaccggttcgccgtcgacaacttgatcgccaacaggggatggaaagcaatcaagcttcccatgctcgccgacgggaagtacatcctgcgacaggagatcatcgcactccacagcgcacacaaccaaggcggggcccagctgtatccgaactgcattcagatcaaggtcgttggtggcaagggaagcgcggtgcccaaccagaactttgatctcaacaaggggtacacatccgatcacccgggacttcggttcaacctgtggcaaccattcaacaattacaccattcccggtcctgaggtctggaagggagttgtggttgcgagcaatggtacaacgaacagcaccacaaatctcaccaacaacaccggcaccggttttgcgaacagcactatggccactggtgaaacaaggaccgagaggagttttatgacacttaccgcatcacattcagacactggcgtccccgccaaatctcatactgtggctgtaagctggacaacatccgccgccgttgttgggtctccgattagcgttaccacaactttcagttcctttaccacaacaccggttccgacgaactctaccggtgcttatctctaccggtacaagtga

SEQ ID NO: 150 - protein, 339 aa, Humicola insolens
MAHPWARCVYTAIWLAASASGHSRVWSVSVNGRYQGPGVDDYLRAPPSDSPVVDLDSPTLNCNVNGNKPVPGFVEVSAGDSLEWKWYYINPYNPSDMIIAAEHRGPIITYITNYTDGQPQGAVWTKIDHEGYDPVTDRFAVDNLIANRGWKAIKLPMLADGKYILRQEIIALHSAHNQGGAQLYPNCIQIKVVGGKGSAVPNQNFDLNKGYTSDHPGLRFNLWQPFNNYTIPGPEVWKGVVVASNGTTNSTTNLTNNTGTGFANSTMATGETRTERSFMTLTASHSDTGVPAKSHTVAVSWTTSAAVVGSPISVTTTFSSFTTTPVPTNSTGAYLYRYK

SEQ ID NO: 151 - DNA, 1005 bp, Malbranchea cinnamomea
  source 1..1005: mol_type = genomic DNA
atgtcaccctccttcaagtccactgccatcctcggagccgttgctctggccgcccgcgtgcgcgcccacggctacgtgtctggaatcgtcgttgacggtgcttaccatggcggttacatcgtcgacaagtacccctacatgcccaacccacccgatgtggtcggctggtcgactacggccacggacctgggcttcgtcgcccctgacgcctttggcgacccggacatcatctgccaccgggacggtgcccccggtgccatccacgccaaagtcaacgccggtgccaccatcgagctgcagtggaacacctggcccgaaagccaccacgggcccgtcatcgactacctggctaactgcaacggtgactgctcgtccgtcgacaagacctcgctcaagttcttcaagatcagcgaggccggcctaaacgacggctccaacgcccccggccagtgggcgtccgacgatctcattgccaacaacaacagctggactgtgaccatccccaagtcgatcgccccgggcaactacgtgctgcgccacgagatcatcgccctgcacagcgccggcaaccagaatggcgcgcagaactacccccagtgcttcaacctcgagatcaccagcaacggcagcgacaacccggagggcgtgctgggaaccgagctgtacaaggccgacgacccgggcattctgttcaacatctaccagcccatggactcgtacccgattcccggccctgctctctacaccggcggctcttctccctcccctaatccgcccacctctacccagtcgcctgtgccccagcccacccagtctcccccatcgggcagcaaccccggcaacggcaacggcgacgacgacaacgacaacggcaacgagaccccatccccgtctctccccgtcgagatccctgacgacctgacctcgcgcgagctactccttgtggcccaggagatcattgcccgtctgcttgagctgcagaatcagctggtcgtctcgaactaa

SEQ ID NO: 152 - protein, 334 aa, Malbranchea cinnamomea
MSPSFKSTAILGAVALAARVRAHGYVSGIVVDGAYHGGYIVDKYPYMPNPPDVVGWSTTATDLGFVAPDAFGDPDIICHRDGAPGAIHAKVNAGATIELQWNTWPESHHGPVIDYLANCNGDCSSVDKTSLKFFKISEAGLNDGSNAPGQWASDDLIANNNSWTVTIPKSIAPGNYVLRHEIIALHSAGNQNGAQNYPQCFNLEITSNGSDNPEGVLGTELYKADDPGILFNIYQPMDSYPIPGPALYTGGSSPSPNPPTSTQSPVPQPTQSPPSGSNPGNGNGDDDNDNGNETPSPSLPVEIPDDLTSRELLLVAQEIIARLLELQNQLVVSN

SEQ ID NO: 153 - DNA, 1101 bp, Talaromyces leycettanus
  source 1..1101: mol_type = genomic DNA
atgcatcaacacttccgatacactgcgctcctgacagcgttgctgtcagcatcaacccgagtcgcatcccacggccatgtcagcaacattgtcattaatggcgttccctatcaaggatgggatatcgattccatgccctacgagtcagacccaccagtggttgtcgcctgggagacacctaacacgtcaaacggtttcattaccccggatcagtacggtacgagtgatattatctgccatctgaacgcaaccaacgcaaagggccatgccgtcgttgctgccggagacaagatcagcattcaatggactgcctggcccagctcccaccacggccctgtcatcagctacctggccaactgtggcgccagctgtgagacagtcgacaaaacgacgttgcaattctttaagatcgacaacatcggtttcatagatgactcttcccccccaggcatctgggcagccgatcaattggaagcaaacaacaacacctggctcgtggagatccccccgaccatcgctccaggatactacgtcctgcgcaacgagatcatcgccctacacggtgcagagaatcaggatggcgcccagaactatccgcagtgcttcaatctgcaggtcaccggctcgggtaccgataaacccgccggcgttcttggaactcagctctattctcccactgacccgggcattctcgtgaacatttacacgagcctttcgacctacatcgtccccggtccaaccccgtacagtggttgggtgtccgtcgtgcagtctagctctgctatcaccgcttctggaaccccggtgacgggcactggcggagttagcccaaccacggctgctactacgacttcttcttctcactccacgacttctactactaccgggcccactgtaacctcgactagccacactactaccactactactcctactaccctcagaaccacgactacaactgcagctggtggtggtgcgacacagaccgtctacggccaatgcggcggtagtggttggactggcgcaactgcctgcgcagccggagctacttgcagcactctgaatccctactatgcccaatgccttcctactggtgcttga

SEQ ID NO: 154 - protein, 366 aa, Talaromyces leycettanus
MHQHFRYTALLTALLSASTRVASHGHVSNIVINGVPYQGWDIDSMPYESDPPVVVAWETPNTSNGFITPDQYGTSDIICHLNATNAKGHAVVAAGDKISIQWTAWPSSHHGPVISYLANCGASCETVDKTTLQFFKIDNIGFIDDSSPPGIWAADQLEANNNTWLVEIPPTIAPGYYVLRNEIIALHGAENQDGAQNYPQCFNLQVTGSGTDKPAGVLGTQLYSPTDPGILVNIYTSLSTYIVPGPTPYSGWVSVVQSSSAITASGTPVTGTGGVSPTTAATTTSSSHSTTSTTTGPTVTSTSHTTTTTTPTTLRTTTTTAAGGGATQTVYGQCGGSGWTGATACAAGATCSTLNPYYAQCLPTGA

SEQ ID NO: 155 - DNA, 1091 bp, Chaetomium thermophilum
  misc_feature 522..524: note = n is a, c, g, or t
  source 1..1091: mol_type = genomic DNA
atgccttccttcgcttcgaagactctcatttctgccctcgccggcgctgccagcgtcgccgctcacggccacgtcaagaacttcgtcatcaacggtctgtcgtaccaggcctacgacccgaccgtcttcccgtacatgcagaaccctcccatcgtcgccggctggacggcctccaacactgacaacggcttcgtgggccccgagtcctactcgagccccgatatcatctgccacaagtcggccacgaacgccaagggccatgccgtcatcaaggccggtgactctgtctacatccagtgggacacctggcccgagtcgcaccacggcccggtcatcgactacctcgccagctgcggcagcgccggctgcgagacggtcgacaagacccagctcgagttcttcaagatcgccgaggccggtctgattgacggctcccaggctcccggaaagtgggctgccgatcagctcatcgcccagaacaactcgtggctggtcaccatccccgagaatatcaagccgctnnnggctcctacgtcctccgccacgagatcatcgccctgcacagcgctggccagaccaacggtgcccagaactaccccgtctgcatcaacctcgaggtcactggtggcggcagcgacgttccctcgggtgtcaagggtactgagctctacaagcccaccgaccccggcatcctcatcaacatctaccagtcgctctcgaactacaccatccctggccctgctctgatgcccggcgccaagccagtcacccagcacacctcagccatcatcggcagcaccaccgccatcactggcaccgccaccgctgctccggccgcgccgacctcgaccgccgctgccatcaccaccagctctgctaatgccaaccccgccccgaccaccacccgcggcaacgccaaccccgtcccgactaccaccctccgcacgagcaccatcgctcctcagcccactgctgcccccatccagaccccgacctccagcgtcggccggcccccgcgcccgacccgctgccctggtctggacaacttcaagcgcgctcgtcgccacgctcgtgaccttgctgcccactaa

SEQ ID NO: 156 - protein, 364 aa, Chaetomium thermophilum
  REGION 174..176: note = misc_feature - Xaa can be any naturally occurring amino acid
MPSFASKTLISALAGAASVAAHGHVKNFVINGLSYQAYDPTVFPYMQNPPIVAGWTASNTDNGFVGPESYSSPDIICHKSATNAKGHAVIKAGDSVYIQWDTWPESHHGPVIDYLASCGSAGCETVDKTQLEFFKIAEAGLIDGSQAPGKWAADQLIAQNNSWLVTIPENIKPXXXGSYVLRHEIIALHSAGQTNGAQNYPVCINLEVTGGGSDVPSGVKGTELYKPTDPGILINIYQSLSNYTIPGPALMPGAKPVTQHTSAIIGSTTAITGTATAAPAAPTSTAAAITTSSANANPAPTTTRGNANPVPTTTLRTSTIAPQPTAAPIQTPTSSVGRPPRPTRCPGLDNFKRARRHARDLAAH

SEQ ID NO: 157 - DNA, 1089 bp, Trichoderma ressei
  source 1..1089: mol_type = genomic DNA
atgatccagaagctttccaacctccttgtcaccgcactggcggtggctactggcgttgtcggacatggacatattaatgacattgtcatcaacggggtgtggtatcaggcctatgatcctacaacgtttccatacgagtcaaacccccccatagtagtgggctggacggctgccgaccttgacaacggtacgtgatcctcatctctatctgtacaacgctcatgctaatccaactcaataggcttcgtttcacccgacgcataccaaaaccctgacatcatctgccacaagaatgctacgaatgccaaggggcacgcgtctgtcaaggccggagacactattctcttccagtgggtgccagttccatggccgcaccctggtcccattgtcgactacctggccaactgcaatggtgactgcgagaccgttgacaagacgacgcttgagttcttcaagatcgatggcgttggtctcctcagcggcggggatccgggcacctgggcctcagacgtgctgatctccaacaacaacacctgggtcgtcaagatccccgacaatcttgcgccaggcaattacgtgctccgccacgagatcatcgcgttacacagcgccgggcaggcaaacggcgctcagaactacccccagtgcttcaacattgccgtctcaggctcgggttctctgcagcccagcggcgttctagggaccgacctctatcacgcgacggaccctggtgttctcatcaacatctacaccagcccgctcaactacatcatccctggacctaccgtggtatcaggcctgccaacgagtgttgcccaggggagctccgccgcgacggccaccgccagcgccactgttcctggaggcggtagcggcccgaccagcagaaccacgacaacggcgaggacgacgcaggcctcaagcaggcccagctctacgcctcccgcaaccacgtcggcacctgctggcggcccaacccagactctgtacggccagtgtggtggcagcggttacagcgggcctactcgatgcgcgccgccagccacttgctctaccttgaacccctactacgcccagtgccttaactag

SEQ ID NO: 158 - protein, 344 aa, Trichoderma ressei
MIQKLSNLLVTALAVATGVVGHGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTLNPYYAQCLN

SEQ ID NO: 159 - DNA, 759 bp, Acrophialophora fusispora
  source 1..759: mol_type = genomic DNA
atgcgcatagaagctatcacaggcctcgtgctggcctcggccggtgcagtgtctgcccatggctgggtcgatgtctgggctattggcggcaagaactacacaggcttcaaccccacggtggcgccatgggtcccggatcagggcaccattgcgtggccggcctggaacaccgacacaggaccggtgtacagcaaggacgtcaacaccacagacatcatctgctcaatcaatgccaccaacgccaagatctactccgaccccatcgccgctgggaacgtcatcaacctgcactggacggtgtggccagactcacaccacgggcccatcctgtcgtacctggccgcgtgcaacggcgactgcgccaaggccgacaagaccaagctcaagtggttcaagattgcccatgccggtcaaatcagcctgggcaccggcggcggccaggttggctactgggccagcgacaagctgcaagacgacaacggcacctggcccgtcaccattccggcctccatcaagcccggcaattacgtgctgcggaacgagattattgccctccattcggcgtacgacgtcggcgccgcccagctctacccgcagtgcgttaatatcaagatcacgggcaacggccgcgtcacccctgccggcgtggtgggaaccaagctctacaaggagaccgatcctggcctgcattataacatctataacgacgagtctaagcctgtctatcagatccccggcccggccttgtgtaagtgctaa

SEQ ID NO: 160 - protein, 252 aa, Acrophialophora fusispora
MRIEAITGLVLASAGAVSAHGWVDVWAIGGKNYTGFNPTVAPWVPDQGTIAWPAWNTDTGPVYSKDVNTTDIICSINATNAKIYSDPIAAGNVINLHWTVWPDSHHGPILSYLAACNGDCAKADKTKLKWFKIAHAGQISLGTGGGQVGYWASDKLQDDNGTWPVTIPASIKPGNYVLRNEIIALHSAYDVGAAQLYPQCVNIKITGNGRVTPAGVVGTKLYKETDPGLHYNIYNDESKPVYQIPGPALCKC

SEQ ID NO: 161 - DNA, 1035 bp, Corynascus sepedonium
  source 1..1035: mol_type = genomic DNA
atgtctaagacttctgctctccttgctggcctaacgggcgcggccctcgtcgctgcccacgggcacgtcagccatatcattgtcaacggtgtctactatgagaactacgaccccacgacacactggtaccagcccaacccaccaacagtcatcggctggacggcagcccagcaggacaacggcttcatcgagcccaacaactttggcacgtcggacatcatctgccacaagagcggttctccaggcggcggtcacgctaccgtcgctgcgggcgacaagatcaacatcgtctggactccggagtggcccgactcccatatcggcccggtcattgactacctggctgcctgcaacggtgactgcgagaccgtaaacaaggagtcgctgcgcttctttaagattgacggggccggctatgacaaggccgctggccgctgggccgccgagactctgcgccagaacggcaacagctggctcgtccagatcccgtctgaccttaaggctggcaactacgtgctccgccacgaaatcatcgccctccacggcgctggaagcgccaacggtgctcaagcctacccgcagtgcatcaaccttcgcgtgacgggcggcggcagcagcgtgcccagcggcgtggccggcacctcgctctacaaagcctccgacgcaggcatcctcttcaacccctacgtcgcctctcccgattacccggtcccaggcccggcgctcattgctggtgccgccagctctatcgtacagagcacgtcggcagtgaccgctaccgcctcggccaccgctcccggtggcggcggcgccaaccccaaccctacgcccaccaccacctcctcgagcaatcccgccccaagcaccaccctcaggacaaccacctcggccgcgcaaaccacgcccccgcctaccaatggcaacgtccagacaaagtacggtcagtgtggtggtagggactggagcggcccaacggcgtgcgcggctggttccagctgctcggtgctcaacgactggtactcccagtgcgtgtaa

SEQ ID NO: 162 - protein, 344 aa, Corynascus sepedonium
MSKTSALLAGLTGAALVAAHGHVSHIIVNGVYYENYDPTTHWYQPNPPTVIGWTAAQQDNGFIEPNNFGTSDIICHKSGSPGGGHATVAAGDKINIVWTPEWPDSHIGPVIDYLAACNGDCETVNKESLRFFKIDGAGYDKAAGRWAAETLRQNGNSWLVQIPSDLKAGNYVLRHEIIALHGAGSANGAQAYPQCINLRVTGGGSSVPSGVAGTSLYKASDAGILFNPYVASPDYPVPGPALIAGAASSIVQSTSAVTATASATAPGGGGANPNPTPTTTSSSNPAPSTTLRTTTSAAQTTPPPTNGNVQTKYGQCGGRDWSGPTACAAGSSCSVLNDWYSQCV

SEQ ID NO: 163 - DNA, 1044 bp, Corynascus sepedonium
  source 1..1044: mol_type = genomic DNA
atgccttcttctacctccaagggtcttttctccgccctcatgggcgcggcgtcggttgccgcccatggtcatgtcaccaacattgtcatcaacggtgtgtcgtaccagaactacgacccgaccagcttcccttacatgcagaacccgccgacggttgttggctggacggcaagcaacactgataacggcttcgtcgctcctgatgcgtttgctagcggcgacatcatctgccacagggacgccaccaatgctggtggtcatgccgtcgttgctgctggtgacaaggtcttcatccagtgggatacctggcctgagtcgcaccatggccccgtccttgattacctcgccagctgcggtgacgccggctgcgaaacggtcgacaagaacactctcgagttcttcaagatcggcgaggctggcctgatcgacggcagcagtgctcccggcaagtgggcgtcggaccagctgattgagaacaataactcgtggatggttcagatccctgccaaccttgcgcccggaaactatgtgctgcggcatgagattattgctttgcacagcgctgggcaagctaacggtgcccaaaactacccccagtgcttcaacctgcaagttaccggctccggcacggacaagcctgccggtgtgctcggcaccgagctctacactcccaccgacgccggcatcttggccaacatctacacctcgcctgttcagtacgagattcctggcccggctctgatctcgggcgcttcggccgttgaacagtcctcctcggctatcaccgcctccgccagcgctgagaccggctccgccacagcaccccctgccggctctgccacggccgcccccaccactaccactaccacggctggctcggatgctagcgctacgccctcgtcctcgtccagctctggtgcgagcaccaccgccgagcccaccccttcggctactactaccgccggcggcagcaccccgcgcccgacccggtgccctggcctgaagcgccgccgccacgcccgtgatgtcaagctcgccctctaa

SEQ ID NO: 164 - protein, 347 aa, Corynascus sepedonium
MPSSTSKGLFSALMGAASVAAHGHVTNIVINGVSYQNYDPTSFPYMQNPPTVVGWTASNTDNGFVAPDAFASGDIICHRDATNAGGHAVVAAGDKVFIQWDTWPESHHGPVLDYLASCGDAGCETVDKNTLEFFKIGEAGLIDGSSAPGKWASDQLIENNNSWMVQIPANLAPGNYVLRHEIIALHSAGQANGAQNYPQCFNLQVTGSGTDKPAGVLGTELYTPTDAGILANIYTSPVQYEIPGPALISGASAVEQSSSAITASASAETGSATAPPAGSATAAPTTTTTTAGSDASATPSSSSSSGASTTAEPTPSATTTAGGSTPRPTRCPGLKRRRHARDVKLAL

SEQ ID NO: 165 - DNA, 1029 bp, Myceliophthora thermophila
  source 1..1029: mol_type = genomic DNA
atgtccaaggcctctgctctcctcgctggcctgacgggcgcggccctcgtcgctgcacatggccacgtcagccacatcgtcgtcaacggcgtctactacaggaactacgaccccacgacagactggtaccagcccaacccgccaacagtcatcggctggacggcagccgatcaggataatggcttcgttgaacccaacagctttggcacgccagatatcatctgccacaagagcgccacccccggcggcggccacgctaccgttgctgccggagacaagatcaacatcgtctggacccccgagtggcccgaatcccacatcggccccgtcattgactacctagccgcctgcaacggtgactgcgagaccgtcgacaagtcgtcgctgcgctggttcaagattgacggcgccggctacgacaaggccgccggccgctgggccgccgacgctctgcgcgccaacggcaacagctggctcgtccagatcccgtcggatctcaaggccggcaactacgtcctccgccacgagatcatcgccctccacggtgctcagagccccaacggcgcccaggcctacccgcagtgcatcaacctccgcgtcaccggcggcggcagcaacctgcccagcggcgtcgccggcacctcgctgtacaaggcgaccgacccgggcatcctcttcaacccctacgtctcctccccggattacaccgtccccggcccggccctcattgccggcgccgccagctcgatcgcccagagcacgtcggtcgccactgccaccggcacggccaccgttcccggcggcggcggcgccaaccctaccgccaccaccaccgccgccacctccgccgccccgagcaccaccctgaggacgaccactacctcggccgcgcagactaccgccccgccctccggcgatgtgcagaccaagtacggccagtgtggtggcaacggatggacgggcccgacggtgtgcgcccccggctcgagctgctccgtcctcaacgagtggtactcccagtgtttgtaa

SEQ ID NO: 166 - protein, 342 aa, Myceliophthora thermophila
MSKASALLAGLTGAALVAAHGHVSHIVVNGVYYRNYDPTTDWYQPNPPTVIGWTAADQDNGFVEPNSFGTPDIICHKSATPGGGHATVAAGDKINIVWTPEWPESHIGPVIDYLAACNGDCETVDKSSLRWFKIDGAGYDKAAGRWAADALRANGNSWLVQIPSDLKAGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 167 - DNA, 765 bp, Talaromyces emersonii
  source 1..765: mol_type = genomic DNA
atgctgtcttcgaaggctcctgtcacccttgcctttgcaggcctcgctggccttctgtccgccccactggtcaaggcccatggttttgtccagggcattgtcatcggtgaccaattctacagcgggtacatcgtcaacgagttcccctacgaatccaacccaccccccgtcatcggctgggccacgacagccaccgacctgggcttcgtcgacggcactgaataccaaggaccagacatcatctgccaccggaatgcgacgcccgcgctgctgacagcccccgtggccgccggcggcaccgtcgagctgcagtggacgccctggccgtccagccaccacgggccggtcatcacgtacctggccaactgcaacggcaactgctcgaccgtcgacaagacgcagctggagttcttcaagatcgaccagtcgggcctgatcaacgacactgacccgccgggcacctgggcgtccgacaacctcatcgccaacaacaacagctggaccgtgaccatccccagcaccctcgagccgggcaactacgtgctgcgccacgagatcatcgccctgcactcggcgggcaacaaagacggcgcccagaactacccccagtgcatcaacatcgaggtcacgggcggcggctcggtcgagccgacgggcacgctgggcgaggatctctaccacgacacggacccgggcattctgatcgacatttacgagccgattgcgacgtataccattccaggaccgcctgagccgacgttctag

SEQ ID NO: 168 - protein, 254 aa, Talaromyces emersonii
MLSSKAPVTLAFAGLAGLLSAPLVKAHGFVQGIVIGDQFYSGYIVNEFPYESNPPPVIGWATTATDLGFVDGTEYQGPDIICHRNATPALLTAPVAAGGTVELQWTPWPSSHHGPVITYLANCNGNCSTVDKTQLEFFKIDQSGLINDTDPPGTWASDNLIANNNSWTVTIPSTLEPGNYVLRHEIIALHSAGNKDGAQNYPQCINIEVTGGGSVEPTGTLGEDLYHDTDPGILIDIYEPIATYTIPGPPEPTF

SEQ ID NO: 169 - DNA, 3060 bp, Aspergillus fumigatus
  source 1..3060: mol_type = genomic DNA
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtttctgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggagcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccaacttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggtttcatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 170 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 171 - DNA, 40 bp, synthetic construct
  misc_feature 1..40: note = Artificial DNA Primer
  source 1..40: mol_type = other DNA
cacaactggggatccatgactttgtccaagatcacttcca

SEQ ID NO: 172 - DNA, 40 bp, synthetic construct
  misc_feature 1..40: note = Artificial DNA Primer
  source 1..40: mol_type = other DNA
ggcctccgcggccgcttaagcgttgaacagtgcaggacca

SEQ ID NO: 173 - DNA, 40 bp, synthetic construct; identical to SEQ ID NO: 171
  misc_feature 1..40: note = Artificial DNA Primer
  source 1..40: mol_type = other DNA

SEQ ID NO: 174 - DNA, 40 bp, synthetic construct; identical to SEQ ID NO: 172
  misc_feature 1..40: note = Artificial DNA Primer
  source 1..40: mol_type = other DNA

SEQ ID NO: 175 - DNA, 40 bp, synthetic construct
  misc_feature 1..40: note = Artificial DNA Primer
  source 1..40: mol_type = other DNA
cacaactggggatccatgctgtcttcgacgactcgcaccc

SEQ ID NO: 176 - DNA, 40 bp, synthetic construct
  misc_feature 1..40: note = Artificial DNA Primer
  source 1..40: mol_type = other DNA
ggcctccgcggccgcctagaacgtcggctcaggcggcccc

SEQ ID NO: 177 - DNA, 27 bp, synthetic construct
  misc_feature 1..27: note = Artificial DNA Primer
  source 1..27: mol_type = other DNA
ctggggatccatgtccttttccaagat

SEQ ID NO: 178 - DNA, 29 bp, synthetic construct
  misc_feature 1..29: note = Artificial DNA Primer
  source 1..29: mol_type = other DNA
ctccgcggccgcttaaccagtatacagag

SEQ ID NO: 179 - DNA, 25 bp, synthetic construct
  misc_feature 1..25: note = Artificial DNA Primer
  source 1..25: mol_type = other DNA
actcaatttacctctatccacactt

SEQ ID NO: 180 - DNA, 25 bp, synthetic construct
  misc_feature 1..25: note = Artificial DNA Primer
  source 1..25: mol_type = other DNA
gaattgtgagcggataacaatttca

SEQ ID NO: 181 - DNA, 36 bp, synthetic construct
  misc_feature 1..36: note = Artificial DNA Primer
  source 1..36: mol_type = other DNA
cggactgcgcaccatgctgtcttcgacgactcgcac

SEQ ID NO: 182 - DNA, 35 bp, synthetic construct
  misc_feature 1..35: note = Artificial DNA Primer
  source 1..35: mol_type = other DNA
tcgccacggagcttatcgacttcttctagaacgtc

SEQ ID NO: 183 - DNA, 49 bp, synthetic construct
  misc_feature 1..49: note = Artificial DNA Primer
  source 1..49: mol_type = other DNA
acaagaatactgatcctggcatctggtttgacatctactcggatctgag

SEQ ID NO: 184 - DNA, 49 bp, synthetic construct
  misc_feature 1..49: note = Artificial DNA Primer
  source 1..49: mol_type = other DNA
ctcagatccgagtagatgtcaaaccagatgccaggatcagtattcttgt

SEQ ID NO: 185 - DNA, 45 bp, synthetic construct
  misc_feature 1..45: note = Artificial DNA Primer
  source 1..45: mol_type = other DNA
atcatcgcccttcactctgcgtttaacctgaacggcgcgcagaac

SEQ ID NO: 186 - DNA, 45 bp, synthetic construct
  misc_feature 1..45: note = Artificial DNA Primer
  source 1..45: mol_type = other DNA
gttctgcgcgccgttcaggttaaacgcagagtgaagggcgatgat

SEQ ID NO: 187 - DNA, 46 bp, synthetic construct
  misc_feature 1..46: note = Artificial DNA Primer
  source 1..46: mol_type = other DNA
gaagtttgtcaagatcgccgctaagggcttgatcgacggctccaac

SEQ ID NO: 188 - DNA, 46 bp, synthetic construct
  misc_feature 1..46: note = Artificial DNA Primer
  source 1..46: mol_type = other DNA
gttggagccgtcgatcaagcccttagcggcgatcttgacaaacttc

SEQ ID NO: 189 - DNA, 48 bp, synthetic construct
  misc_feature 1..48: note = Artificial DNA Primer
  source 1..48: mol_type = other DNA
cttgttaaccaatacccctacatggaaaaccctcccgacaccattgcc

SEQ ID NO: 190 - DNA, 48 bp, synthetic construct
  misc_feature 1..48: note = Artificial DNA Primer
  source 1..48: mol_type = other DNA
ggcaatggtgtcgggagggttttccatgtaggggtattggttaacaag

SEQ ID NO: 191 - DNA, 42 bp, synthetic construct
  misc_feature 1..42: note = Artificial DNA Primer
  source 1..42: mol_type = other DNA
ctcccgacaccattgcctgggccaccaccgccaccgacctcg

SEQ ID NO: 192 - DNA, 42 bp, synthetic construct
  misc_feature 1..42: note = Artificial DNA Primer
  source 1..42: mol_type = other DNA
cgaggtcggtggcggtggtggcccaggcaatggtgtcgggag

SEQ ID NO: 193 - DNA, 46 bp, synthetic construct
  misc_feature 1..46: note = Artificial DNA Primer
  source 1..46: mol_type = other DNA
acagatcgaattccagtggacgaagtggccagagtctcaccatgga

SEQ ID NO: 194 - DNA, 46 bp, synthetic construct; identical to SEQ ID NO: 193
  misc_feature 1..46: note = Artificial DNA Primer
  source 1..46: mol_type = other DNA

SEQ ID NO: 195 - DNA, 37 bp, synthetic construct
  misc_feature 1..37: note = Artificial DNA Primer
  source 1..37: mol_type = other DNA
ccagtggacgaagtggccaaagtctcaccatggaccg

SEQ ID NO: 196 - DNA, 37 bp, synthetic construct
  misc_feature 1..37: note = Artificial DNA Primer
  source 1..37: mol_type = other DNA
cggtccatggtgagactttggccacttcgtccactgg

SEQ ID NO: 197 - DNA, 47 bp, synthetic construct
  misc_feature 1..47: note = Artificial DNA Primer
  source 1..47: mol_type = other DNA
ctggcatctggtttgacatctacggcgatctgagcggtggataccct

SEQ ID NO: 198 - DNA, 47 bp, synthetic construct
  misc_feature 1..47: note = Artificial DNA Primer
  source 1..47: mol_type = other DNA
agggtatccaccgctcagatcgccgtagatgtcaaaccagatgccag

SEQ ID NO: 199 - DNA, 45 bp, synthetic construct; identical to SEQ ID NO: 185
  misc_feature 1..45: note = Artificial DNA Primer
  source 1..45: mol_type = other DNA

SEQ ID NO: 200 - DNA, 45 bp, synthetic construct; identical to SEQ ID NO: 186
  misc_feature 1..45: note = Artificial DNA Primer
  source 1..45: mol_type = other DNA

SEQ ID NO: 201 - DNA, 44 bp, synthetic construct
  misc_feature 1..44: note = Artificial DNA Primer
  source 1..44: mol_type = other DNA
cacgagatcatcgcccttcacaccgcgggtaacctgaacggcgc

SEQ ID NO: 202 - DNA, 44 bp, synthetic construct
  misc_feature 1..44: note = Artificial DNA Primer
  source 1..44: mol_type = other DNA
gcgccgttcaggttacccgcggtgtgaagggcgatgatctcgtg

SEQ ID NO: 203 - DNA, 45 bp, synthetic construct; identical to SEQ ID NO: 185
  misc_feature 1..45: note = Artificial DNA Primer
  source 1..45: mol_type = other DNA

SEQ ID NO: 204 - DNA, 44 bp, synthetic construct
  misc_feature 1..44: note = Artificial DNA Primer
  source 1..44: mol_type = other DNA
cggctccaacccacctggtatctgggcttccgatgaactgatcg

SEQ ID NO: 205 - DNA, 45 bp, synthetic construct; identical to SEQ ID NO: 185
  misc_feature 1..45: note = Artificial DNA Primer
  source 1..45: mol_type = other DNA

SEQ ID NO: 206 - DNA, 44 bp, synthetic construct; identical to SEQ ID NO: 204
  misc_feature 1..44: note = Artificial DNA Primer
  source 1..44: mol_type = other DNA

SEQ ID NO: 207 - DNA, 45 bp, synthetic construct
  misc_feature 1..45: note = Artificial DNA Primer
  source 1..45: mol_type = other DNA
cggcaccggctaccagaccccggatattatctgccacagagacgc

SEQ ID NO: 208 - DNA, 45 bp, synthetic construct; identical to SEQ ID NO: 185
  misc_feature 1..45: note = Artificial DNA Primer
  source 1..45: mol_type = other DNA

SEQ ID NO: 209 - DNA, 44 bp, synthetic construct; identical to SEQ ID NO: 204
  misc_feature 1..44: note = Artificial DNA Primer
  source 1..44: mol_type = other DNA

SEQ ID NO: 210 - DNA, 45 bp, synthetic construct
  misc_feature 1..45: note = Artificial DNA Primer
  source 1..45: mol_type = other DNA
ccagtgtttcaacatccaaatcaccggtcctggcagtgctcaggg

SEQ ID NO: 211 - DNA, 36 bp, synthetic construct
  misc_feature 1..36: note = Artificial DNA Primer
  source 1..36: mol_type = other DNA
ccattcctgcctgctatgcccccggaaactacgtcc

SEQ ID NO: 212 - DNA, 33 bp, synthetic construct
  misc_feature 1..33: note = Artificial DNA Primer
  source 1..33: mol_type = other DNA
cctggtcctgcactgttcaactgctaagcggcc

SEQ ID NO: 213 - DNA, 43 bp, synthetic construct
  misc_feature 1..43: note = Artificial DNA Primer
  source 1..43: mol_type = other DNA
tggaccgtcaccattcccaactgcgtcgcccccggcaactacg

SEQ ID NO: 214 - DNA, 43 bp, synthetic construct
  misc_feature 1..43: note = Artificial DNA Primer
  source 1..43: mol_type = other DNA
cgtagttgccgggggcgacgcagttgggaatggtgacggtcca

SEQ ID NO: 215 - DNA, 45 bp, synthetic construct
  misc_feature 1..45: note = Artificial DNA Primer
  source 1..45: mol_type = other DNA
ccggggccgcctgagccgacgtgctaggcggccgcggaggccacc

SEQ ID NO: 216 - DNA, 45 bp, synthetic construct
  misc_feature 1..45: note = Artificial DNA Primer
  source 1..45: mol_type = other DNA
ggtggcctccgcggccgcctagcacgtcggctcaggcggccccgg

SEQ ID NO: 217 - DNA, 27 bp, synthetic construct
  misc_feature 1..27: note = Artificial DNA Primer
  misc_feature 10: note = N=DEOXYURIDINE
  source 1..27: mol_type = other DNA
atgcagcgcngccataaccatgagtga

SEQ ID NO: 218 - DNA, 28 bp, synthetic construct
  misc_feature 1..28: note = Artificial DNA Primer
  misc_feature 10: note = N=DEOXYURIDINE
  source 1..28: mol_type = other DNA
agcgctgcanaattctcttactgtcatg

SEQ ID NO: 219 - DNA, 36 bp, synthetic construct
  misc_feature 1..36: note = Artificial DNA Primer
  misc_feature 8: note = N=DEOXYURIDINE
  source 1..36: mol_type = other DNA
aattaagncctcagcgtgatttaaaacgccattgct

SEQ ID NO: 220 - DNA, 41 bp, synthetic construct
  misc_feature 1..41: note = Artificial DNA Primer
  misc_feature 8: note = N=DEOXYURIDINE
  source 1..41: mol_type = other DNA
acttaatnaaaccctcagcgcagttaggttggtgttcttct

SEQ ID NO: 221 - DNA, 29 bp, synthetic construct
  misc_feature 1..29: note = Artificial DNA Primer
  misc_feature 11: note = N=DEOXYURIDINE
  source 1..29: mol_type = other DNA
agctcaagganacctacagttattcgaaa

SEQ ID NO: 222 - DNA, 35 bp, synthetic construct
  misc_feature 1..35: note = Artificial DNA Primer
  misc_feature 11: note = N=DEOXYURIDINE
  source 1..35: mol_type = other DNA
atccttgagcngtttcctgtgtgaaattgttatcc

SEQ ID NO: 223 - DNA, 37 bp, synthetic construct
  misc_feature 1..37: note = Artificial DNA Primer
  misc_feature 9: note = N=DEOXYURIDINE
  source 1..37: mol_type = other DNA
atctcctcngctggtctggttaagccagccccgacac

SEQ ID NO: 224 - DNA, 27 bp, synthetic construct
  misc_feature 1..27: note = Artificial DNA Primer
  misc_feature 9: note = N=DEOXYURIDINE
  source 1..27: mol_type = other DNA
agaggaganaatactctgcgctccgcc

SEQ ID NO: 225 - DNA, 32 bp, synthetic construct
  misc_feature 1..32: note = Artificial DNA Primer
  misc_feature 9: note = N=DEOXYURIDINE
  source 1..32: mol_type = other DNA
gggtttaancctcacacaggaaacagctatga

SEQ ID NO: 226 - DNA, 46 bp, synthetic construct
  misc_feature 1..46: note = Artificial DNA Primer
  misc_feature 12: note = N=DEOXYURIDINE
  source 1..46: mol_type = other DNA
agtgtctgcgancgctctcactgcccccagttgtgtatatagagga

SEQ ID NO: 227 - DNA, 36 bp, synthetic construct
  misc_feature 1..36: note = Artificial DNA Primer
  misc_feature 12: note = N=DEOXYURIDINE
  source 1..36: mol_type = other DNA
atcgcagacacngctggcggtagacaatcaatccat

SEQ ID NO: 228 - DNA, 32 bp, synthetic construct
  misc_feature 1..32: note = Artificial DNA Primer
  misc_feature 9: note = N=DEOXYURIDINE
  source 1..32: mol_type = other DNA
ggacttaanggatctaagatgagctcatggct

SEQ ID NO: 229 - DNA, 24 bp, synthetic construct
  misc_feature 1..24: note = Artificial DNA Primer
  source 1..24: mol_type = other DNA
acgccattgctatgatgcttgaag

SEQ ID NO: 230 - DNA, 21 bp, synthetic construct
  misc_feature 1..21: note = Artificial DNA Primer
  source 1..21: mol_type = other DNA
tggtgaggtgctatcgtcctt

SEQ ID NO: 231 - DNA, 21 bp, synthetic construct
  misc_feature 1..21: note = Artificial DNA Primer
  source 1..21: mol_type = other DNA
cttcctgtaggtgcaccgaag

SEQ ID NO: 232 - DNA, 21 bp, synthetic construct
  misc_feature 1..21: note = Artificial DNA Primer
  source 1..21: mol_type = other DNA
acagaacgatatcggacctcg

SEQ ID NO: 233 - DNA, 23 bp, synthetic construct
  misc_feature 1..23: note = Artificial DNA Primer
  source 1..23: mol_type = other DNA
tcgttatgttaagtcttctatca

SEQ ID NO: 234 - DNA, 21 bp, synthetic construct
  misc_feature 1..21: note = Artificial DNA Primer
  source 1..21: mol_type = other DNA
agagctcgaagttcctccgag

SEQ ID NO: 235 - DNA, 22 bp, synthetic construct
  misc_feature 1..22: note = Artificial DNA Primer
  source 1..22: mol_type = other DNA
tatcacgaggccctttcgtctc

SEQ ID NO: 236 - DNA, 22 bp, synthetic construct
  misc_feature 1..22: note = Artificial DNA Primer
  source 1..22: mol_type = other DNA
tccgtcggctcctctccttcgt

SEQ ID NO: 237 - DNA, 24 bp, synthetic construct
  misc_feature 1..24: note = Artificial DNA Primer
  source 1..24: mol_type = other DNA
tgcatatcctctgacagtatatga

SEQ ID NO: 238 - DNA, 23 bp, synthetic construct
  misc_feature 1..23: note = Artificial DNA Primer
  source 1..23: mol_type = other DNA
cagtgaagagggcagtcgatagt

SEQ ID NO: 239 - DNA, 22 bp, synthetic construct
  misc_feature 1..22: note = Artificial DNA Primer
  source 1..22: mol_type = other DNA
acgaggaacatggctatctgga

SEQ ID NO: 240 - DNA, 23 bp, synthetic construct
  misc_feature 1..23: note = Artificial DNA Primer
  source 1..23: mol_type = other DNA
tcagctcattctgggaggtggga

SEQ ID NO: 241 - DNA, 22 bp, synthetic construct
  misc_feature 1..22: note = Artificial DNA Primer
  source 1..22: mol_type = other DNA
actccaggatcctttaaatcca

SEQ ID NO: 242 - DNA, 21 bp, synthetic construct
  misc_feature 1..21: note = Artificial DNA Primer
  source 1..21: mol_type = other DNA
actggcaagggatgccatgct

SEQ ID NO: 243 - DNA, 22 bp, synthetic construct
  misc_feature 1..22: note = Artificial DNA Primer
  source 1..22: mol_type = other DNA
tgatcatataaccaattgccct

SEQ ID NO: 244 - DNA, 22 bp, synthetic construct
  misc_feature 1..22: note = Artificial DNA Primer
  source 1..22: mol_type = other DNA
agttgtgtatatagaggattga

SEQ ID NO: 245 - DNA, 23 bp, synthetic construct
  misc_feature 1..23: note = Artificial DNA Primer
  source 1..23: mol_type = other DNA
tggtccttcgctcgtgatgtgga

SEQ ID NO: 246 - DNA, 20 bp, synthetic construct
  misc_feature 1..20: note = Artificial DNA Primer
  source 1..20: mol_type = other DNA
agtcctcagcgttaccggca

SEQ ID NO: 247 - DNA, 20 bp, synthetic construct
  misc_feature 1..20: note = Artificial DNA Primer
  source 1..20: mol_type = other DNA
accctcagctgtgtccggga

SEQ ID NO: 248 - DNA, 21 bp, synthetic construct
  misc_feature 1..21: note = Artificial DNA Primer
  source 1..21: mol_type = other DNA
tggtatgtgaacgccagtctg

SEQ ID NO: 249 - DNA, 28 bp, synthetic construct
  misc_feature 1..28: note = Artificial DNA Primer
  misc_feature 8: note = N=DEOXYURIDINE
  source 1..28: mol_type = other DNA
agagcganatgtccttttccaagataat

SEQ ID NO: 250 - DNA, 49 bp, synthetic construct
  misc_feature 1..49: note = Artificial DNA Primer
  misc_feature 8: note = N=DEOXYURIDINE
  source 1..49: mol_type = other DNA
tctgcganttagtgatggtggtgatgatgaccagtatacagaggaggac

SEQ ID NO: 251 - DNA, 28 bp, synthetic construct
  misc_feature 1..28: note = Artificial DNA Primer
  misc_feature 8: note = N=DEOXYURIDINE
  source 1..28: mol_type = other DNA
agagcganatgctgtcttcgacgactcg

SEQ ID NO: 252 - DNA, 49 bp, synthetic construct
  misc_feature 1..49: note = Artificial DNA Primer
  misc_feature 8: note = N=DEOXYURIDINE
  source 1..49: mol_type = other DNA
tctgcganctagtgatggtggtgatgatggaacgtcggctcaggcggcc

SEQ ID NO: 253 - DNA, 33 bp, synthetic construct
  misc_feature 1..33: note = Artificial DNA Primer
  misc_feature 8: note = N=DEOXYURIDINE
  source 1..33: mol_type = other DNA
agagcganatgtctgttgctaagtttgctggtg

SEQ ID NO: 254 - DNA, 49 bp, synthetic construct
  misc_feature 1..49: note = Artificial DNA Primer
  misc_feature 8: note = N=DEOXYURIDINE
  source 1..49: mol_type = other DNA
tctgcganttagtgatggtggtgatgatgggcggagaggtcacgggcgt

SEQ ID NO: 255 - DNA, 32 bp, synthetic construct
  misc_feature 1..32: note = Artificial DNA Primer
  misc_feature 8: note = N=DEOXYURIDINE
  source 1..32: mol_type = other DNA
agagcganatgccttctactaaagtcgctgcc

SEQ ID NO: 256 - DNA, 49 bp, synthetic construct
  misc_feature 1..49: note = Artificial DNA Primer
  misc_feature 8: note = N=DEOXYURIDINE
  source 1..49: mol_type = other DNA
tctgcgantcagtgatggtggtgatgatgaaggacagtagtggtgatga

SEQ ID NO: 257 - DNA, 40 bp, synthetic construct
  misc_feature 1..40: note = Artificial DNA Primer
  misc_feature 8: note = N=DEOXYURIDINE
  source 1..40: mol_type = other DNA
agagcganatgccttctttcgcctccaagactctcctttc

SEQ ID NO: 258 - DNA, 49 bp, synthetic construct
  misc_feature 1..49: note = Artificial DNA Primer
  misc_feature 8: note = N=DEOXYURIDINE
  source 1..49: mol_type = other DNA
tctgcgantcagtgatggtggtgatgatggtttgcctcctcagcccctc

SEQ ID NO: 259 - DNA, 20 bp, synthetic construct
  misc_feature 1..20: note = Artificial DNA Primer
  source 1..20: mol_type = other DNA
cccagttatcaactaccttg

SEQ ID NO: 260 - DNA, 20 bp, synthetic construct
  misc_feature 1..20: note = Artificial DNA Primer
  source 1..20: mol_type = other DNA
ctcaatttacctctatccac

SEQ ID NO: 261 - DNA, 20 bp, synthetic construct
  misc_feature 1..20: note = Artificial DNA Primer
  source 1..20: mol_type = other DNA
tataaccaattgccctcatc

SEQ ID NO: 262 - DNA, 20 bp, synthetic construct
  misc_feature 1..20: note = Artificial DNA Primer
  source 1..20: mol_type = other DNA
gcaccgtcgagctgcagtgg

SEQ ID NO: 263 - DNA, 20 bp, synthetic construct
  misc_feature 1..20: note = Artificial DNA Primer
  source 1..20: mol_type = other DNA
ccttgccaactgcaatggtg

SEQ ID NO: 264 - DNA, 28 bp, synthetic construct
  misc_feature 1..28: note = Artificial DNA Primer
  source 1..28: mol_type = other DNA
ggactgcgcaccatgccttctactaaag

SEQ ID NO: 265 - DNA, 34 bp, synthetic construct
  misc_feature 1..34: note = Artificial DNA Primer
  source 1..34: mol_type = other DNA
gccacggagcttaattaatcaaaggacagtagtg

SEQ ID NO: 266 - DNA, 20 bp, synthetic construct
  misc_feature 1..20: note = Artificial DNA Primer
  source 1..20: mol_type = other DNA
caatggcaattgttctaccg

SEQ ID NO: 267 - DNA, 20 bp, synthetic construct
  misc_feature 1..20: note = Artificial DNA Primer
  source 1..20: mol_type = other DNA
cgacggcagctcggcgcccg

SEQ ID NO: 268 - DNA, 31 bp, synthetic construct
  misc_feature 1..31: note = Artificial DNA Primer
  source 1..31: mol_type = other DNA
gcatttatcagggttattgtctcatgagcgg

SEQ ID NO: 269 - DNA, 26 bp, synthetic construct
  misc_feature 1..26: note = Artificial DNA Primer
  source 1..26: mol_type = other DNA
gctgataaatctggagccggtgagcg

SEQ ID NO: 270 - DNA, 30 bp, synthetic construct
  misc_feature 1..30: note = Artificial DNA Primer
  source 1..30: mol_type = other DNA
ccagaccagcagaggagataatactctgcg

SEQ ID NO: 271 - DNA, 32 bp, synthetic construct
  misc_feature 1..32: note = Artificial DNA Primer
  source 1..32: mol_type = other DNA
caaggatacctacagttattcgaaacctcctg

SEQ ID NO: 272 - DNA, 49 bp, synthetic construct
  misc_feature 1..49: note = Artificial DNA Primer
  source 1..49: mol_type = other DNA
gtggctggccatggcttcgttatcaacatcgtgattgatggtaaaaagt

SEQ ID NO: 273 - DNA, 33 bp, synthetic construct
  misc_feature 1..33: note = Artificial DNA Primer
  source 1..33: mol_type = other DNA
aacgaagccatggccagccactagagaagcaga

SEQ ID NO: 274 - DNA, 49 bp, synthetic construct
  misc_feature 1..49: note = Artificial DNA Primer
  source 1..49: mol_type = other DNA
gttatggcgggtatctagtgaacatctatccatacatgtccaatcctcc

SEQ ID NO: 275 - DNA, 35 bp, synthetic construct
  misc_feature 1..35: note = Artificial DNA Primer
  source 1..35: mol_type = other DNA
gttcactagatacccgccataactgtcgattgtca

SEQ ID NO: 276 - DNA, 49 bp, synthetic construct
  misc_feature 1..49: note = Artificial DNA Primer
  source 1..49: mol_type = other DNA
gttatggcgggtatctagtgaacgtctatccatacatgtccaatcctcc

SEQ ID NO: 277 - DNA, 35 bp, synthetic construct; identical to SEQ ID NO: 275
  misc_feature 1..35: note = Artificial DNA Primer
  source 1..35: mol_type = other DNA

SEQ ID NO: 278 - DNA, 49 bp, synthetic construct
  misc_feature 1..49: note = Artificial DNA Primer
  source 1..49: mol_type = other DNA
tgctccgtgcaatggtgattgtaggactgtggataagacccaattagaa

SEQ ID NO: 279 - DNA, 35 bp, synthetic construct
  misc_feature 1..35: note = Artificial DNA Primer
  source 1..35: mol_type = other DNA
acaatcaccattgcacggagcaaggtagttgataa

SEQ ID NO: 280 - DNA, 51 bp, synthetic construct
  misc_feature 1..51: note = Artificial DNA Primer
  source 1..51: mol_type = other DNA
attagaattcttcaaaattgccgaggagggtctcatcaatgatgacaatcc

SEQ ID NO: 281 - DNA, 37 bp, synthetic construct
  misc_feature 1..37: note = Artificial DNA Primer
  source 1..37: mol_type = other DNA
ctcggcaattttgaagaattctaattgggtcttatcc

SEQ ID NO: 282 - DNA, 51 bp, synthetic construct
  misc_feature 1..51: note = Artificial DNA Primer
  source 1..51: mol_type = other DNA
attagaattcttcaaaattgccgagaaaggtctcatcaatgatgacaatcc

SEQ ID NO: 283 - DNA, 37 bp, synthetic construct; identical to SEQ ID NO: 281
  misc_feature 1..37: note = Artificial DNA Primer
  source 1..37: mol_type = other DNA

SEQ ID NO: 284 - DNA, 51 bp, synthetic construct
  misc_feature 1..51: note = Artificial DNA Primer
  source 1..51: mol_type = other DNA
attagaattcttcaaaattgccgagctgggtctcatcaatgatgacaatcc

SEQ ID NO: 285 - DNA, 37 bp, synthetic construct; identical to SEQ ID NO: 281
  misc_feature 1..37: note = Artificial DNA Primer
  source 1..37: mol_type = other DNA

SEQ ID NO: 286 - DNA, 48 bp, synthetic construct
  misc_feature 1..48: note = Artificial DNA Primer
  source 1..48: mol_type = other DNA
gatagcagccaacaacagctgggtcgtcaccattccaaccacaattgc

SEQ ID NO: 287 - DNA, 35 bp, synthetic construct
  misc_feature 1..35: note = Artificial DNA Primer
  source 1..35: mol_type = other DNA
ccagctgttgttggctgctatcagattgtctgaag

SEQ ID NO: 288 - DNA, 48 bp, synthetic construct
  misc_feature 1..48: note = Artificial DNA Primer
  source 1..48: mol_type = other DNA
gatagcagccaacaacagctgggaggtcaccattccaaccacaattgc

SEQ ID NO: 289 - DNA, 35 bp, synthetic construct; identical to SEQ ID NO: 287
  misc_feature 1..35: note = Artificial DNA Primer
  source 1..35: mol_type = other DNA

SEQ ID NO: 290 - DNA, 47 bp, synthetic construct
  misc_feature 1..47: note = Artificial DNA Primer
  source 1..47: mol_type = other DNA
ggcatgagattattgctcttcacaaagctcagaaccaggatggtgcc

SEQ ID NO: 291 - DNA, 36 bp, synthetic construct
  misc_feature 1..36: note = Artificial DNA Primer
  source 1..36: mol_type = other DNA
gtgaagagcaataatctcatgcctcagaacatagtt

SEQ ID NO: 292 - DNA, 47 bp, synthetic construct
  misc_feature 1..47: note = Artificial DNA Primer
  source 1..47: mol_type = other DNA
ggcatgagattattgctcttcacttcgctcagaaccaggatggtgcc

SEQ ID NO: 293 - DNA, 36 bp, synthetic construct; identical to SEQ ID NO: 291
  misc_feature 1..36: note = Artificial DNA Primer
  source 1..36: mol_type = other DNA

SEQ ID NO: 294 - DNA, 47 bp, synthetic construct
  misc_feature 1..47: note = Artificial DNA Primer
  source 1..47: mol_type = other DNA
ggcatgagattattgctcttcacactgctcagaaccaggatggtgcc

SEQ ID NO: 295 - DNA, 36 bp, synthetic construct; identical to SEQ ID NO: 291
  misc_feature 1..36: note = Artificial DNA Primer
  source 1..36: mol_type = other DNA

SEQ ID NO: 296 - DNA, 47 bp, synthetic construct
  misc_feature 1..47: note = Artificial DNA Primer
  source 1..47: mol_type = other DNA
ggcatgagattattgctcttcactatgctcagaaccaggatggtgcc

SEQ ID NO: 297 - DNA, 36 bp, synthetic construct; identical to SEQ ID NO: 291
  misc_feature 1..36: note = Artificial DNA Primer
  source 1..36: mol_type = other DNA

SEQ ID NO: 298 - DNA, 46 bp, synthetic construct
  misc_feature 1..46: note = Artificial DNA Primer
  source 1..46: mol_type = other DNA
gcccagaactatccccagtgcgtcaatctgcaggtcactggaggtg

SEQ ID NO: 299 - DNA, 32 bp, synthetic construct
  misc_feature 1..32: note = Artificial DNA Primer
  source 1..32: mol_type = other DNA
gcactggggatagttctgggcaccatcctggt

SEQ ID NO: 300 - DNA, 46 bp, synthetic construct
  misc_feature 1..46: note = Artificial DNA Primer
  source 1..46: mol_type = other DNA
tggaggtggttctgataaccctgagggaactcttggaacggcactc

SEQ ID NO: 301 - DNA, 33 bp, synthetic construct
  misc_feature 1..33: note = Artificial DNA Primer
  source 1..33: mol_type = other DNA
agggttatcagaaccacctccagtgacctgcag

SEQ ID NO: 302 - DNA, 46 bp, synthetic construct
  misc_feature 1..46: note = Artificial DNA Primer
  source 1..46: mol_type = other DNA
cctgctggaactcttggaacgtgcctctaccacgataccgatcctg

SEQ ID NO: 303 - DNA, 33 bp, synthetic construct
  misc_feature 1..33: note = Artificial DNA Primer
  source 1..33: mol_type = other DNA
cgttccaagagttccagcagggttatcagaacc

SEQ ID NO: 304 - DNA, 46 bp, synthetic construct
  misc_feature 1..46: note = Artificial DNA Primer
  source 1..46: mol_type = other DNA
cctgctggaactcttggaacggagctctaccacgataccgatcctg

SEQ ID NO: 305 - DNA, 33 bp, synthetic construct; identical to SEQ ID NO: 303
  misc_feature 1..33: note = Artificial DNA Primer
  source 1..33: mol_type = other DNA

SEQ ID NO: 306 - DNA, 46 bp, synthetic construct
  misc_feature 1..46: note = Artificial DNA Primer
  source 1..46: mol_type = other DNA
cctgctggaactcttggaacgcagctctaccacgataccgatcctg

SEQ ID NO: 307 - DNA, 33 bp, synthetic construct; identical to SEQ ID NO: 303
  misc_feature 1..33: note = Artificial DNA Primer
  source 1..33: mol_type = other DNA

SEQ ID NO: 308 - DNA, 47 bp, synthetic construct
  misc_feature 1..47: note = Artificial DNA Primer
  source 1..47: mol_type = other DNA
catccctggtcctcctctgtataggggtcatcatcaccaccatcact

SEQ ID NO: 309 - DNA, 35 bp, synthetic construct
  misc_feature 1..35: note = Artificial DNA Primer
  source 1..35: mol_type = other DNA
atacagaggaggaccagggatgatatagctggaaa

SEQ ID NO: 310 - DNA, 47 bp, synthetic construct
  misc_feature 1..47: note = Artificial DNA Primer
  source 1..47: mol_type = other DNA
ctacagcgggtacatcgtcaacgtcttcccctacgaatccaacccac

SEQ ID NO: 311 - DNA, 33 bp, synthetic construct
  misc_feature 1..33: note = Artificial DNA Primer
  source 1..33: mol_type = other DNA
gttgacgatgtacccgctgtagctgttgggagt

SEQ ID NO: 312 - DNA, 44 bp, synthetic construct
  misc_feature 1..44: note = Artificial DNA Primer
  source 1..44: mol_type = other DNA
gtcgacggcacaggataccaacagccggacatcatctgccaccg

SEQ ID NO: 313 - DNA, 31 bp, synthetic construct
  misc_feature 1..31: note = Artificial DNA Primer
  source 1..31: mol_type = other DNA
ttggtatcctgtgccgtcgacgaagcccagg

SEQ ID NO: 314 - DNA, 44 bp, synthetic construct
  misc_feature 1..44: note = Artificial DNA Primer
  source 1..44: mol_type = other DNA
cgccgtgcaacggcaactgccgcaccgtcgacaagacgacgctg

SEQ ID NO: 315 - DNA, 31 bp, synthetic construct
  misc_feature 1..31: note = Artificial DNA Primer
  source 1..31: mol_type = other DNA
gcagttgccgttgcacggcgccaggtaggtg

SEQ ID NO: 316 - DNA, 47 bp, synthetic construct
  misc_feature 1..47: note = Artificial DNA Primer
  source 1..47: mol_type = other DNA
cgtcgatggtagcgagtatgctcaggccgacatcatttgccacaaga

SEQ ID NO: 317 - DNA, 34 bp, synthetic construct
  misc_feature 1..34: note = Artificial DNA Primer
  source 1..34: mol_type = other DNA
agcatactcgctaccatcgacgaaacccaagtcg

SEQ ID NO: 318 - DNA, 47 bp, synthetic construct
  misc_feature 1..47: note = Artificial DNA Primer
  source 1..47: mol_type = other DNA
cgtcgatggtagcgagtatgctaccgccgacatcatttgccacaaga

SEQ ID NO: 319 - DNA, 34 bp, synthetic construct; identical to SEQ ID NO: 317
  misc_feature 1..34: note = Artificial DNA Primer
  source 1..34: mol_type = other DNA

SEQ ID NO: 320 - DNA, 48 bp, synthetic construct
  misc_feature 1..48: note = Artificial DNA Primer
  source 1..48: mol_type = other DNA
ctcgagtttttcaagattgacgagtgcggtctcatcaacgacgacgac

SEQ ID NO: 321 - DNA, 35 bp, synthetic construct
  misc_feature 1..35: note = Artificial DNA Primer
  source 1..35: mol_type = other DNA
ctcgtcaatcttgaaaaactcgaggtcggtcttgg

SEQ ID NO: 322 - DNA, 48 bp, synthetic construct
  misc_feature 1..48: note = Artificial DNA Primer
  source 1..48: mol_type = other DNA
ctcgagtttttcaagattgacgagggtggtctcatcaacgacgacgac

SEQ ID NO: 323 - DNA, 35 bp, synthetic construct; identical to SEQ ID NO: 321
  misc_feature 1..35: note = Artificial DNA Primer
  source 1..35: mol_type = other DNA

SEQ ID NO: 324 - DNA, 45 bp, synthetic construct
  misc_feature 1..45: note = Artificial DNA Primer
  source 1..45: mol_type = other DNA
acgacgacgacgtccccggtatctgggccagtgataacttgatcg

SEQ ID NO: 325 - DNA, 31 bp, synthetic construct
  misc_feature 1..31: note = Artificial DNA Primer
  source 1..31: mol_type = other DNA
accggggacgtcgtcgtcgttgatgagaccg

SEQ ID NO: 326 - DNA, 47 bp, synthetic construct
  misc_feature 1..47: note = Artificial DNA Primer
  source 1..47: mol_type = other DNA
gatcgccaacaacaacagctgggtcgtgaccatcccctctgacattg

SEQ ID NO: 327 - DNA, 35 bp, synthetic construct
  misc_feature 1..35: note = Artificial DNA Primer
  source 1..35: mol_type = other DNA
ccagctgttgttgttggcgatcaagttatcactgg

SEQ ID NO: 328 - DNA, 47 bp, synthetic construct
  misc_feature 1..47: note = Artificial DNA Primer
  source 1..47: mol_type = other DNA
gatcgccaacaacaacagctggttcgtgaccatcccctctgacattg

SEQ ID NO: 329 - DNA, 35 bp, synthetic construct; identical to SEQ ID NO: 327
  misc_feature 1..35: note = Artificial DNA Primer
  source 1..35: mol_type = other DNA

SEQ ID NO: 330 - DNA, 45 bp, synthetic construct
  misc_feature 1..45: note = Artificial DNA Primer
  source 1..45: mol_type = other DNA
tggactgtgaccatcccctctcgtattgcggctggcaactacgtc

SEQ ID NO: 331 - DNA, 33 bp, synthetic construct
  misc_feature 1..33: note = Artificial DNA Primer
  source 1..33: mol_type = other DNA
agaggggatggtcacagtccagctgttgttgtt

SEQ ID NO: 332 - DNA, 48 bp, synthetic construct
  misc_feature 1..48: note = Artificial DNA Primer
  source 1..48: mol_type = other DNA
gtcacgaaatcattgcccttcacaaggctggtaacaaggatggtgctc

SEQ ID NO: 333 - DNA, 34 bp, synthetic construct
  misc_feature 1..34: note = Artificial DNA Primer
  source 1..34: mol_type = other DNA
gtgaagggcaatgatttcgtgacggaggacgtag

SEQ ID NO: 334 - DNA, 48 bp, synthetic construct
  misc_feature 1..48: note = Artificial DNA Primer
  source 1..48: mol_type = other DNA
gtcacgaaatcattgcccttcacaccgctggtaacaaggatggtgctc

SEQ ID NO: 335 - DNA, 34 bp, synthetic construct; identical to SEQ ID NO: 333
  misc_feature 1..34: note = Artificial DNA Primer
  source 1..34: mol_type = other DNA

SEQ ID NO: 336 - DNA, 48 bp, synthetic construct
  misc_feature 1..48: note = Artificial DNA Primer
  source 1..48: mol_type = other DNA
gtcacgaaatcattgcccttcactacgctggtaacaaggatggtgctc

SEQ ID NO: 337 - DNA, 34 bp, synthetic construct; identical to SEQ ID NO: 333
  misc_feature 1..34: note = Artificial DNA Primer
  source 1..34: mol_type = other DNA

SEQ ID NO: 338 - DNA, 45 bp, synthetic construct
  misc_feature 1..45: note = Artificial DNA Primer
  source 1..45: mol_type = other DNA
gctcagaactaccctcagtgcatcaacttgaaggtcactggcggc

SEQ ID NO: 339 - DNA, 33 bp, synthetic construct
  misc_feature 1..33: note = Artificial DNA Primer
  source 1..33: mol_type = other DNA
gcactgagggtagttctgagcaccatccttgtt

SEQ ID NO: 340 - DNA, 45 bp, synthetic construct
  misc_feature 1..45: note = Artificial DNA Primer
  source 1..45: mol_type = other DNA
gctcagaactaccctcagtgcgtcaacttgaaggtcactggcggc

SEQ ID NO: 341 - DNA, 33 bp, synthetic construct; identical to SEQ ID NO: 339
  misc_feature 1..33: note = Artificial DNA Primer
  source 1..33: mol_type = other DNA

SEQ ID NO: 342 - DNA, 46 bp, synthetic construct
  misc_feature 1..46: note = Artificial DNA Primer
  source 1..46: mol_type = other DNA
ccttctggcactgctggtgagatgctgtacaaggacaccgatgctg

SEQ ID NO: 343 - DNA, 32 bp, synthetic construct
  misc_feature 1..32: note = Artificial DNA Primer
  source 1..32: mol_type = other DNA
ctcaccagcagtgccagaaggagcgagatcac

SEQ ID NO: 344 - DNA, 46 bp, synthetic construct
  misc_feature 1..46: note = Artificial DNA Primer
  source 1..46: mol_type = other DNA
ccttctggcactgctggtgagcagctgtacaaggacaccgatgctg

SEQ ID NO: 345 - DNA, 32 bp, synthetic construct; identical to SEQ ID NO: 343
  misc_feature 1..32: note = Artificial DNA Primer
  source 1..32: mol_type = other DNA

SEQ ID NO: 346 - DNA, 45 bp, synthetic construct
  misc_feature 1..45: note = Artificial DNA Primer
  source 1..45: mol_type = other DNA
actgctggtgagagcctgtaccgtgacaccgatgctggtatcctc

SEQ ID NO: 347 - DNA, 32 bp, synthetic construct
  misc_feature 1..32: note = Artificial DNA Primer
  source 1..32: mol_type = other DNA
gtacaggctctcaccagcagtgccagaaggag

SEQ ID NO: 348 - DNA, 47 bp, synthetic construct
  misc_feature 1..47: note = Artificial DNA Primer
  source 1..47: mol_type = other DNA
ctcctacgatattcccggacctcccatgtacaacgctacctccagct

SEQ ID NO: 349 - DNA, 34 bp, synthetic construct
  misc_feature 1..34: note = Artificial DNA Primer
  source 1..34: mol_type = other DNA
aggtccgggaatatcgtaggaggaaagagactgg

SEQ ID NO: 350 - DNA, 52 bp, synthetic construct
  misc_feature 1..52: note = Artificial DNA Primer
  source 1..52: mol_type = other DNA
caaaacatcgttatcgacggtaaattttaagcagtgatgcatccattattaa

SEQ ID NO: 351 - DNA, 36 bp, synthetic construct
  misc_feature 1..36: note = Artificial DNA Primer
  source 1..36: mol_type = other DNA
tttaccgtcgataacgatgttttgcacaaaaccatg

SEQ ID NO: 352 - DNA, 48 bp, synthetic construct
  misc_feature 1..48: note = Artificial DNA Primer
  source 1..48: mol_type = other DNA
agttactctggataccttgtgaatatcttcccctacgagtccaaccca

SEQ ID NO: 353 - DNA, 38 bp, synthetic construct
  misc_feature 1..38: note = Artificial DNA Primer
  source 1..38: mol_type = other DNA
attcacaaggtatccagagtaactgatttttttgtaag

SEQ ID NO: 354 - DNA, 48 bp, synthetic construct
  misc_feature 1..48: note = Artificial DNA Primer
  source 1..48: mol_type = other DNA
agttactctggataccttgtgaatgtcttcccctacgagtccaaccca

SEQ ID NO: 355 - DNA, 38 bp, synthetic construct; identical to SEQ ID NO: 353
  misc_feature 1..38: note = Artificial DNA Primer
  source 1..38: mol_type = other DNA

SEQ ID NO: 356 - DNA, 47 bp, synthetic construct
  misc_feature 1..47: note = Artificial DNA Primer
  source 1..47: mol_type = other DNA
tgtgaatcagttcccctacgagcttaacccaccagctgttattgggt

SEQ ID NO: 357 - DNA, 34 bp, synthetic construct
  misc_feature 1..34: note = Artificial DNA Primer
  source 1..34: mol_type = other DNA
ctcgtaggggaactgattcacaaggtatccagag

SEQ ID NO: 358 - DNA, 45 bp, synthetic construct
  misc_feature 1..45: note = Artificial DNA Primer
  source 1..45: mol_type = other DNA
ccaccagctgttattgggtggtgcacaactgcaaccgacctggga

SEQ ID NO: 359 - DNA, 32 bp, synthetic construct
  misc_feature 1..32: note = Artificial DNA Primer
  source 1..32: mol_type = other DNA
ccacccaataacagctggtgggttggactcgt

SEQ ID NO: 360 - DNA, 45 bp, synthetic construct
  misc_feature 1..45: note = Artificial DNA Primer
  source 1..45: mol_type = other DNA
ccaccagctgttattgggtgggagacaactgcaaccgacctggga

SEQ ID NO: 361 - DNA, 32 bp, synthetic construct; identical to SEQ ID NO: 359
  misc_feature 1..32: note = Artificial DNA Primer
  source 1..32: mol_type = other DNA

SEQ ID NO: 362 - DNA, 49 bp, synthetic construct
  misc_feature 1..49: note = Artificial DNA Primer
  source 1..49: mol_type = other DNA
ctagactttgtcaagattgaccaatgcggtttgatcgacgatactaccc

SEQ ID NO: 363 - DNA, 37 bp, synthetic construct
  misc_feature 1..37: note = Artificial DNA Primer
  source 1..37: mol_type = other DNA
ttggtcaatcttgacaaagtctagcttagtcttatcc

SEQ ID NO: 364 - DNA, 45 bp, synthetic construct
  misc_feature 1..45: note = Artificial DNA Primer
  source 1..45: mol_type = other DNA
acgatactacccccccgggtatctgggcttccgacaaacttatcg

SEQ ID NO: 365 - DNA, 32 bp, synthetic construct
  misc_feature 1..32: note = Artificial DNA Primer
  source 1..32: mol_type = other DNA
acccgggggggtagtatcgtcgatcaaaccac

SEQ ID NO: 366 - DNA, 45 bp, synthetic construct
  misc_feature 1..45: note = Artificial DNA Primer
  source 1..45: mol_type = other DNA
gctgccaacaacagctggacttgcactatcccctccaccatcgcg

SEQ ID NO: 367 - DNA, 34 bp, synthetic construct
  misc_feature 1..34: note = Artificial DNA Primer
  source 1..34: mol_type = other DNA
agtccagctgttgttggcagcgataagtttgtcg

SEQ ID NO: 368 - DNA, 45 bp, synthetic construct
  misc_feature 1..45: note = Artificial DNA Primer
  source 1..45: mol_type = other DNA
gctgccaacaacagctggactcttactatcccctccaccatcgcg

SEQ ID NO: 369 - DNA, 34 bp, synthetic construct; identical to SEQ ID NO: 367
  misc_feature 1..34: note = Artificial DNA Primer
  source 1..34: mol_type = other DNA

SEQ ID NO: 370 - DNA, 46 bp, synthetic construct
  misc_feature 1..46: note = Artificial DNA Primer
  source 1..46: mol_type = other DNA
ccaacaacagctggactgtaactcttccctccaccatcgcgcctgg

SEQ ID NO: 371 - DNA, 35 bp, synthetic construct
  misc_feature 1..35: note = Artificial DNA Primer
  source 1..35: mol_type = other DNA
agttacagtccagctgttgttggcagcgataagtt

SEQ ID NO: 372 - DNA, 47 bp, synthetic construct
  misc_feature 1..47: note = Artificial DNA Primer
  source 1..47: mol_type = other DNA
ctggactgtaactatcccctcccgcatcgcgcctggaaactacgttt

SEQ ID NO: 373 - DNA, 33 bp, synthetic construct
  misc_feature 1..33: note = Artificial DNA Primer
  source 1..33: mol_type = other DNA
ggaggggatagttacagtccagctgttgttggc

SEQ ID NO: 374 - DNA, 46 bp, synthetic construct
  misc_feature 1..46: note = Artificial DNA Primer
  source 1..46: mol_type = other DNA
gccacgaaatcattgctcttcacaaggctggaaacgcagacggtgc

SEQ ID NO: 375 - DNA, 35 bp, synthetic construct
  misc_feature 1..35: note = Artificial DNA Primer
  source 1..35: mol_type = other DNA
gtgaagagcaatgatttcgtggcgcaaaacgtagt

SEQ ID NO: 376 - DNA, 46 bp, synthetic construct
  misc_feature 1..46: note = Artificial DNA Primer
  source 1..46: mol_type = other DNA
gccacgaaatcattgctcttcactttgctggaaacgcagacggtgc

SEQ ID NO: 377 - DNA, 35 bp, synthetic construct; identical to SEQ ID NO: 375
  misc_feature 1..35: note = Artificial DNA Primer
  source 1..35: mol_type = other DNA

SEQ ID NO: 378 - DNA, 46 bp, synthetic construct
  misc_feature 1..46: note = Artificial DNA Primer
  source 1..46: mol_type = other DNA
gccacgaaatcattgctcttcacaccgctggaaacgcagacggtgc

SEQ ID NO: 379 - DNA, 35 bp, synthetic construct; identical to SEQ ID NO: 375
  misc_feature 1..35: note = Artificial DNA Primer
  source 1..35: mol_type = other DNA

SEQ ID NO: 380 - DNA, 46 bp, synthetic construct
  misc_feature 1..46: note = Artificial DNA Primer
  source 1..46: mol_type = other DNA
gccacgaaatcattgctcttcactacgctggaaacgcagacggtgc

SEQ ID NO: 381 - DNA, 35 bp, synthetic construct; identical to SEQ ID NO: 375
  misc_feature 1..35: note = Artificial DNA Primer
  source 1..35: mol_type = other DNA

SEQ ID NO: 382 - DNA, 46 bp, synthetic construct
  misc_feature 1..46: note = Artificial DNA Primer
  source 1..46: mol_type = other DNA
tgcccaaaactaccctcaatgcgtcaacttggagatcaccggcagc

SEQ ID NO: 383 - DNA, 32 bp, synthetic construct
  misc_feature 1..32: note = Artificial DNA Primer
  source 1..32: mol_type = other DNA
gcattgagggtagttttgggcaccgtctgcgt

SEQ ID NO: 384 - DNA, 44 bp, synthetic construct
  misc_feature 1..44: note = Artificial DNA Primer
  source 1..44: mol_type = other DNA
gcagcggaaccgccgctcccgagggtaccgctggcgaaaagctc

SEQ ID NO: 385 - DNA, 30 bp, synthetic construct
  misc_feature 1..30: note = Artificial DNA Primer
  source 1..30: mol_type = other DNA
gggagcggcggttccgctgccggtgatctc

SEQ ID NO: 386 - DNA, 46 bp, synthetic construct
  misc_feature 1..46: note = Artificial DNA Primer
  source 1..46: mol_type = other DNA
accgctggcgaaaagctctaccgctctactgaccccggtatcttgg

SEQ ID NO: 387 - DNA, 32 bp, synthetic construct
  misc_feature 1..32: note = Artificial DNA Primer
  source 1..32: mol_type = other DNA
gtagagcttttcgccagcggtaccagagggag

SEQ ID NO: 388 - DNA, 45 bp, synthetic construct
  misc_feature 1..45: note = Artificial DNA Primer
  source 1..45: mol_type = other DNA
gacctacgttattcccggaccacccctgtggagcggtgctgccaa

SEQ ID NO: 389 - DNA, 34 bp, synthetic construct
  misc_feature 1..34: note = Artificial DNA Primer
  source 1..34: mol_type = other DNA
tggtccgggaataacgtaggtcgacaaggattgg

SEQ ID NO: 390 - DNA, 46 bp, synthetic construct
  misc_feature 1..46: note = Artificial DNA Primer
  source 1..46: mol_type = other DNA
taccagggttacgatccgaccatcttcccttacatgcagaacccgc

SEQ ID NO: 391 - DNA, 31 bp, synthetic construct
  misc_feature 1..31: note = Artificial DNA Primer
  source 1..31: mol_type = other DNA
ggtcggatcgtaaccctggtacgagaccccg

SEQ ID NO: 392 - DNA, 46 bp, synthetic construct
  misc_feature 1..46: note = Artificial DNA Primer
  source 1..46: mol_type = other DNA
taccagggttacgatccgaccgtcttcccttacatgcagaacccgc

SEQ ID NO: 393 - DNA, 31 bp, synthetic construct; identical to SEQ ID NO: 391
  misc_feature 1..31: note = Artificial DNA Primer
  source 1..31: mol_type = other DNA

SEQ ID NO: 394 - DNA, 44 bp, synthetic construct
  misc_feature 1..44: note = Artificial DNA Primer
  source 1..44: mol_type = other DNA
ccgacctccttcccttacatgctcaacccgcccatcgtggtcgg

SEQ ID NO: 395 - DNA, 33 bp, synthetic construct
  misc_feature 1..33: note = Artificial DNA Primer
  source 1..33: mol_type = other DNA
catgtaagggaaggaggtcggatcgtaaccctg

SEQ ID NO: 396 - DNA, 45 bp, synthetic construct
  misc_feature 1..45: note = Artificial DNA Primer
  source 1..45: mol_type = other DNA
ttgccccggatgccttcgccaccggcgatatcatctgccacaaga

SEQ ID NO: 397 - DNA, 31 bp, synthetic construct
  misc_feature 1..31: note = Artificial DNA Primer
  source 1..31: mol_type = other DNA
ggcgaaggcatccggggcaacaaagccgttg

SEQ ID NO: 398 - DNA, 45 bp, synthetic construct
  misc_feature 1..45: note = Artificial DNA Primer
  source 1..45: mol_type = other DNA
cgagttcttcaagatcgacgagtgcggcctggtcgacggcagctc

SEQ ID NO: 399 - DNA, 34 bp, synthetic construct
  misc_feature 1..34: note = Artificial DNA Primer
  source 1..34: mol_type = other DNA
ctcgtcgatcttgaagaactcgagcttggtcttg

SEQ ID NO: 400 - DNA, 45 bp, synthetic construct
  misc_feature 1..45: note = Artificial DNA Primer
  source 1..45: mol_type = other DNA
cgagttcttcaagatcgacgagggcggcctggtcgacggcagctc

SEQ ID NO: 401 - DNA, 34 bp, synthetic construct; identical to SEQ ID NO: 399
  misc_feature 1..34: note = Artificial DNA Primer
  source 1..34: mol_type = other DNA

SEQ ID NO: 402 - DNA, 43 bp, synthetic construct
  misc_feature 1..43: note = Artificial DNA Primer
  source 1..43: mol_type = other DNA
acggcagctcggcgcccggtatctggggctccgaccagctcat

SEQ ID NO: 403 - DNA, 28 bp, synthetic construct
  misc_feature 1..28: note = Artificial DNA Primer
  source 1..28: mol_type = other DNA
accgggcgccgagctgccgtcgaccagg

SEQ ID NO: 404 - DNA, 44 bp, synthetic construct
  misc_feature 1..44: note = Artificial DNA Primer
  source 1..44: mol_type = other DNA
gccaacaacaactcgtggctcctcgagatcccgcccaccatcgc

SEQ ID NO: 405 - DNA, 32 bp, synthetic construct
  misc_feature 1..32: note = Artificial DNA Primer
  source 1..32: mol_type = other DNA
gagccacgagttgttgttggcgatgagctggt

SEQ ID NO: 406 - DNA, 43 bp, synthetic construct
  misc_feature 1..43: note = Artificial DNA Primer
  source 1..43: mol_type = other DNA
cctacaccgtcccggggccgccgctcatctccggcgccgtcag

SEQ ID NO: 407 - DNA, 29 bp, synthetic construct
  misc_feature 1..29: note = Artificial DNA Primer
  source 1..29: mol_type = other DNA
cggccccgggacggtgtaggtgatcgggg

SEQ ID NO: 408 - DNA, 884 bp, Talaromyces thermophilus
  source 1..884: mol_type = genomic DNA
atgaaagcccccagcgctgcgtcgatcctccttcctttccttgcgagcatcacccgtacctctgcccacgggttcgtctccaatatcgtcattaatggtgtctcgtatcggggctggctccccaatgaagacccctataaacctgagcccccgattggcgtgggttgggagacgcccaacctgagcaacggcttcgtgacgcccgaagaagcgttgaccgatgcgatcgtctgccacaaggaggccaagccggcccgcggctatgccagcgtcgcagccggcgacaagatctatatccaatggcagccgattccatggccggagtctcaccatggtgcgttagactctctcattgttttgcagtcaagcctcgcccaactgacaacattctcttccaaggacccgtcctggactatctggccccttgcaacggcgactgccagaacgtcaacaagtccagcctggagtttttcaagatcgacggcaaaggactcatcgacggctcctccccgccgggcttctgggccgacgacgaacttatcgccaacggcaacggctggctggtccagatccccgaggacatcaagccgggcaactacgtgctgcgacatgagatcatcgccttgcatgagggattcaaccagaacggcgcccagctgtatccccagtgcttcaacctgcagattacgggatctggcaccgttgagccggagggaacgcccgccacggagctgtattcgcccaccgacccgggcattctggtcgacatctacaaccccttgagcacgtatgtggtgccgggccccacgctcatcccacaggcggttgagatcgaacagtcttcgtcggcggttacggcgacagggacgccaacgccggcttaa

SEQ ID NO: 409 - protein, 272 aa, Talaromyces thermophilus
MKAPSAASILLPFLASITRTSAHGFVSNIVINGVSYRGWLPNEDPYKPEPPIGVGWETPNLSNGFVTPEEALTDAIVCHKEAKPARGYASVAAGDKIYIQWQPIPWPESHHGPVLDYLAPCNGDCQNVNKSSLEFFKIDGKGLIDGSSPPGFWADDELIANGNGWLVQIPEDIKPGNYVLRHEIIALHEGFNQNGAQLYPQCFNLQITGSGTVEPEGTPATELYSPTDPGILVDIYNPLSTYVVPGPTLIPQAVEIEQSSSAVTATGTPTPA

SEQ ID NO: 410 - DNA, 885 bp, Talaromyces thermophilus
  source 1..885: mol_type = genomic DNA
atgaagggctccagcgctgcgtcggtgcttcttgctctcctcgcgggcattacccgtacctctgcgcacgggtatgtctccaacattgttgtcaacggcgtctactatcgaggctggctccccggcgaagacccctataaccctgaccccccgatcggcgtgggctgggagacgcccaacctgggcaacggcttcgtgacgcctgaagaagcgtcgaccgatgccatcatctgccacaaggaggccaagccggcccgcggccatgccaccgtgaaagccggcgacaagatctacatccaatggcagccgatcccctggccggagtcccaccacggtgcgtagcatttccttgagactctgattgttgcattccagtctcacccactaacaatacttctaggccccgtcctcgactatctggccgcttgcaacggcgactgcgagaccgtcgacaagaccagcctgcggttcttcaagatctccaacaagggtctcatcgacggctcttccccgccgggctactgggctgacgatcagctcatcgagaacggtaacggatggctggttcagattcccgaggacatcaagccgggcaactacgtgctgcgacacgagatcatcgctttgcacgcagcgggcaacccgaacggcgcccagctgtatccgcagtgcttcaacctgcatattacgggttccggcaccgtcgagccgcagggaataccagccaccgagctgtactcgcccgatgacccgggcattctgatcaacatctaccagcccttgaccacgtatgaggtgccgggcccgacgcccatcccacaggcggttgagattgagcagtcttcgtccgcgattaccgcgactggaacgccaacgccggcatga

SEQ ID NO: 411 - protein, 272 aa, Talaromyces thermophilus
MKGSSAASVLLALLAGITRTSAHGYVSNIVVNGVYYRGWLPGEDPYNPDPPIGVGWETPNLGNGFVTPEEASTDAIICHKEAKPARGHATVKAGDKIYIQWQPIPWPESHHGPVLDYLAACNGDCETVDKTSLRFFKISNKGLIDGSSPPGYWADDQLIENGNGWLVQIPEDIKPGNYVLRHEIIALHAAGNPNGAQLYPQCFNLHITGSGTVEPQGIPATELYSPDDPGILINIYQPLTTYEVPGPTPIPQAVEIEQSSSAITATGTPTPA

(19) United States
(12) Patent Application Publication
(10) Pub. No.: US 2024/0110169 A1
(43) Pub. Date: Apr. 4, 2024

(54) GH61 VARIANTS AND POLYNUCLEOTIDES ENCODING SAME

(71) Applicants: Novozymes A/S, Bagsvaerd (DK); Novozymes, Inc., Davis, CA (US)

(72) Inventors: Janine Lin, Davis, CA (US); Doreen Bohan, Fairfield, CA (US); Michelle Maranta, Davis, CA (US); Leslie Beresford, Roseville, CA (US); Michael Lamsa, Woodland, CA (US); Matt Sweeney, Sacramento, CA (US); Mark David Wogulis, Davis, CA (US); Elizabeth Znameroski, Davis, CA (US); Frank Winther Rasmussen, Roskilde (DK)

(73) Assignees: Novozymes A/S, Bagsvaerd (DK); Novozymes, Inc., Davis, CA (US)

(21) Appl. No.: 18/524,366

(22) Filed: Nov. 30, 2023

Related U.S. Application Data
(62) Division of application No. 17/714,461, filed on Apr. 6, 2022, now Pat. No. 11,891,637.
Application No. 17/714,461 is a division of application No. 16/269,212, filed on Feb. 6, 2019, now Pat. No. 11,345,905.
Application No. 16/269,212 is a division of application No. 14/395,984, filed on Oct. 21, 2014, now Pat. No. 10,227,579.
(60) Provisional application No. 61/639,648, filed on Apr. 27, 2012.

(51) Int. Cl. C12N 9/42; C11D 3/386; C12N 9/02; C12P 19/02; C12P 19/14; D21C 5/00; D21H 17/00

(52) U.S. Cl. CPC C12N 9/2437; C11D 3/38645; C12N 9/0071; C12N 9/2434; C12P 19/02; C12P 19/14; C12Y 302/01021; D21C 5/005; D21H 17/005; C12P 2203/00; C12Y 114/00; C12Y 302/01004

(57) ABSTRACT

The present invention relates to GH61 polypeptide variants. The present invention also relates to polynucleotides encoding the variants; nucleic acid constructs, vectors, and host cells comprising the polynucleotides; and methods of using the variants.

### CROSS-REFERENCE TO RELATED APPLICATIONS

This application is a divisional application of U.S. patent application Ser. No. 17/714,461, filed on Apr. 6, 2022, which is a divisional application of U.S. patent application Ser. No. 16/269,212, filed on Feb. 6, 2019, issued U.S. Pat. No. 11,345,905, granted on May 31, 2022, which is a divisional application of U.S. patent application Ser. No. 14/395,984, filed on Oct. 21, 2014, issued U.S. Pat. No. 10,227,579, granted on Mar. 12, 2019 which is a 35 U.S.C. § 371 national application of PCT/US2013/038477, filed on Apr. 26, 2013, which claims priority from U.S. Provisional Application Ser. No. 61/639,648, filed on Apr. 27, 2012. The contents of these applications are fully incorporated herein by reference.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to GH61 polypeptide variants, polynucleotides encoding the variants, methods of producing the variants, and methods of using the variants.

### Description of the Related Art

Cellulose is a polymer of the simple sugar glucose covalently linked by beta-1,4-bonds. Many microorganisms produce enzymes that hydrolyze beta-linked glucans. These enzymes include endoglucanases, cellobiohydrolases, and beta-glucosidases. Endoglucanases digest the cellulose polymer at random locations, opening it to attack by cellobiohydrolases. Cellobiohydrolases sequentially release molecules of cellobiose from the ends of the cellulose polymer. Cellobiose is a water-soluble beta-1,4-linked dimer of glucose. Beta-glucosidases hydrolyze cellobiose to glucose.

The conversion of lignocellulosic feedstocks into ethanol has the advantages of the ready availability of large amounts of feedstock, the desirability of avoiding burning or land filling the materials, and the cleanliness of the ethanol fuel. Wood, agricultural residues, herbaceous crops, and municipal solid wastes have been considered as feedstocks for ethanol production. These materials primarily consist of cellulose, hemicellulose, and lignin. Once the lignocellulose is converted to fermentable sugars, e.g., glucose, the fermentable sugars can easily be fermented by yeast into ethanol.

WO 2005/074647, WO 2008/148131, and WO 2011/035027 disclose isolated GH61 polypeptides having cellulolytic enhancing activity and the polynucleotides thereof from Thielavia terrestris. WO 2005/074656 and WO 2010/065830 disclose isolated GH61 polypeptides having cellulolytic enhancing activity and the polynucleotides thereof from Thermoascus aurantiacus. WO 2007/089290 and WO 2012/149344 disclose isolated GH61 polypeptides having cellulolytic enhancing activity and the polynucleotides thereof from Trichoderma reesei. WO 2009/085935, WO 2009/085859, WO 2009/085864, and WO 2009/085868 disclose isolated GH61 polypeptides having cellulolytic enhancing activity and the polynucleotides thereof from Myceliophthora thermophila. WO 2010/138754 discloses an isolated GH61 polypeptide having cellulolytic enhancing activity and the polynucleotide thereof from Aspergillus fumigatus. WO 2011/005867 discloses an isolated GH61 polypeptide having cellulolytic enhancing activity and the polynucleotide thereof from Penicillium pinophilum. WO 2011/039319 discloses an isolated GH61 polypeptide having cellulolytic enhancing activity and the polynucleotide thereof from Thermoascus sp. WO 2011/041397 discloses an isolated GH61 polypeptide having cellulolytic enhancing activity and the polynucleotide thereof from Penicillium sp. WO 2011/041504 discloses isolated GH61 polypeptides having cellulolytic enhancing activity and the polynucleotides thereof from Thermoascus crustaceus. WO 2012/030799 discloses isolated GH61 polypeptides having cellulolytic enhancing activity and the polynucleotides thereof from Aspergillus aculeatus. WO 2012/113340 discloses isolated GH61 polypeptides having cellulolytic enhancing activity and the polynucleotides thereof from Thermomyces lanuginosus. WO 2012/122477 discloses isolated GH61 polypeptides having cellulolytic enhancing activity and the polynucleotides thereof from Aurantiporus alborubescens, Trichophaea saccata, and Penicillium thomii. WO 2012/135659 discloses an isolated GH61 polypeptide having cellulolytic enhancing activity and the polynucleotide thereof from Talaromyces stipitatus. WO 2012/146171 discloses isolated GH61 polypeptides having cellulolytic enhancing activity and the polynucleotides thereof from Humicola insolens. WO 2012/101206 discloses isolated GH61 polypeptides having cellulolytic enhancing activity and the polynucleotides thereof from Malbranchea cinnamomea, Talaromyces leycettanus, and Chaetomium thermophilum. WO 2013/043910 discloses isolated GH61 polypeptides having cellulolytic enhancing activity and the polynucleotides thereof from Acrophialophora fusispora and Corynascus sepedonium.

WO 2012/044835 and WO 2012/044836 disclose GH61 polypeptide variants having cellulolytic enhancing activity with improved thermal activity and thermostability.

There is a need in the art for GH61 polypeptides having cellulolytic enhancing activity with increased thermostability as a component of enzyme compositions for use in the degradation of lignocellulose at high temperatures.

The present invention provides GH61 polypeptide variants with increased thermostability.

### SUMMARY OF THE INVENTION

The present invention relates to isolated GH61 polypeptide variants, comprising a substitution at one or more (e.g., several) positions corresponding to positions 26, 32, 34, 40, 41, 42, 47, 56, 72, 102, 123, 138, 149, 152, 163, 164, 166, 169, 186, 200, 207, 213, 219, 222, 234, 246, 249, and 250 of the mature polypeptide of SEQ ID NO: 30, wherein the variants have cellulolytic enhancing activity.

The present invention also relates to isolated polynucleotides encoding the variants; nucleic acid constructs, vectors, and host cells comprising the polynucleotides; and methods of producing the variants.

The present invention also relates to processes for degrading or converting a cellulosic material, comprising: treating the cellulosic material with an enzyme composition in the presence of a GH61 polypeptide variant of the present invention. In one aspect, the processes further comprise recovering the degraded or converted cellulosic material.

The present invention also relates to processes of producing a fermentation product, comprising: (a) saccharifying a cellulosic material with an enzyme composition in the presence of a GH61 polypeptide variant of the present invention; (b) fermenting the saccharified cellulosic material with one or more (e.g., several) fermenting microorganisms to produce the fermentation product; and (c) recovering the fermentation product from the fermentation.

The present invention also relates to processes of fermenting a cellulosic material, comprising: fermenting the cellulosic material with one or more (e.g., several) fermenting microorganisms, wherein the cellulosic material is saccharified with an enzyme composition in the presence of a GH61 polypeptide variant of the present invention. In one aspect, the fermenting of the cellulosic material produces a fermentation product. In another aspect, the processes further comprise recovering the fermentation product from the fermentation.

### BRIEF DESCRIPTION OF THE FIGURES

FIG. 1 shows the genomic DNA sequence (SEQ ID NO: 29) and the deduced amino acid sequence (SEQ ID NO: 30) of an Aspergillus fumigatus gene encoding a GH61B polypeptide having cellulolytic enhancing activity.

FIG. 2 shows a restriction map of pMMar44.

FIG. 3 shows a restriction map of pMMar49.

FIG. 4 shows a restriction map of pMMar45.

FIG. 5 shows a restriction map of pDFng113.

FIG. 6 shows a restriction map of pDFng153-4.

FIG. 7 shows a restriction map of pDFng154-17.

FIG. 8 shows a restriction map of pDFng155-33.

FIG. 9 shows a restriction map of pDFng156-37.

FIG. 10 shows a restriction map of pDFng157-51.

FIG. 11 shows a restriction map of pBGMH16.

### DEFINITIONS

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. For purposes of the present invention, acetylxylan esterase activity is determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. For purposes of the present invention, alpha-L-arabinofuranosidase activity is determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, CA, USA).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. For purposes of the present invention, alpha-glucuronidase activity is determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. For purposes of the present invention, beta-glucosidase activity is determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. For purposes of the present invention, beta-xylosidase activity is preferably determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside as substrate in 100 mM sodium citrate containing 0.01% TWEEN® 20.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity is preferably determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68).

For purposes of the present invention, cellulolytic enzyme activity is determined by measuring the increase in hydrolysis of a cellulosic material by cellulolytic enzyme(s) under the following conditions: 1-50 mg of cellulolytic enzyme protein/g of cellulose in PCS (or other pretreated cellulosic material) for 3-7 days at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0, compared to a control hydrolysis without addition of cellulolytic enzyme protein. Typical conditions are 1 ml reactions, washed or unwashed pretreated corn stover (PCS), 5% insoluble solids, 50 mM sodium acetate pH 5, 1 mM MnSO₄, 50° C., 55° C., or 60° C., 72 hours, sugar analysis by AMINEX® HPX-87H column (Bio-Rad Laboratories, Inc., Hercules, CA, USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In a preferred aspect, the cellulosic material is any biomass material. In another preferred aspect, the cellulosic material is lignocellulose, which comprises cellulose, hemicelluloses, and lignin.

In one aspect, the cellulosic material is agricultural residue. In another aspect, the cellulosic material is herbaceous material (including energy crops). In another aspect, the cellulosic material is municipal solid waste. In another aspect, the cellulosic material is pulp and paper mill residue. In another aspect, the cellulosic material is waste paper. In another aspect, the cellulosic material is wood (including forestry residue).

In another aspect, the cellulosic material is arundo. In another aspect, the cellulosic material is bagasse. In another aspect, the cellulosic material is bamboo. In another aspect, the cellulosic material is corn cob. In another aspect, the cellulosic material is corn fiber. In another aspect, the cellulosic material is corn stover. In another aspect, the cellulosic material is miscanthus. In another aspect, the cellulosic material is orange peel. In another aspect, the cellulosic material is rice straw. In another aspect, the cellulosic material is switchgrass. In another aspect, the cellulosic material is wheat straw.

In another aspect, the cellulosic material is aspen. In another aspect, the cellulosic material is eucalyptus. In another aspect, the cellulosic material is fir. In another aspect, the cellulosic material is pine. In another aspect, the cellulosic material is poplar. In another aspect, the cellulosic material is spruce. In another aspect, the cellulosic material is willow.

In another aspect, the cellulosic material is algal cellulose. In another aspect, the cellulosic material is bacterial cellulose. In another aspect, the cellulosic material is cotton linter. In another aspect, the cellulosic material is filter paper. In another aspect, the cellulosic material is microcrystalline cellulose. In another aspect, the cellulosic material is phosphoric-acid treated cellulose.

In another aspect, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art, as described herein. In a preferred aspect, the cellulosic material is pretreated.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Family 61 glycoside hydrolase: The term “Family 61 glycoside hydrolase” or “Family GH61” or “GH61” means a polypeptide falling into the glycoside hydrolase Family 61 according to Henrissat, 1991, Biochem. J. 280: 309-316, and Henrissat, and Bairoch, 1996, Biochem. J. 316: 695-696. The enzymes in this family were originally classified as a glycoside hydrolase family based on measurement of very weak endo-1,4-beta-D-glucanase activity in one family member. The GH61s have recently been classified as lytic polysaccharide monooxygenases (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061).

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase (FAE) is also known as ferulic acid esterase, hydroxycinnamoyl esterase, FAE-III, cinnamoyl ester hydrolase, FAEA, cinnAE, FAE-I, or FAE-II. For purposes of the present invention, feruloyl esterase activity is determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Fragment: The term “fragment” means a polypeptide having one or more (e.g., several) amino acids absent from the amino and/or carboxyl terminus of the mature polypeptide thereof, wherein the fragment has cellulolytic enhancing activity. In one aspect, a fragment contains at least 85% of the amino acid residues, e.g., at least 90% of the amino acid residues or at least 95% of the amino acid residues of the mature polypeptide of a GH61 polypeptide.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure &Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. Such an improved property includes, but is not limited to, increased thermostability.

Increased thermostability: The term “increased thermostability” means a higher retention of cellulolytic enhancing activity of a GH61 polypeptide variant after a period of incubation at a temperature relative to the parent. The increased thermostability of the variant relative to the parent can be assessed, for example, under conditions of one or more (e.g., several) temperatures. For example, the one or more (e.g., several) temperatures can be any temperature or temperatures in the range of 45° C. to 95° C., e.g., 45, 50, 55, 60, 65, 70, 75, 80, 85, or 95° C. (or in between, e.g., 62° C., 68° C., 72° C., etc.) at one or more (e.g., several) pHs in the range of 3 to 9, e.g., 3.0, 3.5, 4.0, 4.5, 5.0, 5.5, 6.0, 6.5, 7.0, 7.5, 8.0, 8.5, or 9.0 (or in between) for a suitable period (time) of incubation, e.g., 1 minute, 5 minutes, 10 minutes, 15 minutes, 20 minutes, 25 minutes, 30 minutes, 45 minutes, or 60 minutes (or in between, e.g., 23 minutes, 37 minutes, etc.), such that the variant retains residual activity. However, longer periods of incubation can also be used. The term “increased thermostability” can be used interchangeably with “improved thermostability”.

The increased thermostability of the variant relative to the parent can be determined by differential scanning calorimetry (DSC) using methods standard in the art (see, for example, Sturtevant, 1987, Annual Review of Physical Chemistry 38: 463-488; Example 9). The increased thermostability of the variant relative to the parent can also be determined using protein thermal unfolding analysis (see, for example, Example 10 herein). The increased thermostability of the variant relative to the parent can also be determined using any enzyme assay known in the art for GH61 polypeptides having cellulolytic enhancing activity to measure residual activity after a temperature treatment. See for example, WO 2005/074647, WO 2008/148131 WO 2005/074656, WO 2010/065830, WO 2007/089290, WO 2009/085935, WO 2009/085859, WO 2009/085864, WO 2009/085868, and WO 2008/151043, which are incorporated herein by reference. Alternatively, the increased thermostability of the variant relative to the parent can be determined using any application assay for the variant where the performance of the variant is compared to the parent. For example, the application assays described in Example 5 can be used.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 20 to 326 of SEQ ID NO: 2 based on the SignalP 3.0 program (Bendtsen et al., 2004, J. Mol. Biol. 340: 783-795) that predicts amino acids 1 to 19 of SEQ ID NO: 2 are a signal peptide. In another aspect, the mature polypeptide is amino acids 18 to 239 of SEQ ID NO: 4 based on the SignalP program that predicts amino acids 1 to 17 of SEQ ID NO: 4 are a signal peptide. In another aspect, the mature polypeptide is amino acids 20 to 258 of SEQ ID NO: 6 based on the SignalP program that predicts amino acids 1 to 19 of SEQ ID NO: 6 are a signal peptide. In another aspect, the mature polypeptide is amino acids 19 to 226 of SEQ ID NO: 8 based on the SignalP program that predicts amino acids 1 to 18 of SEQ ID NO: 8 are a signal peptide. In another aspect, the mature polypeptide is amino acids 20 to 304 of SEQ ID NO: 10 based on the SignalP program that predicts amino acids 1 to 19 of SEQ ID NO: 10 are a signal peptide. In another aspect, the mature polypeptide is amino acids 16 to 317 of SEQ ID NO: 12 based on the SignalP program that predicts amino acids 1 to 15 of SEQ ID NO: 12 are a signal peptide. In another aspect, the mature polypeptide is amino acids 22 to 249 of SEQ ID NO: 14 based on the SignalP program that predicts amino acids 1 to 21 of SEQ ID NO: 14 are a signal peptide. In another aspect, the mature polypeptide is amino acids 20 to 249 of SEQ ID NO: 16 based on the SignalP program that predicts amino acids 1 to 19 of SEQ ID NO: 16 are a signal peptide. In another aspect, the mature polypeptide is amino acids 18 to 232 of SEQ ID NO: 18 based on the SignalP program that predicts amino acids 1 to 17 of SEQ ID NO: 18 are a signal peptide. In another aspect, the mature polypeptide is amino acids 16 to 235 of SEQ ID NO: 20 based on the SignalP program that predicts amino acids 1 to 15 of SEQ ID NO: 20 are a signal peptide. In another aspect, the mature polypeptide is amino acids 19 to 323 of SEQ ID NO: 22 based on the SignalP program that predicts amino acids 1 to 18 of SEQ ID NO: 22 are a signal peptide. In another aspect, the mature polypeptide is amino acids 16 to 310 of SEQ ID NO: 24 based on the SignalP program that predicts amino acids 1 to 15 of SEQ ID NO: 24 are a signal peptide. In another aspect, the mature polypeptide is amino acids 20 to 246 of SEQ ID NO: 26 based on the SignalP program that predicts amino acids 1 to 19 of SEQ ID NO: 26 are a signal peptide. In another aspect, the mature polypeptide is amino acids 22 to 354 of SEQ ID NO: 28 based on the SignalP program that predicts amino acids 1 to 21 of SEQ ID NO: 28 are a signal peptide. In another aspect, the mature polypeptide is amino acids 22 to 250 of SEQ ID NO: 30 based on the SignalP program that predicts amino acids 1 to 21 of SEQ ID NO: 30 are a signal peptide. In another aspect, the mature polypeptide is amino acids 22 to 322 of SEQ ID NO: 32 based on the SignalP program that predicts amino acids 1 to 21 of SEQ ID NO: 32 are a signal peptide. In another aspect, the mature polypeptide is amino acids 24 to 444 of SEQ ID NO: 34 based on the SignalP program that predicts amino acids 1 to 23 of SEQ ID NO: 34 are a signal peptide. In another aspect, the mature polypeptide is amino acids 26 to 253 of SEQ ID NO: 36 based on the SignalP program that predicts amino acids 1 to 25 of SEQ ID NO: 36 are a signal peptide. In another aspect, the mature polypeptide is amino acids 18 to 246 of SEQ ID NO: 38 based on the SignalP program that predicts amino acids 1 to 17 of SEQ ID NO: 38 are a signal peptide. In another aspect, the mature polypeptide is amino acids 20 to 334 of SEQ ID NO: 40 based on the SignalP program that predicts amino acids 1 to 19 of SEQ ID NO: 40 are a signal peptide. In another aspect, the mature polypeptide is amino acids 18 to 227 of SEQ ID NO: 42 based on the SignalP program that predicts amino acids 1 to 17 of SEQ ID NO: 42 are a signal peptide. In another aspect, the mature polypeptide is amino acids 20 to 223 of SEQ ID NO: 44 based on the SignalP program that predicts amino acids 1 to 19 of SEQ ID NO: 44 are a signal peptide. In another aspect, the mature polypeptide is amino acids 22 to 368 of SEQ ID NO: 46 based on the SignalP program that predicts amino acids 1 to 21 of SEQ ID NO: 46 are a signal peptide. In another aspect, the mature polypeptide is amino acids 25 to 330 of SEQ ID NO: 48 based on the SignalP program that predicts amino acids 1 to 24 of SEQ ID NO: 48 are a signal peptide. In another aspect, the mature polypeptide is amino acids 17 to 236 of SEQ ID NO: 50 based on the SignalP program that predicts amino acids 1 to 16 of SEQ ID NO: 50 are a signal peptide. In another aspect, the mature polypeptide is amino acids 19 to 250 of SEQ ID NO: 52 based on the SignalP program that predicts amino acids 1 to 18 of SEQ ID NO: 52 are a signal peptide. In another aspect, the mature polypeptide is amino acids 23 to 478 of SEQ ID NO: 54 based on the SignalP program that predicts amino acids 1 to 22 of SEQ ID NO: 54 are a signal peptide. In another aspect, the mature polypeptide is amino acids 17 to 230 of SEQ ID NO: 56 based on the SignalP program that predicts amino acids 1 to 16 of SEQ ID NO: 56 are a signal peptide. In another aspect, the mature polypeptide is amino acids 20 to 257 of SEQ ID NO: 58 based on the SignalP program that predicts amino acids 1 to 19 of SEQ ID NO: 58 are a signal peptide. In another aspect, the mature polypeptide is amino acids 23 to 251 of SEQ ID NO: 60 based on the SignalP program that predicts amino acids 1 to 22 of SEQ ID NO: 60 are a signal peptide. In another aspect, the mature polypeptide is amino acids 19 to 349 of SEQ ID NO: 62 based on the SignalP program that predicts amino acids 1 to 18 of SEQ ID NO: 62 are a signal peptide. In another aspect, the mature polypeptide is amino acids 24 to 436 of SEQ ID NO: 64 based on the SignalP program that predicts amino acids 1 to 23 of SEQ ID NO: 64 are a signal peptide. In another aspect, the mature polypeptide is amino acids 21 to 344 of SEQ ID NO: 66 based on the SignalP program that predicts amino acids 1 to 23 of SEQ ID NO: 66 are a signal peptide. In another aspect, the mature polypeptide is amino acids 26 to 400 of SEQ ID NO: 68 based on the SignalP program that predicts amino acids 1 to 25 of SEQ ID NO: 68 are a signal peptide. In another aspect, the mature polypeptide is amino acids 21 to 389 of SEQ ID NO: 70 based on the SignalP program that predicts amino acids 1 to 20 of SEQ ID NO: 70 are a signal peptide. In another aspect, the mature polypeptide is amino acids 22 to 406 of SEQ ID NO: 72 based on the SignalP program that predicts amino acids 1 to 21 of SEQ ID NO: 72 are a signal peptide. In another aspect, the mature polypeptide is amino acids 20 to 427 of SEQ ID NO: 74 based on the SignalP program that predicts amino acids 1 to 19 of SEQ ID NO: 74 are a signal peptide. In another aspect, the mature polypeptide is amino acids 18 to 267 of SEQ ID NO: 76 based on the SignalP program that predicts amino acids 1 to 17 of SEQ ID NO: 76 are a signal peptide. In another aspect, the mature polypeptide is amino acids 21 to 273 of SEQ ID NO: 78 based on the SignalP program that predicts amino acids 1 to 20 of SEQ ID NO: 78 are a signal peptide. In another aspect, the mature polypeptide is amino acids 21 to 322 of SEQ ID NO: 80 based on the SignalP program that predicts amino acids 1 to 20 of SEQ ID NO: 80 are a signal peptide. In another aspect, the mature polypeptide is amino acids 18 to 234 of SEQ ID NO: 82 based on the SignalP program that predicts amino acids 1 to 17 of SEQ ID NO: 82 are a signal peptide. In another aspect, the mature polypeptide is amino acids 24 to 233 of SEQ ID NO: 84 based on the SignalP program that predicts amino acids 1 to 23 of SEQ ID NO: 84 are a signal peptide. In another aspect, the mature polypeptide is amino acids 17 to 237 of SEQ ID NO: 86 based on the SignalP program that predicts amino acids 1 to 16 of SEQ ID NO: 86 are a signal peptide. In another aspect, the mature polypeptide is amino acids 20 to 484 of SEQ ID NO: 88 based on the SignalP program that predicts amino acids 1 to 19 of SEQ ID NO: 88 are a signal peptide. In another aspect, the mature polypeptide is amino acids 22 to 320 of SEQ ID NO: 90 based on the SignalP program that predicts amino acids 1 to 21 of SEQ ID NO: 90 are a signal peptide. In another aspect, the mature polypeptide is amino acids 23 to 272 of SEQ ID NO: 92 based on the SignalP program that predicts amino acids 1 to 22 of SEQ ID NO: 92 are a signal peptide. In another aspect, the mature polypeptide is amino acids 22 to 327 of SEQ ID NO: 94 based on the SignalP program that predicts amino acids 1 to 21 of SEQ ID NO: 94 are a signal peptide. In another aspect, the mature polypeptide is amino acids 23 to 274 of SEQ ID NO: 96 based on the SignalP program that predicts amino acids 1 to 22 of SEQ ID NO: 96 are a signal peptide. In another aspect, the mature polypeptide is amino acids 18 to 227 of SEQ ID NO: 98 based on the SignalP program that predicts amino acids 1 to 17 of SEQ ID NO: 98 are a signal peptide. In another aspect, the mature polypeptide is amino acids 17 to 257 of SEQ ID NO: 100 based on the SignalP program that predicts amino acids 1 to 16 of SEQ ID NO: 100 are a signal peptide. In another aspect, the mature polypeptide is amino acids 20 to 246 of SEQ ID NO: 102 based on the SignalP program that predicts amino acids 1 to 19 of SEQ ID NO: 102 are a signal peptide. In another aspect, the mature polypeptide is amino acids 28 to 265 of SEQ ID NO: 104 based on the SignalP program that predicts amino acids 1 to 27 of SEQ ID NO: 104 are a signal peptide. In another aspect, the mature polypeptide is amino acids 16 to 310 of SEQ ID NO: 106 based on the SignalP program that predicts amino acids 1 to 15 of SEQ ID NO: 106 are a signal peptide. In another aspect, the mature polypeptide is amino acids 21 to 354 of SEQ ID NO: 108 based on the SignalP program that predicts amino acids 1 to 20 of SEQ ID NO: 108 are a signal peptide. In another aspect, the mature polypeptide is amino acids 22 to 267 of SEQ ID NO: 110 based on the SignalP program that predicts amino acids 1 to 21 of SEQ ID NO: 110 are a signal peptide. In another aspect, the mature polypeptide is amino acids 16 to 237 of SEQ ID NO: 112 based on the SignalP program that predicts amino acids 1 to 15 of SEQ ID NO: 112 are a signal peptide. In another aspect, the mature polypeptide is amino acids 20 to 234 of SEQ ID NO: 114 based on the SignalP program that predicts amino acids 1 to 19 of SEQ ID NO: 114 are a signal peptide. In another aspect, the mature polypeptide is amino acids 18 to 226 of SEQ ID NO: 116 based on the SignalP program that predicts amino acids 1 to 17 of SEQ ID NO: 116 are a signal peptide. In another aspect, the mature polypeptide is amino acids 17 to 231 of SEQ ID NO: 118 based on the SignalP program that predicts amino acids 1 to 16 of SEQ ID NO: 118 are a signal peptide. In another aspect, the mature polypeptide is amino acids 22 to 248 of SEQ ID NO: 120 based on the SignalP program that predicts amino acids 1 to 21 of SEQ ID NO: 120 are a signal peptide. In another aspect, the mature polypeptide is amino acids 18 to 233 of SEQ ID NO: 122 based on the SignalP program that predicts amino acids 1 to 17 of SEQ ID NO: 122 are a signal peptide. In another aspect, the mature polypeptide is amino acids 21 to 243 of SEQ ID NO: 124 based on the SignalP program that predicts amino acids 1 to 20 of SEQ ID NO: 124 are a signal peptide. In another aspect, the mature polypeptide is amino acids 21 to 363 of SEQ ID NO: 126 based on the SignalP program that predicts amino acids 1 to 20 of SEQ ID NO: 126 are a signal peptide. In another aspect, the mature polypeptide is amino acids 20 to 296 of SEQ ID NO: 128 based on the SignalP program that predicts amino acids 1 to 19 of SEQ ID NO: 128 are a signal peptide. In another aspect, the mature polypeptide is amino acids 16 to 318 of SEQ ID NO: 130 based on the SignalP program that predicts amino acids 1 to 15 of SEQ ID NO: 130 are a signal peptide. In another aspect, the mature polypeptide is amino acids 19 to 259 of SEQ ID NO: 132 based on the SignalP program that predicts amino acids 1 to 18 of SEQ ID NO: 132 are a signal peptide. In another aspect, the mature polypeptide is amino acids 20 to 325 of SEQ ID NO: 134 based on the SignalP program that predicts amino acids 1 to 19 of SEQ ID NO: 134 are a signal peptide. In another aspect, the mature polypeptide is amino acids 19 to 298 of SEQ ID NO: 136 based on the SignalP program that predicts amino acids 1 to 18 of SEQ ID NO: 136 are a signal peptide. In another aspect, the mature polypeptide is amino acids 20 to 298 of SEQ ID NO: 138 based on the SignalP program that predicts amino acids 1 to 19 of SEQ ID NO: 138 are a signal peptide. In another aspect, the mature polypeptide is amino acids 22 to 344 of SEQ ID NO: 140 based on the SignalP program that predicts amino acids 1 to 21 of SEQ ID NO: 140 are a signal peptide. In another aspect, the mature polypeptide is amino acids 20 to 330 of SEQ ID NO: 142 based on the SignalP program that predicts amino acids 1 to 19 of SEQ ID NO: 142 are a signal peptide. In another aspect, the mature polypeptide is amino acids 19 to 216 of SEQ ID NO: 144 based on the SignalP program that predicts amino acids 1 to 18 of SEQ ID NO: 144 are a signal peptide. In another aspect, the mature polypeptide is amino acids 18 to 490 of SEQ ID NO: 146 based on the SignalP program that predicts amino acids 1 to 17 of SEQ ID NO: 146 are a signal peptide. In another aspect, the mature polypeptide is amino acids 21 to 306 of SEQ ID NO: 148 based on the SignalP program that predicts amino acids 1 to 20 of SEQ ID NO: 148 are a signal peptide. In another aspect, the mature polypeptide is amino acids 22 to 339 of SEQ ID NO: 150 based on the SignalP program that predicts amino acids 1 to 21 of SEQ ID NO: 150 are a signal peptide. In another aspect, the mature polypeptide is amino acids 23 to 334 of SEQ ID NO: 152 based on the SignalP program that predicts amino acids 1 to 22 of SEQ ID NO: 152 are a signal peptide. In another aspect, the mature polypeptide is amino acids 24 to 366 of SEQ ID NO: 154 based on the SignalP program that predicts amino acids 1 to 23 of SEQ ID NO: 154 are a signal peptide. In another aspect, the mature polypeptide is amino acids 21 to 364 of SEQ ID NO: 156 based on the SignalP program that predicts amino acids 1 to 20 of SEQ ID NO: 156 are a signal peptide. In another aspect, the mature polypeptide is amino acids 22 to 344 of SEQ ID NO: 158 based on the SignalP program that predicts amino acids 1 to 21 of SEQ ID NO: 158 are a signal peptide. In another aspect, the mature polypeptide is amino acids 20 to 252 of SEQ ID NO: 160 based on the SignalP program that predicts amino acids 1 to 19 of SEQ ID NO: 160 are a signal peptide. In another aspect, the mature polypeptide is amino acids 20 to 344 of SEQ ID NO: 162 based on the SignalP program that predicts amino acids 1 to 19 of SEQ ID NO: 162 are a signal peptide. In another aspect, the mature polypeptide is amino acids 22 to 347 of SEQ ID NO: 164 based on the SignalP program that predicts amino acids 1 to 21 of SEQ ID NO: 164 are a signal peptide. In another aspect, the mature polypeptide is amino acids 20 to 342 of SEQ ID NO: 166 based on the SignalP program that predicts amino acids 1 to 19 of SEQ ID NO: 166 are a signal peptide. In another aspect, the mature polypeptide is amino acids 27 to 254 of SEQ ID NO: 168 based on the SignalP program that predicts amino acids 1 to 26 of SEQ ID NO: 168 are a signal peptide. In another aspect, the mature polypeptide is amino acids 23 to 272 of SEQ ID NO: 409 based on the SignalP program that predicts amino acids 1 to 22 of SEQ ID NO: 409 are a signal peptide. In another aspect, the mature polypeptide is amino acids 23 to 272 of SEQ ID NO: 411 based on the SignalP program that predicts amino acids 1 to 22 of SEQ ID NO: 411 are a signal peptide. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having cellulolytic enhancing activity. In one aspect, the mature polypeptide coding sequence is nucleotides 388 to 1332 of SEQ ID NO: 1 based on the SignalP 3.0 program (Bendtsen et al., 2004, supra) that predicts nucleotides 330 to 387 of SEQ ID NO: 1 encode a signal peptide. In another aspect, the mature polypeptide coding sequence is nucleotides 98 to 821 of SEQ ID NO: 3 based on the SignalP program that predicts nucleotides 47 to 97 of SEQ ID NO: 3 encode a signal peptide. In another aspect, the mature polypeptide coding sequence is nucleotides 126 to 978 of SEQ ID NO: 5 based on the SignalP program that predicts nucleotides 69 to 125 of SEQ ID NO: 5 encode a signal peptide. In another aspect, the mature polypeptide coding sequence is nucleotides 55 to 678 of SEQ ID NO: 7 or the genomic DNA sequence thereof based on the SignalP program that predicts nucleotides 1 to 54 of SEQ ID NO: 7 encode a signal peptide. In another aspect, the mature polypeptide coding sequence is nucleotides 58 to 912 of SEQ ID NO: 9 or the genomic DNA sequence thereof based on the SignalP program that predicts nucleotides 1 to 57 of SEQ ID NO: 9 encode a signal peptide. In another aspect, the mature polypeptide coding sequence is nucleotides 46 to 951 of SEQ ID NO: 11 or the genomic DNA sequence thereof based on the SignalP program that predicts nucleotides 1 to 45 of SEQ ID NO: 11 encode a signal peptide. In another aspect, the mature polypeptide coding sequence is nucleotides 64 to 796 of SEQ ID NO: 13 based on the SignalP program that predicts nucleotides 1 to 63 of SEQ ID NO: 13 encode a signal peptide. In another aspect, the mature polypeptide coding sequence is nucleotides 77 to 766 of SEQ ID NO: 15 based on the SignalP program that predicts nucleotides 20 to 76 of SEQ ID NO: 15 or the genomic DNA sequence thereof encode a signal peptide. In another aspect, the mature polypeptide coding sequence is nucleotides 52 to 921 of SEQ ID NO: 17 based on the SignalP program that predicts nucleotides 1 to 51 of SEQ ID NO: 17 encode a signal peptide. In another aspect, the mature polypeptide coding sequence is nucleotides 46 to 851 of SEQ ID NO: 19 based on the SignalP program that predicts nucleotides 1 to 45 of SEQ ID NO: 19 encode a signal peptide. In another aspect, the mature polypeptide coding sequence is nucleotides 55 to 1239 of SEQ ID NO: 21 based on the SignalP program that predicts nucleotides 1 to 54 of SEQ ID NO: 21 encode a signal peptide. In another aspect, the mature polypeptide coding sequence is nucleotides 46 to 1250 of SEQ ID NO: 23 based on the SignalP program that predicts nucleotides 1 to 45 of SEQ ID NO: 23 encode a signal peptide. In another aspect, the mature polypeptide coding sequence is nucleotides 58 to 811 of SEQ ID NO: 25 based on the SignalP program that predicts nucleotides 1 to 57 of SEQ ID NO: 25 encode a signal peptide. In another aspect, the mature polypeptide coding sequence is nucleotides 64 to 1112 of SEQ ID NO: 27 based on the SignalP program that predicts nucleotides 1 to 63 of SEQ ID NO: 27 encode a signal peptide. In another aspect, the mature polypeptide coding sequence is nucleotides 64 to 859 of SEQ ID NO: 29 based on the SignalP program that predicts nucleotides 1 to 63 of SEQ ID NO: 29 encode a signal peptide. In another aspect, the mature polypeptide coding sequence is nucleotides 64 to 1018 of SEQ ID NO: 31 based on the SignalP program that predicts nucleotides 1 to 63 of SEQ ID NO: 31 encode a signal peptide. In another aspect, the mature polypeptide coding sequence is nucleotides 70 to 1483 of SEQ ID NO: 33 based on the SignalP program that predicts nucleotides 1 to 69 of SEQ ID NO: 33 encode a signal peptide. In another aspect, the mature polypeptide coding sequence is nucleotides 76 to 832 of SEQ ID NO: 35 based on the SignalP program that predicts nucleotides 1 to 75 of SEQ ID NO: 35 encode a signal peptide. In another aspect, the mature polypeptide coding sequence is nucleotides 52 to 875 of SEQ ID NO: 37 based on the SignalP program that predicts nucleotides 1 to 51 of SEQ ID NO: 37 encode a signal peptide. In another aspect, the mature polypeptide coding sequence is nucleotides 58 to 1250 of SEQ ID NO: 39 based on the SignalP program that predicts nucleotides 1 to 57 of SEQ ID NO: 39 encode a signal peptide. In another aspect, the mature polypeptide coding sequence is nucleotides 52 to 795 of SEQ ID NO: 41 based on the SignalP program that predicts nucleotides 1 to 51 of SEQ ID NO: 41 encode a signal peptide. In another aspect, the mature polypeptide coding sequence is nucleotides 58 to 974 of SEQ ID NO: 43 based on the SignalP program that predicts nucleotides 1 to 57 of SEQ ID NO: 43 encode a signal peptide. In another aspect, the mature polypeptide coding sequence is nucleotides 64 to 1104 of SEQ ID NO: 45 based on the SignalP program that predicts nucleotides 1 to 63 of SEQ ID NO: 45 encode a signal peptide. In another aspect, the mature polypeptide coding sequence is nucleotides 73 to 990 of SEQ ID NO: 47 based on the SignalP program that predicts nucleotides 1 to 72 of SEQ ID NO: 47 encode a signal peptide. In another aspect, the mature polypeptide coding sequence is nucleotides 49 to 1218 of SEQ ID NO: 49 based on the SignalP program that predicts nucleotides 1 to 48 of SEQ ID NO: 49 encode a signal peptide. In another aspect, the mature polypeptide coding sequence is nucleotides 55 to 930 of SEQ ID NO: 51 based on the SignalP program that predicts nucleotides 1 to 54 of SEQ ID NO: 51 encode a signal peptide. In another aspect, the mature polypeptide coding sequence is nucleotides 67 to 1581 of SEQ ID NO: 53 based on the SignalP program that predicts nucleotides 1 to 66 of SEQ ID NO: 53 encode a signal peptide. In another aspect, the mature polypeptide coding sequence is nucleotides 49 to 865 of SEQ ID NO: 55 based on the SignalP program that predicts nucleotides 1 to 48 of SEQ ID NO: 55 encode a signal peptide. In another aspect, the mature polypeptide coding sequence is nucleotides 58 to 1065 of SEQ ID NO: 57 based on the SignalP program that predicts nucleotides 1 to 57 of SEQ ID NO: 57 encode a signal peptide. In another aspect, the mature polypeptide coding sequence is nucleotides 67 to 868 of SEQ ID NO: 59 based on the SignalP program that predicts nucleotides 1 to 66 of SEQ ID NO: 59 encode a signal peptide. In another aspect, the mature polypeptide coding sequence is nucleotides 55 to 1099 of SEQ ID NO: 61 based on the SignalP program that predicts nucleotides 1 to 54 of SEQ ID NO: 61 encode a signal peptide. In another aspect, the mature polypeptide coding sequence is nucleotides 70 to 1483 of SEQ ID NO: 63 based on the SignalP program that predicts nucleotides 1 to 69 of SEQ ID NO: 63 encode a signal peptide. In another aspect, the mature polypeptide coding sequence is nucleotides 61 to 1032 of SEQ ID NO: 65 based on the SignalP program that predicts nucleotides 1 to 60 of SEQ ID NO: 65 encode a signal peptide. In another aspect, the mature polypeptide coding sequence is nucleotides 76 to 1200 of SEQ ID NO: 67 based on the SignalP program that predicts nucleotides 1 to 75 of SEQ ID NO: 67 encode a signal peptide. In another aspect, the mature polypeptide coding sequence is nucleotides 61 to 1167 of SEQ ID NO: 69 based on the SignalP program that predicts nucleotides 1 to 60 of SEQ ID NO: 69 encode a signal peptide. In another aspect, the mature polypeptide coding sequence is nucleotides 64 to 1218 of SEQ ID NO: 71 based on the SignalP program that predicts nucleotides 1 to 63 of SEQ ID NO: 71 encode a signal peptide. In another aspect, the mature polypeptide coding sequence is nucleotides 58 to 1281 of SEQ ID NO: 73 based on the SignalP program that predicts nucleotides 1 to 57 of SEQ ID NO: 73 encode a signal peptide. In another aspect, the mature polypeptide coding sequence is nucleotides 52 to 801 of SEQ ID NO: 75 based on the SignalP program that predicts nucleotides 1 to 51 of SEQ ID NO: 75 encode a signal peptide. In another aspect, the mature polypeptide coding sequence is nucleotides 61 to 819 of SEQ ID NO: 77 based on the SignalP program that predicts nucleotides 1 to 60 of SEQ ID NO: 77 encode a signal peptide. In another aspect, the mature polypeptide coding sequence is nucleotides 61 to 966 of SEQ ID NO: 79 based on the SignalP program that predicts nucleotides 1 to 60 of SEQ ID NO: 79 encode a signal peptide. In another aspect, the mature polypeptide coding sequence is nucleotides 52 to 702 of SEQ ID NO: 81 or the genomic DNA sequence thereof based on the SignalP program that predicts nucleotides 1 to 51 of SEQ ID NO: 81 encode a signal peptide. In another aspect, the mature polypeptide coding sequence is nucleotides 70 to 699 of SEQ ID NO: 83 or the genomic DNA sequence thereof based on the SignalP program that predicts nucleotides 1 to 69 of SEQ ID NO: 83 encode a signal peptide. In another aspect, the mature polypeptide coding sequence is nucleotides 49 to 711 of SEQ ID NO: 85 or the genomic DNA sequence thereof based on the SignalP program that predicts nucleotides 1 to 48 of SEQ ID NO: 85 encode a signal peptide. In another aspect, the mature polypeptide coding sequence is nucleotides 76 to 1452 of SEQ ID NO: 87 based on the SignalP program that predicts nucleotides 1 to 75 of SEQ ID NO: 87 encode a signal peptide. or In another aspect, the mature polypeptide coding sequence is nucleotides 64 to 1018 of SEQ ID NO: 89 based on the SignalP program that predicts nucleotides 1 to 63 of SEQ ID NO: 89 encode a signal peptide. In another aspect, the mature polypeptide coding sequence is nucleotides 67 to 869 of SEQ ID NO: 91 based on the SignalP program that predicts nucleotides 1 to 66 of SEQ ID NO: 91 encode a signal peptide. In another aspect, the mature polypeptide coding sequence is nucleotides 64 to 1036 of SEQ ID NO: 93 based on the SignalP program that predicts nucleotides 1 to 63 of SEQ ID NO: 93 encode a signal peptide. In another aspect, the mature polypeptide coding sequence is nucleotides 67 to 878 of SEQ ID NO: 95 based on the SignalP program that predicts nucleotides 1 to 66 of SEQ ID NO: 95 encode a signal peptide. In another aspect, the mature polypeptide coding sequence is nucleotides 52 to 818 of SEQ ID NO: 97 based on the SignalP program that predicts nucleotides 1 to 51 of SEQ ID NO: 97 encode a signal peptide. In another aspect, the mature polypeptide coding sequence is nucleotides 49 to 1117 of SEQ ID NO: 99 based on the SignalP program that predicts nucleotides 1 to 48 of SEQ ID NO: 99 encode a signal peptide. In another aspect, the mature polypeptide coding sequence is nucleotides 58 to 875 of SEQ ID NO: 101 based on the SignalP program that predicts nucleotides 1 to 57 of SEQ ID NO: 101 encode a signal peptide. In another aspect, the mature polypeptide coding sequence is nucleotides 82 to 1064 of SEQ ID NO: 103 based on the SignalP program that predicts nucleotides 1 to 81 of SEQ ID NO: 103 encode a signal peptide. In another aspect, the mature polypeptide coding sequence is nucleotides 46 to 1032 of SEQ ID NO: 105 based on the SignalP program that predicts nucleotides 1 to 45 of SEQ ID NO: 105 encode a signal peptide. In another aspect, the mature polypeptide coding sequence is nucleotides 61 to 1062 of SEQ ID NO: 107 based on the SignalP program that predicts nucleotides 1 to 60 of SEQ ID NO: 107 encode a signal peptide. In another aspect, the mature polypeptide coding sequence is nucleotides 64 to 801 of SEQ ID NO: 109 based on the SignalP program that predicts nucleotides 1 to 63 of SEQ ID NO: 109 encode a signal peptide. In another aspect, the mature polypeptide coding sequence is nucleotides 46 to 840 of SEQ ID NO: 111 based on the SignalP program that predicts nucleotides 1 to 45 of SEQ ID NO: 111 encode a signal peptide. In another aspect, the mature polypeptide coding sequence is nucleotides 58 to 702 of SEQ ID NO: 113 based on the SignalP program that predicts nucleotides 1 to 57 of SEQ ID NO: 113 encode a signal peptide. In another aspect, the mature polypeptide coding sequence is nucleotides 52 to 750 of SEQ ID NO: 115 based on the SignalP program that predicts nucleotides 1 to 51 of SEQ ID NO: 115 encode a signal peptide. In another aspect, the mature polypeptide coding sequence is nucleotides 49 to 851 of SEQ ID NO: 117 based on the SignalP program that predicts nucleotides 1 to 48 of SEQ ID NO: 117 encode a signal peptide. In another aspect, the mature polypeptide coding sequence is nucleotides 64 to 860 of SEQ ID NO: 119 based on the SignalP program that predicts nucleotides 1 to 63 of SEQ ID NO: 119 encode a signal peptide. In another aspect, the mature polypeptide coding sequence is nucleotides 52 to 830 of SEQ ID NO: 121 based on the SignalP program that predicts nucleotides 1 to 51 of SEQ ID NO: 121 encode a signal peptide. In another aspect, the mature polypeptide coding sequence is nucleotides 61 to 925 of SEQ ID NO: 123 based on the SignalP program that predicts nucleotides 1 to 60 of SEQ ID NO: 123 encode a signal peptide. In another aspect, the mature polypeptide coding sequence is nucleotides 61 to 1089 of SEQ ID NO: 125 based on the SignalP program that predicts nucleotides 1 to 60 of SEQ ID NO: 125 encode a signal peptide. In another aspect, the mature polypeptide coding sequence is nucleotides 58 to 1083 of SEQ ID NO: 127 based on the SignalP program (that predicts nucleotides 1 to 57 of SEQ ID NO: 127 encode a signal peptide. In another aspect, the mature polypeptide coding sequence is nucleotides 46 to 1029 of SEQ ID NO: 129 based on the SignalP program that predicts nucleotides 1 to 45 of SEQ ID NO: 129 encode a signal peptide. In another aspect, the mature polypeptide coding sequence is nucleotides 55 to 1110 of SEQ ID NO: 131 based on the SignalP program that predicts nucleotides 1 to 54 of SEQ ID NO: 131 encode a signal peptide. In another aspect, the mature polypeptide coding sequence is nucleotides 58 to 1100 of SEQ ID NO: 133 based on the SignalP program that predicts nucleotides 1 to 57 of SEQ ID NO: 133 encode a signal peptide. In another aspect, the mature polypeptide coding sequence is nucleotides 55 to 1036 of SEQ ID NO: 135 based on the SignalP program that predicts nucleotides 1 to 54 of SEQ ID NO: 135 encode a signal peptide. In another aspect, the mature polypeptide coding sequence is nucleotides 58 to 1022 of SEQ ID NO: 137 based on the SignalP program that predicts nucleotides 1 to 57 of SEQ ID NO: 137 encode a signal peptide. In another aspect, the mature polypeptide coding sequence is nucleotides 64 to 1032 of SEQ ID NO: 139 based on the SignalP program that predicts nucleotides 1 to 63 of SEQ ID NO: 139 encode a signal peptide. In another aspect, the mature polypeptide coding sequence is nucleotides 58 to 1054 of SEQ ID NO: 141 based on the SignalP program that predicts nucleotides 1 to 57 of SEQ ID NO: 141 encode a signal peptide. In another aspect, the mature polypeptide coding sequence is nucleotides 55 to 769 of SEQ ID NO: 143 based on the SignalP program that predicts nucleotides 1 to 54 of SEQ ID NO: 143 encode a signal peptide. In another aspect, the mature polypeptide coding sequence is nucleotides 52 to 1533 of SEQ ID NO: 145 based on the SignalP program that predicts nucleotides 1 to 51 of SEQ ID NO: 145 encode a signal peptide. In another aspect, the mature polypeptide coding sequence is nucleotides 61 to 918 of SEQ ID NO: 147 based on the SignalP program that predicts nucleotides 1 to 60 of SEQ ID NO: 147 encode a signal peptide. In another aspect, the mature polypeptide coding sequence is nucleotides 64 to 1089 of SEQ ID NO: 149 based on the SignalP program that predicts nucleotides 1 to 63 of SEQ ID NO: 149 encode a signal peptide. In another aspect, the mature polypeptide coding sequence is nucleotides 67 to 1002 of SEQ ID NO: 151 based on the SignalP program that predicts nucleotides 1 to 66 of SEQ ID NO: 151 encode a signal peptide. In another aspect, the mature polypeptide coding sequence is nucleotides 70 to 1098 of SEQ ID NO: 153 based on the SignalP program that predicts nucleotides 1 to 69 of SEQ ID NO: 153 encode a signal peptide. In another aspect, the mature polypeptide coding sequence is nucleotides 61 to 1088 of SEQ ID NO: 155 based on the SignalP program that predicts nucleotides 1 to 60 of SEQ ID NO: 155 encode a signal peptide. In another aspect, the mature polypeptide coding sequence is nucleotides 64 to 1086 of SEQ ID NO: 157 based on the SignalP program that predicts nucleotides 1 to 63 of SEQ ID NO: 157 encode a signal peptide. In another aspect, the mature polypeptide coding sequence is nucleotides 58 to 756 of SEQ ID NO: 159 based on the SignalP program that predicts nucleotides 1 to 57 of SEQ ID NO: 159 encode a signal peptide. In another aspect, the mature polypeptide coding sequence is nucleotides 58 to 1032 of SEQ ID NO: 161 based on the SignalP program that predicts nucleotides 1 to 57 of SEQ ID NO: 161 encode a signal peptide. In another aspect, the mature polypeptide coding sequence is nucleotides 64 to 1041 of SEQ ID NO: 163 based on the SignalP program that predicts nucleotides 1 to 63 of SEQ ID NO: 163 encode a signal peptide. In another aspect, the mature polypeptide coding sequence is nucleotides 58 to 1026 of SEQ ID NO: 165 based on the SignalP program that predicts nucleotides 1 to 57 of SEQ ID NO: 165 encode a signal peptide. In another aspect, the mature polypeptide coding sequence is nucleotides 79 to 762 of SEQ ID NO: 167 based on the SignalP program that predicts nucleotides 1 to 78 of SEQ ID NO: 167 encode a signal peptide. In another aspect, the mature polypeptide coding sequence is nucleotides 67 to 881 of SEQ ID NO: 408 based on the SignalP program that predicts nucleotides 1 to 66 of SEQ ID NO: 408 encode a signal peptide. In another aspect, the mature polypeptide coding sequence is nucleotides 67 to 882 of SEQ ID NO: 410 based on the SignalP program that predicts nucleotides 1 to 66 of SEQ ID NO: 410 encode a signal peptide. The term “mature polypeptide coding sequence” herein shall be understood to include the cDNA sequence of the genomic DNA sequence or the genomic DNA sequence of the cDNA sequence.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent GH61 polypeptide: The term “parent” or “parent GH61 polypeptide” means a GH61 polypeptide to which an alteration, i.e., a substitution, insertion, and/or deletion, at one or more (e.g., several) positions, is made to produce the GH61 polypeptide variants of the present invention. The parent may be a naturally occurring (wild-type) polypeptide or a variant or fragment thereof.

Polypeptide having cellulolytic enhancing activity: The term “polypeptide having cellulolytic enhancing activity” means a GH61 polypeptide or variant thereof that catalyzes the enhancement of the hydrolysis of a cellulosic material by enzyme having cellulolytic activity, i.e., a cellulase. For purposes of the present invention, cellulolytic enhancing activity is determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of a GH61 polypeptide or variant thereof for 1-7 days at a suitable temperature, such as 40° C.-80° C., e.g., 50° C., 55° C., 60° C., 65° C., or 70° C., and a suitable pH, such as 4-9, e.g., 4.5, 5.0, 5.5, 6.0, 6.5, 7.0, 7.5, 8.0, or 8.5, compared to a control hydrolysis with equal total protein loading without cellulolytic enhancing activity (1-50 mg of cellulolytic protein/g of cellulose in PCS) In one aspect, GH61 polypeptide enhancing activity is determined using a mixture of CELLUCLAST® 1.5 L (Novozymes A/S, Bagsværd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 02/095014) of cellulase protein loading is used as the source of the cellulolytic activity.

Another assay for determining the cellulolytic enhancing activity of a GH61 polypeptide or variant thereof is to incubate the GH61 polypeptide or variant with 0.5% phosphoric acid swollen cellulose (PASO), 100 mM sodium acetate pH 5, 1 mM MnSO₄, 0.1% gallic acid, 0.025 mg/ml of Aspergillus fumigatus beta-glucosidase, and 0.01% TRITON® X100 for 24-96 hours at 40° C. followed by determination of the glucose released from the PASO.

The GH61 polypeptides or variants thereof having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Pretreated corn stover: The term “PCS” or “Pretreated Corn Stover” means a cellulosic material derived from corn stover by treatment with heat and dilute sulfuric acid, alkaline pretreatment, neutral pretreatment, or any pretreatment known in the art.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence, wherein the subsequence encodes a fragment having cellulolytic enhancing activity. In one aspect, a subsequence contains at least 85% of the nucleotides, e.g., at least 90% of the nucleotides or at least 95% of the nucleotides of the mature polypeptide coding sequence of a GH61 polypeptide.

Variant: The term “variant” means a polypeptide having cellulolytic enhancing activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the cellulolytic enhancing activity of their parent GH61 polypeptides.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

Wild-type GH61 polypeptide: The term “wild-type” GH61 polypeptide means a GH61 polypeptide naturally produced by a microorganism, such as a bacterium, yeast, or filamentous fungus found in nature.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the processes of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrmann et al., 1997, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. The most common total xylanolytic activity assay is based on production of reducing sugars from polymeric 4-O-methyl glucuronoxylan as described in Bailey, Biely, Poutanen, 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 (4-(1,1,3,3-tetramethylbutyl)phenyl-polyethylene glycol) and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

For purposes of the present invention, xylan degrading activity is determined by measuring the increase in hydrolysis of birchwood xylan (Sigma Chemical Co., Inc., St. Louis, MO, USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, Anal. Biochem 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. For purposes of the present invention, xylanase activity is determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

### Conventions for Designation of Variants

For purposes of the present invention, the mature polypeptide disclosed in SEQ ID NO: 30 is used to determine the corresponding amino acid residue in another GH61 polypeptide. The amino acid sequence of another GH61 polypeptide is aligned with the mature polypeptide disclosed in SEQ ID NO: 30, and based on the alignment, the amino acid position number corresponding to any amino acid residue in the mature polypeptide disclosed in SEQ ID NO: 30 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are a gap open penalty of 10, a gap extension penalty of 0.5, and the EBLOSUM62 (EMBOSS version of BLOSUM62) substitution matrix. Numbering of the amino acid positions is based on the full-length polypeptide (e.g., including the signal peptide) of SEQ ID NO: 30 wherein position 1 is the first amino acid of the signal peptide (i.e., Met) and position 22 is His of SEQ ID NO: 30.

For example, the position corresponding to position 105 of the Aspergillus fumigatus GH61 polypeptide (SEQ ID NO: 30) is position 109 in the Penicillium emersonii GH61 polypeptide (SEQ ID NO: 36), position 105 in the Thermoascus aurantiacus GH61 polypeptide (SEQ ID NO: 14), and position 103 in the Aspergillus aculeatus GH61 polypeptide (SEQ ID NO: 68); the position corresponding to position 188 of the Aspergillus fumigatus GH61 polypeptide is position 192 in the Penicillium emersonii GH61 polypeptide, position 188 in the Thermoascus aurantiacus GH61 polypeptide, and position 186 in the Aspergillus aculeatus GH61 polypeptide; the position corresponding to position 154 of the Aspergillus fumigatus GH61 polypeptide is position 152 in the Aspergillus aculeatus GH61 polypeptide; and the position corresponding to position 189 of the Aspergillus fumigatus GH61 polypeptide is position 193 in the Penicillium emersonii GH61 polypeptide and position 187 in the Aspergillus aculeatus GH61 polypeptide.

Identification of the corresponding amino acid residue in another GH61 polypeptide can be determined by an alignment of multiple polypeptide sequences using several computer programs including, but not limited to MUSCLE (multiple sequence comparison by log-expectation; version 3.5 or later; Edgar, 2004, Nucleic Acids Research 32: 1792-1797); MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When another GH61 polypeptide has diverged from the mature polypeptide of SEQ ID NO: 30 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the GH61 polypeptide variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple substitutions. Variants comprising multiple substitutions are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different substitutions. Where different substitutions can be introduced at a position, the different substitutions are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to isolated GH61 polypeptide variants, comprising a substitution at one or more (e.g., several) positions corresponding to positions 26, 32, 34, 40, 41, 42, 47, 56, 72, 102, 123, 138, 149, 152, 163, 164, 166, 169, 186, 200, 207, 213, 219, 222, 234, 246, 249, and 250 of the mature polypeptide of SEQ ID NO: 30, wherein the variants have cellulolytic enhancing activity.

### Variants

In an embodiment, the variant has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, to the amino acid sequence of the parent GH61 polypeptide.

In another embodiment, the variant has at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411.

In one aspect, the number of substitutions in the variants of the present invention is 1-28, e.g., 1, 2, 3, 4, 5, 6, 7, 8, 9, 10, 11, 12, 13, 14, 15, 16, 17, 18, 19, 20, 21, 22, 23, 24, 25, 26, 27, and 28 substitutions.

In another aspect, a variant comprises a substitution at one or more (e.g., several) positions corresponding to positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at two positions corresponding to any of positions 26, 32, 34, 40, 234, 246, 249, and 250. In another aspect, a variant comprises a substitution at three positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at four positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at five positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at six positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at seven positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at eight positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at nine positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at ten positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at eleven positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at twelve positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at thirteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at fourteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at fifteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at sixteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at seventeen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at eighteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at nineteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at twenty positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at twenty-one positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at twenty-two positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at twenty-three positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at twenty-four positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at twenty-five positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at twenty-six positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at twenty-seven positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250. In another aspect, a variant comprises a substitution at each position corresponding to positions 26, 32, 34, 40, 41, 42, 47, 56, 72, 102, 123, 138, 149, 152, 163, 164, 166, 169, 186, 200, 207, 213, 219, 222, 234, 246, 249, and 250.

In another aspect, the variant comprises or consists of a substitution at a position corresponding to position 26. In another aspect, the amino acid at a position corresponding to position 26 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ile. In another aspect, the variant comprises or consists of the substitution S26I of the mature polypeptide of SEQ ID NO: 30.

In another aspect, the variant comprises or consists of a substitution at a position corresponding to position 32. In another aspect, the amino acid at a position corresponding to position 32 is substituted with Ala, Arg, Asn, Asp, Cys, Gin, Glu, Gly, His, Ile, Leu, Lys, Met, Phe, Pro, Ser, Thr, Trp, Tyr, or Val, preferably with Glu or Ser. In another aspect, the variant comprises or consists of the substitution G32E or G32S of the mature polypeptide of SEQ ID NO: 30.

In another aspect, the variant comprises or consists of a substitution at a position corresponding to position 34. In another aspect, the amino acid at a position corresponding to position 34 is substituted with Ala, Arg, Asn, Asp, Cys, Gin, Glu, Gly, His, Ile, Leu, Lys, Met, Phe, Pro, Ser, Thr, Trp, Tyr, or Val, preferably with Phe. In another aspect, the variant comprises or consists of the substitution Y34F of the mature polypeptide of SEQ ID NO: 30.

In another aspect, the variant comprises or consists of a substitution at a position corresponding to position 40. In another aspect, the amino acid at a position corresponding to position 40 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ala. In another aspect, the variant comprises or consists of the substitution V40A of the mature polypeptide of SEQ ID NO: 30.

In another aspect, the variant comprises or consists of a substitution at a position corresponding to position 41. In another aspect, the amino acid at a position corresponding to position 41 is substituted with Ala, Arg, Asn, Asp, Cys, Gin, Glu, Gly, His, Ile, Leu, Lys, Met, Phe, Pro, Ser, Thr, Trp, Tyr, or Val, preferably with Thr. In another aspect, the variant comprises or consists of the substitution N41T of the mature polypeptide of SEQ ID NO: 30.

In another aspect, the variant comprises or consists of a substitution at a position corresponding to position 42. In another aspect, the amino acid at a position corresponding to position 42 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ile, Glu, or Val. In another aspect, the variant comprises or consists of the substitution Q42I, Q42E, or Q42V of the mature polypeptide of SEQ ID NO: 30.

In another aspect, the variant comprises or consists of a substitution at a position corresponding to position 47. In another aspect, the amino acid at a position corresponding to position 47 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu, Leu, or Arg. In another aspect, the variant comprises or consists of the substitution S47E, S47L, or S47R of the mature polypeptide of SEQ ID NO: 30.

In another aspect, the variant comprises or consists of a substitution at a position corresponding to position 56. In another aspect, the amino acid at a position corresponding to position 56 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys, Glu, or Thr. In another aspect, the variant comprises or consists of the substitution S56C, S56E, or S56T of the mature polypeptide of SEQ ID NO: 30.

In another aspect, the variant comprises or consists of a substitution at a position corresponding to position 72. In another aspect, the amino acid at a position corresponding to position 72 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gln or Thr. In another aspect, the variant comprises or consists of the substitution S72Q or S72T of the mature polypeptide of SEQ ID NO: 30.

In another aspect, the variant comprises or consists of a substitution at a position corresponding to position 102. In another aspect, the amino acid at a position corresponding to position 102 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys or Pro. In another aspect, the variant comprises or consists of the substitution T102K or T102P of the mature polypeptide of SEQ ID NO: 30.

In another aspect, the variant comprises or consists of a substitution at a position corresponding to position 123. In another aspect, the amino acid at a position corresponding to position 123 is substituted with Ala, Arg, Asn, Asp, Cys, Gln, Glu, Gly, His, Ile, Leu, Lys, Met, Phe, Pro, Ser, Thr, Trp, Tyr, or Val, preferably with Arg. In another aspect, the variant comprises or consists of the substitution A123R of the mature polypeptide of SEQ ID NO: 30.

In another aspect, the variant comprises or consists of a substitution at a position corresponding to position 138. In another aspect, the amino acid at a position corresponding to position 138 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys, Glu, Gly, Lys, Leu, or Met. In another aspect, the variant comprises or consists of the substitution Q138C, Q138E, Q138G, Q138K, Q138L, or Q138M of the mature polypeptide of SEQ ID NO: 30.

In another aspect, the variant comprises or consists of a substitution at a position corresponding to position 149. In another aspect, the amino acid at a position corresponding to position 149 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile. In another aspect, the variant comprises or consists of the substitution V149I of the mature polypeptide of SEQ ID NO: 30.

In another aspect, the variant comprises or consists of a substitution at a position corresponding to position 152. In another aspect, the amino acid at a position corresponding to position 152 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another aspect, the variant comprises or consists of the substitution D152S of the mature polypeptide of SEQ ID NO: 30.

In another aspect, the variant comprises or consists of a substitution at a position corresponding to position 163. In another aspect, the amino acid at a position corresponding to position 163 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu, Phe, or Val. In another aspect, the variant comprises or consists of the substitution T163E, T163F, or T163V of the mature polypeptide of SEQ ID NO: 30.

In another aspect, the variant comprises or consists of a substitution at a position corresponding to position 164. In another aspect, the amino acid at a position corresponding to position 164 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys or Leu. In another aspect, the variant comprises or consists of the substitution V164C or V164L of the mature polypeptide of SEQ ID NO: 30.

In another aspect, the variant comprises or consists of a substitution at a position corresponding to position 166. In another aspect, the amino acid at a position corresponding to position 166 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution I166L of the mature polypeptide of SEQ ID NO: 30.

In another aspect, the variant comprises or consists of a substitution at a position corresponding to position 169. In another aspect, the amino acid at a position corresponding to position 169 is substituted with Ala, Arg, Asn, Asp, Cys, Gln, Glu, Gly, His, Ile, Leu, Lys, Met, Phe, Pro, Ser, Thr, Trp, Tyr, or Val, preferably with Arg or Cys. In another aspect, the variant comprises or consists of the substitution S169R or S169C of the mature polypeptide of SEQ ID NO: 30. In another aspect, the variant comprises or consists of the substitution S173C of the mature polypeptide of SEQ ID NO: 36. The position in the Penicillium sp. (emersonii) GH61 mature polypeptide corresponding to position 169 in the A. fumigatus GH61 mature polypeptide is position 173.

In another aspect, the variant comprises or consists of a substitution at a position corresponding to position 186. In another aspect, the amino acid at a position corresponding to position 186 is substituted with Ala, Arg, Asn, Asp, Cys, Gin, Glu, Gly, His, Ile, Leu, Lys, Met, Phe, Pro, Ser, Thr, Trp, Tyr, or Val, preferably with Phe, Lys, Thr, or Tyr. In another aspect, the variant comprises or consists of the substitution S186F, S186K, S186T, or S186Y of the mature polypeptide of SEQ ID NO: 30.

In another aspect, the variant comprises or consists of a substitution at a position corresponding to position 200. In another aspect, the amino acid at a position corresponding to position 200 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ile or Val. In another aspect, the variant comprises or consists of the substitution F200I or F200V of the mature polypeptide of SEQ ID NO: 30.

In another aspect, the variant comprises or consists of a substitution at a position corresponding to position 207. In another aspect, the amino acid at a position corresponding to position 207 is substituted with Ala, Arg, Asn, Asp, Cys, Gin, Glu, Gly, His, Ile, Leu, Lys, Met, Phe, Pro, Ser, Thr, Trp, Tyr, or Val, preferably with Pro. In another aspect, the variant comprises or consists of the substitution G207P of the mature polypeptide of SEQ ID NO: 30.

In another aspect, the variant comprises or consists of a substitution at a position corresponding to position 213. In another aspect, the amino acid at a position corresponding to position 213 is substituted with Ala, Arg, Asn, Asp, Cys, Gin, Glu, Gly, His, Ile, Leu, Lys, Met, Phe, Pro, Ser, Thr, Trp, Tyr, or Val, preferably with Glu. In another aspect, the variant comprises or consists of the substitution S213E of the mature polypeptide of SEQ ID NO: 30.

In another aspect, the variant comprises or consists of a substitution at a position corresponding to position 219. In another aspect, the amino acid at a position corresponding to position 219 is substituted with Ala, Arg, Asn, Asp, Cys, Gin, Glu, Gly, His, Ile, Leu, Lys, Met, Phe, Pro, Ser, Thr, Trp, Tyr, or Val, preferably with Glu, Met, Gin, or Cys. In another aspect, the variant comprises or consists of the substitution S219E, S219M, S219Q, or S219C of the mature polypeptide of SEQ ID NO: 30.

In another aspect, the variant comprises or consists of a substitution at a position corresponding to position 222. In another aspect, the amino acid at a position corresponding to position 222 is substituted with Ala, Arg, Asn, Asp, Cys, Gin, Glu, Gly, His, Ile, Leu, Lys, Met, Phe, Pro, Ser, Thr, Trp, Tyr, or Val, preferably with Arg. In another aspect, the variant comprises or consists of the substitution K222R of the mature polypeptide of SEQ ID NO: 30.

In another aspect, the variant comprises or consists of a substitution at a position corresponding to position 234. In another aspect, the amino acid at a position corresponding to position 234 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gly or Lys. In another aspect, the variant comprises or consists of the substitution S234G or S234K of the mature polypeptide of SEQ ID NO: 30.

In another aspect, the variant comprises or consists of a substitution at a position corresponding to position 246. In another aspect, the amino acid at a position corresponding to position 246 is substituted with Ala, Arg, Asn, Asp, Cys, Gin, Glu, Gly, His, Ile, Leu, Lys, Met, Phe, Pro, Ser, Thr, Trp, Tyr, or Val, preferably with Pro. In another aspect, the variant comprises or consists of the substitution A246P of the mature polypeptide of SEQ ID NO: 30.

In another aspect, the variant comprises or consists of a substitution at a position corresponding to position 249. In another aspect, the amino acid at a position corresponding to position 249 is substituted with Ala, Arg, Asn, Asp, Cys, Gin, Glu, Gly, His, Ile, Leu, Lys, Met, Phe, Pro, Ser, Thr, Trp, Tyr, or Val, preferably with Gin, Arg, or Cys. In another aspect, the variant comprises or consists of the substitution N249Q, N249R, or N2490 of the mature polypeptide of SEQ ID NO: 30. In another aspect, the variant comprises or consists of the substitution F253C of the mature polypeptide of SEQ ID NO: 36. The position in the Penicillium sp. (emersonii) GH61 mature polypeptide corresponding to position 249 in the A. fumigatus GH61 mature polypeptide is position 253.

In another aspect, the variant comprises or consists of a substitution at a position corresponding to position 250. In another aspect, the amino acid at a position corresponding to position 250 is substituted with Ala, Arg, Asn, Asp, Cys, Gin, Glu, Gly, His, Ile, Leu, Lys, Met, Phe, Pro, Ser, Thr, Trp, Tyr, or Val, preferably with Cys. In another aspect, the variant comprises or consists of the substitution A250C of the mature polypeptide of SEQ ID NO: 30.

In another aspect, the variant comprises or consists of one or more (e.g., several) substitutions selected from the group consisting of S26I; G32E,S; Y34F; V40A; N41T; Q42I,E,V; S47E,L,R; S56C,E,T; S72Q,T; T102K,P; A123R; Q138C,E,G,K,L,M; V149I; D152S; T163E,F,V; V164C,L; I166L; S169R,C; S186F,K,T,Y; F200I,V; G207P; S213E; S219E,M,Q,C; K222R; S234G,K; A246P; N249Q,R,C; and A250C; or the one or more (e.g., several) substitutions selected from the group consisting of S26I; G32E,S; Y34F; V40A; N41T; Q42I,E,V; S47E,L,R; S56C,E,T; S72Q,T; T102K,P; A123R; Q138C,E,G,K,L,M; V149I; D152S; T163E,F,V; V164C,L; I166L; S169R,C; S186F,K,T,Y; F200I,V; G207P; S213E; S219E,M,Q,C; K222R; S234G,K; A246P; N249Q,R,C; and A250C at positions corresponding to the mature polypeptide of SEQ ID NO: 30 in other GH61 polypeptides described herein.

In each of the aspects below, the variant comprises or consists of the one or more (e.g., several) substitutions described below at positions corresponding to SEQ ID NO: 30 in other GH61 polypeptides described herein.

In another aspect, the variant comprises or consists of the substitutions S173C+F253C of the mature polypeptide of SEQ ID NO: 36. In another aspect, the variant comprises or consists of the substitutions L111V+D152S+M155L+A162W+Q138K+K229W of the mature polypeptide of SEQ ID NO: 30. In another aspect, the variant comprises or consists of the substitutions L111V+D152S+M155L+A162W+547E+K229W of the mature polypeptide of SEQ ID NO: 30. In another aspect, the variant comprises or consists of the substitutions L111V+D152S+M155L+A162W+S56A+K229W of the mature polypeptide of SEQ ID NO: 30. In another aspect, the variant comprises or consists of the substitutions L111V+D152S+M155L+A162W+T102K+K229W of the mature polypeptide of SEQ ID NO: 30. In another aspect, the variant comprises or consists of the substitutions L111V+D152S+M155L+A162W+S186T+K229W of the mature polypeptide of SEQ ID NO: 30. In another aspect, the variant comprises or consists of the substitutions L111V+D152S+M155L+A162W+K229W+S234G of the mature polypeptide of SEQ ID NO: 30. In another aspect, the variant comprises or consists of the substitutions L111V+D152S+M155L+A162W+T102K+E105K+K229W of the mature polypeptide of SEQ ID NO: 30. In another aspect, the variant comprises or consists of the substitutions L111V+D152S+M155L+A162W+Q138K+G188F+K229W of the mature polypeptide of SEQ ID NO: 30. In another aspect, the variant comprises or consists of the substitutions L111V+D152S+M155L+A162W+Q138K+V149I+G188F+K229W of the mature polypeptide of SEQ ID NO: 30. In another aspect, the variant comprises or consists of the substitutions L111V+D152S+M155L+A162W+S169C+G188F+K229W+A250C of the mature polypeptide of SEQ ID NO: 30. In another aspect, the variant comprises or consists of the substitutions L111V+D152S+M155L+A162W+S72T+Q138K+V149I+G188F+K229W of the mature polypeptide of SEQ ID NO: 30. In another aspect, the variant comprises or consists of the substitutions L111V+D152S+M155L+A162W+Q138K+V149I+G188F+G207P+K229W of the mature polypeptide of SEQ ID NO: 30.

The variants may further comprise one or more additional alterations, e.g., substitutions, insertions, or deletions at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

The variants of the present invention may further or even further comprise a substitution at one or more (e.g., several) positions corresponding to positions 111, 152, 155, and 162 of the mature polypeptide of SEQ ID NO: 30, wherein the variants have cellulolytic enhancing activity (WO 2012/044835).

In one aspect, the number of additional substitutions above in the variants of the present invention is 1-4, such as 1, 2, 3, or 4 substitutions.

In another aspect, the variant further or even further comprises a substitution at one or more (e.g., several) positions corresponding to positions 111, 152, 155, and 162. In another aspect, the variant further or even further comprises a substitution at two positions corresponding to any of positions 111, 152, 155, and 162. In another aspect, the variant further or even further comprises a substitution at three positions corresponding to any of positions 111, 152, 155, and 162. In another aspect, the variant further or even further comprises a substitution at each position corresponding to positions 111, 152, 155, and 162.

In another aspect, the variant further or even further comprises a substitution at a position corresponding to position 111. In another aspect, the amino acid at a position corresponding to position 111 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val. In another aspect, the variant further or even further comprises the substitution L111V of the mature polypeptide of SEQ ID NO: 30.

In another aspect, the variant further or even further comprises a substitution at a position corresponding to position 152. In another aspect, the amino acid at a position corresponding to position 152 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another aspect, the variant further or even further comprises the substitution D152S of the mature polypeptide of SEQ ID NO: 30.

In another aspect, the variant further or even further comprises a substitution at a position corresponding to position 155. In another aspect, the amino acid at a position corresponding to position 155 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant further or even further comprises the substitution M155L of the mature polypeptide of SEQ ID NO: 30.

In another aspect, the variant further or even further comprises a substitution at a position corresponding to position 162. In another aspect, the amino acid at a position corresponding to position 162 is substituted with Ala, Arg, Asn, Asp, Cys, Gln, Glu, Gly, His, Ile, Leu, Lys, Met, Phe, Pro, Ser, Thr, Trp, Tyr, or Val, preferably with Trp. In another aspect, the variant further or even further comprises the substitution A162W of the mature polypeptide of SEQ ID NO: 30.

In another aspect, the variant further or even further comprises one or more (e.g., several) substitutions selected from the group consisting of L111V, D152S, M155L, and A162W of the mature polypeptide of SEQ ID NO: 30, or the one or more (e.g., several) substitutions selected from the group consisting of L111V, D152S, M155L, and A162W at positions corresponding to the mature polypeptide of SEQ ID NO: 30 in other GH61 polypeptides described herein.

The variants of the present invention may further or even further comprise a substitution at one or more (e.g., several) positions corresponding to positions 96, 98, 200, 202, and 204 of the mature polypeptide of SEQ ID NO: 30, wherein the variants have cellulolytic enhancing activity (WO 2012/044836).

In one aspect, the number of additional substitutions above in the variants of the present invention is 1-5, such as 1, 2, 3, 4, or 5 substitutions.

In another aspect, the variant further or even further comprises a substitution at one or more (e.g., several) positions corresponding to positions 96, 98, 200, 202, and 204. In another aspect, the variant further or even further comprises a substitution at two positions corresponding to any of positions 96, 98, 200, 202, and 204. In another aspect, the variant further or even further comprises a substitution at three positions corresponding to any of positions 96, 98, 200, 202, and 204. In another aspect, the variant further or even further comprises a substitution at four positions corresponding to any of positions 96, 98, 200, 202, and 204. In another aspect, the variant further or even further comprises a substitution at each position corresponding to positions 96, 98, 200, 202, and 204.

In another aspect, the variant further or even further comprises a substitution at a position corresponding to position 96. In another aspect, the amino acid at a position corresponding to position 96 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val. In another aspect, the variant further or even further comprises the substitution I96V of the mature polypeptide of SEQ ID NO: 30.

In another aspect, the variant further or even further comprises a substitution at a position corresponding to position 98. In another aspect, the amino acid at a position corresponding to position 98 is substituted with Ala, Arg, Asn, Asp, Cys, Gin, Glu, Gly, His, Ile, Leu, Lys, Met, Phe, Pro, Ser, Thr, Trp, Tyr, or Val, preferably with Leu. In another aspect, the variant further or even further comprises the substitution F98L of the mature polypeptide of SEQ ID NO: 30.

In another aspect, the variant further or even further comprises a substitution at a position corresponding to position 200. In another aspect, the amino acid at a position corresponding to position 200 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ile. In another aspect, the variant further or even further comprises the substitution F200I of the mature polypeptide of SEQ ID NO: 30.

In another aspect, the variant further or even further comprises a substitution at a position corresponding to position 202. In another aspect, the amino acid at a position corresponding to position 202 is substituted with Ala, Arg, Asn, Asp, Cys, Gin, Glu, Gly, His, Ile, Leu, Lys, Met, Phe, Pro, Ser, Thr, Trp, Tyr, or Val, preferably with Leu. In another aspect, the variant further or even further comprises the substitution I202L of the mature polypeptide of SEQ ID NO: 30.

In another aspect, the variant further or even further comprises a substitution at a position corresponding to position 204. In another aspect, the amino acid at a position corresponding to position 204 is substituted with Ala, Arg, Asn, Asp, Cys, Gin, Glu, Gly, His, Ile, Leu, Lys, Met, Phe, Pro, Ser, Thr, Trp, Tyr, or Val, preferably with Val. In another aspect, the variant further or even further comprises the substitution I204V of the mature polypeptide of SEQ ID NO: 30.

In another aspect, the variant further or even further comprises one or more (e.g., several) substitutions selected from the group consisting of I96V, F98L, F200I, I202L, and I204V, or the one or more (e.g., several) substitutions selected from the group consisting of I96V, F98L, F200I, I202L, and I204V at positions corresponding to SEQ ID NO: 30 in other GH61 polypeptides described herein.

The variants of the present invention may further or even further comprise a substitution at one or more (e.g., several) positions corresponding to positions 105, 154, 188, 189, 216, and 229 of the mature polypeptide of SEQ ID NO: 30, wherein the variants have cellulolytic enhancing activity.

In one aspect, the number of additional substitutions above in the variants of the present invention is 1-6, e.g., 1, 2, 3, 4, 5, or 6 substitutions.

In another aspect, a variant further or even further comprises a substitution at one or more (e.g., several) positions corresponding to positions 105, 154, 188, 189, 216, and 229. In another aspect, a variant further or even further comprises a substitution at two positions corresponding to any of positions 105, 154, 188, 189, 216, and 229. In another aspect, a variant further or even further comprises a substitution at three positions corresponding to any of positions 105, 154, 188, 189, 216, and 229. In another aspect, a variant further or even further comprises a substitution at four positions corresponding to any of positions 105, 154, 188, 189, 216, and 229. In another aspect, a variant further or even further comprises a substitution at five positions corresponding to any of positions 105, 154, 188, 189, 216, and 229. In another aspect, a variant further or even further comprises a substitution at each position corresponding to positions 105, 154, 188, 189, 216, and 229.

In another aspect, the variant further or even further comprises a substitution at a position corresponding to position 105. In another aspect, the amino acid at a position corresponding to position 105 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro or Lys. In another aspect, the variant further or even further comprises of the substitution E105P or E105K of the mature polypeptide of SEQ ID NO: 30.

In another aspect, the variant further or even further comprises a substitution at a position corresponding to position 154. In another aspect, the amino acid at a position corresponding to position 154 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant further or even further comprises the substitution E154L of the mature polypeptide of SEQ ID NO: 30.

In another aspect, the variant further or even further comprises a substitution at a position corresponding to position 188. In another aspect, the amino acid at a position corresponding to position 188 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala or Trp. In another aspect, the variant further or even further comprises the substitution G188A or G188W of the mature polypeptide of SEQ ID NO: 30.

In another aspect, the variant further or even further comprises a substitution at a position corresponding to position 189. In another aspect, the amino acid at a position corresponding to position 189 is substituted with Ala, Arg, Asn, Asp, Cys, Gin, Glu, Gly, His, Ile, Leu, Lys, Met, Phe, Pro, Ser, Thr, Trp, Tyr, or Val, preferably with Lys. In another aspect, the variant further or even further comprises the substitution N189K of the mature polypeptide of SEQ ID NO: 30.

In another aspect, the variant further or even further comprises a substitution at a position corresponding to position 216. In another aspect, the amino acid at a position corresponding to position 216 is substituted with Ala, Arg, Asn, Asp, Cys, Gin, Glu, Gly, His, Ile, Leu, Lys, Met, Phe, Pro, Ser, Thr, Trp, Tyr, or Val, preferably with Leu or Tyr. In another aspect, the variant further or even further comprises the substitution A216L or A216Y of the mature polypeptide of SEQ ID NO: 30.

In another aspect, the variant further or even further comprises a substitution at a position corresponding to position 229. In another aspect, the amino acid at a position corresponding to position 229 is substituted with Ala, Arg, Asn, Asp, Cys, Gln, Glu, Gly, His, Ile, Leu, Lys, Met, Phe, Pro, Ser, Thr, Trp, Tyr, or Val, preferably with Trp, His, Ile, or Tyr. In another aspect, the variant further or even further comprises the substitution A229W, A229H, A229I, or A229Y of the mature polypeptide of SEQ ID NO: 30.

In another aspect, the variant further or even further comprises one or more (e.g., several) substitutions selected from the group consisting of E105P,K; E154L; G188A,W; N189K; A216L,Y; and A229W,H,I,Y, of the mature polypeptide of SEQ ID NO: 30, or the one or more (e.g., several) substitutions selected from the group consisting of E105P,K; E154L; G188A,W; N189K; A216L,Y; and A229W,H,I,Y at positions corresponding to the mature polypeptide of SEQ ID NO: 30 in other GH61 polypeptides described herein.

The variants may consist of at least 85% of the amino acid residues, e.g., at least 90% of the amino acid residues or at least 95% of the amino acid residues of the mature polypeptides of the corresponding parent GH61 polypeptides.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for cellulolytic enhancing activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. Essential amino acids in GH61 polypeptides correspond to positions 22, 107, 194, and/or 196 of the mature polypeptide of SEQ ID NO: 30.

In an embodiment, the variants have increased thermostability compared to their parent GH61 polypeptides.

In one aspect, the thermostability of the variant relative to the parent is determined at pH 3.0 and 45° C. In another aspect, the thermostability of the variant relative to the parent is determined at pH 3.0 and 50° C. In another aspect, the thermostability of the variant relative to the parent is determined at pH 3.0 and 55° C. In another aspect, the thermostability of the variant relative to the parent is determined at pH 3.0 and 60° C. In another aspect, the thermostability of the variant relative to the parent is determined at pH 3.0 and 62° C. In another aspect, the thermostability of the variant relative to the parent is determined at pH 3.0 and 65° C. In another aspect, the thermostability of the variant relative to the parent is determined at pH 3.0 and 68° C. In another aspect, the thermostability of the variant relative to the parent is determined at pH 3.0 and 70° C. In another aspect, the thermostability of the variant relative to the parent is determined at pH 3.0 and 72° C. In another aspect, the thermostability of the variant relative to the parent is determined at pH 3.0 and 75° C. In another aspect, the thermostability of the variant relative to the parent is determined at pH 3.0 and 80° C. In another aspect, the thermostability of the variant relative to the parent is determined at pH 3.0 and 85° C. In another aspect, the thermostability of the variant relative to the parent is determined at pH 3.0 and 90° C. In another aspect, the thermostability of the variant relative to the parent is determined at pH 3.0 and 95° C.

In another aspect, the thermostability of the variant relative to the parent is determined at pH 3.5 and 45° C. In another aspect, the thermostability of the variant relative to the parent is determined at pH 3.5 and 50° C. In another aspect, the thermostability of the variant relative to the parent is determined at pH 3.5 and 55° C. In another aspect, the thermostability of the variant relative to the parent is determined at pH 3.5 and 60° C. In another aspect, the thermostability of the variant relative to the parent is determined at pH 3.5 and 62° C. In another aspect, the thermostability of the variant relative to the parent is determined at pH 3.5 and 65° C. In another aspect, the thermostability of the variant relative to the parent is determined at pH 3.5 and 68° C. In another aspect, the thermostability of the variant relative to the parent is determined at pH 3.5 and 70° C. In another aspect, the thermostability of the variant relative to the parent is determined at pH 3.5 and 72° C. In another aspect, the thermostability of the variant relative to the parent is determined at pH 3.5 and 75° C. In another aspect, the thermostability of the variant relative to the parent is determined at pH 3.5 and 80° C. In another aspect, the thermostability of the variant relative to the parent is determined at pH 3.5 and 85° C. In another aspect, the thermostability of the variant relative to the parent is determined at pH 3.5 and 90° C. In another aspect, the thermostability of the variant relative to the parent is determined at pH 3.5 and 95° C.

In another aspect, the thermostability of the variant relative to the parent is determined at pH 4.0 and 45° C. In another aspect, the thermostability of the variant relative to the parent is determined at pH 4.0 and 50° C. In another aspect, the thermostability of the variant relative to the parent is determined at pH 4.0 and 55° C. In another aspect, the thermostability of the variant relative to the parent is determined at pH 4.0 and 60° C. In another aspect, the thermostability of the variant relative to the parent is determined at pH 4.0 and 62° C. In another aspect, the thermostability of the variant relative to the parent is determined at pH 4.0 and 65° C. In another aspect, the thermostability of the variant relative to the parent is determined at pH 4.0 and 68° C. In another aspect, the thermostability of the variant relative to the parent is determined at pH 4.0 and 70° C. In another aspect, the thermostability of the variant relative to the parent is determined at pH 4.0 and 72° C. In another aspect, the thermostability of the variant relative to the parent is determined at pH 4.0 and 75° C. In another aspect, the thermostability of the variant relative to the parent is determined at pH 4.0 and 80° C. In another aspect, the thermostability of the variant relative to the parent is determined at pH 4.0 and 85° C. In another aspect, the thermostability of the variant relative to the parent is determined at pH 4.0 and 90° C. In another aspect, the thermostability of the variant relative to the parent is determined at pH 4.0 and 95° C.

In another aspect, the thermostability of the variant relative to the parent is determined at pH 4.5 and 45° C. In another aspect, the thermostability of the variant relative to the parent is determined at pH 4.5 and 50° C. In another aspect, the thermostability of the variant relative to the parent is determined at pH 4.5 and 55° C. In another aspect, the thermostability of the variant relative to the parent is determined at pH 4.5 and 60° C. In another aspect, the thermostability of the variant relative to the parent is determined at pH 4.5 and 62° C. In another aspect, the thermostability of the variant relative to the parent is determined at pH 4.5 and 65° C. In another aspect, the thermostability of the variant relative to the parent is determined at pH 4.5 and 68° C. In another aspect, the thermostability of the variant relative to the parent is determined at pH 4.5 and 70° C. In another aspect, the thermostability of the variant relative to the parent is determined at pH 4.5 and 72° C. In another aspect, the thermostability of the variant relative to the parent is determined at pH 4.5 and 75° C. In another aspect, the thermostability of the variant relative to the parent is determined at pH 4.5 and 80° C. In another aspect, the thermostability of the variant relative to the parent is determined at pH 4.5 and 85° C. In another aspect, the thermostability of the variant relative to the parent is determined at pH 4.5 and 90° C. In another aspect, the thermostability of the variant relative to the parent is determined at pH 4.5 and 95° C.

In another aspect, the thermostability of the variant relative to the parent is determined at pH 5.0 and 45° C. In another aspect, the thermostability of the variant relative to the parent is determined at pH 5.0 and 50° C. In another aspect, the thermostability of the variant relative to the parent is determined at pH 5.0 and 55° C. In another aspect, the thermostability of the variant relative to the parent is determined at pH 5.0 and 60° C. In another aspect, the thermostability of the variant relative to the parent is determined at pH 5.0 and 62° C. In another aspect, the thermostability of the variant relative to the parent is determined at pH 5.0 and 65° C. In another aspect, the thermostability of the variant relative to the parent is determined at pH 5.0 and 68° C. In another aspect, the thermostability of the variant relative to the parent is determined at pH 5.0 and 70° C. In another aspect, the thermostability of the variant relative to the parent is determined at pH 5.0 and 72° C. In another aspect, the thermostability of the variant relative to the parent is determined at pH 5.0 and 75° C. In another aspect, the thermostability of the variant relative to the parent is determined at pH 5.0 and 80° C. In another aspect, the thermostability of the variant relative to the parent is determined at pH 5.0 and 85° C. In another aspect, the thermostability of the variant relative to the parent is determined at pH 5.0 and 90° C. In another aspect, the thermostability of the variant relative to the parent is determined at pH 5.0 and 95° C.

In another aspect, the thermostability of the variant relative to the parent is determined at pH 5.5 and 45° C. In another aspect, the thermostability of the variant relative to the parent is determined at pH 5.5 and 50° C. In another aspect, the thermostability of the variant relative to the parent is determined at pH 5.5 and 55° C. In another aspect, the thermostability of the variant relative to the parent is determined at pH 5.5 and 60° C. In another aspect, the thermostability of the variant relative to the parent is determined at pH 5.5 and 62° C. In another aspect, the thermostability of the variant relative to the parent is determined at pH 5.5 and 65° C. In another aspect, the thermostability of the variant relative to the parent is determined at pH 5.5 and 68° C. In another aspect, the thermostability of the variant relative to the parent is determined at pH 5.5 and 70° C. In another aspect, the thermostability of the variant relative to the parent is determined at pH 5.5 and 72° C. In another aspect, the thermostability of the variant relative to the parent is determined at pH 5.5 and 75° C. In another aspect, the thermostability of the variant relative to the parent is determined at pH 5.5 and 80° C. In another aspect, the thermostability of the variant relative to the parent is determined at pH 5.5 and 85° C. In another aspect, the thermostability of the variant relative to the parent is determined at pH 5.5 and 90° C. In another aspect, the thermostability of the variant relative to the parent is determined at pH 5.5 and 95° C.

In another aspect, the thermostability of the variant relative to the parent is determined at pH 6.0 and 45° C. In another aspect, the thermostability of the variant relative to the parent is determined at pH 6.0 and 50° C. In another aspect, the thermostability of the variant relative to the parent is determined at pH 6.0 and 55° C. In another aspect, the thermostability of the variant relative to the parent is determined at pH 6.0 and 60° C. In another aspect, the thermostability of the variant relative to the parent is determined at pH 6.0 and 62° C. In another aspect, the thermostability of the variant relative to the parent is determined at pH 6.0 and 65° C. In another aspect, the thermostability of the variant relative to the parent is determined at pH 6.0 and 68° C. In another aspect, the thermostability of the variant relative to the parent is determined at pH 6.0 and 70° C. In another aspect, the thermostability of the variant relative to the parent is determined at pH 6.0 and 72° C. In another aspect, the thermostability of the variant relative to the parent is determined at pH 6.0 and 75° C. In another aspect, the thermostability of the variant relative to the parent is determined at pH 6.0 and 80° C. In another aspect, the thermostability of the variant relative to the parent is determined at pH 6.0 and 85° C. In another aspect, the thermostability of the variant relative to the parent is determined at pH 6.0 and 90° C. In another aspect, the thermostability of the variant relative to the parent is determined at pH 6.0 and 95° C.

In another aspect, the thermostability of the variant relative to the parent is determined at pH 6.5 and 45° C. In another aspect, the thermostability of the variant relative to the parent is determined at pH 6.5 and 50° C. In another aspect, the thermostability of the variant relative to the parent is determined at pH 6.5 and 55° C. In another aspect, the thermostability of the variant relative to the parent is determined at pH 6.5 and 60° C. In another aspect, the thermostability of the variant relative to the parent is determined at pH 6.5 and 62° C. In another aspect, the thermostability of the variant relative to the parent is determined at pH 6.5 and 65° C. In another aspect, the thermostability of the variant relative to the parent is determined at pH 6.5 and 68° C. In another aspect, the thermostability of the variant relative to the parent is determined at pH 6.5 and 70° C. In another aspect, the thermostability of the variant relative to the parent is determined at pH 6.5 and 72° C. In another aspect, the thermostability of the variant relative to the parent is determined at pH 6.5 and 75° C. In another aspect, the thermostability of the variant relative to the parent is determined at pH 6.5 and 80° C. In another aspect, the thermostability of the variant relative to the parent is determined at pH 6.5 and 85° C. In another aspect, the thermostability of the variant relative to the parent is determined at pH 6.5 and 90° C. In another aspect, the thermostability of the variant relative to the parent is determined at pH 6.5 and 95° C.

In another aspect, the thermostability of the variant relative to the parent is determined at pH 7.0 and 45° C. In another aspect, the thermostability of the variant relative to the parent is determined at pH 7.0 and 50° C. In another aspect, the thermostability of the variant relative to the parent is determined at pH 7.0 and 55° C. In another aspect, the thermostability of the variant relative to the parent is determined at pH 7.0 and 60° C. In another aspect, the thermostability of the variant relative to the parent is determined at pH 7.0 and 62° C. In another aspect, the thermostability of the variant relative to the parent is determined at pH 7.0 and 65° C. In another aspect, the thermostability of the variant relative to the parent is determined at pH 7.0 and 68° C. In another aspect, the thermostability of the variant relative to the parent is determined at pH 7.0 and 70° C. In another aspect, the thermostability of the variant relative to the parent is determined at pH 7.0 and 72° C. In another aspect, the thermostability of the variant relative to the parent is determined at pH 7.0 and 75° C. In another aspect, the thermostability of the variant relative to the parent is determined at pH 7.0 and 80° C. In another aspect, the thermostability of the variant relative to the parent is determined at pH 7.0 and 85° C. In another aspect, the thermostability of the variant relative to the parent is determined at pH 7.0 and 90° C. In another aspect, the thermostability of the variant relative to the parent is determined at pH 7.0 and 95° C.

In another aspect, the thermostability of the variant relative to the parent is determined at pH 7.5 and 45° C. In another aspect, the thermostability of the variant relative to the parent is determined at pH 7.5 and 50° C. In another aspect, the thermostability of the variant relative to the parent is determined at pH 7.5 and 55° C. In another aspect, the thermostability of the variant relative to the parent is determined at pH 7.5 and 60° C. In another aspect, the thermostability of the variant relative to the parent is determined at pH 7.5 and 62° C. In another aspect, the thermostability of the variant relative to the parent is determined at pH 7.5 and 65° C. In another aspect, the thermostability of the variant relative to the parent is determined at pH 7.5 and 68° C. In another aspect, the thermostability of the variant relative to the parent is determined at pH 7.5 and 70° C. In another aspect, the thermostability of the variant relative to the parent is determined at pH 7.5 and 72° C. In another aspect, the thermostability of the variant relative to the parent is determined at pH 7.5 and 75° C. In another aspect, the thermostability of the variant relative to the parent is determined at pH 7.5 and 80° C. In another aspect, the thermostability of the variant relative to the parent is determined at pH 7.5 and 85° C. In another aspect, the thermostability of the variant relative to the parent is determined at pH 7.5 and 90° C. In another aspect, the thermostability of the variant relative to the parent is determined at pH 7.5 and 95° C.

In another aspect, the thermostability of the variant relative to the parent is determined at pH 8.0 and 45° C. In another aspect, the thermostability of the variant relative to the parent is determined at pH 8.0 and 50° C. In another aspect, the thermostability of the variant relative to the parent is determined at pH 8.0 and 55° C. In another aspect, the thermostability of the variant relative to the parent is determined at pH 8.0 and 60° C. In another aspect, the thermostability of the variant relative to the parent is determined at pH 8.0 and 62° C. In another aspect, the thermostability of the variant relative to the parent is determined at pH 8.0 and 65° C. In another aspect, the thermostability of the variant relative to the parent is determined at pH 8.0 and 68° C. In another aspect, the thermostability of the variant relative to the parent is determined at pH 8.0 and 70° C. In another aspect, the thermostability of the variant relative to the parent is determined at pH 8.0 and 72° C. In another aspect, the thermostability of the variant relative to the parent is determined at pH 8.0 and 75° C. In another aspect, the thermostability of the variant relative to the parent is determined at pH 8.0 and 80° C. In another aspect, the thermostability of the variant relative to the parent is determined at pH 8.0 and 85° C. In another aspect, the thermostability of the variant relative to the parent is determined at pH 8.0 and 90° C. In another aspect, the thermostability of the variant relative to the parent is determined at pH 8.0 and 95° C.

In another aspect, the thermostability of the variant relative to the parent is determined at pH 8.5 and 45° C. In another aspect, the thermostability of the variant relative to the parent is determined at pH 8.5 and 50° C. In another aspect, the thermostability of the variant relative to the parent is determined at pH 8.5 and 55° C. In another aspect, the thermostability of the variant relative to the parent is determined at pH 8.5 and 60° C. In another aspect, the thermostability of the variant relative to the parent is determined at pH 8.5 and 62° C. In another aspect, the thermostability of the variant relative to the parent is determined at pH 8.5 and 65° C. In another aspect, the thermostability of the variant relative to the parent is determined at pH 8.5 and 68° C. In another aspect, the thermostability of the variant relative to the parent is determined at pH 8.5 and 70° C. In another aspect, the thermostability of the variant relative to the parent is determined at pH 8.5 and 72° C. In another aspect, the thermostability of the variant relative to the parent is determined at pH 8.5 and 75° C. In another aspect, the thermostability of the variant relative to the parent is determined at pH 8.5 and 80° C. In another aspect, the thermostability of the variant relative to the parent is determined at pH 8.5 and 85° C. In another aspect, the thermostability of the variant relative to the parent is determined at pH 8.5 and 90° C. In another aspect, the thermostability of the variant relative to the parent is determined at pH 8.5 and 95° C.

In another aspect, the thermostability of the variant relative to the parent is determined at pH 9.0 and 45° C. In another aspect, the thermostability of the variant relative to the parent is determined at pH 9.0 and 50° C. In another aspect, the thermostability of the variant relative to the parent is determined at pH 9.0 and 55° C. In another aspect, the thermostability of the variant relative to the parent is determined at pH 9.0 and 60° C. In another aspect, the thermostability of the variant relative to the parent is determined at pH 9.0 and 62° C. In another aspect, the thermostability of the variant relative to the parent is determined at pH 9.0 and 65° C. In another aspect, the thermostability of the variant relative to the parent is determined at pH 9.0 and 68° C. In another aspect, the thermostability of the variant relative to the parent is determined at pH 9.0 and 70° C. In another aspect, the thermostability of the variant relative to the parent is determined at pH 9.0 and 72° C. In another aspect, the thermostability of the variant relative to the parent is determined at pH 9.0 and 75° C. In another aspect, the thermostability of the variant relative to the parent is determined at pH 9.0 and 80° C. In another aspect, the thermostability of the variant relative to the parent is determined at pH 9.0 and 85° C. In another aspect, the thermostability of the variant relative to the parent is determined at pH 9.0 and 90° C. In another aspect, the thermostability of the variant relative to the parent is determined at pH 9.0 and 95° C.

In each of the aspects above, the thermostability of the variant relative to the parent can be determined by incubating the variant and parent for 1 minute. In each of the aspects above, the thermostability of the variant relative to the parent can be determined by incubating the variant and parent for 5 minutes. In each of the aspects above, the thermostability of the variant relative to the parent can be determined by incubating the variant and parent for 10 minutes. In each of the aspects above, the thermostability of the variant relative to the parent can be determined by incubating the variant and parent for 15 minutes. In each of the aspects above, the thermostability of the variant relative to the parent can be determined by incubating the variant and parent for 20 minutes. In each of the aspects above, the thermostability of the variant relative to the parent can be determined by incubating the variant and parent for 25 minutes. In each of the aspects above, the thermostability of the variant relative to the parent can be determined by incubating the variant and parent for 30 minutes. In each of the aspects above, the thermostability of the variant relative to the parent can be determined by incubating the variant and parent for 45 minutes. In each of the aspects above, the thermostability of the variant relative to the parent can be determined by incubating the variant and parent for 60 minutes. A time period longer than 60 minutes can also be used.

In one aspect, the thermostability of the variant having cellulolytic enhancing activity is increased at least 1.01-fold, e.g., at least 1.05-fold, at least 1.1-fold, at least 1.2-fold, at least 1.3-fold, at least 1.4-fold, at least 1.5-fold, at least 1.8-fold, at least 2-fold, at least 5-fold, at least 10-fold, at least 15-fold, at least 20-fold, at least 25-fold, at least 30-fold, at least 50-fold, at least 75-fold, or at least 100-fold compared to more thermostable than the parent.

### Parent GH61 Polypeptides

The parent GH61 polypeptide may be any GH61 polypeptide having cellulolytic enhancing activity.

The parent GH61 polypeptide may be (a) a polypeptide having at least 60% sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411; (b) a polypeptide encoded by a polynucleotide that hybridizes under at least low stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410, or (ii) the full-length complement of (i); or (c) a polypeptide encoded by a polynucleotide having at least 60% sequence identity to the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410.

In one aspect, the parent has a sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have cellulolytic enhancing activity.

In one embodiment, the amino acid sequence of the parent differs by up to 10 amino acids, e.g., 1, 2, 3, 4, 5, 6, 7, 8, 9, or 10 from the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411.

In another embodiment, the parent comprises or consists of the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411.

In another embodiment, the parent comprises or consists of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411.

In another embodiment, the parent is a fragment containing at least 85% of the amino acid residues, e.g., at least 90% of the amino acid residues or at least 95% of the amino acid residues of the mature polypeptide of a GH61 polypeptide.

In another embodiment, the parent is an allelic variant of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411.

In another aspect, the parent is encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410, or the full-length complements thereof (Sambrook et al., 1989, Molecular Cloning, A Laboratory Manual, 2d edition, Cold Spring Harbor, New York).

The polynucleotide of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410, or subsequences thereof, as well as the polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411, or fragments thereof, may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410, or subsequences thereof, the carrier material is used in a Southern blot.

For purposes of the present invention, hybridization indicates that the polynucleotide hybridizes to a labeled nucleic acid probe corresponding to (i) SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410; (ii) the mature polypeptide coding sequence thereof; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410.

In another embodiment, the nucleic acid probe is a polynucleotide that encodes the polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411; the mature polypeptide thereof; or a fragment thereof.

In another embodiment, the nucleic acid probe is SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410.

In another aspect, the parent is encoded by a polynucleotide having a sequence identity to the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

The parent may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one embodiment, the parent is secreted extracellularly.

The parent may be a bacterial GH61 polypeptide. For example, the parent may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces GH61 polypeptide, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma GH61 polypeptide.

In one embodiment, the parent is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis GH61 polypeptide.

The parent may be a fungal GH61 polypeptide. For example, the parent may be a yeast GH61 polypeptide such as a Candida, Kluyveromyces, Pichia, Saccharomyces, Schizosaccharomyces, or Yarrowia GH61 polypeptide; or a filamentous fungal GH61 polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptosphaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria GH61 polypeptide.

In another embodiment, the parent is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis GH61 polypeptide.

In another embodiment, the parent is an Acremonium cellulolyticus, Aspergillus aculeatus, Aspergillus awamori, Aspergillus foetidus, Aspergillus fumigatus, Aspergillus japonicus, Aspergillus lentulus, Aspergillus nidulans, Aspergillus niger, Aspergillus oryzae, Aspergillus terreus, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fennellia nivea, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium emersonii, Penicillium funiculosum, Penicillium pinophilum, Penicillium purpurogenum, Phanerochaete chrysosporium, Talaromyces leycettanus, Thermoascus aurantiacus, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride GH61 polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

### Preparation of Variants

The present invention also relates to methods for obtaining a GH61 polypeptide variant having cellulolytic enhancing activity, comprising: (a) introducing into a parent GH61 polypeptide a substitution at one or more (e.g., several) positions corresponding to positions 26, 32, 34, 40, 41, 42, 47, 56, 72, 102, 123, 138, 149, 152, 163, 164, 166, 169, 186, 200, 207, 213, 219, 222, 234, 246, 249, and 250 of the mature polypeptide of SEQ ID NO: 30, wherein the variant has cellulolytic enhancing activity; and optionally (b) recovering the variant. In one aspect, the methods further or even further comprise introducing into the parent GH61 polypeptide a substitution at one or more (e.g., several) positions corresponding to positions 111, 152, 155, and 162 of the mature polypeptide of SEQ ID NO: 30, wherein the variant has cellulolytic enhancing activity. In another aspect, the methods further or even further comprise introducing into the parent GH61 polypeptide a substitution at one or more (e.g., several) positions corresponding to positions 96, 98, 200, 202, and 204 of the mature polypeptide of SEQ ID NO: 30, wherein the variant has cellulolytic enhancing activity. In another aspect, the methods further or even further comprise introducing into the parent GH61 polypeptide a substitution at one or more (e.g., several) positions corresponding to positions 105, 154, 188, 189, 216, and 229 of the mature polypeptide of SEQ ID NO: 30, wherein the variant has cellulolytic enhancing activity,

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent. Any site-directed mutagenesis procedure can be used in the present invention. There are many commercial kits available that can be used to prepare variants.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Site-saturation mutagenesis systematically replaces a polypeptide coding sequence with sequences encoding all 19 amino acids at one or more (e.g., several) specific positions (Parikh and Matsumura, 2005, J. Mol. Biol. 352: 621-628).

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to isolated polynucleotides encoding GH61 polypeptide variants of the present invention.

### Nucleic Acid Constructs

The present invention also relates to nucleic acid constructs comprising a polynucleotide encoding a GH61 polypeptide variant of the present invention operably linked to one or more control sequences that direct the expression of the coding sequence in a suitable host cell under conditions compatible with the control sequences.

The polynucleotide may be manipulated in a variety of ways to provide for expression of a GH61 polypeptide variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide recognized by a host cell for expression of a polynucleotide encoding a variant of the present invention. The promoter contains transcriptional control sequences that mediate the expression of the GH61 polypeptide variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the GH61 polypeptide variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the GH61 polypeptide variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the GH61 polypeptide variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a GH61 polypeptide variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCI B 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a GH61 polypeptide variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the GH61 polypeptide variant and the signal peptide sequence is positioned next to the N-terminus of the propeptide sequence.

It may also be desirable to add regulatory sequences that regulate expression of the GH61 polypeptide variant relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked to the regulatory sequence.

### Expression Vectors

The present invention also relates to recombinant expression vectors comprising a polynucleotide encoding a GH61 polypeptide variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system. The vector preferably contains an element(s) that permits integration of the vector into the host cell's genome or autonomous replication of the vector in the cell independent of the genome.

For integration into the host cell genome, the vector may rely on the polynucleotide's sequence encoding the GH61 polypeptide variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a GH61 polypeptide variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a GH61 polypeptide variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a GH61 polypeptide variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma. The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp. 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a GH61 polypeptide variant, comprising: (a) cultivating a recombinant host cell of the present invention under conditions conducive for production of the variant; and optionally (b) recovering the variant.

The host cells are cultivated in a nutrient medium suitable for production of the GH61 polypeptide variant using methods known in the art. For example, the cells may be cultivated by multi-well plates such as 24, 48, or 96 well plates, shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The GH61 polypeptide variant may be detected using methods known in the art that are specific for the variant. These detection methods include, but are not limited to, use of specific antibodies, formation of an enzyme product, or disappearance of an enzyme substrate. For example, an enzyme assay may be used to determine the activity of the variant. A specific assay for GH61 proteins is to incubate the GH61 polypeptide variants with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X100 for 24-96 hours at 40° C. followed by an assay of this reaction to determine the glucose released from the PASC. See the assay described in Example 5.

The GH61 polypeptide variants may be recovered using methods known in the art. For example, the variant may be recovered from the nutrient medium by conventional procedures including, but not limited to, collection, centrifugation, filtration, extraction, spray-drying, evaporation, or precipitation. In one aspect, a whole fermentation broth comprising a variant of the present invention is recovered.

The GH61 polypeptide variants may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the GH61 polypeptide variant is not recovered, but rather a host cell of the present invention expressing the variant is used as a source of the variant.

### Fermentation Broth Formulations or Cell Compositions

The present invention also relates to a fermentation broth formulation or a cell composition comprising a variant of the present invention. The fermentation broth product further comprises additional ingredients used in the fermentation process, such as, for example, cells (including, the host cells containing the gene encoding the variant of the present invention which are used to produce the variant), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The fermentation broth formulations or cell compositions may further comprise multiple enzymatic activities, such as one or more (e.g., several) enzymes selected from the group consisting of a cellulase, a hemicellulase, an esterase, an expansin, a laccase, a ligninolytic enzyme, a pectinase, a peroxidase, a protease, and a swollenin. The fermentation broth formulations or cell compositions may also comprise one or more (e.g., several) enzymes selected from the group consisting of a hydrolase, an isomerase, a ligase, a lyase, an oxidoreductase, or a transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis (e.g., expression of cellulase and/or glucosidase enzyme(s)). In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

Examples are given below of preferred uses of the compositions of the present invention. The dosage of the composition and other conditions under which the composition is used may be determined on the basis of methods known in the art.

### Enzyme Compositions

The present invention also relates to compositions comprising a variant of the present invention. Preferably, the compositions are enriched in such a variant. The term “enriched” indicates that the cellulolytic enhancing activity of the composition has been increased, e.g., with an enrichment factor of at least 1.1.

The compositions may comprise a variant of the present invention as the major enzymatic component, e.g., a mono-component composition. Alternatively, the compositions may comprise multiple enzymatic activities, such as one or more (e.g., several) enzymes selected from the group consisting of a cellulase, a hemicellulase, a GH61 polypeptide, an esterase, an expansin, a laccase, a ligninolytic enzyme, a pectinase, a peroxidase, a protease, and a swollenin. The compositions may also comprise one or more (e.g., several) enzymes selected from the group consisting of a hydrolase, an isomerase, a ligase, a lyase, an oxidoreductase, or a transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

### Uses

The present invention is also directed to the following processes for using the GH61 polypeptide variants having cellulolytic enhancing activity, or compositions thereof.

The present invention also relates to processes for degrading or converting a cellulosic material, comprising: treating the cellulosic material with an enzyme composition in the presence of a GH61 polypeptide variant of the present invention. In one aspect, the processes further comprise recovering the degraded or converted cellulosic material. Soluble products of degradation or conversion of the cellulosic material can be separated from insoluble cellulosic material using a method known in the art such as, for example, centrifugation, filtration, or gravity settling.

The present invention also relates to processes of producing a fermentation product, comprising: (a) saccharifying a cellulosic material with an enzyme composition in the presence of a GH61 polypeptide variant of the present invention; (b) fermenting the saccharified cellulosic material with one or more (e.g., several) fermenting microorganisms to produce the fermentation product; and (c) recovering the fermentation product from the fermentation.

The present invention also relates to processes of fermenting a cellulosic material, comprising: fermenting the cellulosic material with one or more (e.g., several) fermenting microorganisms, wherein the cellulosic material is saccharified with an enzyme composition in the presence of a GH61 polypeptide variant of the present invention. In one aspect, the fermenting of the cellulosic material produces a fermentation product. In another aspect, the processes further comprise recovering the fermentation product from the fermentation.

The processes of the present invention can be used to saccharify the cellulosic material to fermentable sugars and to convert the fermentable sugars to many useful fermentation products, e.g., fuel (ethanol, n-butanol, isobutanol, biodiesel, jet fuel) and/or platform chemicals (e.g., acids, alcohols, ketones, gases, oils, and the like). The production of a desired fermentation product from the cellulosic material typically involves pretreatment, enzymatic hydrolysis (saccharification), and fermentation.

The processing of the cellulosic material according to the present invention can be accomplished using methods conventional in the art. Moreover, the processes of the present invention can be implemented using any conventional biomass processing apparatus configured to operate in accordance with the invention.

Hydrolysis (saccharification) and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF); and direct microbial conversion (DMC), also sometimes called consolidated bioprocessing (CBP). SHF uses separate process steps to first enzymatically hydrolyze the cellulosic material to fermentable sugars, e.g., glucose, cellobiose, and pentose monomers, and then ferment the fermentable sugars to ethanol. In SSF, the enzymatic hydrolysis of the cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, DC, 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (e.g., several) steps where the same organism is used to produce the enzymes for conversion of the cellulosic material to fermentable sugars and to convert the fermentable sugars into a final product (Lynd et al., 2002, Microbiol. Mol. Biol. Reviews 66: 506-577). It is understood herein that any method known in the art comprising pretreatment, enzymatic hydrolysis (saccharification), fermentation, or a combination thereof, can be used in the practicing the processes of the present invention.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

Pretreatment. In practicing the processes of the present invention, any pretreatment process known in the art can be used to disrupt plant cell wall components of the cellulosic material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technol. 100: 10-18; Mosier et al., 2005, Bioresource Technol. 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40).

The cellulosic material can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

The cellulosic material can be pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

Steam Pretreatment. In steam pretreatment, the cellulosic material is heated to disrupt the plant cell wall components, including lignin, hemicellulose, and cellulose to make the cellulose and other fractions, e.g., hemicellulose, accessible to enzymes. The cellulosic material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

Chemical Pretreatment: The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, supra; Schell et al., 2004, Bioresource Technology 91: 179-188; Lee et al., 1999, Adv. Biochem. Eng. Biotechnol. 65: 93-115).

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment.

Lime pretreatment is performed with calcium oxide or calcium hydroxide at temperatures of 85-150° C. and residence times from 1 hour to several days (Wyman et al., 2005, Bioresource Technol. 96: 1959-1966; Mosier et al., 2005, Bioresource Technol. 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technol. 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the cellulosic material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, Bioresource Technology 96: 673-686, and U.S. Published Application 2002/0164730.

In one aspect, the chemical pretreatment is preferably carried out as a dilute acid treatment, and more preferably as a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one aspect, the acid concentration is in the range from preferably 0.01 to 10 wt. % acid, e.g., 0.05 to 5 wt. % acid or 0.1 to 2 wt. % acid. The acid is contacted with the cellulosic material and held at a temperature in the range of preferably 140-200° C., e.g., 165-190° C., for periods ranging from 1 to 60 minutes.

In another aspect, pretreatment takes place in an aqueous slurry. In preferred aspects, the cellulosic material is present during pretreatment in amounts preferably between 10-80 wt %, e.g., 20-70 wt % or 30-60 wt %, such as around 40 wt %. The pretreated cellulosic material can be unwashed or washed using any method known in the art, e.g., washed with water.

Mechanical Pretreatment or Physical Pretreatment: The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The cellulosic material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperature in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, in a preferred aspect, the cellulosic material is subjected to physical (mechanical) or chemical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from the cellulosic material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, DC, 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, DC, chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

Saccharification. In the hydrolysis step, also known as saccharification, the cellulosic material, e.g., pretreated, is hydrolyzed to break down cellulose and/or hemicellulose to fermentable sugars, such as glucose, cellobiose, xylose, xylulose, arabinose, mannose, galactose, and/or soluble oligosaccharides. The hydrolysis is performed enzymatically by an enzyme composition in the presence of a GH61 polypeptide variant of the present invention. The enzymes of the compositions can be added simultaneously or sequentially.

Enzymatic hydrolysis is preferably carried out in a suitable aqueous environment under conditions that can be readily determined by one skilled in the art. In one aspect, hydrolysis is performed under conditions suitable for the activity of the enzyme(s), i.e., optimal for the enzyme(s). The hydrolysis can be carried out as a fed batch or continuous process where the cellulosic material is fed gradually to, for example, an enzyme containing hydrolysis solution.

The saccharification is generally performed in stirred-tank reactors or fermentors under controlled pH, temperature, and mixing conditions. Suitable process time, temperature and pH conditions can readily be determined by one skilled in the art. For example, the saccharification can last up to 200 hours, but is typically performed for preferably about 12 to about 120 hours, e.g., about 16 to about 72 hours or about 24 to about 48 hours. The temperature is in the range of preferably about 25° C. to about 70° C., e.g., about 30° C. to about 65° C., about 40° C. to about 60° C., or about 50° C. to about 55° C. The pH is in the range of preferably about 3 to about 8, e.g., about 3.5 to about 7, about 4 to about 6, or about 5 to about 5.5. The dry solids content is in the range of preferably about 5 to about 50 wt %, e.g., about 10 to about 40 wt % or about 20 to about 30 wt %.

The enzyme compositions can comprise any protein useful in degrading the cellulosic material.

In one aspect, the enzyme composition comprises or further comprises one or more (e.g., several) proteins selected from the group consisting of a cellulase, a GH61 polypeptide having cellulolytic enhancing activity, a hemicellulase, an esterase, an expansin, a ligninolytic enzyme, an oxidoreductase, a pectinase, a protease, and a swollenin. In another aspect, the cellulase is preferably one or more (e.g., several) enzymes selected from the group consisting of an endoglucanase, a cellobiohydrolase, and a beta-glucosidase. In another aspect, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. In another aspect, the oxidoreductase is preferably one or more (e.g., several) enzymes selected from the group consisting of a catalase, a laccase, and a peroxidase.

In another aspect, the enzyme composition comprises one or more (e.g., several) cellulolytic enzymes. In another aspect, the enzyme composition comprises or further comprises one or more (e.g., several) hemicellulolytic enzymes. In another aspect, the enzyme composition comprises one or more (e.g., several) cellulolytic enzymes and one or more (e.g., several) hemicellulolytic enzymes. In another aspect, the enzyme composition comprises one or more (e.g., several) enzymes selected from the group of cellulolytic enzymes and hemicellulolytic enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises a GH61 polypeptide having cellulolytic enhancing activity. In another aspect, the enzyme composition comprises an endoglucanase and a GH61 polypeptide having cellulolytic enhancing activity. In another aspect, the enzyme composition comprises a cellobiohydrolase and a GH61 polypeptide having cellulolytic enhancing activity. In another aspect, the enzyme composition comprises a beta-glucosidase and a GH61 polypeptide having cellulolytic enhancing activity. In another aspect, the enzyme composition comprises an endoglucanase and a cellobiohydrolase. In another aspect, the enzyme composition comprises an endoglucanase and a cellobiohydrolase I, a cellobiohydrolase II, or a combination of a cellobiohydrolase I and a cellobiohydrolase II. In another aspect, the enzyme composition comprises an endoglucanase and a beta-glucosidase. In another aspect, the enzyme composition comprises a beta-glucosidase and a cellobiohydrolase. In another aspect, the enzyme composition comprises a beta-glucosidase and a cellobiohydrolase I, a cellobiohydrolase II, or a combination of a cellobiohydrolase I and a cellobiohydrolase II In another aspect, the enzyme composition comprises an endoglucanase, a GH61 polypeptide having cellulolytic enhancing activity, and a cellobiohydrolase. In another aspect, the enzyme composition comprises an endoglucanase, a GH61 polypeptide having cellulolytic enhancing activity, and a cellobiohydrolase I, a cellobiohydrolase II, or a combination of a cellobiohydrolase I and a cellobiohydrolase II. In another aspect, the enzyme composition comprises an endoglucanase, a beta-glucosidase, and a GH61 polypeptide having cellulolytic enhancing activity. In another aspect, the enzyme composition comprises a beta-glucosidase, a GH61 polypeptide having cellulolytic enhancing activity, and a cellobiohydrolase. In another aspect, the enzyme composition comprises a beta-glucosidase, a GH61 polypeptide having cellulolytic enhancing activity, and a cellobiohydrolase I, a cellobiohydrolase II, or a combination of a cellobiohydrolase I and a cellobiohydrolase II. In another aspect, the enzyme composition comprises an endoglucanase, a beta-glucosidase, and a cellobiohydrolase. In another aspect, the enzyme composition comprises an endoglucanase, a beta-glucosidase, and a cellobiohydrolase I, a cellobiohydrolase II, or a combination of a cellobiohydrolase I and a cellobiohydrolase II. In another aspect, the enzyme composition comprises an endoglucanase, a cellobiohydrolase, a beta-glucosidase, and a GH61 polypeptide having cellulolytic enhancing activity. In another aspect, the enzyme composition comprises an endoglucanase, a beta-glucosidase, a GH61 polypeptide having cellulolytic enhancing activity, and a cellobiohydrolase I, a cellobiohydrolase II, or a combination of a cellobiohydrolase I and a cellobiohydrolase II.

In another aspect, the enzyme composition comprises an acetylmannan esterase. In another aspect, the enzyme composition comprises an acetylxylan esterase. In another aspect, the enzyme composition comprises an arabinanase (e.g., alpha-L-arabinanase). In another aspect, the enzyme composition comprises an arabinofuranosidase (e.g., alpha-L-arabinofuranosidase). In another aspect, the enzyme composition comprises a coumaric acid esterase. In another aspect, the enzyme composition comprises a feruloyl esterase. In another aspect, the enzyme composition comprises a galactosidase (e.g., alpha-galactosidase and/or beta-galactosidase). In another aspect, the enzyme composition comprises a glucuronidase (e.g., alpha-D-glucuronidase). In another aspect, the enzyme composition comprises a glucuronoyl esterase. In another aspect, the enzyme composition comprises a mannanase. In another aspect, the enzyme composition comprises a mannosidase (e.g., beta-mannosidase). In another aspect, the enzyme composition comprises a xylanase. In a preferred aspect, the xylanase is a Family xylanase. In another preferred aspect, the xylanase is a Family 11 xylanase. In another aspect, the enzyme composition comprises a xylosidase (e.g., beta-xylosidase).

In another aspect, the enzyme composition comprises an esterase. In another aspect, the enzyme composition comprises an expansin. In another aspect, the enzyme composition comprises a ligninolytic enzyme. In a preferred aspect, the ligninolytic enzyme is a manganese peroxidase. In another preferred aspect, the ligninolytic enzyme is a lignin peroxidase. In another preferred aspect, the ligninolytic enzyme is a H₂O₂-producing enzyme. In another aspect, the enzyme composition comprises a pectinase. In another aspect, the enzyme composition comprises an oxidoreductase. In another preferred aspect, the oxidoreductase is a catalase. In another preferred aspect, the oxidoreductase is a laccase. In another preferred aspect, the oxidoreductase is a peroxidase. In another aspect, the enzyme composition comprises a protease. In another aspect, the enzyme composition comprises a swollenin.

In the processes of the present invention, the enzyme(s) can be added prior to or during saccharification, saccharification and fermentation, or fermentation.

One or more (e.g., several) components of the enzyme composition may be native proteins, recombinant proteins, or a combination of native proteins and recombinant proteins. For example, one or more (e.g., several) components may be native proteins of a cell, which is used as a host cell to express recombinantly one or more (e.g., several) other components of the enzyme composition. It is understood herein that the recombinant proteins may be heterologous (e.g., foreign) and native to the host cell. One or more (e.g., several) components of the enzyme composition may be produced as monocomponents, which are then combined to form the enzyme composition. The enzyme composition may be a combination of multicomponent and monocomponent protein preparations.

The enzymes used in the processes of the present invention may be in any form suitable for use, such as, for example, a fermentation broth formulation or a cell composition, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The optimum amounts of the enzymes and the GH61 polypeptide variants depend on several factors including, but not limited to, the mixture of cellulolytic enzymes and/or hemicellulolytic enzymes, the cellulosic material, the concentration of cellulosic material, the pretreatment(s) of the cellulosic material, temperature, time, pH, and inclusion of a fermenting organism (e.g., for Simultaneous Saccharification and Fermentation).

In one aspect, an effective amount of cellulolytic or hemicellulolytic enzyme to the cellulosic material is about 0.5 to about 50 mg, e.g., about 0.5 to about 40 mg, about 0.5 to about 25 mg, about 0.75 to about 20 mg, about 0.75 to about 15 mg, about 0.5 to about 10 mg, or about 2.5 to about 10 mg per g of the cellulosic material.

In another aspect, an effective amount of a GH61 polypeptide variant to the cellulosic material is about 0.01 to about 50.0 mg, e.g., about 0.01 to about 40 mg, about 0.01 to about 30 mg, about 0.01 to about 20 mg, about 0.01 to about 10 mg, about 0.01 to about 5 mg, about 0.025 to about 1.5 mg, about 0.05 to about 1.25 mg, about 0.075 to about 1.25 mg, about 0.1 to about 1.25 mg, about 0.15 to about 1.25 mg, or about 0.25 to about 1.0 mg per g of the cellulosic material.

In another aspect, an effective amount of a GH61 polypeptide variant to cellulolytic or hemicellulolytic enzyme is about 0.005 to about 1.0 g, e.g., about 0.01 to about 1.0 g, about 0.15 to about 0.75 g, about 0.15 to about 0.5 g, about 0.1 to about 0.5 g, about 0.1 to about 0.25 g, or about 0.05 to about 0.2 g per g of cellulolytic or hemicellulolytic enzyme.

The polypeptides having cellulolytic enzyme activity or hemicellulolytic enzyme activity as well as other proteins/polypeptides useful in the degradation of the cellulosic material (collectively hereinafter “polypeptides having enzyme activity”) can be derived or obtained from any suitable origin, including, archaeal, bacterial, fungal, yeast, plant, or animal origin. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained by, for example, site-directed mutagenesis or shuffling.

A polypeptide having enzyme activity may be a bacterial polypeptide. For example, the polypeptide may be a Gram-positive bacterial polypeptide such as a Bacillus, Streptococcus, Streptomyces, Staphylococcus, Enterococcus, Lactobacillus, Lactococcus, Clostridium, Geobacillus, Caldicellulosiruptor, Acidothermus, Thermobifidia, or Oceanobacillus polypeptide having enzyme activity, or a Gram-negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having enzyme activity.

The polypeptide having enzyme activity may also be a fungal polypeptide, and more preferably a yeast polypeptide such as a Candida, Kluyveromyces, Pichia, Saccharomyces, Schizosaccharomyces, or Yarrowia polypeptide having enzyme activity; or more preferably a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptosphaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide having enzyme activity.

In one aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having enzyme activity.

In another aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, Trichoderma viride, or Trichophaea saccata polypeptide having enzyme activity.

Chemically modified or protein engineered mutants of polypeptides having enzyme activity may also be used.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host is preferably a heterologous host (enzyme is foreign to host), but the host may under certain conditions also be a homologous host (enzyme is native to host). Monocomponent cellulolytic proteins may also be prepared by purifying such a protein from a fermentation broth.

In one aspect, the one or more (e.g., several) cellulolytic enzymes comprise a commercial cellulolytic enzyme preparation. Examples of commercial cellulolytic enzyme preparations suitable for use in the present invention include, for example, CELLIC® CTec (Novozymes A/S), CELLIC® CTec2 (Novozymes A/S), CELLIC® CTec3 (Novozymes A/S), CELLUCLAST™ (Novozymes A/S), NOVOZYM™ 188 (Novozymes A/S), SPEZYME™ CP (Genencor Int.), ACCELERASE™ TRIO (DuPont), FILTRASE® NL (DSM); METHAPLUS® S/L 100 (DSM), ROHAMENT™ 7069 W (Rohm GmbH), or ALTERNAFUEL® CMAX3™ (Dyadic International, Inc.). The cellulase enzymes are added in amounts effective from about 0.001 to about 5.0 wt. % of solids, e.g., about 0.025 to about 4.0 wt. % of solids or about 0.005 to about 2.0 wt. % of solids.

Examples of bacterial endoglucanases that can be used in the processes of the present invention, include, but are not limited to, Acidothermus cellulolyticus endoglucanase (WO 91/05039; WO 93/15186; U.S. Pat. No. 5,275,944; WO 96/02551; U.S. Pat. No. 5,536,655; WO 00/70031; WO 05/093050), Erwinia carotovora endoglucanase (Saarilahti et al., 1990, Gene 90: 9-14), Thermobifida fusca endoglucanase III (WO 05/093050), and Thermobifida fusca endoglucanase V (WO 05/093050).

Examples of fungal endoglucanases that can be used in the present invention, include, but are not limited to, Trichoderma reesei endoglucanase I (Penttila et al., 1986, Gene 45: 253-263, Trichoderma reesei Cel7B endoglucanase I (GenBank: M15665), Trichoderma reesei endoglucanase II (Saloheimo et al., 1988, Gene 63:11-22), Trichoderma reesei Cel5A endoglucanase II (GenBank: M19373), Trichoderma reesei endoglucanase III (Okada et al., 1988, Appl. Environ. Microbiol. 64: 555-563, GenBank: AB003694), Trichoderma reesei endoglucanase V (Saloheimo et al., 1994, Molecular Microbiology 13: 219-228, GenBank: Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Fusarium oxysporum endoglucanase (GenBank: L29381), Humicola grisea var. thermoidea endoglucanase (Gen Bank:AB003107), Melanocarpus albomyces endoglucanase (GenBank: MAL515703), Neurospora crassa endoglucanase (GenBank: XM_324477), Humicola insolens endoglucanase V, Myceliophthora thermophila CBS 117.65 endoglucanase, Thermoascus aurantiacus endoglucanase I (GenBank: AF487830), and Trichoderma reesei strain No. VTT-D-80133 endoglucanase (GenBank: M15665).

Examples of cellobiohydrolases useful in the present invention include, but are not limited to, Aspergillus aculeatus cellobiohydrolase II (WO 2011/059740), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Penicillium occitanis cellobiohydrolase I (GenBank: AY690482), Talaromyces emersonii cellobiohydrolase I (GenBank: AF439936), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

Examples of beta-glucosidases useful in the present invention include, but are not limited to, beta-glucosidases from Aspergillus aculeatus (Kawaguchi et al., 1996, Gene 173: 287-288), Aspergillus fumigatus (WO 2005/047499), Aspergillus niger (Dan et al., 2000, J. Biol. Chem. 275: 4973-4980), Aspergillus oryzae (WO 02/095014), Penicillium brasilianum IBT 20888 (WO 2007/019442 and WO 2010/088387), Thielavia terrestris (WO 2011/035029), and Trichophaea saccata (WO 2007/019442).

The beta-glucosidase may be a fusion protein. In one aspect, the beta-glucosidase is an Aspergillus oryzae beta-glucosidase variant BG fusion protein (WO 2008/057637) or an Aspergillus oryzae beta-glucosidase fusion protein (WO 2008/057637).

Other useful endoglucanases, cellobiohydrolases, and beta-glucosidases are disclosed in numerous Glycosyl Hydrolase families using the classification according to Henrissat, 1991, Biochem. J. 280: 309-316, and Henrissat and Bairoch, 1996, Biochem. J. 316: 695-696.

Other cellulolytic enzymes that may be used in the present invention are described in WO 98/13465, WO 98/015619, WO 98/015633, WO 99/06574, WO 99/10481, WO 99/025847, WO 99/031255, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. Nos. 5,457,046, 5,648,263, and 5,686,593.

In the processes of the present invention, any GH61 polypeptide having cellulolytic enhancing activity can be used as a component of the enzyme composition.

Examples of GH61 polypeptides useful in the processes of the present invention include, but are not limited to, GH61 polypeptides from Thielavia terrestris (WO 2005/074647, WO 2008/148131, and WO 2011/035027), Thermoascus aurantiacus (WO 2005/074656 and WO 2010/065830), Trichoderma reesei (WO 2007/089290), Myceliophthora thermophila (WO 2009/085935, WO 2009/085859, WO 2009/085864, and WO 2009/085868), Aspergillus fumigatus (WO 2010/138754), Penicillium pinophilum (WO 2011/005867), Thermoascus sp. (WO 2011/039319), Penicillium sp. (WO 2011/041397), Thermoascus crustaceous (WO 2011/041504), Aspergillus aculeatus (WO 2012/125925), Thermomyces lanuginosus (WO 2012/113340, WO 12/129699, and WO 2012/130964), Aurantiporus alborubescens (WO 2012/122477), Trichophaea saccata (WO 2012/122477), Penicillium thomii (WO 2012/122477), Talaromyces stipitatus (WO 2012/135659), Humicola insolens (WO 2012/146171), Malbranchea cinnamomea (WO 2012/101206), Talaromyces leycettanus (WO 2012/101206), and Chaetomium thermophilum (WO 2012/101206).

In one aspect, the GH61 polypeptide variant and GH61 polypeptide having cellulolytic enhancing activity is used in the presence of a soluble activating divalent metal cation according to WO 2008/151043, e.g., manganese or copper.

In another aspect, the GH61 polypeptide variant and GH61 polypeptide having cellulolytic enhancing activity is used in the presence of a dioxy compound, a bicyclic compound, a heterocyclic compound, a nitrogen-containing compound, a quinone compound, a sulfur-containing compound, or a liquor obtained from a pretreated cellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

In one aspect, such a compound is added at a molar ratio of the compound to glucosyl units of cellulose of about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻². In another aspect, an effective amount of such a compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about μM to about 10 mM, about 5 μM to about 5 mM, or about 0.1 mM to about 1 mM.

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc., under conditions as described herein, and the soluble contents thereof. A liquor for cellulolytic enhancement of a GH61 polypeptide can be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and a GH61 polypeptide during hydrolysis of a cellulosic substrate by a cellulase preparation. The liquor can be separated from the treated material using a method standard in the art, such as filtration, sedimentation, or centrifugation.

In one aspect, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, or about 10⁻³ to about 10⁻² g per g of cellulose.

In one aspect, the one or more (e.g., several) hemicellulolytic enzymes comprise a commercial hemicellulolytic enzyme preparation. Examples of commercial hemicellulolytic enzyme preparations suitable for use in the present invention include, for example, SHEARZYME™ (Novozymes A/S), CELLIC® HTec (Novozymes A/S), CELLIC® HTec2 (Novozymes A/S), CELLIC® HTec3 (Novozymes A/S), VISCOZYME® (Novozymes A/S), ULTRAFLO® (Novozymes A/S), PULPZYME® HC (Novozymes A/S), MULTIFECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK), ALTERNA FUEL 100P (Dyadic), and ALTERNA FUEL 200P (Dyadic).

Examples of xylanases useful in the processes of the present invention include, but are not limited to, xylanases from Aspergillus aculeatus (GeneSeqP:AAR63790; WO 94/21785), Aspergillus fumigatus (WO 2006/078256), Penicillium pinophilum (WO 2011/041405), Penicillium sp. (WO 2010/126772), Talaromyces lanuginosus GH11 (WO 2012/130965), Talaromyces thermophilus GH11 (WO 2012/13095), Thielavia terrestris NRRL 8126 (WO 2009/079210), and Trichophaea saccata GH10 (WO 2011/057083).

Examples of beta-xylosidases useful in the processes of the present invention include, but are not limited to, beta-xylosidases from Neurospora crassa (SwissProt:Q7SOW4), Trichoderma reesei (UniProtKB/TrEMBL:Q92458), Talaromyces emersonii (SwissProt:Q8X212), and Talaromyces thermophilus GH11 (WO 2012/13095).

Examples of acetylxylan esterases useful in the processes of the present invention include, but are not limited to, acetylxylan esterases from Aspergillus aculeatus (WO 2010/108918), Chaetomium globosum (UniProt:Q2GWX4), Chaetomium gracile (GeneSeqP:AAB82124), Humicola insolens DSM 1800 (WO 2009/073709), Hypocrea jecorina (WO 2005/001036), Myceliophtera thermophila (WO 2010/014880), Neurospora crassa (UniProt: q7s259), Phaeosphaeria nodorum (UniProt:Q0UHJ1), and Thielavia terrestris NRRL 8126 (WO 2009/042846).

Examples of feruloyl esterases (ferulic acid esterases) useful in the processes of the present invention include, but are not limited to, feruloyl esterases form Humicola insolens DSM 1800 (WO 2009/076122), Neosartorya fischeri (UniProt:A1D9T4), Neurospora crassa (UniProt:Q9HGR3), Penicillium aurantiogriseum (WO 2009/127729), and Thielavia terrestris (WO 2010/053838 and WO 2010/065448).

Examples of arabinofuranosidases useful in the processes of the present invention include, but are not limited to, arabinofuranosidases from Aspergillus niger (GeneSeqP:AAR94170), Humicola insolens DSM 1800 (WO 2006/114094 and WO 2009/073383), and M. giganteus (WO 2006/114094).

Examples of alpha-glucuronidases useful in the processes of the present invention include, but are not limited to, alpha-glucuronidases from Aspergillus clavatus (UniProt:alcc12), Aspergillus fumigatus (SwissProt:Q4WW45), Aspergillus niger (UniProt:Q96WX9), Aspergillus terreus (Swiss Prot: Q0CJP9), Humicola insolens (WO 2010/014706), Penicillium aurantiogriseum (WO 2009/068565), Talaromyces emersonii (UniProt:Q8X211), and Trichoderma reesei (UniProt:Q99024).

Examples of oxidoreductases useful in the processes of the present invention include, but are not limited to, Aspergillus fumigatus catalase, Aspergillus lentilus catalase, Aspergillus niger catalase, Aspergillus oryzae catalase, Humicola insolens catalase, Neurospora crassa catalase, Penicillium emersonii catalase, Scytalidium thermophilum catalase, Talaromyces stipitatus catalase, Thermoascus aurantiacus catalase, Coprinus cinereus laccase, Myceliophthora thermophila laccase, Polyporus pinsitus laccase, Pycnoporus cinnabarinus laccase, Rhizoctonia solani laccase, Streptomyces coelicolor laccase, Coprinus cinereus peroxidase, Soy peroxidase, and Royal palm peroxidase.

The polypeptides having enzyme activity used in the processes of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, C A, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, N Y, 1986).

The fermentation can be any method of cultivation of a cell resulting in the expression or isolation of an enzyme or protein. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

Fermentation. The fermentable sugars obtained from the hydrolyzed cellulosic material can be fermented by one or more (e.g., several) fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In the fermentation step, sugars, released from the cellulosic material as a result of the pretreatment and enzymatic hydrolysis steps, are fermented to a product, e.g., ethanol, by a fermenting organism, such as yeast. Hydrolysis (saccharification) and fermentation can be separate or simultaneous.

Any suitable hydrolyzed cellulosic material can be used in the fermentation step in practicing the present invention. The material is generally selected based on economics, i.e., costs per equivalent sugar potential, and recalcitrance to enzymatic conversion.

The term “fermentation medium” is understood herein to refer to a medium before the fermenting microorganism(s) is(are) added, such as, a medium resulting from a saccharification process, as well as a medium used in a simultaneous saccharification and fermentation process (SSF).

“Fermenting microorganism” refers to any microorganism, including bacterial and fungal organisms, suitable for use in a desired fermentation process to produce a fermentation product. The fermenting organism can be hexose and/or pentose fermenting organisms, or a combination thereof. Both hexose and pentose fermenting organisms are well known in the art. Suitable fermenting microorganisms are able to ferment, i.e., convert, sugars, such as glucose, xylose, xylulose, arabinose, maltose, mannose, galactose, and/or oligosaccharides, directly or indirectly into the desired fermentation product. Examples of bacterial and fungal fermenting organisms producing ethanol are described by Lin et al., 2006, Appl. Microbiol. Biotechnol. 69: 627-642.

Examples of fermenting microorganisms that can ferment hexose sugars include bacterial and fungal organisms, such as yeast. Yeast include strains of Candida, Kluyveromyces, and Saccharomyces, e.g., Candida sonorensis, Kluyveromyces marxianus, and Saccharomyces cerevisiae.

Examples of fermenting organisms that can ferment pentose sugars in their native state include bacterial and fungal organisms, such as some yeast. Xylose fermenting yeast include strains of Candida, preferably C. sheatae or C. sonorensis; and strains of Pichia, e.g., P. stipitis, such as P. stipitis CBS 5773. Pentose fermenting yeast include strains of Pachysolen, preferably P. tannophilus. Organisms not capable of fermenting pentose sugars, such as xylose and arabinose, may be genetically modified to do so by methods known in the art.

Examples of bacteria that can efficiently ferment hexose and pentose to ethanol include, for example, Bacillus coagulans, Clostridium acetobutylicum, Clostridium thermocellum, Clostridium phytofermentans, Geobacillus sp., Thermoanaerobacter saccharolyticum, and Zymomonas mobilis (Philippidis, 1996, supra).

Other fermenting organisms include strains of Bacillus, such as Bacillus coagulans; Candida, such as C. sonorensis, C. methanosorbosa, C. diddensiae, C. parapsilosis, C. naedodendra, C. blankii, C. entomophilia, C. brassicae, C. pseudotropicalis, C. boidinii, C. utilis, and C. scehatae; Clostridium, such as C. acetobutylicum, C. thermocellum, and C. phytofermentans; E. coli, especially E. coli strains that have been genetically modified to improve the yield of ethanol; Geobacillus sp.; Hansenula, such as Hansenula anomala; Klebsiella, such as K. oxytoca; Kluyveromyces, such as K. marxianus, K. lactis, K. thermotolerans, and K. fragilis; Schizosaccharomyces, such as S. pombe; Thermoanaerobacter, such as Thermoanaerobacter saccharolyticum; and Zymomonas, such as Zymomonas mobilis.

Commercially available yeast suitable for ethanol production include, e.g., BIOFERM™ AFT and XR (NABC—North American Bioproducts Corporation, GA, USA), ETHANOL RED™ yeast (Fermentis/Lesaffre, USA), FALI™ (Fleischmann's Yeast, USA), FERMIOL™ (DSM Specialties), GERT STRAND™ (Gert Strand AB, Sweden), and SUPERSTART™ and THERMOSACC™ fresh yeast (Ethanol Technology, WI, USA).

In an aspect, the fermenting microorganism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (co-fermentation) (Chen and Ho, 1993, Appl. Biochem. Biotechnol. 39-40: 135-147; Ho et al., 1998, Appl. Environ. Microbiol. 64: 1852-1859; Kotter and Ciriacy, 1993, Appl. Microbiol. Biotechnol. 38: 776-783; Walfridsson et al., 1995, Appl. Environ. Microbiol. 61: 4184-4190; Kuyper et al., 2004, FEMS Yeast Research 4: 655-664; Beall et al., 1991, Biotech. Bioeng. 38: 296-303; Ingram et al., 1998, Biotechnol. Bioeng. 58: 204-214; Zhang et al., 1995, Science 267: 240-243; Deanda et al., 1996, Appl. Environ. Microbiol. 62: 4465-4470; WO 03/062430).

It is well known in the art that the organisms described above can also be used to produce other substances, as described herein.

The fermenting microorganism is typically added to the degraded cellulosic material or hydrolysate and the fermentation is performed for about 8 to about 96 hours, e.g., about 24 to about 60 hours. The temperature is typically between about 26° C. to about 60° C., e.g., about 32° C. or 50° C., and about pH 3 to about pH 8, e.g., pH 4-5, 6, or 7.

In one aspect, the yeast and/or another microorganism are applied to the degraded cellulosic material and the fermentation is performed for about 12 to about 96 hours, such as typically 24-60 hours. In another aspect, the temperature is preferably between about 20° C. to about 60° C., e.g., about 25° C. to about 50° C., about 32° C. to about 50° C., or about 32° C. to about 50° C., and the pH is generally from about pH 3 to about pH 7, e.g., about pH 4 to about pH 7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

A fermentation stimulator can be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

Fermentation products: A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g. pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide. The fermentation product can also be protein as a high value product.

In a preferred aspect, the fermentation product is an alcohol. It will be understood that the term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603.

In another preferred aspect, the fermentation product is an alkane. The alkane may be an unbranched or a branched alkane. The alkane can be, but is not limited to, pentane, hexane, heptane, octane, nonane, decane, undecane, or dodecane.

In another preferred aspect, the fermentation product is a cycloalkane. The cycloalkane can be, but is not limited to, cyclopentane, cyclohexane, cycloheptane, or cyclooctane.

In another preferred aspect, the fermentation product is an alkene. The alkene may be an unbranched or a branched alkene. The alkene can be, but is not limited to, pentene, hexene, heptene, or octene.

In another preferred aspect, the fermentation product is an amino acid. The organic acid can be, but is not limited to, aspartic acid, glutamic acid, glycine, lysine, serine, or threonine. See, for example, Richard and Margaritis, 2004, Biotechnology and Bioengineering 87(4): 501-515.

In another preferred aspect, the fermentation product is a gas. The gas can be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another preferred aspect, the fermentation product is isoprene.

In another preferred aspect, the fermentation product is a ketone. It will be understood that the term “ketone” encompasses a substance that contains one or more ketone moieties. The ketone can be, but is not limited to, acetone.

In another preferred aspect, the fermentation product is an organic acid. The organic acid can be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another preferred aspect, the fermentation product is polyketide.

Recovery. The fermentation product(s) can be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

### Detergent Compositions

The present invention also relates to detergent compositions comprising a GH61 polypeptide variant of the present invention and a surfactant. A GH61 polypeptide variant of the present invention may be added to and thus become a component of a detergent composition.

The detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. In one aspect, the present invention also relates to methods for cleaning or washing a hard surface or laundry, the method comprising contacting the hard surface or the laundry with a detergent composition of the present invention.

In a specific aspect, the present invention provides a detergent additive comprising a GH61 polypeptide variant of the invention. The detergent additive as well as the detergent composition may comprise one or more (e.g., several) enzymes selected from the group consisting of an amylase, arabinase, cutinase, carbohydrase, cellulase, galactanase, laccase, lipase, mannanase, oxidase, pectinase, peroxidase, protease, and xylanase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and PCT/DK98/00299.

Commercially available cellulases include CELLUZYME™, and CAREZYME™ (Novozymes A/S), CLAZINASE™, and PURADAX HA™ (Genencor International Inc.), and KAC-500(B)™ (Kao Corporation).

Proteases: Suitable proteases include those of animal, vegetable or microbial origin. Microbial origin is preferred. Chemically modified or protein engineered mutants are included. The protease may be a serine protease or a metalloprotease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g., of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235, and 274.

Preferred commercially available protease enzymes include ALCALASE™, SAVINASE™ PRIMASE™, DURALASE™, ESPERASE™, and KANNASE™ (Novozymes A/S), MAXATASE™, MAXACAL™, MAXAPEM™, PROPERASE™, PURAFECT™, PURAFECT OXP™, FN2™, and FN3™ (Genencor International Inc.).

Lipases: Suitable lipases include those of bacterial or fungal origin. Chemically modified or protein engineered mutants are included. Examples of useful lipases include lipases from Humicola (synonym Thermomyces), e.g., from H. lanuginosa (T. lanuginosus) as described in EP 258 068 and EP 305 216 or from H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131: 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include LIPOLASE™ and LIPOLASE ULTRA™ (Novozymes A/S).

Amylases: Suitable amylases (a and/or 13) include those of bacterial or fungal origin.

Chemically modified or protein engineered mutants are included. Amylases include, for example, α-amylases obtained from Bacillus, e.g., a special strain of Bacillus licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are DURAMYL™, TERMAMYL™, FUNGAMYL™ and BAN™ (Novozymes A/S), RAPIDASE™ and PURASTAR™ (from Genencor International Inc.).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include GUARDZYME™ (Novozymes A/S).

The detergent enzyme(s) may be included in a detergent composition by adding separate additives containing one or more (e.g., several) enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The detergent composition of the invention may be in any convenient form, e.g., a bar, a tablet, a powder, a granule, a paste, or a liquid. A liquid detergent may be aqueous, typically containing up to 70% water and 0-30% organic solvent, or non-aqueous.

The detergent composition comprises one or more (e.g., several) surfactants, which may be non-ionic including semi-polar and/or anionic and/or cationic and/or zwitterionic. The surfactants are typically present at a level of from 0.1% to 60% by weight.

When included therein the detergent will usually contain from about 1% to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid, or soap.

When included therein the detergent will usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

The detergent may contain 0-65% of a detergent builder or complexing agent such as zeolite, diphosphate, triphosphate, phosphonate, carbonate, citrate, nitrilotriacetic acid, ethylenediaminetetraacetic acid, diethylenetriaminepentaacetic acid, alkyl- or alkenylsuccinic acid, soluble silicates, or layered silicates (e.g., SKS-6 from Hoechst).

The detergent may comprise one or more (e.g., several) polymers. Examples are carboxymethylcellulose, poly(vinylpyrrolidone), poly (ethylene glycol), poly(vinyl alcohol), poly(vinylpyridine-N-oxide), poly(vinylimidazole), polycarboxylates such as polyacrylates, maleic/acrylic acid copolymers, and lauryl methacrylate/acrylic acid copolymers.

The detergent may contain a bleaching system which may comprise a H₂O₂ source such as perborate or percarbonate which may be combined with a peracid-forming bleach activator such as tetraacetylethylenediamine or nonanoyloxybenzenesulfonate. Alternatively, the bleaching system may comprise peroxyacids of, for example, the amide, imide, or sulfone type.

The enzyme(s) of the detergent composition of the invention may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in, for example, WO 92/19709 and WO 92/19708.

The detergent may also contain other conventional detergent ingredients such as, e.g., fabric conditioners including clays, foam boosters, suds suppressors, anti-corrosion agents, soil-suspending agents, anti-soil redeposition agents, dyes, bactericides, optical brighteners, hydrotropes, tarnish inhibitors, or perfumes.

In the detergent compositions, any enzyme may be added in an amount corresponding to 0.01-100 mg of enzyme protein per liter of wash liquor, preferably 0.05-5 mg of enzyme protein per liter of wash liquor, in particular 0.1-1 mg of enzyme protein per liter of wash liquor.

In the detergent compositions, a GH61 polypeptide variant of the present invention having cellulolytic enhancing activity may be added in an amount corresponding to 0.001-100 mg of protein, preferably 0.005-50 mg of protein, more preferably 0.01-25 mg of protein, even more preferably 0.05-10 mg of protein, most preferably 0.05-5 mg of protein, and even most preferably 0.01-1 mg of protein per liter of wash liquor.

A GH61 polypeptide variant having cellulolytic enhancing activity of the present invention may also be incorporated in the detergent formulations disclosed in WO 97/07202, which is hereby incorporated by reference.

### Plants

The present invention also relates to isolated plants, e.g., a transgenic plant, plant part, or plant cell, comprising a polynucleotide of the present invention so as to express and produce a GH61 polypeptide variant in recoverable quantities. The variant may be recovered from the plant or plant part. Alternatively, the plant or plant part containing the variant may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing a variant may be constructed in accordance with methods known in the art. In short, the plant or plant cell is constructed by incorporating one or more expression constructs encoding a variant into the plant host genome or chloroplast genome and propagating the resulting modified plant or plant cell into a transgenic plant or plant cell.

The expression construct is conveniently a nucleic acid construct that comprises a polynucleotide encoding a variant operably linked with appropriate regulatory sequences required for expression of the polynucleotide in the plant or plant part of choice. Furthermore, the expression construct may comprise a selectable marker useful for identifying plant cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences (Sticklen, 2008, Nature Reviews 9: 433-443), is determined, for example, on the basis of when, where, and how the variant is desired to be expressed. For instance, the expression of the gene encoding a variant may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a variant in the plant. For instance, the promoter enhancer element may be an intron that is placed between the promoter and the polynucleotide encoding a variant. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase.

In addition to direct transformation of a particular plant genotype with a construct of the present invention, transgenic plants may be made by crossing a plant having the construct to a second plant lacking the construct. For example, a construct encoding a variant can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention. Crossing results in the introduction of a transgene into a plant line by cross pollinating a starting line with a donor plant line. Non-limiting examples of such steps are described in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a variant of the present invention comprising: (a) cultivating a transgenic plant or a plant cell comprising a polynucleotide encoding the variant under conditions conducive for production of the variant; and optionally (b) recovering the variant.

The present invention is further described by the following examples that should not be construed as limiting the scope of the invention.

### EXAMPLES

### Strains

Aspergillus oryzae strain PFJO218 (amy⁻, alp⁻, Npl⁻, CPA⁻, KA⁻, pyrG⁻, ku70⁻; U.S. Patent Application 20100221783) was used as an expression host for the GH61 polypeptide variants.

Aspergillus oryzae strain COLs1300 was also used as an expression host for GH61 polypeptide variants. A. niger COLs1300 (amyA, amyB, amyC, alpA, nprA, kusA, niiA, amdS+) was created from A. oryzae PFJ0220 (EP 2 147 107 B1) by deleting the promoter and 5′ part of both the nitrite reductase (niiA) gene and nitrate reductase (niaD) gene.

### Media and Reagents

AMG trace metals solution was composed of 14.3 g of ZnSO₄·7H₂O, 2.5 g of CuSO₄·5H₂O, 0.5 g of NiCl₂·6H₂O, 13.8 g of FeSO₄·7H₂O, 8.5 g of MnSO₄·H₂O, 3 g of citric acid, and deionized water to 1 liter.

COLs1300 protoplasting cultivating medium was composed of 100 ml of sucrose medium and 1 ml of 1 M urea.

COLs1300 protoplasting solution was composed of 80 mg of GLUCANEX® (Novozymes A/S, Bagsvaerd, Denmark), 0.5 mg/ml of chitinase (Sigma Chemical Co., Inc., St. Louis, MO, USA), 10 ml of 1.2 M MgSO₄, and 100 μl of 1 M NaH₂PO₄ pH 5.8.

COVE-N-Gly plates were composed of 50 ml of COVE salt solution, 218 g of sorbitol, 10 g of glycerol, 2.02 g of KNO₃, 25 g of Noble agar, and deionized water to 1 liter.

COVE-N-Gly plates with 10 mM uridine were composed of 50 ml of COVE salt solution, 218 g of sorbitol, 10 g of glycerol, 2.02 g of KNO₃, 25 g of Noble agar, and deionized water to 1 liter; uridine was then added at a concentration of 10 mM to individual plates.

COVE salt solution was composed of 26 g of KCl, 26 g of MgSO₄·7H₂O, 76 g of KH₂PO₄, 50 ml of COVE trace elements solution, and deionized water to 1 liter.

COVE trace elements solution was composed of 40 mg of Na₂B₄O₇·10H₂O, 0.4 g of CuSO₄·5H₂O, 1.2 g of FeSO₄·7H₂O, 0.7 g of MnSO₄·H₂O, 0.8 g of Na₂MoO₂·2H₂O, 10 g of ZnSO₄·7H₂O, and deionized water to 1 liter.

LB agar were composed of 5 g of starch (Merck, Whitehouse Station, NJ, USA), 37 g of LB agar (Sigma Chemical Co., Inc., St. Louis, MO, USA), and deionized water to 1 liter.

LB+Amp agar plates were composed of LB agar supplemented with 150 μg of ampicillin per ml.

M400 medium was composed of 50 g of maltodextrin, 2 g of MgSO₄·7H₂O, 2 g of KH₂PO₄, 4 g of citric acid, 8 g of yeast extract, 2 g of urea, 0.5 ml of AMG trace metals solution, 0.5 g of CaCl₂, and deionized water to 1 liter; adjusted with NaOH to pH 6. After pH adjustment 0.7 ml of antifoam was added.

Magnificent broth was composed of 50 g of Magnificent Broth powder (MacConnell Research Corp. San Diego, CA, USA) and deionized water to 1 liter.

MaltV1 medium was composed of 20 g of maltose, 10 g of Bacto Peptone, 1 g of yeast extract, 1.45 g of (NH₄)₂SO₄, 2.08 g of KH₂PO₄, 0.28 g of CaCl₂, 0.42 g of MgSO₄·7H₂O, 0.42 ml of Trichoderma trace metals solution, 0.48 g of citric acid, 19.52 g of 2-(N-morpholino)ethanesulfonic acid (MES), and deionized water to 1 liter; adjusted with NaOH to pH 5.5.

MDU2BP medium (pH 5.0) was composed of 135 g of maltose, 3 g of MgSO₄·7H₂O, 3 g of NaCl, 6 g of K₂SO₄, 36 g of KH₂PO₄, 21 g of yeast extract, 6 g of urea, 1.5 ml of AMG trace metals solution, and deionized water up to 1 liter.

PEG solution was composed of 6 g of polyethylene glycol 4000 (PEG 4000), 100 μl of 1 M Tris pH 7.5, 100 μl of 1 M CaCl₂, and deionized water to 10 ml.

Protoplasting cultivation medium was composed of 92 ml of transformation sucrose medium, 2 ml of 1 M uridine, 1 ml of 1 M NaNO₃, and 10 ml of YP medium.

Protoplasting solution was composed of 15 ml of 1.2 M MgSO₄, 150 μl of 1 M NaH₂PO₄ (pH 5.8), 100 mg of GLUCANEX® (Novozymes A/S, Bagsvaerd, Denmark), and 10 mg of chitinase (Sigma Chemical Co., Inc., St. Louis, MO, USA).

ST solution was composed of 1.5 ml of 2 M sorbitol, 500 μl of 1 M Tris pH 7.5, and deionized water to 5 ml.

STC solution was composed of 60 ml of 2 M sorbitol, 1 ml of 1 M Tris pH 7.5, 1 ml of 1 M CaCl₂, and deionized water to 100 ml.

Sucrose medium was composed of 20 ml of COVE salt solution, 342 g of sucrose, and deionized water to 1 liter.

Sucrose agar plates were composed of 20 ml of Trichoderma trace metals solution, 20 g of Noble agar, 342 g of sucrose, and deionized water to 1 liter.

TAE buffer was composed of 40 mM 2-amino-2-hydroxymethyl-propane-1,3-diol, 20 mM glacial acetic acid, and 2 mM ethylenediaminetetraacetic acid at pH 8.0.

TBE buffer was composed of 10.8 g of Tris base, 5.5 g of boric acid, and 0.74 g of EDTA (pH 8) in deionized water to 1 liter.

TE buffer was composed of 10 mM Tris-0.1 mM EDTA pH 8.

Top agar was composed of 500 ml of sucrose medium, 5 g of low melting agarose, and 10 ml of 20 mM Tris pH 7.5.

Transformation sucrose medium was composed of 70 ml of 1 M sucrose and 20 ml of COVE salt solution.

Trichoderma trace metals solution was composed of 216 g of FeCl₃·6H₂O, 58 g of ZnSO₄·7H₂O, 27 g of MnSO₄·H₂O, 10 g of CuSO₄·5H₂O, 2.4 g of H₃BO₃, 336 g of citric acid, and deionized water to 1 liter.

2XYT agar plates were composed of 16 g of tryptone, 10 g of yeast extract, 5 g of NaCl, 15 g of Bacto agar, and deionized water to 1 liter.

2XYT+Amp agar plates were composed of 2XYT agar supplemented with 100 μg of ampicillin per ml.

YP medium was composed of 10 g of Bacto yeast extract, 20 g of Bacto peptone, and deionized water to 1 liter.

### Example 1: Construction of Expression Vectors pMMar44, pMMar49, pMMar45, and pDFng113

Plasmid pMMar44 was constructed as described below for expression of the Aspergillus fumigatus GH61B polypeptide, and generation of mutant gene libraries. Additionally, plasmids pMMar49, pMMar45, and pDFng113 were constructed as described below for expression of the Aspergillus fumigatus GH61B polypeptide variants (WO 2012/044835), Penicillium sp. (emersonii) GH61A polypeptide (hereinafter Penicillium emersonii GH61A polypeptide), and Thermoascus aurantiacus GH61A polypeptide, respectively, and generation of variants.

Plasmid pENI2376 (U.S. Patent Application 20060234340) containing the AMA sequence for autonomous maintenance in Aspergillus was digested with Barn HI and Not I to linearize the plasmid and remove an 8 bp fragment. The digested plasmid was purified using a PCR Purification Kit (QIAGEN Inc., Valencia, CA, USA).

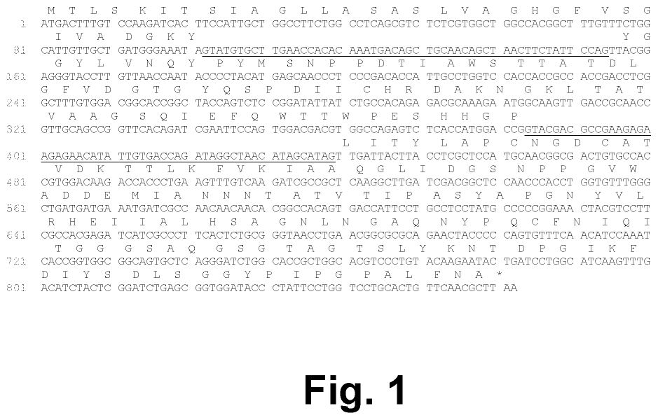

The Aspergillus fumigatus GH61B polypeptide coding sequence (FIG. 1; SEQ ID NO: 29 [genomic DNA sequence] and SEQ ID NO: 30 [deduced amino acid sequence]), mutated Aspergillus fumigatus GH61B polypeptide coding sequence (WO 2012/044835), Penicillium emersonii GH61A polypeptide coding sequence (SEQ ID NO: 35 [genomic DNA sequence] and SEQ ID NO: 36 [deduced amino acid sequence]), and Thermoascus aurantiacus GH61A polypeptide coding sequence (SEQ ID NO: 13 [genomic DNA sequence] and SEQ ID NO: 14 [deduced amino acid sequence]) were amplified from source plasmids described below using the primers shown in Table 1. Bold letters represent coding sequence. The remaining sequences are homologous to insertion sites of pENI2376 for expression of the GH61 polypeptide coding sequences.

TABLE 1

| GH61 | | | | |
|---|---|---|---|---|
| Polypeptide | Source | | | |
| origin | Template | Plasmid | Primer ID | Primer Sequence |
| Aspergillus | pAG43 (WO | pMMar44 | AspfuGH61Bp | CACAACTGGGGATCCATGACT |
| fumigatus | 2010/138754) | | ENI2376F_2 | TTGTCCAAGATCACTTCCA |
| GH61B | | | | (SEQ ID NO: 171) |
| | | | AspfuGH61Bp | GGCCTCCGCGGCCGCTTAAG |
| | | | ENI2376R_2 | CGTTGAACAGTGCAGGACCA |
| | | | | (SEQ ID NO: 172) |
| Mutated | pTH230 (WO | pMMar49 | AfumGH61SD | CACAACTGGGGATCCATGACT |
| Aspergillus | 2012/044835) | | MB3pENI3376 | TTGTCCAAGATCACTTCCA |
| fumigatus | | | F | (SEQ ID NO: 173) |
| GH61B | | | AfumGH61SD | GGCCTCCGCGGCCGCTTAAG |
| | | | MB3pENI3376 | CGTTGAACAGTGCAGGACCA |
| | | | R | (SEQ ID NO: 174) |
| Penicillium | pDM286 | pMMar45 | PenemGH61p | CACAACTGGGGATCCATGCTG |
| emersonii | | | ENI2376F | TCTTCGACGACTCGCACCC |
| GH61A | | | | (SEQ ID NO: 175) |
| | | | PenemGH61p | GGCCTCCGCGGCCGCCTAGA |
| | | | ENI2376R | ACGTCGGCTCAGGCGGCCCC |
| | | | | (SEQ ID NO: 176) |
| Thermoascus | pDZA2 | pDFng113 | TaGH61aBaM | CTGGGGATCCATGTCCTTTTC |
| aurantiacus | (WO | | HItagF | CAAGAT (SEQ ID NO: 177) |
| GH61A | 2005/074656) | | TaGH61aNcoI | CTCCGCGGCCGCTTAACCAGT |
| | | | tagR | ATACAGAG (SEQ ID NO: 178) |

Construction of plasmid pMMar44 containing the Aspergillus fumigatus GH61B polypeptide coding sequence is described below. The Aspergillus fumigatus GH61B polypeptide coding sequence was amplified from plasmid pAG43 (WO 2010/138754) using the primers shown in Table 1 with overhangs designed for cloning into plasmid pENI2376.

Fifty picomoles of each of the primers listed in Table 1 were used in a PCR composed of 90 ng of pAG43, 1× ADVANTAGE® 2 PCR Buffer (Clontech Laboratories, Inc., Mountain View, CA, USA), 1 μl of a blend of dATP, dTTP, dGTP, and dCTP, each at 10 mM, and 1× ADVANTAGE® 2 DNA Polymerase Mix (Clontech Laboratories, Inc., Mountain View, CA, USA) in a final volume of 50 μl. The amplification was performed using an EPPENDORF® MASTERCYCLER® 5333 (Eppendorf Scientific, Inc., Westbury, NY, USA) programmed for 1 cycle at 95° C. for 1 minute; 30 cycles each at 95° C. for 30 seconds, 60° C. for 30 seconds, and 72° C. for 1 minute; and a final elongation at 72° C. for 10 minutes. The heat block then went to a 4° C. soak cycle.

The reaction product was isolated by 1.0% agarose gel electrophoresis using TBE buffer where an approximately 862 bp PCR product band was excised from the gel and extracted using a QIAQUICK® Gel Extraction Kit (QIAGEN Inc., Valencia, CA, USA).

The homologous ends of the 862 bp PCR product and the digested pENI2376 were joined together using an IN-FUSION™ ADVANTAGE® PCR Cloning Kit (Clontech Laboratories, Inc., Mountain View, CA, USA). A total of 63 ng of the 862 bp PCR product and 200 ng of the Barn HI/NotI digested pENI2376 were used in a reaction composed of 4 μl of 5× IN-FUSION™ reaction buffer (Clontech Laboratories, Inc., Mountain View, CA, USA) and 2 μl of IN-FUSION™ enzyme (Clontech Laboratories, Inc., Mountain View, CA, USA) in a final volume of 20 μl. The reaction was incubated for 15 minutes at 37° C., followed by 15 minutes at 50° C., and then placed on ice. The reaction volume was increased to 100 μl with TE buffer and 2 μl of the reaction were transformed into E. coli XL10-GOLD® Super Competent Cells (Stratagene, La Jolla, CA, USA) according to the manufacturer's instructions. E. coli transformants were selected on 2XYT+Amp agar plates. Plasmid DNA from several of the resulting E. coli transformants was prepared using a BIOROBOT® 9600 (QIAGEN Inc., Valencia, CA, USA). The Aspergillus fumigatus GH61B polypeptide coding sequence insert was confirmed by DNA sequencing using a Model 3130xL Genetic Analyzer (Applied Biosystems®, Life Technologies, Grand Island, NY, USA) and dye-terminator chemistry from a BigDye® Terminator v3.1 Cycle Sequencing Kit (Applied Biosystems®, Life Technologies). Sequencing primers used for verification of the gene insert and sequence are shown below.

Primer 996271:
(SEQ ID NO: 179)
ACTCAATTTACCTCTATCCACACTT
Primer pALLO2 3′:
(SEQ ID NO: 180)
GAATTGTGAGCGGATAACAATTTCA

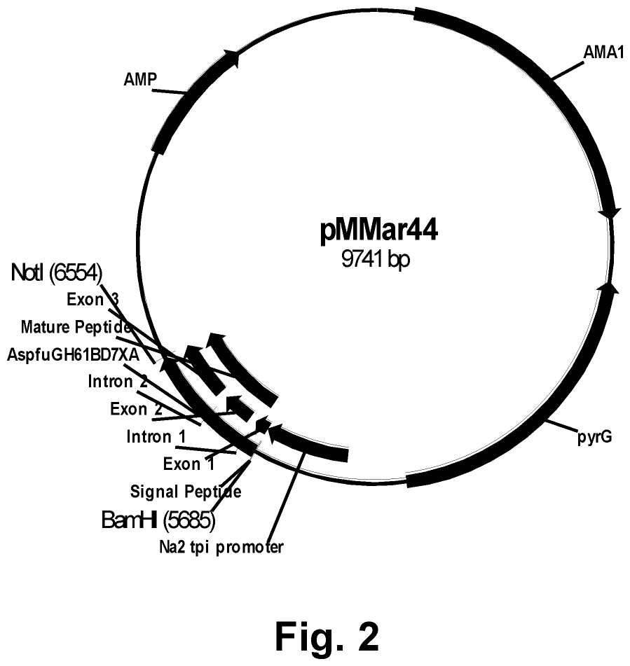

A plasmid containing the correct A. fumigatus GH61B polypeptide coding sequence was selected and designated pMMar44 (FIG. 2).

Construction of plasmid pMMar49 containing eight base-pair changes resulted in four amino acid mutations of the Aspergillus fumigatus GH61B polypeptide (WO 2012/044835) is described below. The mutated Aspergillus fumigatus GH61B polypeptide coding sequence (WO 2012/044835) was amplified from plasmid pTH230 using the primers shown in Table 1 with overhangs designed for cloning into plasmid pENI2376.

Fifty picomoles of each of the primers listed in Table 1 were used in a PCR composed of 100 ng of pTH230, 1× ADVANTAGE® 2 PCR Buffer (Clontech Laboratories, Inc., Mountain View, CA, USA), 1 μl of a blend of dATP, dTTP, dGTP, and dCTP, each at 10 mM, and 1× ADVANTAGE® 2 DNA Polymerase Mix (Clontech Laboratories, Inc., Mountain View, CA, USA) in a final volume of 50 μl. The amplification was performed using an EPPENDORF® MASTERCYCLER® 5333 programmed for 1 cycle at 95° C. for 1 minute; 30 cycles each at 95° C. for 30 seconds, 60° C. for 30 seconds, and 72° C. for 1 minute; and a final elongation at 72° C. for 7 minutes. The heat block then went to a 4° C. soak cycle.

The reaction product was isolated by 1.0% agarose gel electrophoresis using TBE buffer where an approximately 862 bp PCR product band was excised from the gel and extracted using a QIAQUICK® Gel Extraction Kit.

The homologous ends of the 862 bp PCR product and the digested pENI2376 were joined together using an IN-FUSION™ ADVANTAGE® PCR Cloning Kit. A total of 90 ng of the 862 bp PCR product and 220 ng of the Barn HI/Not I digested pENI2376 were used in a reaction composed of 4 μl of 5× IN-FUSION™ reaction buffer and 2 μl of IN-FUSION™ enzyme in a final volume of 20 μl. The reaction was incubated for 15 minutes at 37° C., followed by 15 minutes at 50° C., and then placed on ice. The reaction volume was increased to 100 μl with TE buffer and 2 μl of the reaction were transformed into E. coli XL10-GOLD® Super Competent Cells according to the manufacturer's instructions. E. coli transformants were selected on 2XYT+Amp agar plates. Plasmid DNA from several of the resulting E. coli transformants was prepared using a BIOROBOT® 9600. The mutated Aspergillus fumigatus GH61B polypeptide coding sequence insert was confirmed by DNA sequencing using a Model 3130xL Genetic Analyzer and dye-terminator chemistry from a BigDye® Terminator v3.1 Cycle Sequencing Kit. The sequencing primers 996271 and pALLO2 3′ were used for verification of the gene insert and sequence.

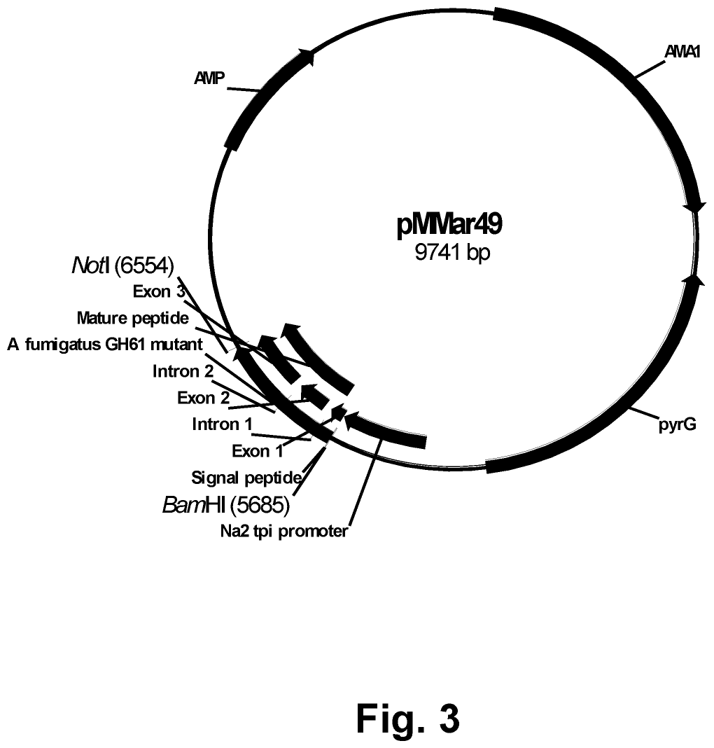

A plasmid containing the correct mutated A. fumigatus GH61B polypeptide coding sequence was selected and designated pMMar49 (FIG. 3).

Construction of plasmid pMMar45 containing the Penicillium emersonii GH61A polypeptide coding sequence is described below. The Penicillium emersonii GH61A polypeptide coding sequence was amplified from plasmid pDM286 containing the Penicillium emersonii GH61A polypeptide coding sequence using the primers shown in Table 1 with overhangs designed for cloning into plasmid pENI2376.

Plasmid pDM286 was constructed according to the following protocol. The P. emersonii GH61A polypeptide gene was amplified from plasmid pGH61D23Y4 (WO 2011/041397) using PHUSION™ High-Fidelity Hot Start DNA Polymerase (Finnzymes Oy, Espoo, Finland) and gene-specific forward and reverse primers shown below. The region in italics represents vector homology to the site of insertion.

Forward primer:
(SEQ ID NO: 181)
5′-CGGACTGCGCACCATGCTGTCTTCGACGACTCGCAC-3′
Reverse primer:
(SEQ ID NO: 182)
5′-TCGCCACGGAGCTTATCGACTTCTTCTAGAACGTC-3′

The amplification reaction contained 30 ng of plasmid pGH61D23Y4, 50 pmoles of each of the primers listed above, 1 μl of a 10 mM blend of dATP, dTTP, dGTP, and dCTP, 1× PHUSION™ High-Fidelity Hot Start DNA Polymerase buffer (Finnzymes Oy, Espoo, Finland) and 1 unit of PHUSION™ High-Fidelity Hot Start DNA Polymerase in a final volume of 50 μl.

The amplification reaction was incubated in an EPPENDORF® MASTERCYCLER® 5333 epgradient S (Eppendorf Scientific, Inc., Westbury, NY, USA) programmed for 1 cycle at 98° C. for 30 seconds; 35 cycles each at 98° C. for 10 seconds, 60° C. for 30 seconds, and 72° C. for 30 seconds, and 1 cycle at 72° C. for 10 minutes.

PCR products were separated by 1% agarose gel electrophoresis using TAE buffer. A 0.87 kb fragment was excised from the gel and extracted using a NUCLEOSPIN® Extract II Kit (Macherey-Nagel, Inc., Bethlehem, PA, USA).

Plasmid pMJ09 (US 2005/0214920 A1) was digested with Nco I and Pac I, and after digestion, the digested vector was isolated by 1.0% agarose gel electrophoresis using TBE buffer where an approximately 7.1 kb fragment was excised from the gel and extracted using a QIAQUICK® Gel Extraction Kit. The 0.87 kb PCR product was inserted into Nco I/Pac I-digested pMJ09 using an IN-FUSION™ ADVANTAGE® PCR Cloning Kit according to the manufacturer's protocol. The IN-FUSION™ reaction was composed of 1× IN-FUSION™ Reaction buffer, 180 ng of Not I/Pac I digested plasmid pMJ09, 108 ng of the 0.87 kb PCR product, and 1 μl of IN-FUSION™ Enzyme in a 10 μl reaction volume. The reaction was incubated for 15 minutes at 37° C. and then for 15 minutes at 50° C. To the reaction 40 μl of TE were added and 2 μl were used to transform ONE SHOT® TOP10 competent cells (Invitrogen, Carlsbad, CA, USA) according to the manufacturer's protocol. E. coli transformants were selected on 2XYT+Amp agar plates. Plasmid DNA from several of the resulting E. coli transformants was prepared using a BIOROBOT® 9600. The insert was confirmed by DNA sequencing using a Model 3130xL Genetic Analyzer and dye-terminator chemistry from a BigDye® Terminator v3.1 Cycle Sequencing Kit. One clone containing the insert with no PCR errors was identified and designated plasmid pDM286. Plasmid pDM286 can be digested with Pme I to generate an approximately 5.4 kb fragment for T. reesei transformation. This 5.4 kb fragment contains the expression cassette [T. reesei Cel7A cellobiohydrolase (CBHI) promoter, P. emersonii glycosyl hydrolase 61A (GH61A) gene, T. reesei Cel7A cellobiohydrolase (CBHI) terminator], and Aspergillus nidulans acetamidase (amdS) gene.

For construction of pMMar45, 50 picomoles of each of the primers listed in Table 1 were used in a PCR composed of 120 ng of pDM286, 1× EXPAND® PCR Buffer (Roche Diagnostics, Inc., Indianapolis, IN, USA), 1 μl of a blend of dATP, dTTP, dGTP, and dCTP, each at 10 mM, and 1× EXPAND® DNA Polymerase Mix (Roche Diagnostics, Inc., Indianapolis, IN, USA) in a final volume of 50 μl. The amplification was performed using an EPPENDORF® MASTERCYCLER® 5333 programmed for 1 cycle at 95° C. for 1 minute; 30 cycles each at 95° C. for 30 seconds, 60° C. for 30 seconds, and 72° C. for 1 minute; and a final elongation at 72° C. for 10 minutes. The heat block then went to a 4° C. soak cycle.

The reaction product was isolated by 1.0% agarose gel electrophoresis using TBE buffer where an approximately 762 bp PCR product band was excised from the gel and extracted using a QIAQUICK® Gel Extraction Kit.

The homologous ends of the 762 bp PCR product and the Barn HI/Not I digested pENI2376 were joined together using an IN-FUSION™ ADVANTAGE® PCR Cloning Kit. A total of 90 ng of the 762 bp PCR product and 200 ng of the digested pENI2376 were used in a reaction composed of 4 μl of 5× IN-FUSION™ reaction buffer and 2 μl of IN-FUSION™ enzyme in a final volume of 20 μl. The reaction was incubated for 15 minutes at 37° C., followed by 15 minutes at 50° C., and then placed on ice. The reaction volume was increased to 100 μl with TE buffer and 2 μl of the reaction were transformed into E. coli XL10-GOLD® Super Competent Cells according to the manufacturer's instructions. E. coli transformants were selected on 2XYT+Amp agar plates. Plasmid DNA from several of the resulting E. coli transformants was prepared using a BIOROBOT® 9600. The P. emersonii GH61A polypeptide coding sequence insert was confirmed by DNA sequencing using a Model 3130xL Genetic Analyzer and dye-terminator chemistry from a BigDye® Terminator v3.1 Cycle Sequencing Kit. The sequencing primers 996271 and pALLO2 3′ were used for verification of the gene insert and sequence.

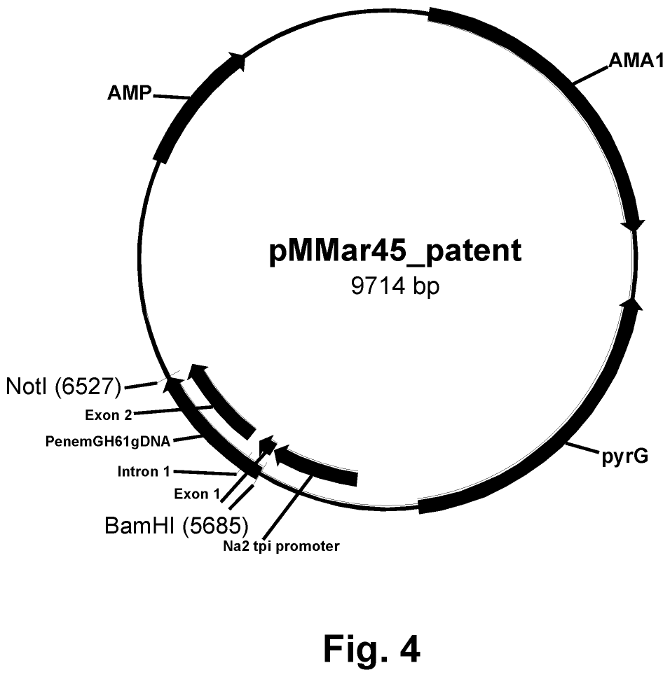

A plasmid containing the correct P. emersonii GH61A polypeptide coding sequence was selected and designated pMMar45 (FIG. 4).

Construction of plasmid pDFng113 containing the Thermoascus aurantiacus GH61A polypeptide coding sequence is described below. The Thermoascus aurantiacus GH61A polypeptide coding sequence was amplified from plasmid pDZA2 (WO 2005/074656) using the primers shown in Table 1 with overhangs designed for cloning into plasmid pENI2376.

Fifty picomoles of each of the primers listed in Table 1 were used in a PCR composed of 100 ng of pDZA2, 1× EXPAND® PCR Buffer, 1 μl of a blend of dATP, dTTP, dGTP, and dCTP, each at 10 mM, and 1× EXPAND® DNA Polymerase Mix in a final volume of 50 μl. The amplification reaction was performed using an EPPENDORF® MASTERCYCLER® 5333 programmed for 1 cycle at 94° C. for 2 minutes; 30 cycles each at 94° C. for 15 seconds, 59.9° C. for seconds, and 72° C. for 1 minute; and a final elongation at 72° C. for 7 minutes. The heat block then went to a 4° C. soak cycle.

The reaction product was isolated by 1.0% agarose gel electrophoresis using TBE buffer where an approximately 822 bp PCR product band was excised from the gel and extracted using a QIAQUICK® Gel Extraction Kit.

The homologous ends of the 822 bp PCR product and the Barn HI/Not I digested pENI2376 were joined together using an IN-FUSION™ ADVANTAGE® PCR Cloning Kit. A total of 37 ng of the 799 bp PCR product and 200 ng of the digested pENI2376 were used in a reaction composed of 4 μl of 5× IN-FUSION™ reaction buffer and 2 μl of IN-FUSION™ enzyme in a final volume of 20 μl. The reaction was incubated for 15 minutes at 37° C., followed by 15 minutes at 50° C., and then placed on ice. The reaction volume was increased to 50 μl with TE buffer and 2 μl of the reaction were transformed into E. coli XL10-GOLD® Ultracompetent Cells (Stratagene, La Jolla, CA, USA) according to the manufacturer's instructions. E. coli transformants were selected on 2XYT+Amp agar plates. Plasmid DNA from several of the resulting E. coli transformants was prepared using a BIOROBOT® 9600. The T. aurantiacus GH61A polypeptide coding sequence insert was confirmed by DNA sequencing using a Model 3130xL Genetic Analyzer and dye-terminator chemistry from a BigDye® Terminator v3.1 Cycle Sequencing Kit. The sequencing primers 996271 and pALLO2 3′ were used for verification of the gene insert and sequence.

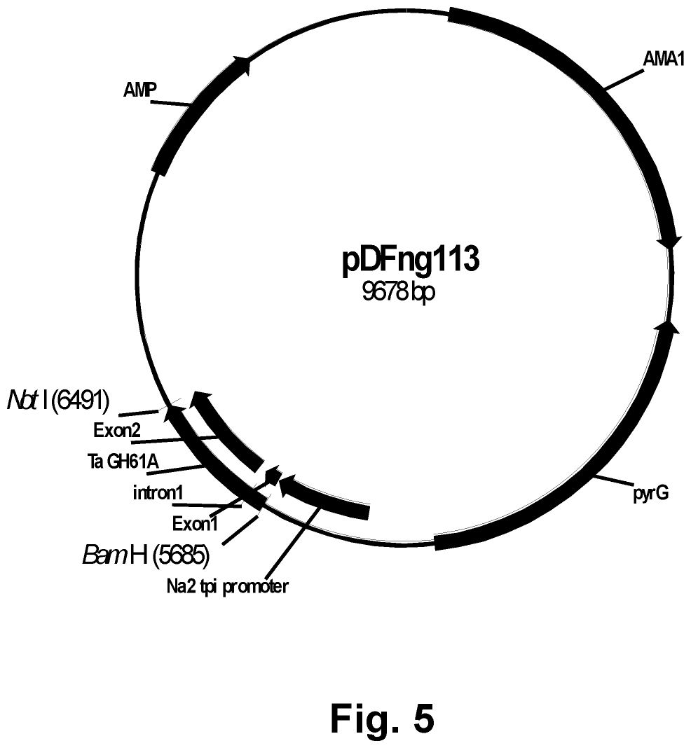

A plasmid containing the correct T. aurantiacus GH61A polypeptide coding sequence was selected and designated pDFng113 (FIG. 5).

### Example 2: Construction of an Aspergillus fumigatus GH61B Polypeptide Site Saturation Library

A site saturation library of the Aspergillus fumigatus GH61B polypeptide coding sequence was synthesized by GeneArt AG (Regensburg, Germany). An average of 16.8 mutations per position was synthesized for a total of 165 residues, excluding the most conserved residues, resulting in a total of 2768 mutants. E. coli DH10B (Invitrogen, Carlsbad, CA, USA) strains containing mutant plasmids with known mutations were arrayed in 96 well plates as 50 μl glycerol stocks, and stored at −80° C.

DNA was generated from a thawed GeneArt plate by using a sterile 96 well replicator to stamp the GeneArt plate onto a 2XYT agar plate containing 100 μg/ml of ampicillin. The agar plate was incubated overnight at 37° C. Resulting colonies from the agar plate were used to inoculate a 96 deep well block with each well containing 1 ml of Magnificent broth supplemented with 400 μg of ampicillin per ml. The block was covered with an airpore breathable lid and then incubated in a humidified box at 37° C. overnight at 350 rpm. The block was centrifuged at 1100×g for 10 minutes and the supernatant discarded. Plasmids were extracted from the cell pellets using a BIOROBOT® 9600.

### Example 3: Expression of the Wild-Type and Variants of the A. fumigatus GH61B Polypeptide and P. emersonii GH61A Polypeptide in Aspergillus oryzae PFJ0218

Aspergillus oryzae PFJO218 was inoculated onto a COVE-N-Gly plate with 10 mM uridine and incubated at 34° C. until confluent. Spores were collected from the plate by washing with 8 ml of 0.01% TWEEN® 20. One ml of the spore suspension was used to inoculate 103 ml of the Protoplasting cultivation medium in a 500 ml polycarbonate shake flask. The shake flask was incubated at 30° C. with agitation at 180 rpm for 17-20 hours. Mycelia were filtered through a funnel lined with MIRACLOTH® (Calbiochem, La Jolla, CA, USA) and washed with 200 ml of 0.6 M MgSO₄. Washed mycelia were resuspended in 15 ml of Protoplasting solution in a 125 ml sterile polycarbonate shake flask and incubated on ice for 5 minutes. One ml of a solution of 12 mg of bovine serum albumin per ml of deionized water was added to the shake flask and the shake flask was then incubated at 37° C. with mixing at 70 rpm for 1-3 hours until protoplasting was complete. The mycelia/protoplast mixture was filtered through a funnel lined with MIRACLOTH® into a 50 ml conical tube and overlayed with 5 ml of ST solution. The 50 ml conical tube was centrifuged at 1050×g for 15 minutes with slow acceleration/deceleration. After centrifugation, the liquid was separated into 3 phases. The interphase which contained the protoplasts was transferred to a new 50 ml conical tube. Two volumes of STC solution were added to the protoplasts followed by a brief centrifugation at 1050×g for 5 minutes. The supernatant was discarded. The protoplasts were washed twice with 20 ml of STC with resuspension of the protoplast pellet, centrifugation at 1050×g for 10 minutes, and decanting of the supernatant each time. After the final decanting, the protoplast pellet was resuspended in STC at a concentration of 1×10⁸/ml. Protoplasts were frozen at −80° C. until transformation.

A 1.3 μl volume of each mutant plasmid was used to transform 3.5 μl of A. oryzae PFJO218 protoplasts with 3.5 μl of PEG solution per well in a 24 well plate. Plasmid pMMar44 or pMMar45 (Table 1) was also transformed as above into A. oryzae PFJO218 protoplasts to provide broth comprising the A. fumigatus or P. emersonii wild-type GH61 polypeptides. The 24 well plate was incubated at 37° C. stationary for 30 minutes followed by addition of 28.6 μl of Transformation sucrose medium containing 10 mM NaNO₃ and 14.3 μl of STC. The 24 well plate was then placed in a humidified box at 37° C. stationary for 7 days. On day 7, 1 ml of MaltV1 medium was added to each well. The plate was returned to the humidified box at 39° C. stationary and incubated for an additional 5 days. At least 550 μl of broth for each variant or the wild-type A. fumigatus or P. emersonii GH61 polypeptide were harvested using a pipette to remove the mycelia mat and aspirate the liquid, for assay using PASC as a substrate. Mutant plasmids resulting in variants with improved thermostability using a PASC assay (Example 5) were transformed again and retested using the protocols described above.

Some of the variants were spore-purified for further characterization. After a 7 day incubation of the transformation and prior to the addition of 1 ml of MaltV1 expression medium, a loop was swiped over the initial growth from the transformation to collect spores in the well. The spores were then streaked onto a COVE-N-Gly plate and incubated at 37° C. for approximately 36 hours. Single individual transformants were excised from the plate and transferred onto fresh COVE-N-Gly plates. The plates were stored at 34° C. until confluent. Once confluent, a loop dipped in 0.01% TWEEN® 20 was swiped over the spores which was then used to inoculate a 24 well plate with each well containing 1 ml of MaltV1 expression medium. The 24 well plate was placed in a humidified box at 39° C. Samples were harvested on the fifth day by removing the mycelia mat and pipetting up the broth.

### Example 4: Preparation of Aspergillus fumigatus Beta-Glucosidase

Aspergillus fumigatus NN055679 Cel3A beta-glucosidase (SEQ ID NO: 169 [DNA sequence] and SEQ ID NO: 170 [deduced amino acid sequence]) was recombinantly prepared according to WO 2005/047499 using Aspergillus oryzae as a host.

The filtered broth was adjusted to pH 8.0 with 20% sodium acetate, which made the solution turbid. To remove the turbidity, the solution was centrifuged (20000×g for 20 minutes), and the supernatant was filtered through a 0.2 μm filtration unit (Nalgene, Rochester, NY, USA). The filtrate was diluted with deionized water to reach the same conductivity as 50 mM Tris/HCl, pH 8.0. The adjusted enzyme solution was applied to a Q SEPHAROSE® Fast Flow column (GE Healthcare, Piscataway, NJ, USA) equilibrated with 50 mM Tris-HCl pH 8.0 and eluted with a linear gradient from 0 to 500 mM sodium chloride. Fractions were pooled and treated with 1% (w/v) activated charcoal to remove color from the beta-glucosidase pool. The charcoal was removed by filtration of the suspension through a 0.2 μm filtration unit (Nalgene, Rochester, NY, USA). The filtrate was adjusted to pH 5.0 with 20% acetic acid and diluted 10 times with deionized water. The adjusted filtrate was applied to a SP SEPHAROSE® Fast Flow column (GE Healthcare, Piscataway, NJ, USA) equilibrated with 10 mM succinic acid pH 5.0 and eluted with a linear gradient from 0 to 500 mM sodium chloride. Protein concentration was determined using a Microplate BCA™ Protein Assay Kit (Thermo Fischer Scientific, Waltham, MA, USA) in which bovine serum albumin was used as a protein standard.

### Example 5: Screening of Aspergillus fumigatus GH61B and Penicillium emersonii GH61A Polypeptide Variant Libraries

Using a BIOMEK® FX Laboratory Automation Workstation (Beckman Coulter, Fullerton, CA, USA) with a DYAD® Thermal Cycler (Bio-Rad Laboratories, Inc., Richmond, CA, USA), 80 μl of each broth sample from the library plates of the Aspergillus fumigatus GH61B variants and parent (wild-type) polypeptide grown in MaltV1 medium (Example 3) were mixed with 20 μl of 1 M sodium acetate-10 mM MnSO₄ pH 5.0 buffer. Depending on the library, the samples were then heat challenged at 62° C., 65° C., 68° C., 72° C., or 75° C. for 20 minutes and compared to ambient temperature controls. After the heat challenge, the broth samples were diluted 1.25, 2.5, 6.25, and 15.625-fold in 2 mM MnSO₄-200 mM sodium acetate pH 5 and 12.5 μl of the dilutions were then transferred to 384-well polypropylene assay plates containing 25 μl of 1% phosphoric acid swollen cellulose (PASC) and 12.5 μl of a cofactor solution (400 mM sodium acetate pH 5, 4 mM MnSO₄, 0.4% gallic acid, 0.1 mg/ml of Aspergillus fumigatus beta-glucosidase, and 0.04% TRITON® X100). The plates were heat-sealed using an ALPS-300™ (Abgene, Epsom, United Kingdom) with a plastic sheet and incubated at 40° C. for 4 days.

Background glucose concentration of the buffer-treated broth samples was determined prior to incubation by performing a glucose assay using the following reagents per liter: 0.9951 g of ATP, 0.5176 g of NAD, 0.5511 g of MgSO₄·7H₂O, 20.9 g of MOPS, 1000 units of hexokinase, 1000 units of glucose-6-phosphate dehydrogenase, and 0.01% TRITON® X-100, pH 7.5. The BIOMEK® FX Laboratory Automation Workstation was used for this assay. Four 2-fold serial dilutions were performed in 384-well polystyrene plates using water as diluent. Five μl of the dilutions were added to a new 384-well polystyrene plate, followed by addition of 60 μl of the above reagents. The plate was incubated at ambient temperature (22° C.±2° C.) for 30 to 45 minutes. Relative fluorescent units (RFU) were determined using a DTX 880 plate reader (Beckman Coulter, Fullerton, CA, USA) with excitation at 360 nm and emission at 465 nm and compared to glucose standards (1 mg/ml and 0.125 mg/ml) diluted in the same plate as the samples. At the end of four days, the 40° C. incubated PASC plates were analyzed for glucose concentration using the glucose assay described above. Any background glucose was subtracted from the appropriate samples and then residual activity was calculated by comparing the glucose released in the PASC assay of the ambient sample treatment to the glucose released in the PASC assay of the heat challenged sample. Only data that fits in the linear part of the curve (defined as less than or equal to 1 mg/ml glucose produced in an assay containing 5 mg/ml PASC) was used in the calculation. The formula for calculating the residual activity of the heat treatment was as follows: (mg/ml glucose produced for heat treated sample/mg/ml glucose produced for ambient treated sample)×100%. Improved variants were those having a higher % residual activity as compared to wild-type A. fumigatus GH61A polypeptide broth from MaltV1 medium in at least one heat treatment condition. MICROSOFT® EXCEL® (Microsoft Corporation, Redmond, WA, USA) was used for all calculations.

### Example 6: Thermostability of Aspergillus fumigatus GH61B Variants Measured by Residual Activity after Heat Treatment

Based on the residual activity ratios as described in Example 5, screening of libraries constructed in the previous Examples generated the results listed in Table 2.

Table 2 shows average % Residual Activity (from 3-5 samples of each variant and the wild-type control) after treatment at 62, 65, or 68° C. The parent Aspergillus fumigatus GH61B polypeptide showed decreased residual activity of 56%, 35%, and 12% when the temperature was increased from 62° C. to 65° C. to 68° C., respectively. The increase in thermostability of the Aspergillus fumigatus GH61B polypeptide variants ranged from 1.02- to 1.3-fold increase when treated at 62° C., 1.06- to 1.7-fold increase when treated at 65° C., and 1.3- to 3.8-fold increase when treated at 68° C. compared to the wild-type A. fumigatus GH61 polypeptide. The results showed that improvements were most significant when treated at 68° C.

TABLE 2
Variants with improved thermostability at 62, 65, or 68° C. treatment

| | Avg % | | Avg % | | Avg % | |
|---|---|---|---|---|---|---|
| | Res. Act. | | Res. Act. | | Res. Act. | |
| | 62° C. | Standard | 65° C. | Standard | 68° C. | Standard |
| Variant | treatment | Deviation | treatment | Deviation | treatment | Deviation |
| Parent | 56% | 13% | 35% | 16% | 12% | 10% |
| (Wild-Type) | | | | | | |
| S26I | 71% | 8% | 59% | 15% | 36% | 17% |
| G32E | 64% | 21% | 50% | 24% | 28% | 20% |
| G32S | 67% | 17% | 50% | 20% | 33% | 15% |
| Y34F | 50% | 6% | 38% | 9% | 25% | 21% |
| V40A | 46% | 10% | 35% | 7% | 22% | 7% |
| N41T | 59% | 21% | 56% | 22% | 41% | 30% |
| Q42E | 60% | 20% | 43% | 15% | 27% | 19% |
| Q42I | 48% | 7% | 50% | 16% | 41% | 33% |
| Q42V | 62% | 22% | 47% | 14% | 28% | 14% |
| S47R | 58% | 13% | 41% | 17% | 18% | 11% |
| S47E | 58% | 11% | 47% | 11% | 22% | 4% |
| S47L | 52% | 13% | 42% | 7% | 18% | 6% |
| S56T | 53% | 8% | 41% | 15% | 19% | 15% |
| S56E | 59% | 9% | 40% | 12% | 22% | 16% |
| S56C | 55% | 10% | 38% | 15% | 16% | 14% |
| S72Q | 57% | 6% | 45% | 8% | 31% | 21% |
| S72T | 60% | 13% | 45% | 7% | 25% | 12% |
| T102K | 60% | 2% | 44% | 10% | 26% | 7% |
| T102P | 55% | 7% | 42% | 9% | 33% | 18% |
| A123R | 52% | 7% | 39% | 9% | 17% | 15% |
| Q138C | 67% | 13% | 51% | 16% | 35% | 15% |
| Q138E | 63% | 15% | 58% | 10% | 46% | 11% |
| Q138K | 69% | 11% | 58% | 8% | 46% | 13% |
| Q138L | 69% | 11% | 56% | 16% | 39% | 17% |
| Q138M | 62% | 12% | 55% | 11% | 34% | 21% |
| Q138G | 53% | 11% | 47% | 13% | 29% | 21% |
| V149I | 53% | 2% | 43% | 4% | 28% | 11% |
| D152S | 64% | 7% | 59% | 8% | 39% | 17% |
| T163V | 60% | 13% | 50% | 5% | 24% | 3% |
| T163F | 61% | 15% | 47% | 8% | 20% | 5% |
| T163E | 59% | 13% | 43% | 8% | 21% | 4% |
| V164C | 58% | 12% | 48% | 7% | 24% | 3% |
| V164L | 56% | 9% | 40% | 5% | 16% | 1% |
| I166L | 59% | 16% | 45% | 11% | 17% | 3% |
| S169R | 45% | 9% | 48% | 14% | 38% | 29% |
| S186K | 47% | 8% | 41% | 9% | 38% | 22% |
| S186F | 46% | 11% | 38% | 11% | 28% | 15% |
| S186T | 46% | 23% | 35% | 25% | 31% | 29% |
| S186Y | 45% | 19% | 37% | 16% | 32% | 20% |
| F200I | 42% | 13% | 40% | 13% | 32% | 15% |
| F200V | 48% | 14% | 45% | 19% | 32% | 31% |
| G207P | 60% | 12% | 40% | 17% | 20% | 14% |
| S213E | 50% | 12% | 35% | 11% | 18% | 8% |
| S219C | 45% | 9% | 35% | 4% | 27% | 6% |
| S219E | 47% | 12% | 39% | 7% | 28% | 8% |
| S219M | 48% | 9% | 42% | 13% | 31% | 18% |
| S219Q | 49% | 12% | 41% | 30% | 31% | 31% |
| K222R | 61% | 14% | 50% | 24% | 35% | 35% |
| S234K | 52% | 22% | 36% | 21% | 17% | 26% |
| S234G | 68% | 13% | 45% | 18% | 27% | 27% |
| A246P | 56% | 7% | 43% | 11% | 26% | 15% |
| N249Q | 67% | 14% | 52% | 16% | 36% | 20% |
| N249R | 61% | 19% | 46% | 20% | 32% | 22% |

### Example 7: Thermostability of Aspergillus fumigatus GH61B Polypeptide Combinatorial Variants

Plasmid pLSBF09-3 was constructed as a template for subsequent Aspergillus fumigatus GH61B combinatorial variants. This plasmid was constructed by performing a single site-directed mutagenesis reaction on pMMar49 (Example 1) using a QUIKCHANGE® II XL Site-Directed Mutagenesis Kit (Stratagene, La Jolla, CA, USA). Two mutagenic primers were designed to insert the desired mutation. The PCR was composed of 125 ng of each primer, approximately 25 ng of template plasmid, 1× QUIKCHANGE® reaction buffer (Stratagene, La Jolla, CA, USA), 3 μl of QUIKSOLUTION® (Stratagene, La Jolla, CA, USA), 1 μl of XL dNTP mix, and 1 μl of 2.5 U/μl Pfu ULTRA™ enzyme (Stratagene, La Jolla, CA, USA) in a final volume of 50 μl. The amplification reaction was performed using an EPPENDORF® MASTERCYCLER® thermocycler programmed for a 95° C. hot start; 1 cycle at 95° C. for 1 minute; 18 cycles each at 95° C. for 50 seconds, 60° C. for 50 seconds, and 68° C. for 10 minutes; and a 4° C. hold. One microliter of Dpn I was directly added to the amplification reaction and incubated at 37° C. for 1 hour. A 2 μl volume of the Dpn I digested reaction was used to transform E. coli XL10-GOLD® Ultracompetent Cells according to the manufacturer's instructions. E. coli transformants were selected on 2XYT+Amp agar plates. One of the clones with the desired mutation was designated pLSBF09-3. Using pLSBF09-3 as a template, two additional combinatorial plasmids were constructed. Plasmids pDFng146 and pDFng148 were each mutagenized as described above. The primers used in these reactions are shown in Table 3.

TABLE 3

| | | Parent | | |
|---|---|---|---|---|
| Plasmid | Mutations | Plasmid | Oligo ID # | Sequence |
| pLSBF09-3 | L111V, | pMMar49 | 615628 | ACAAGAATACTGATCCTGGCATCT |
| | D152S, | | | GGTTTGACATCTACTCGGATCTGA |
| | M155L, | | | G (SEQ ID NO: 183) |
| | A162W, | | 615632 | CTCAGATCCGAGTAGATGTCAAAC |
| | K229W | | | CAGATGCCAGGATCAGTATTCTTG |
| | | | | T (SEQ ID NO: 184) |
| pDFng146 | L111V, | pLSBF09-3 | 1200378 | ATCATCGCCCTTCACTCTGCGTTT |
| | D152S, | | | AACCTGAACGGCGCGCAGAAC |
| | M155L, | | | (SEQ ID NO: 185) |
| | A162W, | | 1200379 | GTTCTGCGCGCCGTTCAGGTTAA |
| | G188F, | | | ACGCAGAGTGAAGGGCGATGAT |
| | K229W | | | (SEQ ID NO: 186) |
| pDFng148 | L111V, | pLSBF09-3 | 1200382 | GAAGTTTGTCAAGATCGCCGCTAA |
| | D152S, | | | GGGCTTGATCGACGGCTCCAAC |
| | M155L, | | | (SEQ ID NO: 187) |
| | A162W, | | 1200383 | GTTGGAGCCGTCGATCAAGCCCT |
| | Q138K, | | | TAGCGGCGATCTTGACAAACTTC |
| | K229W | | | (SEQ ID NO: 188) |

Seven variants (pLSBF15, 17, 18, 20, 22, 53, and pDFNG145) of the Aspergillus fumigatus GH61B polypeptide were constructed by adding a single amino acid mutation on top of pLSBF09-3 or pDFng148 using a QUIKCHANGE® II XL Site-Directed Mutagenesis Kit). The site directed mutagenesis method described above was used in the construction of these mutants as well. Primers used in these reactions are shown in Table 4.

Four additional variants (pLSBF47, 48, 49, and 54) of Aspergillus fumigatus GH61B were constructed via multi-site-directed mutagenesis of pDFng146 or pDFng148 using a QUIKCHANGE® Lightning Multi Site-Directed Mutagenesis Kit (Stratagene, La Jolla, CA, USA). One mutagenic primer was designed for each desired mutation. One hundred ng of each primer (Table 4) were used in a PCR containing approximately 100 ng of template plasmid, 1× QUIKCHANGE® Lightning Multi Buffer (Stratagene, La Jolla, CA, USA), 0.5 μl of QUIKSOLUTION® (Stratagene, La Jolla, CA, USA), 1 μl of dNTP mix, and 1 μl of QUIKCHANGE® Lightning Multi enzyme blend (Stratagene, La Jolla, CA, USA) in a final volume of 25 μl. The amplification reaction was performed using an EPPENDORF® MASTERCYCLER® thermocycler programmed for a 95° C. hot start; 1 cycle at 95° C. for 2 minutes; 30 cycles each at 95° C. for 20 seconds, 55° C. for 30 seconds, and 65° C. for 5 minutes; 1 cycle at 65° C. for 5 minutes, and a 4° C. hold. One microliter of Dpn I was directly added to the amplification reaction and incubated at 37° C. for 5 minutes. A 1.5 μl volume of the Dpn I digested reaction was transformed into E. coli XL10-GOLD® Ultracompetent Cells according to the manufacturer's instructions. E. coli transformants were selected on 2XYT+Amp agar plates. Plasmid DNA from several of the resulting E. coli transformants was prepared using a BIOROBOT® 9600. The insert was confirmed by DNA sequencing using a Model 3130xL Genetic Analyzer and dye-terminator chemistry from a BigDye® Terminator v3.1 Cycle Sequencing Kit. One of the clones with the desired mutations was designated as each plasmid listed below. The A. fumigatus GH61A polypeptide variants were expressed using Aspergillus oryzae PFJO218 as host was performed according to the procedure described in Example 3.

TABLE 4

| | | Parent | | |
|---|---|---|---|---|
| Plasmid | Mutations | Plasmid | Oligo ID # | Sequence |
| pLSBF15 | L111V, | pLSBF09-3 | 1200265 | CTTGTTAACCAATACCCCTACATGGAA |
| | D152S, | | | AACCCTCCCGACACCATTGCC (SEQ ID |
| | M155L, | | | NO: 189) |
| | A162W, | | 1200266 | GGCAATGGTGTCGGGAGGGTTTTCCA |
| | S47E, | | | TGTAGGGGTATTGGTTAACAAG (SEQ |
| | K229W | | | ID NO: 190) |
| pLSBF17 | L111V, | pLSBF09-3 | 1200269 | CTCCCGACACCATTGCCTGGGCCACC |
| | D152S, | | | ACCGCCACCGACCTCG (SEQ ID NO: |
| | M155L | | | 191) |
| | A162W, | | 1200270 | CGAGGTCGGTGGCGGTGGTGGCCCA |
| | S56A, | | | GGCAATGGTGTCGGGAG (SEQ ID NO: |
| | K229W | | | 192) |
| pLSBF18 | L111V, | pLSBF09-3 | 1200271 | ACAGATCGAATTCCAGTGGACGAAGT |
| | D152S, | | | GGCCAGAGTCTCACCATGGA (SEQ ID |
| | M155L | | | NO: 193) |
| | A162W, | | 1200272 | ACAGATCGAATTCCAGTGGACGAAGT |
| | T102K, | | | GGCCAGAGTCTCACCATGGA (SEQ ID |
| | K229W | | | NO: 194) |
| pLSBF20 | L111V, | pLSBF09-3 | 1200275 | CCAGTGGACGAAGTGGCCAAAGTCTC |
| | D152S, | | | ACCATGGACCG (SEQ ID NO: 195) |
| | M155L, | | 1200276 | CGGTCCATGGTGAGACTTTGGCCACT |
| | A162W, | | | TCGTCCACTGG (SEQ ID NO: 196) |
| | T102K, | | | |
| | E105K, | | | |
| | K229W | | | |
| pLSBF22 | L111V, | pLSBF09-3 | 1200279 | CTGGCATCTGGTTTGACATCTACGGC |
| | D152S, | | | GATCTGAGCGGTGGATACCCT (SEQ |
| | M155L | | | ID NO: 197) |
| | A162W, | | 1200280 | AGGGTATCCACCGCTCAGATCGCCGT |
| | K229W, | | | AGATGTCAAACCAGATGCCAG (SEQ ID |
| | S234G | | | NO: 198) |
| pLSBF53 | L111V, | pDFng148 | 1200378 | ATCATCGCCCTTCACTCTGCGTTTAAC |
| | D152S, | | | CTGAACGGCGCGCAGAAC (SEQ ID |
| | M155L, | | | NO: 199) |
| | A162W, | | 1200379 | GTTCTGCGCGCCGTTCAGGTTAAACG |
| | Q138K, | | | CAGAGTGAAGGGCGATGAT (SEQ ID |
| | G188F, | | | NO: 200) |
| | K229W | | | |
| pDFng145 | L111V, | pLSBF09-3 | 1200376 | CACGAGATCATCGCCCTTCACACCGC |
| | D152S, | | | GGGTAACCTGAACGGCGC (SEQ ID |
| | M155L, | | | NO: 201) |
| | A162W, | | 1200377 | GCGCCGTTCAGGTTACCCGCGGTGTG |
| | S186T, | | | AAGGGCGATGATCTCGTG (SEQ ID |
| | K229W | | | NO: 202) |
| pLSBF47 | L111V, | pDFng148 | 1200378 | ATCATCGCCCTTCACTCTGCGTTTAAC |
| | D152S, | | | CTGAACGGCGCGCAGAAC (SEQ ID |
| | M155L, | | | NO: 203) |
| | A162W | | 1201840 | CGGCTCCAACCCACCTGGTATCTGGG |
| | Q138K, | | | CTTCCGATGAACTGATCG (SEQ ID NO: |
| | V149I, | | | 204) |
| | G188F, | | | |
| | K229W | | | |
| pLSBF48 | L111V, | pDFng148 | 1200378 | ATCATCGCCCTTCACTCTGCGTTTAAC |
| | D152S, | | | CTGAACGGCGCGCAGAAC (SEQ ID |
| | M155L, | | | NO: 205) |
| | A162W, | | 1201840 | CGGCTCCAACCCACCTGGTATCTGGG |
| | S72T, | | | CTTCCGATGAACTGATCG (SEQ ID NO: |
| | Q138K, | | | 206) |
| | V149I, | | 1201841 | CGGCACCGGCTACCAGACCCCGGATA |
| | G188F, | | | TTATCTGCCACAGAGACGC (SEQ ID |
| | K229W | | | NO: 207) |
| pLSBF49 | L111V, | pDFng148 | 1200378 | ATCATCGCCCTTCACTCTGCGTTTAAC |
| | D152S, | | | CTGAACGGCGCGCAGAAC (SEQ ID |
| | M155L, | | | NO: 208) |
| | A162W, | | 1201840 | CGGCTCCAACCCACCTGGTATCTGGG |
| | Q138K, | | | CTTCCGATGAACTGATCG (SEQ ID NO: |
| | V149I, | | | 209) |
| | G188F, | | 1201842 | CCAGTGTTTCAACATCCAAATCACCGG |
| | G207P, | | | TCCTGGCAGTGCTCAGGG (SEQ ID |
| | K229W | | | NO: 210) |
| pLSBF54 | L111V, | pDFng146 | 1201386 | CCATTCCTGCCTGCTATGCCCCCGGA |
| | D152S, | | | AACTACGTCC (SEQ ID NO: 211) |
| | M155L, | | 1201390 | CCTGGTCCTGCACTGTTCAACTGCTAA |
| | A162W | | | GCGGCC (SEQ ID NO: 212) |
| | S169C, | | | |
| | G188F, | | | |
| | K229W, | | | |
| | A250C | | | |

Based on the residual activity ratios determined according to Example 5, screening of libraries constructed in the previous Examples generated the results listed in Table 5. Table 5 shows an average % Residual Activity (2-199 samples for each of the combinatorial variants and 34-179 samples for the wild-type GH61 polypeptide after treatment at any of 65° C., 68° C., 72° C., or 75° C.

The parent wild-type A. fumigatus GH61 polypeptide showed decreased residual activity of 40%, 24%, 0% and 0% when the temperature of treatment was increased from 65° C. to 68° C. to 72° C. to 75° C., respectively. The thermostability of the Aspergillus fumigatus GH61B polypeptide combinatorial variants ranged from 0.8-fold (decrease) to 1.4-fold increase at 65° C., no increase to 2-fold increase at 68° C. compared to the wild-type A. fumigatus GH61 polypeptide. Since wild-type GH61 polypeptide has no residual activity at 72° C., the other variant (L111V, D152S, M155L, A162W) was used for comparison at 72° C. and 75° C. In these cases, the variants ranged from no increase to 4.4-fold increase at 72° C., and 4-fold to 33-fold increase at 75° C. The results showed that improvements were most significant at the 75° C. treatment for those measured at 75° C., otherwise they were most significant at 72° C. where the wild-type had no measurable residual activity.

TABLE 5
Aspergillus fumigatus GH61B polypeptide variants with improved
thermostability at 65° C., 68° C., 72° C. or 75° C. treatment

| | Avg | | Avg | | Avg | | Avg | |
|---|---|---|---|---|---|---|---|---|
| | % | | % | | % | | % | |
| | Res. | | Res. | | Res. | | Res. | |
| | Act. | Standard | Act. | Standard | Act. | Standard | Act. | Standard |
| Mutations | 65° C. | Deviation | 68° C. | Deviation | 72° C. | Deviation | 75° C. | Deviation |
| L111V, D152S, | 55% | 16% | 49% | 21% | 10% | 7% | 1% | 7% |
| M155L, A162W | | | | | | | | |
| L111V, D152S, | 51% | 7% | NA | NA | 44% | 1% | 30% | 5% |
| M155L, A162W, | | | | | | | | |
| Q138K, G188F, | | | | | | | | |
| K229W | | | | | | | | |
| L111V, D152S, | 470% | 9% | NA | NA | 42% | 9% | 32% | 12% |
| M155L, A162W, | | | | | | | | |
| Q138K, V149I, | | | | | | | | |
| G188F, G207P, | | | | | | | | |
| K229W | | | | | | | | |
| L111V, D152S, | 50% | 4% | NA | NA | 41% | 6% | 31% | 8% |
| M155L, A162W, | | | | | | | | |
| S72T, Q138K, | | | | | | | | |
| V149I, G188F, | | | | | | | | |
| K229W | | | | | | | | |
| L111V, D152S, | 39% | 12% | NA | NA | 37% | 14% | 33% | 12% |
| M155L, A162W, | | | | | | | | |
| S169C, G188F, | | | | | | | | |
| K229W, A250C | | | | | | | | |
| L111V, D152S, | 49% | 6% | NA | NA | 36% | 19% | 28% | 14% |
| M155L, A162W, | | | | | | | | |
| Q138K, V149I, | | | | | | | | |
| G188F, K229W | | | | | | | | |
| L111V, D152S, | 49% | 12% | 35% | 11% | 26% | 8% | 4% | 5% |
| M155L, A162W, | | | | | | | | |
| Q138K, K229W | | | | | | | | |
| L111V, D152S, | 34% | 15% | 32% | 9% | 20% | 4% | NA | NA |
| M155L, A162W, | | | | | | | | |
| K229W, S234G | | | | | | | | |
| L111V, D152S, | 37% | 24% | 33% | 21% | 19% | 10% | NA | NA |
| M155L, A162W, | | | | | | | | |
| T102K, E105K, | | | | | | | | |
| K229W | | | | | | | | |
| L111V, D152S, | 33% | 14% | 32% | 11% | 18% | 3% | NA | NA |
| M155L, A162W, | | | | | | | | |
| S186T, K229W | | | | | | | | |
| L111V, D152S, | 36% | 16% | 33% | 16% | 18% | 11% | NA | NA |
| M155L, A162W, | | | | | | | | |
| T102K, K229W | | | | | | | | |
| L111V, D152S, | 32% | 21% | 27% | 24% | 11% | 13% | NA | NA |
| M155L, A162W, | | | | | | | | |
| S47E, K229W | | | | | | | | |
| L111V, D152S, | 34% | 28% | 25% | 18% | 11% | 3% | NA | NA |
| M155L, A162W, | | | | | | | | |
| S56A, K229W | | | | | | | | |
| Wild-Type | 40% | 18% | 24% | 21% | 0% | 0% | 0% | 0% |

### Example 8: Purification of Aspergillus fumigatus GH61B and Penicillium emersonii GH61 Polypeptide Variants

Expression and purification of the wild-type Aspergillus fumigatus GH61B and Penicillium emersonii GH61 polypeptides was conducted as previously described in WO 2012/044835.

Strains expressing Aspergillus fumigatus GH61B and Penicillium emersonii GH61 polypeptide variants, generated as described in Examples 7 and 11, were cultured in shake flasks to generate material as described below for purification Following isolation of single colonies, the Aspergillus oryzae PFJO218 transformants were cultured for 4 days at 34° C. on COVE-N-Gly plates in preparation for larger scale fermentation. Spores were recovered from each plate using 0.01% TWEEN® 20. Each spore suspension (500 μl) was inoculated into 25 ml of M400 medium in 125 ml plastic shake flasks. The transformants were fermented for 3 days at 39° C. with agitation at 150 rpm and the broths were collected and filtered using 0.22 μm filters. The filtered culture broths were then concentrated by centrifugal ultrafiltration using VIVACELL® 100 5 kDa MWCO centrifugal concentration devices (Sartorius Stedim, Goettingen, Germany) and then buffer exchanged into 20 mM Tris-HCl pH 8.0.

The concentrated and buffer exchanged Aspergillus fumigatus GH61B and Penicillium emersonii GH61 polypeptide variants were further purified by one of two chromatographic methods. In one method, the concentrated and buffer exchanged broths were then each applied to a MONO Q® HR 16/10 column (GE Healthcare, Piscataway, NJ, USA) equilibrated with 20 mM Tris-HCl pH 8.0. Bound proteins were eluted with a linear gradient of 0-500 mM sodium chloride in 20 mM Tris-HCl pH 8.0. Fractions were analyzed by SDS-PAGE using a CRITERION® Stain-Free Tris-HCl 8-16% SDS-PAGE gel (Bio-Rad Laboratories, Inc., Hercules, CA, USA), pooled based on the abundance of an approximately 25 kDa band, and concentrated using VIVASPIN® kDa MWCO centrifugal concentration devices (GE Healthcare, Buckinghamshire, United Kingdom). Alternatively, the concentrated and desalted broths were then each applied to a HI LOAD® 26/60 SUPERDEX® 75 (GE Healthcare, Piscataway, NJ, USA) size exclusion column equilibrated with 20 mM Tris-HCl pH 8.0 and 150 mM NaCl. Applied proteins were eluted isocratically using 20 mM Tris-HCl pH 8.0 and 150 mM NaCl as the mobile phase. Fractions were analyzed by SDS-PAGE using a CRITERION® Stain-Free Tris-HCl 8-16% SDS-PAGE gel, pooled based on the abundance of an approximately 25 kDa band, and concentrated using VIVASPIN® 5 kDa MWCO centrifugal concentration devices and then buffer exchanged into 20 mM MES pH 6.0.

Protein concentrations were determined using a Microplate BCA™ Protein Assay Kit in which bovine serum albumin was used as a protein standard.

### Example 9: Determination of Tm (Melting Temperature) of the Aspergillus fumigatus Wild-Type GH61B Polypeptide and Aspergillus fumigatus GH61B Polypeptide Variants by Differential Scanning Calorimetry

The thermostabilities of the A. fumigatus wild-type GH61B polypeptide and the Aspergillus fumigatus GH61B polypeptide variants, which were purified as described in Example 8, were determined by Differential Scanning calorimetry (DSC) using a VP Differential Scanning calorimeter (MicroCal Inc., GE Healthcare, Piscataway, NJ, USA). The melting temperature, Tm (° C.), was taken as the top of denaturation peak (major endothermic peak) in thermograms (Cp vs. T) obtained after heating a 1 mg protein per ml solution of the enzyme in 50 mM sodium acetate pH 5.0, 100 μM CuSO₄, or a 1 mg protein per ml solution of the enzyme in 50 mM sodium acetate pH 5.0, 10 mM EDTA pH 5.0, at a constant programmed heating rate. One ml of sample and reference-solutions were degassed at 25° C. using a ThermoVac (MicroCal Inc., GE Healthcare, Piscataway, NJ, USA) prior to loading of sample and reference cells of the calorimeter. Sample and reference (reference: degassed water) solutions were manually loaded into the DSC and thermally pre-equilibrated to 25° C. before the DSC scan was performed from 25° C. to 95° C. at a scan rate of 90 K/hour. Denaturation temperatures were determined at an accuracy of approximately +/−1° C. The results of the thermostability determinations of the A. fumigatus GH61B polypeptide variants are shown in Table 6.

TABLE 6
Melting temperatures (° C.) of the A. fumigatus GH61B
polypeptide and variants of the A. fumigatus GH61B
polypeptide, as determined by differential scanning calorimetry

| | Tm + 100 μm | Tm + 10 mM |
|---|---|---|
| Mutations | CuSO₄ | EDTA pH 5 |
| WT | 69 | 59 |
| S186K | 77 | n.d. |
| S234K | 73 | n.d. |
| L111V, D152S, M155L, | n.d. | 69 |
| A162W, Q138K, K229W | | |

### Example 10: Determination of Tm (Melting Temperature) of the Aspergillus fumigatus Wild-Type GH61B Polypeptide and Aspergillus fumigatus GH61B Polypeptide Variants by Protein Thermal Unfolding Analysis

Protein thermal unfolding of the Aspergillus fumigatus GH61B polypeptide variants was monitored using SYPRO® Orange Protein Stain (Invitrogen, Naerum, Denmark) using a StepOnePlus™ Real-Time PCR System (Applied Biosystems Inc., Foster City, CA, USA). In a 96-well white PCR-plate, 15 μl of a protein sample (prepared as described in Example 8) in 100 mM sodium acetate pH 5.0 was mixed (1:1) with Sypro Orange (resulting concentration=10×; stock solution=5000× in DMSO) in 20 mM EDTA. The plate was sealed with an optical PCR seal. The PCR instrument was set at a scan-rate of 76° C. per hour, starting at 25° C. and finishing at 96° C. Fluorescence was monitored every 20 seconds using a built-in LED blue light for excitation and ROX-filter (610 nm, emission). Tm-values were calculated as the maximum value of the first derivative (dF/dK) (Gregory et al., 2009, J. Biomol. Screen. 14: 700). The results of the thermostability determinations are shown in Table 7.

TABLE 7
Melting temperatures (° C.) of the
A. fumigatus GH61B polypeptide
and variants determined
by thermal unfolding analysis

| Mutations | Tm |
|---|---|
| Wild-Type | 59 |
| Q138E | 61 |
| Q138L | 62 |
| D152S | 65 |

### Example 11: Construction of Penicillium emersonii GH61A Polypeptide Variants

Variant plasmid pLSBF10 was constructed through two sequential single site-directed mutagenesis reactions on pMMar45 (Example 1) using a QUIKCHANGE® 11 XL Site-Directed Mutagenesis Kit. Two mutagenic primers were designed to insert each desired mutation. A total of 125 ng of each primer was used in a PCR containing approximately 25 ng of template plasmid, 1× QUIKCHANGE® reaction buffer, 3 μl of QUIKSOLUTION®, 1 μl of XL dNTP mix (provided in the QUIKCHANGE® 11 XL Site-Directed Mutagenesis Kit), and 1 μl of 2.5 U/μl Pfu Ultra enzyme in a final volume of 50 μl. The PCR was performed using an EPPENDORF® MASTERCYCLER® thermocycler programmed for a 95° C. hot start; 1 cycle at 95° C. for 1 minute; 18 cycles each at 95° C. for 50 seconds, 60° C. for 50 seconds, and 68° C. for 10 minutes; and a 4° C. hold. One microliter of Dpn I was directly added to the amplification reaction and incubated at 37° C. for 1 hour. A 2 μl volume of the Dpn I digested reaction was used to transform E. coli XL10-GOLD® Ultracompetent Cells according to the manufacturer's instructions. E. coli transformants were selected on 2XYT+Amp agar plates. Plasmid DNA from several of the resulting E. coli transformants was prepared using a BIOROBOT® 9600. The insert was confirmed by DNA sequencing using a Model 3130xL Genetic Analyzer and dye-terminator chemistry from a BigDye® Terminator v3.1 Cycle Sequencing Kit. One of the clones with the desired 2 mutations was designated as pLSBF10. The primers used in this reaction can be found below in Table 8.

A summary of the primers used for the site-directed mutagenesis and the variant (S173C, F253C) obtained are shown in Table 8.

The resulting mutant plasmid DNA was prepared using a BIOROBOT® 9600. Each mutant plasmid was sequenced using a 3130xl Genetic Analyzer to verify the substitutions. The sequencing primers 996271 and pALLO2 3′ were used for verification.

TABLE 8

| | | Parent | | |
|---|---|---|---|---|
| Plasmid | Mutation(s) | Plasmid | Oligo ID # | Sequence |
| pLSBF10 | S173C, | pMMar45 | 615942 | TGGACCGTCACCATTCCCAA |
| | F253C | | | CTGCGTCGCCCCCGGCAAC |
| | | | | TACG (SEQ ID NO: 213) |
| | | | 615943 | CGTAGTTGCCGGGGGCGAC |
| | | | | GCAGTTGGGAATGGTGACG |
| | | | | GTCCA (SEQ ID NO: 214) |
| | | | 615944 | CCGGGGCCGCCTGAGCCGA |
| | | | | CGTGCTAGGCGGCCGCGGA |
| | | | | GGCCACC (SEQ ID NO: 215) |
| | | | 615945 | GGTGGCCTCCGCGGCCGCC |
| | | | | TAGCACGTCGGCTCAGGCG |
| | | | | GCCCCGG (SEQ ID NO: 216) |

The P. emersonii GH61A polypeptide variants were expressed using Aspergillus oryzae PFJO218 as host was performed according to the procedure described in Example 3.

### Example 12: Thermostability of the P. emersonii Wild-Type GH61A Polypeptide and P. emersonii GH61A Polypeptide Variant

Based on the residual activity ratios determined according to Example 5, screening of libraries constructed in the previous Examples generated the results listed in Table 9. Table 9 shows an average % Residual Activity (from 2-14 samples each for the combinatorial variants and 14 samples of wild-type after treatment at any of 65° C., 68° C., 72° C. or, 75° C.

The parent wild-type P. emersonii GH61A polypeptide showed decreased residual activity of 45%, 18% and 1% when the temperature of treatment was increased from 65° C. to 72° C. to 75° C., respectively (68° C. was not tested). The thermostability of the P. emersonii GH61A polypeptide combinatorial variant (S173C, F253C) ranged from 0.8-fold (decrease) to 2.2-fold increase at 72° C., and 30-fold compared to the wild-type P. emersonii GH61A polypeptide at 75° C.

TABLE 9
Penicillium emersonii GH61A polypeptide variants with improved
thermostability at 65° C., 68° C., 72° C. or 75° C. treatment

| | Avg | | Avg | | Avg | | Avg | |
|---|---|---|---|---|---|---|---|---|
| | % | | % | | % | | % | |
| | Res. | | Res. | | Res. | | Res. | |
| | Act. | Standard | Act. | Standard | Act. | Standard | Act. | Standard |
| Mutations | 65° C. | Deviation | 68° C. | Deviation | 72° C. | Deviation | 75° C. | Deviation |
| Wild-Type | 45% | 7% | NA | NA | 18% | 7% | 1% | 5% |
| S173C, F253C | 38% | 6% | 49% | 8% | 40% | 19% | 30% | 6% |

### Example 13: Determination of Tm (Melting Temperature) of the P. emersonii Wild-Type GH61A Polypeptide and P. emersonii GH61A Polypeptide Variant by Differential Scanning Calorimetry

The thermostabilities of the P. emersonii wild-type GH61A polypeptide and the P. emersonii GH61A polypeptide variant (S173C, F253C), purified as described in Example 8, were determined by Differential Scanning calorimetry (DSC) using a VP Differential Scanning calorimeter as described in Example 9. The results of the thermostability determination of the P. emersonii GH61A polypeptide variant are shown in Table 10.

TABLE 10
Melting temperatures (° C.) of the P. emersonii wild-type
GH61A polypeptide and P. emersonii GH61A polypeptide
and variant of P. emersonii GH61A polypeptide,
as determined by differential scanning calorimetry

| | Tm + 100 um | Tm + 10 mM |
|---|---|---|
| Enzyme Sample | CuSO4 pH 5 | EDTA pH 5 |
| Wild-Type | 82 | 74 |
| S173C, F253C | 87 | 77 |

### Example 14: Determination of Tm (Melting Temperature) of the P. emersonii Wild-Type GH61A Polypeptide and P. emersonii GH61A Polypeptide Variant by Protein Thermal Unfolding Analysis

Protein thermal unfolding of the Penicillium emersonii GH61A polypeptide variant (S173C, F253C) was monitored using SYPRO® Orange Protein Stain and was performed using a StepOnePlus™ Real-Time PCR System as described as Example 10. The culture broths of P. emersonii GH61A wild-type polypeptide and P. emersonii GH61A polypeptide variant were prepared as described in Example 11. The results of the thermostability determination are shown in Table 11.

TABLE 11
Melting temperature (° C.) of
Penicillium emersonii
GH61A polypeptide variants by
protein thermal unfolding analysis

| Mutations | Tm |
|---|---|
| Wild-Type | 64 |
| S173C, F253C | 69 |

### Example 15: Construction of Expression Vectors pDFng153-4, pDFng154-17, pDFng155-33, pDFng156-37, and pDFng157-51

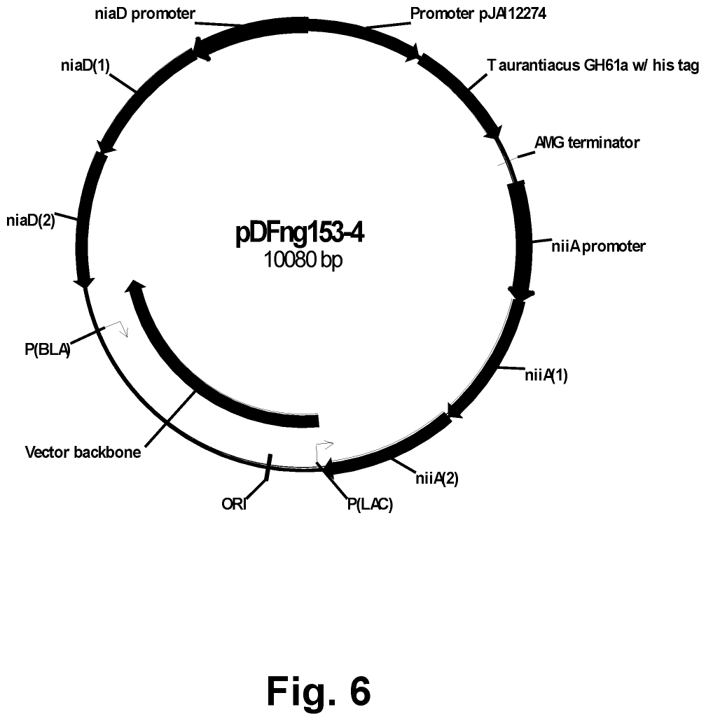

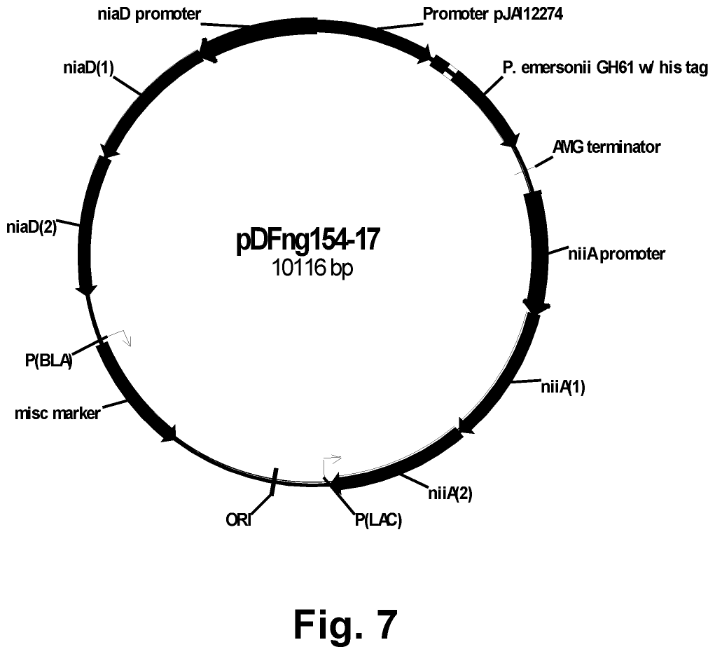

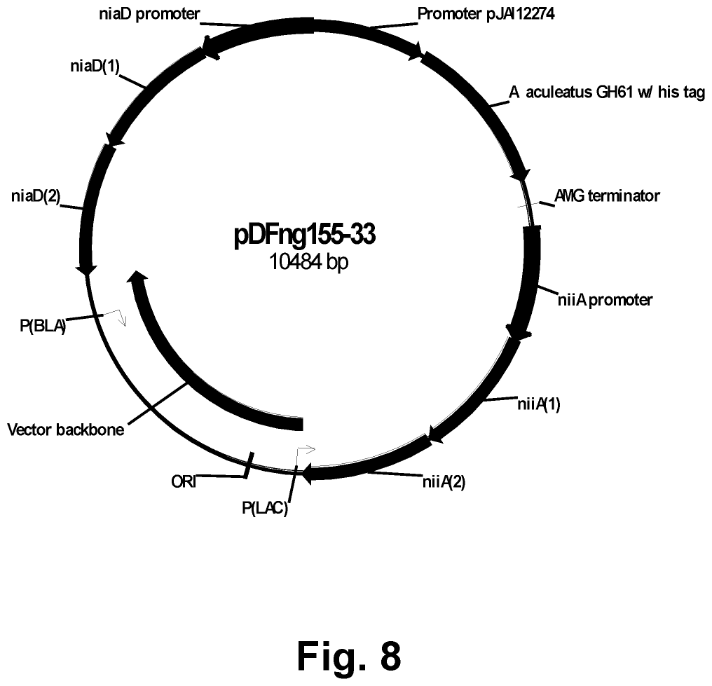

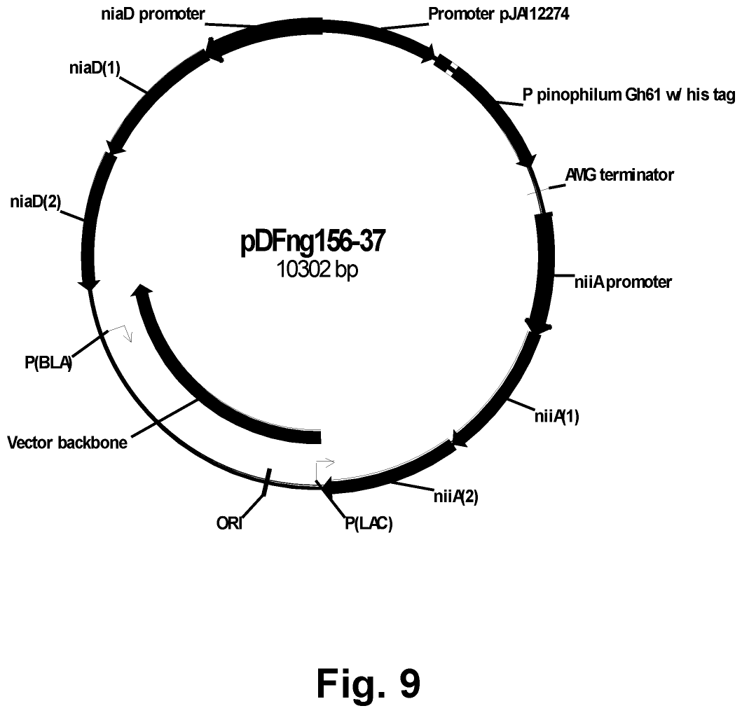

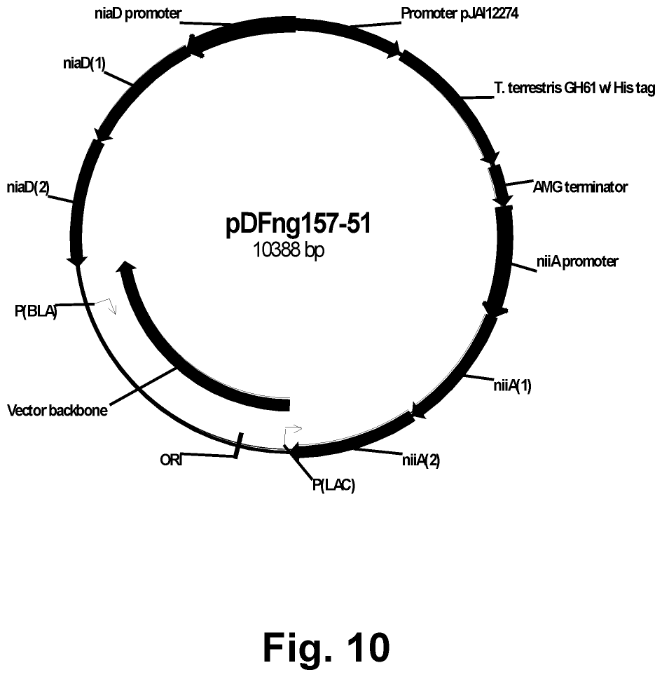

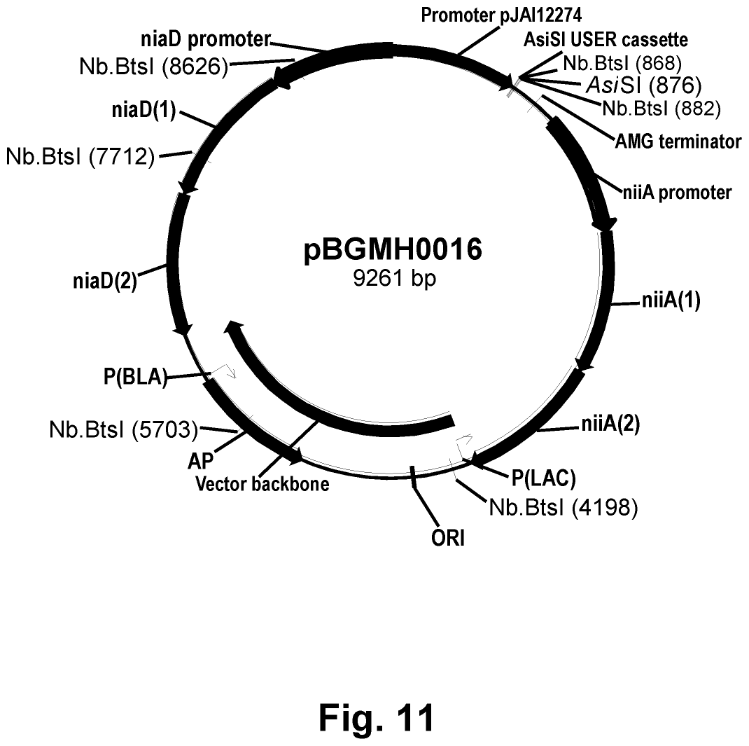

Plasmids pDFng153-4 (FIG. 6), pDFng154-17 (FIG. 7), pDFng155-33 (FIG. 8), and pDFng156-37 (FIG. 9), and pDFng157-51 (FIG. 10) were constructed as described below for expression of the Thermoascus aurantiacus GH61A polypeptide, Penicillium emersonii GH61A polypeptide, Aspergillus aculeatus GH61 polypeptide, Penicillium pinophilum GH61 polypeptide, and Thielavia terrestris GH61 polypeptide, respectively, and generation of the variants listed in Table 12. The plasmids were constructed using plasmid pBGMH16 (FIG. 11).

Plasmid pBGMH16 was constructed according to the protocol described below. A Nb.BtsI recognition site in pUC19 was removed by PCR using the primer pair BGMH24 and BGMH25 shown below followed by the uracil-specific excision reagent USER™ based cloning (New England BioLabs, Ipswich, MA, USA). Plasmid pUC19 is described in Yanisch-Perron et al., 1985, Gene 33(1): 103-19.

Primer BGM H24:
(SEQ ID NO: 217)
ATGCAGCGCUGCCATAACCATGAGTGA
Primer BGMH25:
(SEQ ID NO: 218)
AGCGCTGCAUAATTCTCTTACTGTCATG

Underlined sequence is used in the USER™ assisted fusion of the PCR fragment creating pBGMH13. USER™ (Uracil-Specific Excision Reagent) Enzyme (New England Biolabs, Ipswich, MA, USA) generates a single nucleotide gap at the location of a uracil. USER™ Enzyme is a mixture of Uracil DNA glycosylase (UDG) and the DNA glycosylase-lyase endonuclease VIII. UDG catalyzes the excision of a uracil base, forming an abasic (apyrimidinic) site while leaving the phosphodiester backbone intact. The lyase activity of endonuclease VIII cleaves the phosphodiester backbone at the 3′ and 5′ sides of the basic site so that base-free deoxyribose is released.

The amplification reaction was composed of 100 ng of each primer, 10 ng of pUC19, 1× PfuTurbo® Cₓ Reaction Buffer (Stratagene, La Jolla, CA, USA), 2.5 μl of a blend of dATP, dTTP, dGTP, and dCTP, each at 10 mM, and 2.5 units of PfuTurbo® Cₓ Hot Start DNA Polymerase (Stratagene, La Jolla, CA, USA) in a final volume of 50 μl. The reaction was performed using a EPPENDORF® MASTERCYCLER® 5333 programmed for 1 cycle at 95° C. for 2 minutes; 32 cycles each at 95° C. for 30 seconds, 55° C. for 30 seconds, and 72° C. for 3 minutes; and a final elongation at 72° C. for 7 minutes. Five μl of 10× NEBuffer 4 (New England Biolabs, Inc., Ipswich, MA, USA) and 20 units of Dpn I were added and incubated at 37° C. for 1 hour. The Dpn I was inactivated at 80° C. for 20 minutes. A total of 100 ng of the PCR product and 1 unit of USER™ enzyme in a total volume of 10 μl were incubated 20 minutes at 37° C. followed by 20 minutes at 25° C. Ten μl were transformed into ONE SHOT® TOP10 competent cells. E. coli transformants were selected on LB+Amp agar plates and plasmid DNA was prepared using QIAPREP® Spin Miniprep Kit (QIAGEN Inc., Valencia, CA, USA). The resulting plasmid pBGMH13 was confirmed by sequencing using an Applied Biosystems Model 3730XL Automated DNA Sequencer using version 3.1 BIG-DYE™ terminator chemistry (Applied Biosystems, Inc., Foster City, CA, USA).

Plasmid pBGMH14 contains part of pBGMH13 as vector backbone and a Pac I/Nt.BbvCI USER™ cassette (Hansen et al., 2011, Appl. Environ. Microbiol. 77(9): 3044-51) which is flanked by part of the A. oryzae niaD gene on one side and part of the A. oryzae niiA gene on the other side. The Pac I/Nt.BbvCI USER™ cassette can be linearized with Pac I and Nt.BbvCI and a PCR product with compatible overhangs can be cloned into this site (New England Biolabs, Ipswich, MA, USA).

An Aspergillus oryzae niiA fragment was generated using primers BGMH27 and BGMH29 shown below. The primer pair BGMH28 and BGMH32 shown below was used to amplify the Aspergillus oryzae niaD gene region and primer pair BGMH30 and BGMH31 shown below was used to amplify the plasmid backbone region.

Primer BGMH 27:
(SEQ ID NO: 219)
AATTAAGUCCTCAGCGTGATTTAAAACGCCATTGCT
Primer BGMH 28:
(SEQ ID NO: 220)
ACTTAATUAAACCCTCAGCGCAGTTAGGTTGGTGTTCTTCT
Primer BGMH 29:
(SEQ ID NO: 221)
AGCTCAAGGAUACCTACAGTTATTCGAAA
Primer BGMH 30:
(SEQ ID NO: 222)
ATCCTTGAGCUGTTTCCTGTGTGAAATTGTTATCC
Primer BGMH 31:
(SEQ ID NO: 223)
ATCTCCTCUGCTGGTCTGGTTAAGCCAGCCCCGACAC
Primer BGMH 32:
(SEQ ID NO: 224)
AGAGGAGAUAATACTCTGCGCTCCGCC

Underlined sequence was used in the USER™ assisted fusion of the three fragments. The sequence marked in bold was used to introduce a PacI/Nt.BbvCI USER™ cassette (Hansen et al., 2011, supra) between the niiA and niaD fragments.

Genomic DNA from A. oryzae BECH2 (WO 00/39322) was purified using a FASTDNA™ 2 ml SPIN Kit for Soil (MP Biomedicals, Santa Ana, California, USA).

Each PCR was composed of 100 ng of each primer, template DNA (pBGMH13 or A. oryzae BECH2 genomic DNA), 1× PfuTurbo® Cₓ Reaction Buffer, 2.5 μl of a blend of dATP, dTTP, dGTP, and dCTP, each at 10 mM, and 2.5 units of PfuTurbo® Cₓ Hot Start DNA Polymerase in a final volume of 50 μl. The PCRs were performed using a EPPENDORF® MASTERCYCLER® 5333 programmed for 1 cycle at 95° C. for 2 minutes; 32 cycles each at 95° C. for 30 seconds, 55° C. for 30 seconds, and 72° C. for 4 minutes; and a final elongation at 72° C. for 10 minutes. Where template DNA was a plasmid, 5 μl of 10× NEBuffer 4 and 20 units of Dpn I were added and incubated at 37° C. for 1 hour. The Dpn I was inactivated at 80° C. for 20 minutes. Fifty ng of each of the PCR products and 1 unit of USER™ enzyme in a total volume of 10 μl were incubated for 20 minutes at 37° C. followed by 20 minutes at 25° C. Then 10 μl were transformed into ONE SHOT® TOP10 competent cells. The three fragments were fused by uracil-specific excision reagent based cloning (Nour-Eldin et al., 2010, Methods Mol. Biol. 643: 185-200). E. coli transformants were selected on LB+Amp agar plates and plasmid DNA was prepared using QIAPREP® Spin Miniprep Kit. Plasmid pBGMH14 was confirmed by sequencing using an Applied Biosystems Model 3730XL Automated DNA Sequencer using version 3.1 BIG-DYE™ terminator chemistry. Promoter P13amy is a derivative of the NA2-tpi promoter from pJaL676 (WO 2003/008575). The A. niger AMG terminator used is described by Christensen et al., 1988, Nature Biotechnology 6: 141-1422. The P13amy promoter and AMG terminator were cloned into the Pac I/Nt.BbvCI USER™ cassette in pBGMH14. The primers shown below were designed so that an Asi SI/Nb.BtsI USER™ cassette (Hansen et al., 2011, supra) was introduced between the promoter and terminator.

Primer BGMH 49:
(SEQ ID NO: 225)
GGGTTTAAUCCTCACACAGGAAACAGCTATGA
Primer BGMH 50:
(SEQ ID NO: 226)
AGTGTCTGCGAUCGCTCTCACTGCCCCCAGTTGTGTATATAGAGGA
Primer BGMH 51:
(SEQ ID NO: 227)
ATCGCAGACACUGCTGGCGGTAGACAATCAATCCAT
Primer BGMH 52:
(SEQ ID NO: 228)
GGACTTAAUGGATCTAAGATGAGCTCATGGCT

Underlined sequence was used in the USER™ assisted fusion of the two fragments into a Pac I/Nt.BbvCI digested pBGMH14. The sequence marked in bold was used to introduce a AsiSI/Nb.BtsI USER™ cassette (Hansen et al., 2011, supra) between the promoter and terminator.

The primer pair BGMH49 and BGMH50 was used to amplify promoter P13amy and the primer pair BGMH51 and BGMH52 was used to amplify the AMG terminator. The PCR was composed of 100 ng of each primer, template DNA, 1× PfuTurbo® Cₓ Reaction Buffer, 2.5 μl of a blend of dATP, dTTP, dGTP, and dCTP, each at 10 mM, and 2.5 units of PfuTurbo® Cₓ Hot Start DNA Polymerase in a final volume of 50 μl. The reaction was performed using a EPPENDORF® MASTERCYCLER® 5333 programmed for 1 cycle at 95° C. for 2 minutes; 32 cycles each at 95° C. for 30 seconds, 55° C. for 30 seconds, and 72° C. for 45 seconds; and a final elongation at 72° C. for 3 minutes. Then 5 μl of 10× NEBuffer 4 and 20 units of Dpn I were added and incubated at 37° C. for 1 hour. The Dpn I was inactivated at 80° C. for 20 minutes.

The two fragments were fused into Pac I/Nt.BbvCI digested pBGMH14 by USER™ based cloning method in a reaction composed of 10 ng of Pac I/Nt.BbvCI digested pBGMH14, 50 ng of each of the two PCR products, and 1 unit of USER™ enzyme in a total volume of 10 μl. The reaction was incubated at 37° C. for 20 minutes followed by 20 minutes at 25° C. Then 10 μl were transformed into ONE SHOT® TOP10 competent cells. E. coli transformants were selected on LB+Amp agar plates and plasmid DNA was prepared using QIAPREP® Spin Miniprep Kit. Plasmid pBGMH16 was confirmed by sequencing.

DNA sequence was verified by Sanger sequencing with an Applied Biosystems Model 3730XL Automated DNA Sequencer using version 3.1 BIG-DYE™ terminator chemistry. Sequencing primers used for verification of niiA, niaD, the P13amy promoter, AsiSI/Nb.BtsI USER™ cassette, and AMG terminator sequence in BGMH16 are shown below.

BGMH 36
(SEQ ID NO: 229)
ACGCCATTGCTATGATGCTTGAAG
BGMH 37
(SEQ ID NO: 230)
TGGTGAGGTGCTATCGTCCTT
BGMH 38
(SEQ ID NO: 231)
CTTCCTGTAGGTGCACCGAAG
BGMH 39
(SEQ ID NO: 232)
ACAGAACGATATCGGACCTCG
BGMH 40
(SEQ ID NO: 233)
TCGTTATGTTAAGTCTTCTATCA
BGMH 41
(SEQ ID NO: 234)
AGAGCTCGAAGTTCCTCCGAG
BGMH 42
(SEQ ID NO: 235)
TATCACGAGGCCCTTTCGTCTC
BGMH 43
(SEQ ID NO: 236)
TCCGTCGGCTCCTCTCCTTCGT
BGMH 44
(SEQ ID NO: 237)
TGCATATCCTCTGACAGTATATGA
BGMH 45
(SEQ ID NO: 238)
CAGTGAAGAGGGCAGTCGATAGT
BGMH 46
(SEQ ID NO: 239)
ACGAGGAACATGGCTATCTGGA
BGMH 47
(SEQ ID NO: 240)
TCAGCTCATTCTGGGAGGTGGGA
BGMH 48
(SEQ ID NO: 241)
ACTCCAGGATCCTTTAAATCCA
BGMH 53
(SEQ ID NO: 242)
ACTGGCAAGGGATGCCATGCT
BGMH 54
(SEQ ID NO: 243)
TGATCATATAACCAATTGCCCT
BGMH 55
(SEQ ID NO: 244)
AGTTGTGTATATAGAGGATTGA
BGMH 56
(SEQ ID NO: 245)
TGGTCCTTCGCTCGTGATGTGGA
BGMH 57
(SEQ ID NO: 246)
AGTCCTCAGCGTTACCGGCA
BGMH 58
(SEQ ID NO: 247)
ACCCTCAGCTGTGTCCGGGA
BGMH 59
(SEQ ID NO: 248)
TGGTATGTGAACGCCAGTCTG

Plasmid pBGMH16 contains flanking regions designed to repair the niiA gene and niaD gene in Aspergillus oryzae COLs1300. Plasmid pBGMH16 was digested with Asi Si and Nb. Bts I to linearize the plasmid and create single stranded overhangs so that a PCR product with compatible overhangs can be cloned into this site by USER™ cloning (New England Biolabs, Inc., Ipswich, MA, USA). The digested plasmid was purified using a DNA Purification Kit (QIAGEN Inc., Valencia, CA, USA) according to the manufacturer's instructions.

The T. aurantiacus GH61A polypeptide coding sequence (SEQ ID NO: 13 [genomic DNA sequence] and SEQ ID NO: 14 [deduced amino acid sequence]), P. emersonii GH61A polypeptide coding sequence (SEQ ID NO: 35 [genomic DNA sequence] and SEQ ID NO: 36 [deduced amino acid sequence]), A. aculeatus GH61 polypeptide coding sequence (SEQ ID NO: 67 [genomic DNA sequence] and SEQ ID NO: 68 [deduced amino acid sequence]), P. pinophilum GH61 polypeptide coding sequence (SEQ ID NO: 31 [genomic DNA sequence] and SEQ ID NO: 32 [deduced amino acid sequence]), and T. terrestris GH61 polypeptide coding sequence (SEQ ID NO: 45 [genomic DNA sequence; cDNA was used herein] and SEQ ID NO: 46 [deduced amino acid sequence]) were amplified from source plasmids described below using the primers shown in Table 12. Bold letters represent coding sequence. The single deoxyuridine (U) residue inserted into each primer is the U that is excised from the PCR products using the USER™ enzyme (New England Biolabs, Inc., Ipswich, MA, USA) to obtain overhangs for the insertion site. The underline letters represent a His tag. The remaining sequences are homologous to insertion sites of pBGMH16 for expression of the GH61 polypeptides.

TABLE 12

| | Source | | | |
|---|---|---|---|---|
| GH61 origin | Template | Plasmid | Primer ID | Primer Sequence |
| Thermoascus | pDFng113 | pDFng153-4 | TaGH61_US | AGAGCGA(U)ATGTCCTTTTCCA |
| aurantiacus | Example 1 | | ERtagF | AGATAAT (SEQ ID NO: 249) |
| GH61A | | | TaGH61_US | TCTGCGA(U)TTAGTGATGGTG |
| | | | ER_HIStagR | GTGATGATGACCAGTATACAG |
| | | | | AGGAGGAC (SEQ ID NO: 250) |
| Penicillium | pMMar45 | pDFng154- | PeGH61_US | AGAGCGA(U)ATGCTGTCTTCG |
| emersonii | Example 1 | 17 | ERtagF | ACGACTCG (SEQ ID NO: 251) |
| GH61A | | | PeGH61_US | TCTGCGA(U)CTAGTGATGGTG |
| | | | ER_HIStagR | GTGATGATGGAACGTCGGCTC |
| | | | | AGGCGGCC (SEQ ID NO: 252) |
| Aspergillus | Xyz1566 | pDFng155- | AaGH61_US | AGAGCGA(U)ATGTCTGTTGCT |
| aculeatus | (WO2012/ | 33 | ERtagF | AAGTTTGCTGGTG (SEQ ID NO: |
| GH61 | 030799) | | | 253) |
| | | | AaGH61_US | TCTGCGA(U)TTAGTGATGGTG |
| | | | ER_HIStagR | GTGATGATGGGCGGAGAGGTC |
| | | | | ACGGGCGT (SEQ ID NO: 254) |
| Penicillium | pSMai215 | pDFng156- | PpGH61_US | AGAGCGA(U)ATGCCTTCTACT |
| pinophilum | (Example 15) | 37 | ERtagF | AAAGTCGCTGCC (SEQ ID NO: |
| GH61 | | | | 255) |
| | | | PpGH61_US | TCTGCGA(U)TCAGTGATGGTG |
| | | | ER_HIStagR | GTGATGATGAAGGACAGTAGT |
| | | | | GGTGATGA (SEQ ID NO: 256) |
| Thielavia | pAG68 | pDFng157- | TtGH61_USE | AGAGCGA(U)ATGCCTTCTTTCG |
| terrestris | (WO2011/ | 51 | RtagF | CCTCCAAGACTCTCCTTTC |
| GH61 | 035027) | | | (SEQ ID NO: 257) |
| | | | TtGH61_USE | TCTGCGA(U)TCAGTGATGGTG |
| | | | R_HIStagR | GTGATGATGGTTTGCCTCCTCA |
| | | | | GCCCCTC (SEQ ID NO: 258 ID |
| | | | | NO) |

Construction of plasmid pDFng153-4 containing the Thermoascus aurantiacus GH61A polypeptide coding sequence is described below. The T. aurantiacus GH61A polypeptide coding sequence was amplified from plasmid pDFng113 using the primers shown in Table 12 with overhangs designed for cloning into plasmid pBGMH16. The amplification was composed of 100 ng of each primer listed in Table 12, 30 ng of pDFng113, 1× PfuTurbo® Cₓ Reaction Buffer, 2.5 μl of a blend of dATP, dTTP, dGTP, and dCTP, each at 10 mM, and 2.5 units of PfuTurbo® Cₓ Hot Start DNA Polymerase in a final volume of 50 μl. The PCR was performed using an EPPENDORF® MASTERCYCLER® 5333 programmed for 1 cycle at 95° C. for 2 minutes; 30 cycles each at 95° C. for 30 seconds, 57.7° C. for 30 seconds, and 72° C. for 1.5 minutes; and a final elongation at 72° C. for 10 minutes. The heat block then went to a 10° C. soak cycle.

The PCR solution was analyzed by 0.7% agarose gel electrophoresis using TBE buffer where an approximately 894 bp PCR product band was observed. The PCR solution was then digested with 1 μl of Dpn I and 4.5 μl of NEBuffer 4 at 37° C. overnight and purified using a QIAGEN® Purification Kit according to the manufacturer's instructions.

The homologous ends of the 894 bp PCR product and the Asi SI and Nb.BtsI digested pBGMH16 were joined together in a reaction composed of 10 μl of the PCR containing the 894 bp PCR product, 1 μl of the Asi SI and Nb.BtsI digested plasmid pBGMH16, and 1 μl of USER™ enzyme. The reaction was incubated for 15 minutes at 37° C., followed by 15 minutes at 25° C. Ten μl of the reaction were transformed into E. coli XL10-GOLD® Super Competent Cells according to the manufacturer's instructions. E. coli transformants were selected on 2XYT+Amp agar plates. Plasmid DNA from several of the resulting E. coli transformants was prepared using a BIOROBOT® 9600. The T. aurantiacus GH61A polypeptide coding sequence insert was confirmed by DNA sequencing using a Model 3130xL Genetic Analyzer and dye-terminator chemistry from a BigDye® Terminator v3.1 Cycle Sequencing Kit. The sequencing primers shown below were used for verification of the gene insert and sequence.

Primer TaGH61seqF:
(SEQ ID NO: 259)
CCCAGTTATCAACTACCTTG
Primer pBGMH16seqF:
(SEQ ID NO: 260)
CTCAATTTACCTCTATCCAC
Primer pBGMH16seqR:
(SEQ ID NO: 261)
TATAACCAATTGCCCTCATC

A plasmid containing the correct T. aurantiacus GH61A polypeptide coding sequence was selected and designated pDFng153-4.

Construction of plasmid pDFng154-17 containing the Penicillium emersonii GH61A polypeptide coding sequence is described below. The P. emersonii GH61A polypeptide coding sequence was amplified from plasmid pMMar45 using the primers shown in Table 12 with overhangs designed for cloning into plasmid pBGMH16. The amplification was composed of 100 ng of each primer listed in Table 12, 30 ng of pMMar45, 1× PfuTurbo® Cₓ Reaction Buffer, 2.5 μl of a blend of dATP, dTTP, dGTP, and dCTP, each at 10 mM, and 2.5 units of PfuTurbo® Cₓ Hot Start DNA Polymerase in a final volume of 50 μl. The amplification was performed using an EPPENDORF® MASTERCYCLER® 5333 programmed for 1 cycle at 95° C. for 2 minutes; 30 cycles each at 95° C. for 30 seconds, 64.1° C. for 30 seconds, and 72° C. for 1.5 minutes; and a final elongation at 72° C. for 10 minutes. The heat block then went to a 10° C. soak cycle.

The PCR solution was analyzed by 0.7% agarose gel electrophoresis using TBE buffer where an approximately 930 bp PCR product band was observed. The PCR solution was then digested with 1 μl of Dpn I and 4.5 μl of NEBuffer 4 at 37° C. overnight and purified using a QIAGEN® Purification Kit according to the manufacturer's instructions.

The homologous ends of the 930 bp PCR product and the Asi SI and Nb.BtsI digested pBGMH16 were joined together in a reaction composed of 10 μl of the PCR solution containing the 930 bp PCR product, 1 μl of the Asi SI and Nb.BtsI digested pBGMH16, and 1 μl of USER™ enzyme. The reaction was incubated for 15 minutes at 37° C., followed by 15 minutes at 25° C. Ten μl of the reaction were transformed into E. coli XL10-GOLD® Super Competent Cells according to the manufacturer's instructions. E. coli transformants were selected on 2XYT+Amp agar plates. Plasmid DNA from several of the resulting E. coli transformants was prepared using a BIOROBOT® 9600. The P. emersonii GH61A polypeptide coding sequence insert was confirmed by DNA sequencing using a Model 3130xL Genetic Analyzer and dye-terminator chemistry from a BigDye® Terminator v3.1 Cycle Sequencing Kit. The sequencing primers pBGMH16seqF and pBGMH16seqR and primer PeGH61seqF shown below were used for verification of the gene insert and sequence.

PeGH61seqF:
(SEQ ID NO: 261)
GCACCGTCGAGCTGCAGTGG

A plasmid containing the correct P. emersonii GH61A polypeptide coding sequence was selected and designated pDFng154-17.

Construction of plasmid pDFng155-33 containing the Aspergillus aculeatus GH61A polypeptide coding sequence is described below. The A. aculeatus GH61A polypeptide coding sequence was amplified from plasmid Xyz1566 (WO 2012/030799 Example 3) using primers shown in Table 12 with overhangs designed for cloning into plasmid pBGMH16. The amplification reaction was composed of 100 ng of each primer listed in Table 12, 30 ng of plasmid Xyz1566, 1× PfuTurbo® Cₓ Reaction Buffer, 2.5 μl of a blend of dATP, dTTP, dGTP, and dCTP, each at 10 mM, and 2.5 units of PfuTurbo® Cₓ Hot Start DNA Polymerase in a final volume of 50 μl. The amplification was performed using an EPPENDORF® MASTERCYCLER® 5333 programmed for 1 cycle at 95° C. for 2 minutes; 30 cycles each at 95° C. for 30 seconds, 63.4° C. for 30 seconds, and 72° C. for 1.5 minutes; and a final elongation at 72° C. for 10 minutes. The heat block then went to a 10° C. soak cycle.

The PCR product solution was analyzed by 0.7% agarose gel electrophoresis using TBE buffer where an approximately 1.3 kb PCR product band was observed. The PCR product solution was then digested with 1 μl of Dpn I and 4.5 μl of NEBuffer 4 at 37° C. overnight and purified using a QIAGEN® Purification Kit according to the manufacturer's instructions.

The homologous ends of the 1.3 kb PCR product and the digested pBGMH16 were joined together in a reaction composed of 10 μl of the PCR containing the 1.3 kb PCR product, 1 μl of the digested pBGMH16, and 1 μl of USER™ enzyme. The reaction was incubated for 15 minutes at 37° C., followed by 15 minutes at 25° C. Ten μl of the reaction were transformed into E. coli XL10-GOLD® Super Competent Cells according to the manufacturer's instructions. E. coli transformants were selected on 2XYT+Amp agar plates. Plasmid DNA from several of the resulting E. coli transformants was prepared using a BIOROBOT® 9600. The A. aculeatus GH61A polypeptide coding sequence insert was confirmed by DNA sequencing using a Model 3130xL Genetic Analyzer and dye-terminator chemistry from a BigDye® Terminator v3.1 Cycle Sequencing Kit. The sequencing primers pBGMH16seqF and pBGMH16seqR and primer AaGH61seqF shown below were used for verification of the gene insert and sequence.

Primer AaGH61seqF:
(SEQ ID NO: 263)
CCTTGCCAACTGCAATGGTG

A plasmid containing the correct A. aculeatus GH61A polypeptide coding sequence was selected and designated pDFng155-33.

Plasmid pSMai215 was used to provide the DNA template of the Penicillium pinophilum GH61A gene for construction of pDFng156-37. Plasmid pSMai215 was constructed as described below. Two synthetic oligonucleotide primers shown below were designed to PCR amplify the Penicillium pinophilum GH61A polypeptide gene. An IN-FUSION™ PCR Cloning Kit was used to clone the fragment directly into the expression vector pMJ09 (WO 2005/047499). Bold letters represent coding sequence. The remaining sequence is homologous to the insertion sites of pMJ09.

Forward primer:
(SEQ ID NO: 264)
5′-GGACTGCGCACCATGCCTTCTACTAAAG-3′
Reverse primer:
(SEQ ID NO: 265)
5′-GCCACGGAGCTTAATTAATCAAAGGACAGTAGTG-3′

Fifty picomoles of each of the primers above were used in a PCR composed of 10 ng of pPin7, 1× EXPAND® High Fidelity PCR buffer with MgCl₂ (Roche Diagnostics Corporation, Indianapolis, IN, USA), 0.25 mM each of dATP, dTTP, dGTP, and dCTP, and 2.6 units of EXPAND® High Fidelity Enzyme Mix (Roche Diagnostics Corporation, Indianapolis, IN, USA), in a final volume of 50 μl. The amplification was performed using an EPPENDORF® MASTERCYCLER® 5333 epgradient S programmed for 1 cycle at 98° C. for 3 minute; 30 cycles each at 98° C. for 30 seconds, 59° C. for 30 seconds, and 72° C. for 1 minute; and a final elongation at 72° C. for 15 minutes. The heat block then went to a 4° C. soak cycle. The reaction products were isolated by 1% agarose gel electrophoresis in TAE buffer where an approximately 1.05 kb product band was observed on the gel. The PCR product solution was purified using a MINELUTE® Gel Extraction Kit.

Plasmid pMJ09 (WO 2005/047499) was digested with Nco I and Pac I, isolated by 1.0% agarose gel electrophoresis in TAE buffer, excised from the gel, and extracted using a QIAQUICK® Gel Extraction Kit (QIAGEN Inc., Valencia, CA, USA) according to the manufacturer's instructions.

The 1.05 kb gene fragment and the digested vector were ligated together using an IN-FUSION™ PCR Cloning Kit (Clontech Laboratories Inc., Mountain View, CA, USA) resulting in pSMai215. The ligation reaction (20 μl) was composed of 1× IN-FUSION™ Buffer, 1×BSA, 1 μl of IN-FUSION™ enzyme (diluted 1:10), 100 ng of the gel-purified Nco I/Pac I digested pMJ09, and 42 ng of the purified 1.05 kb PCR product. The reaction was incubated at 37° C. for 15 minutes follow by 50° C. for 15 minutes. After diluting the reaction mix with 50 μl of TE buffer (pH 8), 2.5 μl of the reaction were transformed into E. coli XL10 SOLOPACK® Gold Supercompetent cells. The E. coli transformation reactions were spread onto _{2XYTamp} agar plates. An E. coli transformant containing pSMai215 was detected by restriction digestion and plasmid DNA was prepared using a BIOROBOT® 9600. The Penicillium pinophilum GH61A insert in pSMai215 was confirmed by DNA sequencing.

Construction of plasmid pDFng156-37 containing the Penicillium pinophilum GH61A polypeptide coding sequence is described below. The P. pinophilum GH61A polypeptide coding sequence was amplified from plasmid pSMai215 using the primers shown in Table 12 with overhangs designed for cloning into plasmid pBGMH16. The amplification reaction was composed of 100 ng of each primer listed in Table 12, 30 ng of plasmid pSMai215, 1× PfuTurbo® Cₓ Reaction Buffer, 2.5 μl of a blend of dATP, dTTP, dGTP, and dCTP, each at 10 mM, and 2.5 units of PfuTurbo® Cₓ Hot Start DNA Polymerase in a final volume of 50 μl. The PCR was performed using an EPPENDORF® MASTERCYCLER® 5333 programmed for 1 cycle at 95° C. for 2 minutes; 30 cycles each at 95° C. for 30 seconds, 61.2° C. for 30 seconds, and 72° C. for 1.5 minutes; and a final elongation at 72° C. for 10 minutes. The heat block then went to a 10° C. soak cycle.

The PCR product solution was analyzed by 0.7% agarose gel electrophoresis using TBE buffer where an approximately 1.1 kb PCR product band was observed. The PCR product solution was then digested with 1 μl of Dpn I and 4.5 μl of NEB4 buffer at 37° C. overnight and purified using a QIAGEN® Purification Kit.

The homologous ends of the 1.1 kb PCR product and the digested pBGMH16 were joined together in a reaction composed of 10 μl of the PCR containing the 1.1 kb PCR product, 1 μl of the digested pBGMH16, and 1 μl of USER™ enzyme. The reaction was incubated at 37° C. for 15 minutes, followed by 15 minutes at 25° C. Ten μl of the reaction were transformed into E. coli XL10-GOLD® Super Competent Cells according to the manufacturer's instructions. E. coli transformants were selected on 2XYT+Amp agar plates. Plasmid DNA from several of the resulting E. coli transformants was prepared using a BIOROBOT® 9600. The P. pinophilum GH61A polypeptide coding sequence insert was confirmed by DNA sequencing using a Model 3130xL Genetic Analyzer and dye-terminator chemistry from a BigDye® Terminator v3.1 Cycle Sequencing Kit. The sequencing primers pBGMH16seqF and pBGMH16seqR and primer PpGH61seqF shown below were used for verification of the gene insert and sequence.

Primer PpGH61seqF:
(SEQ ID NO: 266)
CAATGGCAATTGTTCTACCG

A plasmid containing the correct P. pinophilum GH61A polypeptide coding sequence was selected and designated pDFng156-37.

Construction of plasmid pDFng157-51 containing the Thielavia terrestris GH61 polypeptide coding sequence is described below. The T. terrestris GH61 polypeptide coding sequence was amplified from plasmid pAG68 using the primers shown in Table 12 with overhangs designed for cloning into plasmid pBGMH16. The amplification reaction was composed of 100 ng of each primer listed in Table 12, 30 ng of plasmid pAG68, 1× PfuTurbo® Cₓ Reaction Buffer, 2.5 μl of a blend of dATP, dTTP, dGTP, and dCTP, each at 10 mM, and 2.5 units of PfuTurbo® Cₓ Hot Start DNA Polymerase in a final volume of 50 μl. The PCR was performed using an EPPENDORF® MASTERCYCLER® 5333 programmed for 1 cycle at 95° C. for 2 minutes; 30 cycles each at 95° C. for 30 seconds, 61.2° C. for 30 seconds, and 72° C. for 1.5 minutes; and a final elongation at 72° C. for 10 minutes. The heat block then went to a 10° C. soak cycle.

The PCR product solution was analyzed by 0.7% agarose gel electrophoresis using TBE buffer where an approximately 1.2 kb PCR product band was observed. The PCR product solution was then digested with 1 μl of Dpn I and 4.5 μl of NEB4 buffer at 37° C. overnight and purified using a QIAGEN® Purification Kit according to the manufacturer's instructions

The homologous ends of the 1.2 kb PCR product and the digested pBGMH16 were joined together in a reaction composed of 10 μl of the PCR containing the 1.1 kb PCR product, 1 μl of the digested pBGMH16, and 1 μl of USER™ enzyme. The reaction was incubated at 37° C. for 15 minutes, followed by 15 minutes at 25° C. Ten μl of the reaction were transformed into E. coli XL10-GOLD® Super Competent Cells according to the manufacturer's instructions. E. coli transformants were selected on 2XYT+Amp agar plates. Plasmid DNA from several of the resulting E. coli transformants was prepared using a BIOROBOT® 9600. The T. terrestris GH61A polypeptide coding sequence insert was confirmed by DNA sequencing using a Model 3130xL Genetic Analyzer and dye-terminator chemistry from a BigDye® Terminator v3.1 Cycle Sequencing Kit. The sequencing primers pBGMH16seqF and pBGMH16seqR and primer TtGH61seqF shown below were used for verification of the gene insert and sequence.

Primer TtGH61seqF:
(SEQ ID NO: 267)
CGACGGCAGCTCGGCGCCCG

A plasmid containing the correct T. terrestris GH61 polypeptide coding sequence was selected and designated pDFng157-51.

### Example 16: Construction of Thermoascus aurantiacus GH61 Polypeptide Variants

The Thermoascus aurantiacus GH61 polypeptide variants were constructed by SOE-PCR (Splicing by Overhang Extension Polymerase Chain Reaction) with plasmid pDFng153-4. In brief, the first PCR used forward primer EbZn NiaD Fwd and a mutation specific reverse primer (Table 13). The second PCR used reverse primer EbZn NiiA Rev and a mutation specific forward primer (Table 13) containing the sequence coding for the altered amino acid. The mutation specific forward and reverse primers contained 15-20 overlapping nucleotides. The third PCR used the overlapping nucleotides to splice together the fragments produced in the first and second reaction. Finally, using a nested forward primer BGMH110V2F and a nested reverse primer BGMH109V2R, the spliced fragment was amplified by PCR.

Primer EbZn NiaD Fwd:
(SEQ ID NO: 268)
5′-GCATTTATCAGGGTTATTGTCTCATGAGCGG-3′
Primer EbZn NiiA Rev:
(SEQ ID NO: 269)
5′-GCTGATAAATCTGGAGCCGGTGAGCG-3′
Primer BGMH110V2F:
(SEQ ID NO: 270)
5′-CCAGACCAGCAGAGGAGATAATACTCTGCG-3′
Primer BGMH109V2R:
(SEQ ID NO: 271)
5′-CAAGGATACCTACAGTTATTCGAAACCTCCTG-3′

The first PCRs for the T. aurantiacus GH61 polypeptide variants contained 0.2 picomole of the EbZn NiaD Fwd primer, 0.2 picomole of the reverse primer listed in Table 13, 10 ng of template (pDFng153-4), 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient (Eppendorf Scientific, Inc., Westbury, NY, USA) programmed for 1 cycle at 98° C. for 2 minutes; 35 cycles each at 98° C. for 25 seconds, 66° C. for 30 seconds, and 72° C. for 5 minute; and a final elongation at 72° C. for 10 minutes. The heat block then went to a 10° C. hold stage.

The second SOE-PCRs for the T. aurantiacus GH61 variants contained 0.2 picomole of the forward primer listed in Table 13, 0.2 picomole of the EbZn NiiA Rev primer, 10 ng of template (pDFng153-4), 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 25 seconds, 66° C. for seconds, and 72° C. for 5 minutes; and a final elongation at 72° C. for 10 minutes. The heat block then went to a 10° C. hold stage.

Each PCR product was analyzed by 1.0% agarose electrophoresis using TAE buffer where a 4.1 to 5.3 kb (as specified in Table 13) PCR product band was observed indicating proper amplification. The remaining 45 microliters were then treated with 10 units of Dpn I and 1× NEB4 to remove the remaining wild-type template. The reaction was incubated for 4 hours at 37° C. and then purified using a MINELUTE® 96 UF Purification Kit (QIAGEN Inc., Valencia, CA, USA). The purified PCR products were resuspended in deionized water to a final volume equal to 20 μl. The concentration of each fragment was measured using a NanoDrop 2000 (Thermo Scientific, Wilmington, DE, USA).

The third SOE-PCR for the T. aurantiacus GH61 variants contained 50 to 100 ng of each fragment produced in the first and second PCRs, 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 15 seconds, 68° C. for 30 seconds, and 72° C. for 10 minutes; and a final elongation at 72° C. for 10 minutes. The heat block then went to a 10° C. hold stage. Primer BGMH110V2F primer (0.2 picomole) and primer BGMH109V2R (0.2 picomole) were added during the annealing/elongation step of the fifth cycle to allow the overlapping nucleotides to splice before amplification.

The wild-type fragment was produced using conditions similar to the third PCR. The reaction was composed of 10 ng of template (pDFng153-4), 0.2 picomole of primer BGMH110V2F, 0.2 picomole of primer BGMH109V2R, 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 15 seconds, 68° C. for 30 seconds, and 72° C. for 10 minutes; and a final elongation at 72° C. for 10 minutes. The heat block then went to a 10° C. hold stage.

Each SOE-PCR product was analyzed by 1.0% agarose electrophoresis using TAE buffer where an approximately 8 kb PCR product band was observed indicating proper amplification. The remaining 45 μl of each SOE-PCR product was then purified using a MINELUTE® 96 UF Purification Kit. The purified PCR products were resuspended in deionized water to a final volume equal to 20 μl. The concentration of each fragment was measured using a NanoDrop 2000. The entire volume was then transformed into the Aspergillus oryzae COLs1300 strain as described in Example 21.

TABLE 13

| | | | | | PCR |
|---|---|---|---|---|---|
| Template | Amino Acid | | Primer | | fragment |
| Backbone | Substitution | Primer ID | Direction | Primer Sequence | size (kb) |
| Thermoascus | Q26I | 1203249 | Fwd | GTGGCTGGCCATGGCTTCGT | 5.3 |
| aurantiacus | | | | TATCAACATCGTGATTGATG | |
| GH61 | | | | GTAAAAAGT (SEQ ID NO: | |
| | | | | 272) | |
| | | 1203250 | Rev | AACGAAGCCATGGCCAGCC | 4.1 |
| | | | | ACTAGAGAAGCAGA (SEQ ID | |
| | | | | NO: 273) | |
| Thermoascus | Q42I | 1203262 | Fwd | GTTATGGCGGGTATCTAGTG | 5.2 |
| aurantiacus | | | | AACATCTATCCATACATGTCC | |
| GH61 | | | | AATCCTCC (SEQ ID NO: 274) | |
| | | 1203261 | Rev | GTTCACTAGATACCCGCCAT | 4.2 |
| | | | | AACTGTCGATTGTCA (SEQ | |
| | | | | ID NO: 275) | |
| Thermoascus | Q42V | 1203263 | Fwd | GTTATGGCGGGTATCTAGTG | 5.2 |
| aurantiacus | | | | AACGTCTATCCATACATGTC | |
| GH61 | | | | CAATCCTCC (SEQ ID NO: | |
| | | | | 277) | |
| | | 1203261 | Rev | GTTCACTAGATACCCGCCAT | 4.2 |
| | | | | AACTGTCGATTGTCA (SEQ | |
| | | | | ID NO: 278) | |
| Thermoascus | S123R | 1203276 | Fwd | TGCTCCGTGCAATGGTGATT | 4.9 |
| aurantiacus | | | | GTAGGACTGTGGATAAGACC | |
| GH61 | | | | CAATTAGAA (SEQ ID NO: | |
| | | | | 279) | |
| | | 1203277 | Rev | ACAATCACCATTGCACGGAG | |
| | | | | CAAGGTAGTTGATAA (SEQ | |
| | | | | ID NO: 280) | |
| Thermoascus | S138E | 1203280 | Fwd | ATTAGAATTCTTCAAAATTGC | 4.9 |
| aurantiacus | | | | CGAGGAGGGTCTCATCAATG | |
| GH61 | | | | ATGACAATCC (SEQ ID NO: | |
| | | | | 281) | |
| | | 1203279 | Rev | CTCGGCAATTTTGAAGAATT | 4.5 |
| | | | | CTAATTGGGTCTTATCC | |
| | | | | (SEQ ID NO: 282) | |
| Thermoascus | S138K | 1203281 | Fwd | ATTAGAATTCTTCAAAATTGC | |
| aurantiacus | | | | CGAGAAAGGTCTCATCAATG | 4.9 |
| GH61 | | | | ATGACAATCC (SEQ ID NO: | |
| | | | | 283) | |
| | | 1203279 | Rev | CTCGGCAATTTTGAAGAATT | 4.5 |
| | | | | CTAATTGGGTCTTATCC | |
| | | | | (SEQ ID NO: 284) | |
| Thermoascus | S138L | 1203282 | Fwd | ATTAGAATTCTTCAAAATTGC | 4.9 |
| aurantiacus | | | | CGAGCTGGGTCTCATCAATG | |
| GH61 | | | | ATGACAATCC (SEQ ID NO: | |
| | | | | 285) | |
| | | 1203279 | Rev | CTCGGCAATTTTGAAGAATT | 4.5 |
| | | | | CTAATTGGGTCTTATCC | |
| | | | | (SEQ ID NO: 286) | |
| Thermoascus | T163V | 1203285 | Fwd | GATAGCAGCCAACAACAGCT | 4.8 |
| aurantiacus | | | | GGGTCGTCACCATTCCAACC | |
| GH61 | | | | ACAATTGC (SEQ ID NO: 287) | |
| | | 1203286 | Rev | CCAGCTGTTGTTGGCTGCTA | 4.6 |
| | | | | TCAGATTGTCTGAAG (SEQ | |
| | | | | ID NO: 288) | |
| Thermoascus | T163E | 1203288 | Fwd | GATAGCAGCCAACAACAGCT | 4.8 |
| aurantiacus | | | | GGGAGGTCACCATTCCAACC | |
| GH61 | | | | ACAATTGC (SEQ ID NO: 289) | |
| | | 1203289 | Rev | CCAGCTGTTGTTGGCTGCTA | 4.6 |
| | | | | TCAGATTGTCTGAAG (SEQ | |
| | | | | ID NO: 290) | |
| Thermoascus | S186K | 1203297 | Fwd | GGCATGAGATTATTGCTCTT | 4.8 |
| aurantiacus | | | | CACAAAGCTCAGAACCAGGA | |
| GH61 | | | | TGGTGCC (SEQ ID NO: 291) | |
| | | 1203298 | Rev | GTGAAGAGCAATAATCTCAT | 4.6 |
| | | | | GCCTCAGAACATAGTT (SEQ | |
| | | | | ID NO: 292) | |
| Thermoascus | S186F | 1203299 | Fwd | GGCATGAGATTATTGCTCTT | 4.8 |
| aurantiacus | | | | CACTTCGCTCAGAACCAGGA | |
| GH61 | | | | TGGTGCC (SEQ ID NO: 293) | |
| | | 1203298 | Rev | GTGAAGAGCAATAATCTCAT | 4.6 |
| | | | | GCCTCAGAACATAGTT (SEQ | |
| | | | | ID NO: 294) | |
| Thermoascus | S186T | 1203300 | Fwd | GGCATGAGATTATTGCTCTT | 4.8 |
| aurantiacus | | | | CACACTGCTCAGAACCAGGA | |
| GH61 | | | | TGGTGCC (SEQ ID NO: 295) | |
| | | 1203298 | Rev | GTGAAGAGCAATAATCTCAT | 4.6 |
| | | | | GCCTCAGAACATAGTT (SEQ | |
| | | | | ID NO: 296) | |
| Thermoascus | S186Y | 1203301 | Fwd | GGCATGAGATTATTGCTCTT | 4.8 |
| aurantiacus | | | | CACTATGCTCAGAACCAGGA | |
| GH61 | | | | TGGTGCC (SEQ ID NO: 297) | |
| | | 1203298 | Rev | GTGAAGAGCAATAATCTCAT | 4.6 |
| | | | | GCCTCAGAACATAGTT (SEQ | |
| | | | | ID NO: 298) | |
| Thermoascus | I200V | 1203302 | Fwd | GCCCAGAACTATCCCCAGTG | 4.7 |
| aurantiacus | | | | CGTCAATCTGCAGGTCACTG | |
| GH61 | | | | GAGGTG (SEQ ID NO: 299) | |
| | | 1203303 | Rev | GCACTGGGGATAGTTCTGG | 4.7 |
| | | | | GCACCATCCTGGT (SEQ ID | |
| | | | | NO: 300) | |
| Thermoascus | A213E | 1203306 | Fwd | TGGAGGTGGTTCTGATAACC | 4.7 |
| aurantiacus | | | | CTGAGGGAACTCTTGGAACG | |
| GH61 | | | | GCACTC (SEQ ID NO: 301) | |
| | | 1203307 | Rev | AGGGTTATCAGAACCACCTC | 4.7 |
| | | | | CAGTGACCTGCAG (SEQ ID | |
| | | | | NO: 302) | |
| Thermoascus | A219C | 1203308 | Fwd | CCTGCTGGAACTCTTGGAAC | 4.7 |
| aurantiacus | | | | GTGCCTCTACCACGATACCG | |
| GH61 | | | | ATCCTG (SEQ ID NO: 303) | |
| | | 1203309 | Rev | CGTTCCAAGAGTTCCAGCAG | 4.7 |
| | | | | GGTTATCAGAACC (SEQ ID | |
| | | | | NO: 304) | |
| Thermoascus | A219E | 1203310 | Fwd | CCTGCTGGAACTCTTGGAAC | 4.7 |
| aurantiacus | | | | GGAGCTCTACCACGATACCG | |
| GH61 | | | | ATCCTG (SEQ ID NO: 305) | |
| | | 1203309 | Rev | CGTTCCAAGAGTTCCAGCAG | 4.7 |
| | | | | GGTTATCAGAACC (SEQ ID | |
| | | | | NO: 306) | |
| Thermoascus | A219Q | 1203312 | Fwd | CCTGCTGGAACTCTTGGAAC | 4.7 |
| aurantiacus | | | | GCAGCTCTACCACGATACCG | |
| GH61 | | | | ATCCTG (SEQ ID NO: 307) | |
| | | 1203309 | Rev | CGTTCCAAGAGTTCCAGCAG | 4.7 |
| | | | | GGTTATCAGAACC (SEQ ID | |
| | | | | NO: 308) | |
| Thermoascus | T248R | 1203320 | Fwd | CATCCCTGGTCCTCCTCTGT | 4.6 |
| aurantiacus | | | | ATAGGGGTCATCATCACCAC | |
| GH61 | | | | CATCACT (SEQ ID NO: 309) | |
| | | 1203319 | Rev | ATACAGAGGAGGACCAGGG | 4.8 |
| | | | | ATGATATAGCTGGAAA (SEQ | |
| | | | | ID NO: 310) | |

### Example 17: Construction of Penicillium emersonii GH61 Polypeptide Variants

The Penicillium emersonii GH61 polypeptide variants were constructed by SOE-PCR (Splicing by Overhang Extension Polymerase Chain Reaction) with plasmid pDFng154-17. In brief, the first PCR used forward primer EbZn NiaD Fwd and a mutation specific reverse primer (Table 14). The second PCR used reverse primer EbZn NiiA Rev and a mutation specific forward primer (Table 14) containing the sequence coding for the altered amino acid. The mutation specific forward and reverse primers contained 15-20 overlapping nucleotides. The third PCR used the overlapping nucleotides to splice together the fragments produced in the first and second reaction. Finally, using a nested forward primer BGMH110V2F and a nested reverse primer BGMH109V2R, the spliced fragment was amplified by PCR.

The first PCRs for the P. emersonii GH61 polypeptide variants contained 0.2 picomole of the EbZn NiaD Fwd primer, 0.2 picomole of the reverse primer listed in Table 14, 10 ng of template (pDFng154-17), 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 25 seconds, 66° C. for seconds, and 72° C. for 5 minute; and a final elongation at 72° C. for 10 minutes. The heat block then went to a 10° C. hold stage.

The second PCRs for the P. emersonii GH61 variants contained 0.2 picomole of the forward primer listed in Table 14, 0.2 picomole of the EbZn NiiA Rev primer, 10 ng of template (pDFng154-17), 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 25 seconds, 66° C. for seconds, and 72° C. for 5 minutes; and a final elongation at 72° C. for 10 minutes. The heat block then went to a 10° C. hold stage.

Each PCR product was analyzed by 1.0% agarose electrophoresis using TAE buffer where a 4.2 to 5.2 kb (as specified in Table 14) PCR product band was observed indicating proper amplification. The remaining 45 microliters were then treated with 10 units of Dpn I and 1× NEB4 to remove the remaining wild-type template. The reaction was incubated for 4 hours at 37° C. and then purified using a MINELUTE® 96 UF Purification Kit. The purified PCR products were resuspended in deionized water to a final volume equal to 20 μl. The concentration of each fragment was measured using a NanoDrop 2000.

The third SOE-PCR for the P. emersonii GH61 variants contained 50 to 100 ng of each fragment produced in the first and second PCRs, 4 nanomoles each dATP, dTTP, dGTP, and dCTP, 1× ADVANTAGE® GC-Melt Buffer (Clontech Laboratories, Inc., Mountain View, CA, USA), and 2.5 units of ADVANTAGE® GC Genomic LA Polymerase (Clontech Laboratories, Inc., Mountain View, CA, USA) in a final reaction volume of 50 μl. The amplification was performed using an EPPENDORF® MASTERCYCLER® Gradient programmed for 1 cycle at 94° C. for 1 minutes; 35 cycles each at 94° C. for 30 seconds, 66° C. for 30 seconds, and 72° C. for 10 minutes; and a final elongation at 72° C. for 10 minutes. The heat block then went to a 10° C. hold stage. Primer BGMH110V2F primer (0.4 picomole) and primer BGMH109V2R (0.4 picomole) were added during the annealing/elongation step of the fifth cycle to allow for the overlapping nucleotides to splice before amplification.

The wild-type fragment was produced using conditions similar to the third PCR. The reaction was composed of 10 ng of template (pDFng154-17), 0.4 picomole of primer BGMH110V2F, 0.4 picomole of primer BGMH109V2R, 1 nanomole each dATP, dTTP, dGTP, and dCTP, 1× ADVANTAGE® GC-Melt Buffer, and 2.5 units of ADVANTAGE® GC Genomic LA Polymerase in a final reaction volume of 50 μl. The amplification was performed using an EPPENDORF® MASTERCYCLER® Gradient programmed for 1 cycle at 94° C. for 1 minutes; 35 cycles each at 94° C. for 30 seconds, 66° C. for 30 seconds, and 72° C. for 10 minutes; and a final elongation at 72° C. for 10 minutes. The heat block then went to a 10° C. hold stage.

Each SOE-PCR product was analyzed by 1.0% agarose electrophoresis using TAE buffer where an approximately 8 kb PCR product band was observed indicating proper amplification. The remaining 45 μl of each SOE-PCR product was then purified using a MINELUTE® 96 UF Purification Kit. The purified PCR products were resuspended in deionized water to a final volume equal to 20 μl. The concentration of each fragment was measured using a NanoDrop 2000. The entire volume was then transformed into the Aspergillus oryzae COLs1300 strain as described in Example 21.

TABLE 14

| | | | | | PCR |
|---|---|---|---|---|---|
| Template | Amino Acid | | Primer | | fragment |
| Backbone | Substitution | Primer ID | Direction | Primer Sequence | size (kb) |
| Penicillium | S46V | 1203486 | Fwd | CTACAGCGGGTACATCGTCAA | 5.2 |
| emersonii | | | | CGTCTTCCCCTACGAATCCAA | |
| GH61 | | | | CCCAC (SEQ ID NO: 311) | |
| | | 1203484 | Rev | GTTGACGATGTACCCGCTGTA | 4.2 |
| | | | | GCTGTTGGGAGT (SEQ ID NO: | |
| | | | | 312) | |
| Penicillium | G76Q | 1203493 | Fwd | GTCGACGGCACAGGATACCA | 5.1 |
| emersonii | | | | ACAGCCGGACATCATCTGCC | |
| GH61 | | | | ACCG (SEQ ID NO: 313) | |
| | | 1203494 | Rev | TTGGTATCCTGTGCCGTCGAC | 4.3 |
| | | | | GAAGCCCAGG(SEQ ID NO: | |
| | | | | 314) | |
| Penicillium | S127R | 1203496 | Fwd | CGCCGTGCAACGGCAACTGC | 4.9 |
| emersonii | | | | CGCACCGTCGACAAGACGAC | |
| GH61 | | | | GCTG (SEQ ID NO: 315) | |
| | | 1203497 | Rev | GCAGTTGCCGTTGCACGGCG | 4.5 |
| | | | | CCAGGTAGGTG (SEQ ID NO: | |
| | | | | 316) | |

### Example 18: Construction of Aspergillus aculeatus GH61 Polypeptide Variants

The Aspergillus aculeatus GH61 polypeptide variants were constructed by SOE-PCR (Splicing by Overhang Extension Polymerase Chain Reaction) with plasmid pDFng155-33. In brief, the first PCR used forward primer EbZn NiaD Fwd and a mutation specific reverse primer (Table 15). The second PCR used reverse primer EbZn NiiA Rev and a mutation specific forward primer (Table 15) containing the sequence coding for the altered amino acid. The mutation specific forward and reverse primers contained 15-20 overlapping nucleotides. The third PCR used the overlapping nucleotides to splice together the fragments produced in the first and second reaction. Finally, using a nested forward primer BGMH110V2F and a nested reverse primer BGMH109V2R, the spliced fragment was amplified by PCR.

The first PCRs for the A. aculeatus GH61 polypeptide variants contained 0.2 picomole of the EbZn NiaD Fwd primer, 0.2 picomole of the reverse primer listed in Table 15, 10 ng of template (pDFng155-33), 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 25 seconds, 66° C. for seconds, and 72° C. for 5 minutes; and a final elongation at 72° C. for 10 minutes. The heat block then went to a 10° C. hold stage.

The second PCRs for the A. aculeatus GH61 variants contained 0.2 picomole of the forward primer listed in Table 15, 0.2 picomole of the EbZn NiiA Rev primer, 10 ng of template (pDFng155-33), 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 25 seconds, 66° C. for seconds, and 72° C. for 5 minutes; and a final elongation at 72° C. for 10 minutes. The heat block then went to a 10° C. hold stage.

Each PCR product was analyzed by 1.0% agarose electrophoresis using TAE buffer where a 4.6 to 5.1 kb (as specified in Table 15) PCR product band was observed indicating proper amplification. The remaining 45 microliters were then treated with 10 units of Dpn I and 1× NEB4 to remove the remaining wild-type template. The reaction was incubated for 4 hours at 37° C. and then purified using a MINELUTE® 96 UF Purification Kit. The purified PCR products were resuspended in deionized water to a final volume equal to 20 μl. The concentration of each fragment was measured using a NanoDrop 2000.

The third SOE-PCR for the A. aculeatus GH61 variants contained 50 to 100 ng of each fragment produced in the first and second PCRs, 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 15 seconds, 68° C. for 30 seconds, and 72° C. for 10 minutes; and a final elongation at 72° C. for 10 minutes. The heat block then went to a 10° C. hold stage. Primer BGMH110V2F primer (0.2 picomole) and primer BGMH109V2R (0.2 picomole) were added during the annealing/elongation step of the fifth cycle to allow for the overlapping nucleotides to splice before amplification.

The wild-type fragment was produced using conditions similar to the third PCR. The reaction was composed of 10 ng of template (pDFng155-33), 0.2 picomole of primer BGMH110V2F, 0.2 picomole of primer BGMH109V2R, 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 15 seconds, 68° C. for 30 seconds, and 72° C. for 10 minutes; and a final elongation at 72° C. for 10 minutes. The heat block then went to a 10° C. hold stage.

Each SOE-PCR product was analyzed by 1.0% agarose electrophoresis using TAE buffer where an approximately 8 kb PCR product band was observed indicating proper amplification. The remaining 45 μl of each SOE-PCR product was then purified using a MINELUTE® 96 UF Purification Kit. The purified PCR products were resuspended in deionized water to a final volume equal to 20 μl. The concentration of each fragment was measured using a NanoDrop 2000. The entire volume was then transformed into the Aspergillus oryzae COLs1300 strain as described in Example 21.

TABLE 15

| | | | | | PCR |
|---|---|---|---|---|---|
| | | | | | fragment |
| Template | Amino Acid | | Primer | | size |
| Backbone | Substitution | Primer ID | Direction | Primer Sequence | (kb) |
| Aspergillus | D70Q | 1203194 | Fwd | CGTCGATGGTAGCGAGTATG | 5.6 |
| aculeatus | | | | CTCAGGCCGACATCATTTGCC | |
| GH61 | | | | ACAAGA (SEQ ID NO: 317) | |
| | | 1203195 | Rev | AGCATACTCGCTACCATCGAC | 4.1 |
| | | | | GAAACCCAAGTCG (SEQ ID | |
| | | | | NO: 318) | |
| Aspergillus | D70T | 1203196 | Fwd | CGTCGATGGTAGCGAGTATG | 5.6 |
| aculeatus | | | | CTACCGCCGACATCATTTGCC | |
| GH61 | | | | ACAAGA (SEQ ID NO: 319) | |
| | | 1203195 | Rev | AGCATACTCGCTACCATCGAC | 4.1 |
| | | | | GAAACCCAAGTCG (SEQ ID | |
| | | | | NO: 320) | |
| Aspergillus | S136C | 1203202 | Fwd | CTCGAGTTTTTCAAGATTGAC | 5.4 |
| aculeatus | | | | GAGTGCGGTCTCATCAACGA | |
| GH61 | | | | CGACGAC (SEQ ID NO: 321) | |
| | | 1203203 | Rev | CTCGTCAATCTTGAAAAACTC | 4.3 |
| | | | | GAGGTCGGTCTTGG (SEQ ID | |
| | | | | NO: 322) | |
| Aspergillus | S136G | 1203208 | Fwd | CTCGAGTTTTTCAAGATTGAC | 5.4 |
| aculeatus | | | | GAGGGTGGTCTCATCAACGA | |
| GH61 | | | | CGACGAC (SEQ ID NO: 323) | |
| | | 1203203 | Rev | CTCGTCAATCTTGAAAAACTC | 4.3 |
| | | | | GAGGTCGGTCTTGG (SEQ ID | |
| | | | | NO: 324) | |
| Aspergillus | T147I | 1203209 | Fwd | ACGACGACGACGTCCCCGGT | 5.3 |
| aculeatus | | | | ATCTGGGCCAGTGATAACTTG | |
| GH61 | | | | ATCG (SEQ ID NO: 325) | |
| | | 1203210 | Rev | ACCGGGGACGTCGTCGTCGT | 4.4 |
| | | | | TGATGAGACCG (SEQ ID NO: | |
| | | | | 326) | |
| Aspergillus | T161V | 1203211 | Fwd | GATCGCCAACAACAACAGCT | 5.3 |
| aculeatus | | | | GGGTCGTGACCATCCCCTCT | |
| GH61 | | | | GACATTG (SEQ ID NO: 327) | |
| | | 1203212 | Rev | CCAGCTGTTGTTGTTGGCGAT | 4.4 |
| | | | | CAAGTTATCACTGG (SEQ ID | |
| | | | | NO: 328) | |
| Aspergillus | T161F | 1203213 | Fwd | GATCGCCAACAACAACAGCT | 5.3 |
| aculeatus | | | | GGTTCGTGACCATCCCCTCTG | |
| GH61 | | | | ACATTG (SEQ ID NO: 329) | |
| | | 1203212 | Rev | CCAGCTGTTGTTGTTGGCGAT | 4.4 |
| | | | | CAAGTTATCACTGG (SEQ ID | |
| | | | | NO: 330) | |
| Aspergillus | D167R | 1203220 | Fwd | TGGACTGTGACCATCCCCTCT | 5.3 |
| aculeatus | | | | CGTATTGCGGCTGGCAACTA | |
| GH61 | | | | CGTC (SEQ ID NO: 331) | |
| | | 1203221 | Rev | AGAGGGGATGGTCACAGTCC | 4.4 |
| | | | | AGCTGTTGTTGTT (SEQ ID | |
| | | | | NO: 332) | |
| Aspergillus | S184K | 1203222 | Fwd | GTCACGAAATCATTGCCCTTC | 5.2 |
| aculeatus | | | | ACAAGGCTGGTAACAAGGAT | |
| GH61 | | | | GGTGCTC (SEQ ID NO: 333) | |
| | | 1203223 | Rev | GTGAAGGGCAATGATTTCGTG | 4.5 |
| | | | | ACGGAGGACGTAG (SEQ ID | |
| | | | | NO: 334) | |
| Aspergillus | S184T | 1203225 | Fwd | GTCACGAAATCATTGCCCTTC | 5.2 |
| aculeatus | | | | ACACCGCTGGTAACAAGGAT | |
| GH61 | | | | GGTGCTC (SEQ ID NO: 335) | |
| | | 1203223 | Rev | GTGAAGGGCAATGATTTCGTG | 4.5 |
| | | | | ACGGAGGACGTAG (SEQ ID | |
| | | | | NO: 336) | |
| Aspergillus | S184Y | 1203226 | Fwd | GTCACGAAATCATTGCCCTTC | 5.2 |
| aculeatus | | | | ACTACGCTGGTAACAAGGATG | |
| GH61 | | | | GTGCTC (SEQ ID NO: 337) | |
| | | 1203223 | Rev | GTGAAGGGCAATGATTTCGTG | 4.5 |
| | | | | ACGGAGGACGTAG (SEQ ID | |
| | | | | NO: 338) | |
| Aspergillus | L198I | 1203227 | Fwd | GCTCAGAACTACCCTCAGTGC | 5.2 |
| aculeatus | | | | ATCAACTTGAAGGTCACTGGC | |
| GH61 | | | | GGC (SEQ ID NO: 339) | |
| | | 1203228 | Rev | GCACTGAGGGTAGTTCTGAG | 4.5 |
| | | | | CACCATCCTTGTT (SEQ ID | |
| | | | | NO: 340) | |
| Aspergillus | L198V | 1203229 | Fwd | GCTCAGAACTACCCTCAGTGC | 5.2 |
| aculeatus | | | | GTCAACTTGAAGGTCACTGGC | |
| GH61 | | | | GGC (SEQ ID NO: 341) | |
| | | 1203228 | Rev | GCACTGAGGGTAGTTCTGAG | 4.5 |
| | | | | CACCATCCTTGTT (SEQ ID | |
| | | | | NO: 342) | |
| Aspergillus | S217M | 1203237 | Fwd | CCTTCTGGCACTGCTGGTGA | 5.1 |
| aculeatus | | | | GATGCTGTACAAGGACACCG | |
| GH61 | | | | ATGCTG (SEQ ID NO: 343) | |
| | | 1203235 | Rev | CTCACCAGCAGTGCCAGAAG | 4.6 |
| | | | | GAGCGAGATCAC (SEQ ID NO: | |
| | | | | 344) | |
| Aspergillus | S217Q | 1203238 | Fwd | CCTTCTGGCACTGCTGGTGA | 5.1 |
| aculeatus | | | | GCAGCTGTACAAGGACACCG | |
| GH61 | | | | ATGCTG (SEQ ID NO: 345) | |
| | | 1203235 | Rev | CTCACCAGCAGTGCCAGAAG | 4.6 |
| | | | | GAGCGAGATCAC (SEQ ID NO: | |
| | | | | 346) | |
| Aspergillus | K220R | 1203239 | Fwd | ACTGCTGGTGAGAGCCTGTA | 5.1 |
| aculeatus | | | | CCGTGACACCGATGCTGGTA | |
| GH61 | | | | TCCTC (SEQ ID NO: 347) | |
| | | 1203240 | Rev | GTACAGGCTCTCACCAGCAG | 4.6 |
| | | | | TGCCAGAAGGAG (SEQ ID NO: | |
| | | | | 348) | |
| Aspergillus | A243P | 1203244 | Fwd | CTCCTACGATATTCCCGGACC | 5.0 |
| aculeatus | | | | TCCCATGTACAACGCTACCTC | |
| GH61 | | | | CAGCT (SEQ ID NO: 349) | |
| | | 1203245 | Rev | AGGTCCGGGAATATCGTAGG | 4.7 |
| | | | | AGGAAAGAGACTGG (SEQ ID | |
| | | | | NO: 350) | |

### Example 19: Construction of Penicillium pinophilum GH61 Polypeptide Variants

The Penicillium pinophilum GH61 polypeptide variants were constructed by SOE-PCR (Splicing by Overhang Extension Polymerase Chain Reaction) with plasmid pDFng156-37. In brief, the first PCR used forward primer EbZn NiaD Fwd and a mutation specific reverse primer (Table 16). The second PCR used reverse primer EbZn NiiA Rev and a mutation specific forward primer (Table 16) containing the sequence coding for the altered amino acid. The mutation specific forward and reverse primers contained 15-20 overlapping nucleotides. The third PCR used the overlapping nucleotides to splice together the fragments produced in the first and second reaction. Finally, using a nested forward primer BGMH110V2F and a nested reverse primer BGMH109V2R, the spliced fragment was amplified by PCR.

The first PCRs for the P. pinophilum GH61 polypeptide variants contained 0.2 picomole of the EbZn NiaD Fwd primer, 0.2 picomole of the reverse primer listed in Table 16, 10 ng of template (pDFng156-37), 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 25 seconds, 66° C. for 30 seconds, and 72° C. for 5 minutes; and a final elongation at 72° C. for 10 minutes. The heat block then went to a 10° C. hold stage.

The second PCRs for the P. pinophilum GH61 variants contained 0.2 picomole of the forward primer listed in Table 16, 0.2 picomole of the EbZn NiiA Rev primer, 10 ng of template (pDFng156-37), 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 25 seconds, 66° C. for seconds, and 72° C. for 5 minutes; and a final elongation at 72° C. for 10 minutes. The heat block then went to a 10° C. hold stage.

Each PCR product was analyzed by 1.0% agarose electrophoresis using TAE buffer where a 4.1 to 5.5 kb (as specified in Table 16) PCR product band was observed indicating proper amplification. The remaining 45 microliters were then treated with 10 units of Dpn I and 1× NEB4 to remove the remaining wild-type template. The reaction was incubated for 4 hours at 37° C. and then purified using a MINELUTE® 96 UF Purification Kit. The purified PCR products were resuspended in deionized water to a final volume equal to 20 μl. The concentration of each fragment was measured using a NanoDrop 2000.

The third SOE-PCR for the P. pinophilum GH61 variants contained 50 to 100 ng of each fragment produced in the first and second PCRs, 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 15 seconds, 68° C. for 30 seconds, and 72° C. for 10 minutes; and a final elongation at 72° C. for 10 minutes. The heat block then went to a 10° C. hold stage. Primer BGMH110V2F primer (0.2 picomole) and primer BGMH109V2R (0.2 picomole) were added during the annealing/elongation step of the fifth cycle to allow for the overlapping nucleotides to splice before amplification.

The wild-type fragment was produced using conditions similar to the third PCR. The reaction was composed of 10 ng of template (pDFng156-37), 0.2 picomole of primer BGMH110V2F, 0.2 picomole of primer BGMH109V2R, 1 nanomole each dATP, dTTP, dGTP, and dCTP, 1× PHUSION® High-Fidelity Buffer, and 0.7 unit of PHUSION® High-Fidelity DNA Polymerase in a final reaction volume of 50 μl. The amplification was performed using an EPPENDORF® MASTERCYCLER® Gradient programmed for 1 cycle at 98° C. for 2 minutes; 35 cycles each at 98° C. for 15 seconds, 68° C. for 30 seconds, and 72° C. for 10 minutes; and a final elongation at 72° C. for 10 minutes. The heat block then went to a 10° C. hold stage.

Each SOE-PCR product was analyzed by 1.0% agarose electrophoresis using TAE buffer where an approximately 8 kb PCR product band was observed indicating proper amplification. The remaining 45 μl of each SOE-PCR product was then purified using a MINELUTE® 96 UF Purification Kit. The purified PCR products were resuspended in deionized water to a final volume equal to 20 μl. The concentration of each fragment was measured using a NanoDrop 2000. The entire volume was then transformed into the Aspergillus oryzae COLs1300 strain as described in Example 21.

TABLE 16

| | | | | | PCR |
|---|---|---|---|---|---|
| Template | Amino Acid | Primer | Primer | | fragment |
| Backbone | Substitution | ID | Direction | Primer Sequence | size (kb) |
| Penicillium | S34F | 1203326 | Fwd | CAAAACATCGTTATCGACG | 5.5 |
| pinophilum | | | | GTAAATTTTAAGCAGTGAT | |
| GH61 | | | | GCATCCATTATTAA (SEQ | |
| | | | | ID NO: 351) | |
| | | 1203327 | Rev | TTTACCGTCGATAACGATG | 4.1 |
| | | | | TTTTGCACAAAACCATG | |
| | | | | (SEQ ID NO: 352) | |
| Penicillium | Q42I | 1203334 | Fwd | AGTTACTCTGGATACCTTG | 5.4 |
| pinophilum | | | | TGAATATCTTCCCCTACGA | |
| GH61 | | | | GTCCAACCCA (SEQ ID NO: | |
| | | | | 353) | |
| | | 1203333 | Rev | ATTCACAAGGTATCCAGAG | 4.2 |
| | | | | TAACTGATTTTTTTGTAAG | |
| | | | | (SEQ ID NO: 354) | |
| Penicillium | Q42V | 1203335 | Fwd | AGTTACTCTGGATACCTTG | 5.4 |
| pinophilum | | | | TGAATGTCTTCCCCTACGA | |
| GH61 | | | | GTCCAACCCA (SEQ ID NO: | |
| | | | | 355) | |
| | | 1203333 | Rev | ATTCACAAGGTATCCAGAG | 4.2 |
| | | | | TAACTGATTTTTTTGTAAG | |
| | | | | (SEQ ID NO: 356) | |
| Penicillium | S47L | 1203338 | Fwd | TGTGAATCAGTTCCCCTAC | 5.4 |
| pinophilum | | | | GAGCTTAACCCACCAGCT | |
| GH61 | | | | GTTATTGGGT (SEQ ID NO: | |
| | | | | 357) | |
| | | 1203337 | Rev | CTCGTAGGGGAACTGATTC | 4.2 |
| | | | | ACAAGGTATCCAGAG (SEQ | |
| | | | | ID NO: 358) | |
| Penicillium | A56C | 1203340 | Fwd | CCACCAGCTGTTATTGGGT | 5.4 |
| pinophilum | | | | GGTGCACAACTGCAACCG | |
| GH61 | | | | ACCTGGGA (SEQ ID NO: | |
| | | | | 359) | |
| | | 1203341 | Rev | CCACCCAATAACAGCTGGT | 4.2 |
| | | | | GGGTTGGACTCGT (SEQ ID | |
| | | | | NO: 360) | |
| Penicillium | A56E | 1203342 | Fwd | CCACCAGCTGTTATTGGGT | 5.4 |
| pinophilum | | | | GGGAGACAACTGCAACCG | |
| GH61 | | | | ACCTGGGA (SEQ ID NO: | |
| | | | | 361 | |
| | | 1203341 | Rev | CCACCCAATAACAGCTGGT | 4.2 |
| | | | | GGGTTGGACTCGT (SEQ ID | |
| | | | | NO: 362) | |
| Penicillium | G138C | 1203353 | Fwd | CTAGACTTTGTCAAGATTG | 5.1 |
| pinophilum | | | | ACCAATGCGGTTTGATCGA | |
| GH61 | | | | CGATACTACCC (SEQ ID | |
| | | | | NO: 363) | |
| | | 1203354 | Rev | TTGGTCAATCTTGACAAAG | 4.5 |
| | | | | TCTAGCTTAGTCTTATCC | |
| | | | | (SEQ ID NO: 364) | |
| Penicillium | T149I | 1203359 | Fwd | ACGATACTACCCCCCCGG | 5.1 |
| pinophilum | | | | GTATCTGGGCTTCCGACAA | |
| GH61 | | | | ACTTATCG (SEQ ID NO: | |
| | | | | 365) | |
| | | 1203360 | Rev | ACCCGGGGGGGTAGTATC | 4.5 |
| | | | | GTCGATCAAACCAC (SEQ | |
| | | | | ID NO: 366) | |
| Penicillium | V164C | 1203365 | Fwd | GCTGCCAACAACAGCTGG | 5.1 |
| pinophilum | | | | ACTTGCACTATCCCCTCCA | |
| GH61 | | | | CCATCGCG (SEQ ID NO: | |
| | | | | 367) | |
| | | 1203366 | Rev | AGTCCAGCTGTTGTTGGCA | 4.5 |
| | | | | GCGATAAGTTTGTCG (SEQ | |
| | | | | ID NO: 368) | |
| Penicillium | V164L | 1203367 | Fwd | GCTGCCAACAACAGCTGG | 5.1 |
| pinophilum | | | | ACTCTTACTATCCCCTCCA | |
| GH61 | | | | CCATCGCG (SEQ ID NO: | |
| | | | | 369) | |
| | | 1203366 | Rev | AGTCCAGCTGTTGTTGGCA | 4.5 |
| | | | | GCGATAAGTTTGTCG (SEQ | |
| | | | | ID NO: 370) | |
| Penicillium | I166L | 1203368 | Fwd | CCAACAACAGCTGGACTGT | 5.0 |
| pinophilum | | | | AACTCTTCCCTCCACCATC | |
| GH61 | | | | GCGCCTGG (SEQ ID NO: | |
| | | | | 372) | |
| | | 1203369 | Rev | AGTTACAGTCCAGCTGTTG | 4.6 |
| | | | | TTGGCAGCGATAAGTT | |
| | | | | (SEQ ID NO: 372) | |
| Penicillium | T169R | 1203370 | Fwd | CTGGACTGTAACTATCCCC | 5.0 |
| pinophilum | | | | TCCCGCATCGCGCCTGGA | |
| GH61 | | | | AACTACGTTT (SEQ ID NO: | |
| | | | | 373) | |
| | | 1203371 | Rev | GGAGGGGATAGTTACAGT | 4.6 |
| | | | | CCAGCTGTTGTTGGC (SEQ | |
| | | | | ID NO: 374) | |
| Penicillium | S186K | 1203372 | Fwd | GCCACGAAATCATTGCTCT | 5.0 |
| pinophilum | | | | TCACAAGGCTGGAAACGC | |
| GH61 | | | | AGACGGTGC (SEQ ID NO: | |
| | | | | 375) | |
| | | 1203373 | Rev | GTGAAGAGCAATGATTTCG | 4.6 |
| | | | | TGGCGCAAAACGTAGT | |
| | | | | (SEQ ID NO: 376) | |
| Penicillium | S186F | 1203374 | Fwd | GCCACGAAATCATTGCTCT | 5.0 |
| pinophilum | | | | TCACTTTGCTGGAAACGCA | |
| GH61 | | | | GACGGTGC (SEQ ID NO: | |
| | | | | 377) | |
| | | 1203373 | Rev | GTGAAGAGCAATGATTTCG | 4.6 |
| | | | | TGGCGCAAAACGTAGT | |
| | | | | (SEQ ID NO: 378) | |
| Penicillium | S186T | 1203375 | Fwd | GCCACGAAATCATTGCTCT | 5.0 |
| pinophilum | | | | TCACACCGCTGGAAACGC | |
| GH61 | | | | AGACGGTGC (SEQ ID NO: | |
| | | | | 379) | |
| | | 1203373 | Rev | GTGAAGAGCAATGATTTCG | 4.6 |
| | | | | TGGCGCAAAACGTAGT | |
| | | | | (SEQ ID NO: 380) | |
| Penicillium | S186Y | 1203376 | Fwd | GCCACGAAATCATTGCTCT | 5.0 |
| pinophilum | | | | TCACTACGCTGGAAACGCA | |
| GH61 | | | | GACGGTGC (SEQ ID NO: | |
| | | | | 381) | |
| | | 1203373 | Rev | GTGAAGAGCAATGATTTCG | 4.6 |
| | | | | TGGCGCAAAACGTAGT | |
| | | | | (SEQ ID NO: 382) | |
| Penicillium | 1200V | 1203377 | Fwd | TGCCCAAAACTACCCTCAA | 4.9 |
| pinophilum | | | | TGCGTCAACTTGGAGATCA | |
| GH61 | | | | CCGGCAGC (SEQ ID NO: | |
| | | | | 383) | |
| | | 1203378 | Rev | GCATTGAGGGTAGTTTTGG | 4.7 |
| | | | | GCACCGTCTGCGT (SEQ ID | |
| | | | | NO: 384) | |
| Penicillium | S213E | 1203381 | Fwd | GCAGCGGAACCGCCGCTC | 4.9 |
| pinophilum | | | | CCGAGGGTACCGCTGGCG | |
| GH61 | | | | AAAAGCTC (SEQ ID NO: | |
| | | | | 385) | |
| | | 1203382 | Rev | GGGAGCGGCGGTTCCGCT | 4.7 |
| | | | | GCCGGTGATCTC (SEQ ID | |
| | | | | NO: 386) | |
| Penicillium | T222R | 1203388 | Fwd | ACCGCTGGCGAAAAGCTC | 4.9 |
| pinophilum | | | | TACCGCTCTACTGACCCCG | |
| GH61 | | | | GTATCTTGG (SEQ ID NO: | |
| | | | | 387) | |
| | | 1203389 | Rev | GTAGAGCTTTTCGCCAGC | 4.7 |
| | | | | GGTACCAGAGGGAG (SEQ | |
| | | | | ID NO: 388) | |
| Penicillium | T245P | 1203393 | Fwd | GACCTACGTTATTCCCGGA | 4.8 |
| pinophilum | | | | CCACCCCTGTGGAGCGGT | |
| GH61 | | | | GCTGCCAA (SEQ ID NO: | |
| | | | | 389) | |
| | | 1203394 | Rev | TGGTCCGGGAATAACGTA | 4.8 |
| | | | | GGTCGACAAGGATTGG | |
| | | | | (SEQ ID NO: 390) | |

### Example 20: Construction of Thielavia terrestris GH61 Polypeptide Variants

The Thielavia terrestris GH61 polypeptide variants were constructed by SOE-PCR (Splicing by Overhang Extension Polymerase Chain Reaction) with plasmid pDFng157-51. In brief, the first PCR used forward primer EbZn NiaD Fwd and a mutation specific reverse primer (Table 17). The second PCR used reverse primer EbZn NiiA Rev and a mutation specific forward primer (Table 17) containing the sequence coding for the altered amino acid. The mutation specific forward and reverse primers contained 15-20 overlapping nucleotides. The third PCR used the overlapping nucleotides to splice together the fragments produced in the first and second reaction. Finally, using a nested forward primer BGMH110V2F and a nested reverse primer BGMH109V2R, the spliced fragment was amplified by PCR.

The first PCRs for the T. terrestris GH61 polypeptide variants contained 0.2 picomole of the EbZn NiaD Fwd primer, 0.4 picomole of the reverse primer listed in Table 17, 10 ng of template (pDFng157-51), 4 nanomoles each dATP, dTTP, dGTP, and dCTP, 1× ADVANTAGE® GC-Melt Buffer, and 2.5 units of ADVANTAGE® GC Genomic LA Polymerase in a final reaction volume of 50 μl. The amplification was performed using an EPPENDORF® MASTERCYCLER® Gradient programmed for 1 cycle at 94° C. for 1 minute; 35 cycles each at 94° C. for 30 seconds, 66° C. for 30 seconds, and 72° C. for 5 minutes; and a final elongation at 72° C. for 10 minutes. The heat block then went to a 10° C. hold stage.

The second PCRs for the T. terrestris GH61 variants contained 0.2 picomole of the forward primer listed in Table 17, 0.4 picomole of the EbZn NiiA Rev primer, 10 ng of template (pDFng157-51), 4 nanomoles each dATP, dTTP, dGTP, and dCTP, lx Advantage® GC-Melt Buffer, and 2.5 units of Advantage® GC Genomic LA Polymerase in a final reaction volume of 50 μl. The amplification was performed using an EPPENDORF® MASTERCYCLER® Gradient programmed for 1 cycle at 94° C. for 1 minute; 35 cycles each at 94° C. for 30 seconds, 66° C. for 30 seconds, and 72° C. for 5 minutes; and a final elongation at 72° C. for 10 minutes. The heat block then went to a 10° C. hold stage.

Each PCR product was analyzed by 1.0% agarose electrophoresis using TAE buffer where a 4.1 to 5.6 kb (as specified in Table 17) PCR product band was observed indicating proper amplification. The remaining 45 microliters were then treated with 10 units of Dpn I and 1× NEB4 to remove the remaining wild-type template. The reaction was incubated for 4 hours at 37° C. and then purified using a MINELUTE® 96 UF Purification Kit. The purified PCR products were resuspended in deionized water to a final volume equal to 20 μl. The concentration of each fragment was measured using a NanoDrop 2000.

The third SOE-PCR for the T. terrestris GH61 variants contained 50 to 100 ng of each fragment produced in the first and second PCRs, 4 nanomoles each dATP, dTTP, dGTP, and dCTP, 1× Advantage® GC-Melt Buffer, and 2.5 units of Advantage® GC Genomic LA Polymerase in a final reaction volume of 50 μl. The amplification was performed using an EPPENDORF® MASTERCYCLER® Gradient programmed for 1 cycle at 94° C. for 1 minute; 35 cycles each at 94° C. for 30 seconds, 66° C. for 30 seconds, and 72° C. for 10 minutes; and a final elongation at 72° C. for 10 minutes. The heat block then went to a 10° C. hold stage. Primer BGMH110V2F primer (0.4 picomole) and primer BGMH109V2R (0.4 picomole) were added during the annealing/elongation step of the fifth cycle to allow for the overlapping nucleotides to splice before amplification.

The wild-type fragment was produced using conditions similar to the third PCR. The reaction was composed of 10 ng of template (pDFng157-51), 0.4 picomole of primer BGMH110V2F, 0.4 picomole of primer BGMH109V2R, 1 nanomole each dATP, dTTP, dGTP, and dCTP, 1× Advantage® GC-Melt Buffer, and 2.5 units of Advantage® GC Genomic LA Polymerase in a final reaction volume of 50 μl. The amplification was performed using an EPPENDORF® MASTERCYCLER® Gradient programmed for 1 cycle at 94° C. for 1 minute; 35 cycles each at 94° C. for 30 seconds, 66° C. for 30 seconds, and 72° C. for 10 minutes; and a final elongation at 72° C. for 10 minutes. The heat block then went to a 10° C. hold stage.

Each SOE-PCR product solution was analyzed by 1.0% agarose electrophoresis using TAE buffer where an approximately 8 kb PCR product band was observed indicating proper amplification. The remaining 45 μl of each SOE-PCR product was then purified using a MINELUTE® 96 UF Purification Kit. The purified PCR products were resuspended in deionized water to a final volume equal to 20 μl. The concentration of each fragment was measured using a NanoDrop 2000. The entire volume was then transformed into the Aspergillus oryzae COLs1300 strain as described in Example 21.

TABLE 17

| | | | | | PCR |
|---|---|---|---|---|---|
| | | | | | fragment |
| Template | Amino Acid | | Primer | | size |
| Backbone | Substitution | Primer ID | Direction | Primer Sequence | (kb) |
| Thielavia | S42I | 1203409 | Fwd | TACCAGGGTTACGATCCGACCAT | 5.6 |
| terrestris | | | | CTTCCCTTACATGCAGAACCCGC | |
| GH61 | | | | (SEQ ID NO: 391) | |
| | | 1203408 | Rev | GGTCGGATCGTAACCCTGGTACG | 4.1 |
| | | | | AGACCCCG (SEQ ID NO: 392) | |
| Thielavia | S42V | 1203410 | Fwd | TACCAGGGTTACGATCCGACCGT | 5.6 |
| terrestris | | | | CTTCCCTTACATGCAGAACCCGC | |
| GH61 | | | | (SEQ ID NO: 393) | |
| | | 1203408 | Rev | GGTCGGATCGTAACCCTGGTACG | 4.1 |
| | | | | AGACCCCG (SEQ ID NO: 394) | |
| Thielavia | Q47L | 1203413 | Fwd | CCGACCTCCTTCCCTTACATGCT | 5.5 |
| terrestris | | | | CAACCCGCCCATCGTGGTCGG | |
| GH61 | | | | (SEQ ID NO: 395) | |
| | | 1203412 | Rev | CATGTAAGGGAAGGAGGTCGGAT | 4.2 |
| | | | | CGTAACCCTG (SEQ ID NO: 396) | |
| Thielavia | S72T | 1203421 | Fwd | TTGCCCCGGATGCCTTCGCCACC | 5.4 |
| terrestris | | | | GGCGATATCATCTGCCACAAGA | |
| GH61 | | | | (SEQ ID NO: 397) | |
| | | 1203420 | Rev | GGCGAAGGCATCCGGGGCAACA | 4.3 |
| | | | | AAGCCGTTG (SEQ ID NO: 398) | |
| Thielavia | V139C | 1203427 | Fwd | CGAGTTCTTCAAGATCGACGAGT | 5.3 |
| terrestris | | | | GCGGCCTGGTCGACGGCAGCTC | |
| GH61 | | | | (SEQ ID NO: 399) | |
| | | 1203428 | Rev | CTCGTCGATCTTGAAGAACTCGA | 4.4 |
| | | | | GCTTGGTCTTG (SEQ ID NO: 400) | |
| Thielavia | V139G | 1203433 | Fwd | CGAGTTCTTCAAGATCGACGAGG | 5.3 |
| terrestris | | | | GCGGCCTGGTCGACGGCAGCTC | |
| GH61 | | | | (SEQ ID NO: 401) | |
| | | 1203428 | Rev | CTCGTCGATCTTGAAGAACTCGA | 4.4 |
| | | | | GCTTGGTCTTG (SEQ ID NO: 402) | |
| Thielavia | V150I | 1203434 | Fwd | ACGGCAGCTCGGCGCCCGGTAT | 5.2 |
| terrestris | | | | CTGGGGCTCCGACCAGCTCAT | |
| GH61 | | | | (SEQ ID NO: 403) | |
| | | 1203435 | Rev | ACCGGGCGCCGAGCTGCCGTCG | 4.5 |
| | | | | ACCAGG (SEQ ID NO: 404) | |
| Thielavia | V165L | 1203442 | Fwd | GCCAACAACAACTCGTGGCTCCT | 5.2 |
| terrestris | | | | CGAGATCCCGCCCACCATCGC | |
| GH61 | | | | (SEQ ID NO: 405) | |
| | | 1203441 | Rev | GAGCCACGAGTTGTTGTTGGCGA | 4.5 |
| | | | | TGAGCTGGT (SEQ ID NO: 406) | |
| Thielavia | A246P | 1203469 | Fwd | CCTACACCGTCCCGGGGCCGCC | 4.9 |
| terrestris | | | | GCTCATCTCCGGCGCCGTCAG | |
| GH61 | | | | (SEQ ID NO: 407) | |
| | | 1203470 | Rev | CGGCCCCGGGACGGTGTAGGTG | 4.8 |
| | | | | ATCGGGG (SEQ ID NO: 408) | |

### Example 21: Expression of the T. aurantiacus GH61A, P. emersonii GH61A, A. aculeatus GH61, Penicillium pinophilum GH61, and Thielavia terrestris GH61 Polypeptides Variants in Aspergillus oryzae COLs1300

Aspergillus oryzae COLs1300 was inoculated onto a COVE-N-Gly plate containing 10 mM urea and incubated at 34° C. until confluent. Spores were collected from the plate by washing with 10 ml of YP medium. The whole spore suspension was used to inoculate 100 ml of COL1300 protoplasting cultivation medium in a 500 ml polycarbonate shake flask. The shake flask was incubated at 30° C. with agitation at 200 rpm for 16-20 hours. Mycelia were filtered through a funnel lined with MIRACLOTH® and washed with 200 ml of 0.6 M MgSO₄. Washed mycelia were resuspended in 20 ml of COLs1300 protoplasting solution in a 125 ml sterile polycarbonate shake flask and incubated at room temperature for 3 minutes. One ml of a solution of 12 mg of BSA per ml of deionized water was added to the shake flask and the shake flask was then incubated at 37° C. with mixing at 65 rpm for 100-150 minutes until protoplasting was complete. The mycelia/protoplast mixture was filtered through a funnel lined with MIRACLOTH® in to a 50 ml conical tube and overlayed with 5 ml of ST solution. The 50 ml conical tube was centrifuged at 1050×g for 15 minutes with slow acceleration/deceleration. After centrifugation, the liquid was separated in 3 phases. The interphase which contained the protoplasts was transferred to a new 50 ml conical tube. Two volumes of STC solution were added to the protoplasts followed by centrifugation at 1050×g for 5 minutes. The supernatant was discarded and the protoplasts were washed twice with 5 ml of STC solution with resuspension of the protoplast pellet, centrifugation at 1050×g for 5 minutes, and decanting of the supernatant each time. After the final decanting, the protoplast pellet was resuspended in STC solution at a concentration of 5×10 7/ml. Protoplasts were frozen at −80° C. until transformation.

A 15 μl volume of each mutant fragment, as described in Examples 16-20, was used to transform 100 μl of A. oryzae COLs1300 protoplasts in a 15 ml round bottom tube. After an initial incubation at room temperature for 15 minutes, 300 μl of PEG solution was added to the 15 ml round bottom tube containing the transformation mixture. The reaction was incubated for an additional 15 minutes at room temperature. Six ml of melted top agar was added to the reaction and the whole mixture was poured evenly onto a sucrose agar plate supplemented with 10 mM NaNO₃ and left at room temperature until the top agar was set. The plates were incubated at 37° C. for 4-6 days. Resulting transformants were picked using sterile inoculating loops and transferred to plates containing COVE-N-gly medium and incubated at 34° C. for approximately 4 days (until sporulation). Spores were inoculated into a deep well 48 well plate containing 0.5 ml of MDU2BP medium incubated at 34° C. for 3 days, stationary in a humidified box. Samples were harvested on the third day by removing the mycelia mat.

### Example 22: Determination of Tm (Melting Temperature) of T. aurantiacus GH61A, P. emersonii GH61A, A. aculeatus GH61, Penicillium pinophilum GH61, and Thielavia terrestris GH61 Variants by Protein Thermal Unfolding Analysis

Protein thermal unfolding of the Thermoascus aurantiacus GH61A, Penicillium emersonii GH61A, Aspergillus aculeatus GH61, Penicillium pinophilum GH61, Thielavia terrestris GH61 variants was determined by protein thermal unfolding analysis described according to Example 10. The broths of the variants, and wild-type polypeptides thereof were prepared as described in Example 21. Average reading of triplicate broths from one to five transformants for each variant was determined, and the increase in Tm for each variant is shown in Table 18.

TABLE 18
Increase of melting temperatures (° C.) of T. aurantiacus GH61A,
P. emersonii GH61A, A. aculeatus GH61, P. pinophilum
GH61, and T. terrestris GH61 polypeptides
variants comparing to wild-type polypeptide
as determined by protein thermal unfolding analysis

| | | Wild-Type Tm (° C.) |
|---|---|---|
| Wild-Type T. aurantiacus GH61A | | 73.0 |
| | Mutation | Δ° C. |
| T. aurantiacus GH61A | Q26I | 3 |
| T. aurantiacus GH61A | Q42I | 2 |
| T. aurantiacus GH61A | Q42V | 2 |
| T. aurantiacus GH61A | S123R | 2 |
| T. aurantiacus GH61A | S138E | 3 |
| T. aurantiacus GH61A | S138K | 3 |
| T. aurantiacus GH61A | S138L | 3 |
| T. aurantiacus GH61A | T163V | 3 |
| T. aurantiacus GH61A | T163E | 3 |
| T. aurantiacus GH61A | S186K | 3 |
| T. aurantiacus GH61A | S186F | 3 |
| T. aurantiacus GH61A | S186T | 3 |
| T. aurantiacus GH61A | S186Y | 3 |
| T. aurantiacus GH61A | I200V | 3 |
| T. aurantiacus GH61A | A213E | 3 |
| T. aurantiacus GH61A | A219C | 3 |
| T. aurantiacus GH61A | A219E | 3 |
| T. aurantiacus GH61A | A219Q | 3 |
| T. aurantiacus GH61A | T248R | 3 |
| | | Wild-Type Tm (° C.) |
| Wild-Type P. emersonii GH61A | | 67.2 |
| | Mutation | Δ° C. |
| P. emersonii GH61A | S46V | 2 |
| P. emersonii GH61A | G76Q | 1 |
| P. emersonii GH61A | S127R | 1 |
| | | Wild-Type Tm (° C.) |
| Wild-Type A. aculeatus GH61 | | 44.8 |
| | Mutation | Δ° C. |
| A. aculeatus GH61 | D70Q | 1 |
| A. aculeatus GH61 | D70T | 2 |
| A. aculeatus GH61 | S136C | 2 |
| A. aculeatus GH61 | S136G | 1 |
| A. aculeatus GH61 | T147I | 3 |
| A. aculeatus GH61 | T161V | 2 |
| A. aculeatus GH61 | T161F | 2 |
| A. aculeatus GH61 | D167R | 5 |
| A. aculeatus GH61 | S184K | 2 |
| A. aculeatus GH61 | S184T | 2 |
| A. aculeatus GH61 | S184Y | 3 |
| A. aculeatus GH61 | L198I | 2 |
| A. aculeatus GH61 | L198V | 1 |
| A. aculeatus GH61 | S217M | 2 |
| A. aculeatus GH61 | S217Q | 2 |
| A. aculeatus GH61 | K220R | 2 |
| A. aculeatus GH61 | A243P | 3 |
| | | Wild-Type Tm (° C.) |
| Wild-Type P. pinophilum GH61 | | 55.4 |
| | Mutation | Δ° C. |
| P. pinophilum GH61 | S34F | 2 |
| P. pinophilum GH61 | Q42I | 4 |
| P. pinophilum GH61 | Q42V | 4 |
| P. pinophilum GH61 | S47L | 2 |
| P. pinophilum GH61 | A56C | 2 |
| P. pinophilum GH61 | A56E | 2 |
| P. pinophilum GH61 | G138C | 5 |
| P. pinophilum GH61 | T149I | 3 |
| P. pinophilum GH61 | V164C | 4 |
| P. pinophilum GH61 | V164L | 3 |
| P. pinophilum GH61 | I166L | 2 |
| P. pinophilum GH61 | T169R | 2 |
| P. pinophilum GH61 | S186K | 5 |
| P. pinophilum GH61 | S186F | 5 |
| P. pinophilum GH61 | S186T | 2 |
| P. pinophilum GH61 | S186Y | 4 |
| P. pinophilum GH61 | I200V | 2 |
| P. pinophilum GH61 | S213E | 2 |
| P. pinophilum GH61 | T222R | 5 |
| P. pinophilum GH61 | T245P | 3 |
| | | Wild-Type Tm (° C.) |
| Wild-Type T. terrestris GH61 | | 56.1 |
| | Mutation | Δ° C. |
| T. terrestris GH61 | S42I | 2 |
| T. terrestris GH61 | S42V | 2 |
| T. terrestris GH61 | Q47L | 2 |
| T. terrestris GH61 | S72T | 2 |
| T. terrestris GH61 | V139C | 2 |
| T. terrestris GH61 | V139G | 2 |
| T. terrestris GH61 | V150I | 1 |
| T. terrestris GH61 | V165L | 2 |
| T. terrestris GH61 | A246P | 3 |

The present invention is further described by the following numbered paragraphs:
[1] A GH61 polypeptide variant, comprising a substitution at one or more positions corresponding to positions 26, 32, 34, 40, 41, 42, 47, 56, 72, 102, 123, 138, 149, 152, 163, 164, 166, 169, 186, 200, 207, 213, 219, 222, 234, 246, 249, and 250 of the mature polypeptide of SEQ ID NO: 30, wherein the variant has cellulolytic enhancing activity and wherein the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99%, but less than 100%, sequence identity to the amino acid sequence of a parent GH61 polypeptide.
[2] The variant of paragraph 1, wherein the parent GH61 polypeptide is selected from the group consisting of: (a) a polypeptide having at least 60% sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411; (b) a polypeptide encoded by a polynucleotide that hybridizes under at least low stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410, or (ii) the full-length complement of (i); (c) a polypeptide encoded by a polynucleotide having at least 60% sequence identity to the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410; and (d) a fragment of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411, which has cellulolytic enhancing activity.
[3] The variant of paragraph 2, wherein the parent GH61 polypeptide has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, at least 99% or 100% sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411.
[4] The variant of paragraph 2, wherein the parent GH61 polypeptide is encoded by a polynucleotide that hybridizes under low stringency conditions, medium stringency conditions, medium-high stringency conditions, high stringency conditions, or very high stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410 or (ii) the full-length complement of (i).
[5] The variant of paragraph 2, wherein the parent GH61 polypeptide is encoded by a polynucleotide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, at least 99%, or 100% sequence identity to the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 408, or 410.
[6] The variant of paragraph 2, wherein the parent GH61 polypeptide comprises or consists of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411.
[7] The variant of paragraph 2, wherein the parent GH61 polypeptide is a fragment of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411, wherein the fragment has cellulolytic enhancing activity.
[8] The variant of any of paragraphs 1-7, which has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99%, but less than 100% sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411.
[9] The variant of any of paragraphs 2-8, wherein the fragment of the variant consists of at least 85% of the amino acid residues, e.g., at least 90% of the amino acid residues or at least 95% of the amino acid residues of the mature polypeptide of the parent GH61 polypeptide.
[10] The variant of any of paragraphs 1-9, wherein the number of substitutions is 1-29, e.g., 1, 2, 3, 4, 5, 6, 7, 8, 9, 10, 11, 12, 13, 14, 15, 16, 17, 18, 19, 20, 21, 22, 23, 24, 25, 26, 27, and 28 substitutions
[11] The variant of any of paragraphs 1-10, which comprises a substitution at a position corresponding to position 26.
[12] The variant of paragraph 11, wherein the substitution is Ile.
[13] The variant of any of paragraphs 1-12, which comprises a substitution at a position corresponding to position 32.
[14] The variant of paragraph 13, wherein the substitution is Glu or Ser.
[15] The variant of any of paragraphs 1-14, which comprises a substitution at a position corresponding to position 34.
[16] The variant of paragraph 15, wherein the substitution is Phe.
[17] The variant of any of paragraphs 1-16, which comprises a substitution at a position corresponding to position 40.
[18] The variant of paragraph 17, wherein the substitution is Ala.
[19] The variant of any of paragraphs 1-18, which comprises a substitution at a position corresponding to position 41.
[20] The variant of paragraph 19, wherein the substitution is Thr.
[21] The variant of any of paragraphs 1-20, which comprises a substitution at a position corresponding to position 42.
[22] The variant of paragraph 21, wherein the substitution is Ile, Glu, or Val.
[23] The variant of any of paragraphs 1-22, which comprises a substitution at a position corresponding to position 47.
[24] The variant of paragraph 23, wherein the substitution is Glu, Leu, or Arg.
[25] The variant of any of paragraphs 1-24, which comprises a substitution at a position corresponding to position 56.
[26] The variant of paragraph 25, wherein the substitution is Cys, Glu, or Thr.
[27] The variant of any of paragraphs 1-26, which comprises a substitution at a position corresponding to position 72.
[28] The variant of paragraph 27, wherein the substitution is Gln or Thr.
[29] The variant of any of paragraphs 1-28, which comprises a substitution at a position corresponding to position 102.
[30] The variant of paragraph 29, wherein the substitution is Lys or Pro.
[31] The variant of any of paragraphs 1-30, which comprises a substitution at a position corresponding to position 123.
[32] The variant of paragraph 31, wherein the substitution is Arg.
[33] The variant of any of paragraphs 1-32, which comprises a substitution at a position corresponding to position 138.
[34] The variant of paragraph 33, wherein the substitution is Cys, Glu, Gly, Lys, Leu, or Met.
[35] The variant of any of paragraphs 1-34, which comprises a substitution at a position corresponding to position 149.
[36] The variant of paragraph 35, wherein the substitution is Ile.
[37] The variant of any of paragraphs 1-36, which comprises a substitution at a position corresponding to position 152.
[38] The variant of paragraph 37, wherein the substitution is Ser.
[39] The variant of any of paragraphs 1-38, which comprises a substitution at a position corresponding to position 163.
[40] The variant of paragraph 39, wherein the substitution is Glu, Phe, or Val.
[41] The variant of any of paragraphs 1-40, which comprises a substitution at a position corresponding to position 164.
[42] The variant of paragraph 41, wherein the substitution is Cys or Leu.
[43] The variant of any of paragraphs 1-42, which comprises a substitution at a position corresponding to position 166.
[44] The variant of paragraph 43, wherein the substitution is Leu.
[45] The variant of any of paragraphs 1-44, which comprises a substitution at a position corresponding to position 169.
[46] The variant of paragraph 45, wherein the substitution is Arg or Cys.
[47] The variant of any of paragraphs 1-46, which comprises a substitution at a position corresponding to position 173.
[48] The variant of paragraph 47, wherein the substitution is Cys.
[49] The variant of any of paragraphs 1-48, which comprises a substitution at a position corresponding to position 186.
[50] The variant of paragraph 49, wherein the substitution is Phe, Lys, Thr, or Tyr.
[51] The variant of any of paragraphs 1-50, which comprises a substitution at a position corresponding to position 200.
[52] The variant of paragraph 51, wherein the substitution is Ile or Val.
[53] The variant of any of paragraphs 1-52, which comprises a substitution at a position corresponding to position 207.
[54] The variant of paragraph 53, wherein the substitution is Pro.
[55] The variant of any of paragraphs 1-54, which comprises a substitution at a position corresponding to position 213.
[56] The variant of paragraph 55, wherein the substitution is Glu.
[57] The variant of any of paragraphs 1-56, which comprises a substitution at a position corresponding to position 219.
[58] The variant of paragraph 57, wherein the substitution is Glu, Met, Gin, or Cys.
[59] The variant of any of paragraphs 1-58, which comprises a substitution at a position corresponding to position 222.
[60] The variant of paragraph 59, wherein the substitution is Arg.
[61] The variant of any of paragraphs 1-60, which comprises a substitution at a position corresponding to position 234.
[62] The variant of paragraph 61, wherein the substitution is Gly or Lys.
[63] The variant of any of paragraphs 1-62, which comprises a substitution at a position corresponding to position 246.
[64] The variant of paragraph 63, wherein the substitution is Pro.
[65] The variant of any of paragraphs 1-64, which comprises a substitution at a position corresponding to position 249.
[66] The variant of paragraph 65, wherein the substitution is Gin, Arg, or Cys.
[67] The variant of any of paragraphs 1-66, which comprises a substitution at a position corresponding to position 250.
[68] The variant of paragraph 67, wherein the substitution is Cys.
[69] The variant of any of paragraphs 1-68, which comprises a substitution at two positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[70] The variant of any of paragraphs 1-68, which comprises a substitution at three positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[71] The variant of any of paragraphs 1-68, which comprises a substitution at four positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[72] The variant of any of paragraphs 1-68, which comprises a substitution at five positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[73] The variant of any of paragraphs 1-68, which comprises a substitution at six positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[74] The variant of any of paragraphs 1-68, which comprises a substitution at seven positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[75] The variant of any of paragraphs 1-68, which comprises a substitution at eight positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[76] The variant of any of paragraphs 1-68, which comprises a substitution at nine positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[77] The variant of any of paragraphs 1-68, which comprises a substitution at ten positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[78] The variant of any of paragraphs 1-68, which comprises a substitution at eleven positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[79] The variant of any of paragraphs 1-68, which comprises a substitution at twelve positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[80] The variant of any of paragraphs 1-68, which comprises a substitution at thirteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[81] The variant of any of paragraphs 1-68, which comprises a substitution at fourteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[82] The variant of any of paragraphs 1-68, which comprises a substitution at fifteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[83] The variant of any of paragraphs 1-68, which comprises a substitution at sixteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[84] The variant of any of paragraphs 1-68, which comprises a substitution at seventeen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[85] The variant of any of paragraphs 1-68, which comprises a substitution at eighteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[86] The variant of any of paragraphs 1-68, which comprises a substitution at nineteen positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[87] The variant of any of paragraphs 1-68, which comprises a substitution at twenty positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[88] The variant of any of paragraphs 1-68, which comprises a substitution at twenty-one positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[89] The variant of any of paragraphs 1-68, which comprises a substitution at twenty-two positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[90] The variant of any of paragraphs 1-68, which comprises a substitution at twenty-three positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[91] The variant of any of paragraphs 1-68, which comprises a substitution at twenty-four positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[92] The variant of any of paragraphs 1-68, which comprises a substitution at twenty-five positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[93] The variant of any of paragraphs 1-68, which comprises a substitution at twenty-six positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[94] The variant of any of paragraphs 1-68, which comprises a substitution at twenty-seven positions corresponding to any of positions 26, 32, 34, 40, 41, 42, 47, 56, 72, 102, 123, 138, 149, 152, 163, 164, 166, 169, 186, 200, 207, 213, 219, 222, 234, 246, 249, and 250.
[95] The variant of any of paragraphs 1-68, which comprises a substitution at each position corresponding to positions 26, 32, 34, 40, 41, 42, 47, 56, 72, 102, 123, 138, 149, 152, 163, 164, 166, 169, 186, 200, 207, 213, 219, 222, 234, 246, 249, and 250.
[96] The variant of any of paragraphs 1-95, which comprises one or more substitutions selected from the group consisting of S26I; G32E,S; Y34F; V40A; N41T; Q42I,E,V; S47E,L,R; S56C,E,T; S72Q,T; T102K,P; A123R; Q138C,E,G,K,L,M; V149I; D152S; T163E,F,V; V164C,L; I166L; S169R,C; S186F,K,T,Y; F200I,V; G207P; S213E; S219E,M,Q,C; K222R; S234G,K; A246P; N249Q,R,C, and A250C.
[97] The variant of any of paragraphs 1-96, which comprises the substitutions S173C+F253C of the mature polypeptide of SEQ ID NO: 36.
[98] The variant of any of paragraphs 1-96, which comprises the substitutions L111V+D152S+M155L+A162W+Q138K+K229W of the mature polypeptide of SEQ ID NO: 30.
[99] The variant of any of paragraphs 1-96, which comprises the substitutions L111V+D152S+M155L+A162W+547E+K229W of the mature polypeptide of SEQ ID NO: 30.
[100] The variant of any of paragraphs 1-96, which comprises the substitutions L111V+D152S+M155L+A162W+S56A+K229W of the mature polypeptide of SEQ ID NO: 30.
[101] The variant of any of paragraphs 1-96, which comprises the substitutions L111V+D152S+M155L+A162W+T102K+K229W of the mature polypeptide of SEQ ID NO: 30.
[102] The variant of any of paragraphs 1-96, which comprises the substitutions L111V+D152S+M155L+A162W+S186T+K229W of the mature polypeptide of SEQ ID NO: 30.
[103] The variant of any of paragraphs 1-96, which comprises the substitutions L111V+D152S+M155L+A162W+K229W+S234G of the mature polypeptide of SEQ ID NO: 30.
[104] The variant of any of paragraphs 1-96, which comprises the substitutions L111V+D152S+M155L+A162W+T102K+E105K+K229W of the mature polypeptide of SEQ ID NO: 30.
[105] The variant of any of paragraphs 1-96, which comprises the substitutions L111V+D152S+M155L+A162W+Q138K+G188F+K229W of the mature polypeptide of SEQ ID NO: 30.
[106] The variant of any of paragraphs 1-96, which comprises the substitutions L111V+D152S+M155L+A162W+Q138K+V149I+G188F+K229W of the mature polypeptide of SEQ ID NO: 30.
[107] The variant of any of paragraphs 1-96, which comprises the substitutions L111V+D152S+M155L+A162W+S169C+G188F+K229W+A250C of the mature polypeptide of SEQ ID NO: 30.
[108] The variant of any of paragraphs 1-96, which comprises the substitutions L111V+D152S+M155L+A162W+S72T+Q138K+V149I+G188F+K229W of the mature polypeptide of SEQ ID NO: 30.
[109] The variant of any of paragraphs 1-96, which comprises the substitutions L111V+D152S+M155L+A162W+Q138K+V149I+G188F+G207P+K229W of the mature polypeptide of SEQ ID NO: 30
[110] The variant of any of paragraphs 1-109, which further comprises a substitution at positions corresponding to positions 111, 152, 155, and 162 of the mature polypeptide of SEQ ID NO: 30, wherein the variant has cellulolytic enhancing activity.
[111] The variant of paragraph 110, wherein the number of additional substitutions is 1-4, e.g., such as 1, 2, 3, or 4 substitutions.
[112] The variant of paragraph 110 or 111, which comprises a substitution at a position corresponding to position 111.
[113] The variant of paragraph 112, wherein the substitution is Val.
[114] The variant of any of paragraphs 110-113, which comprises a substitution at a position corresponding to position 152.
[115] The variant of paragraph 114, wherein the substitution is Ser.
[116] The variant of any of paragraphs 110-115, which comprises a substitution at a position corresponding to position 155.
[117] The variant of paragraph 116, wherein the substitution is Leu.
[118] The variant of any of paragraphs 110-117, which comprises a substitution at a position corresponding to position 162.
[119] The variant of paragraph 118, wherein the substitution is Trp.
[120] The variant of any of paragraphs 110-119, which comprises a substitution at two positions corresponding to any of positions 111, 152, 155, and 162.
[121] The variant of any of paragraphs 110-119, which comprises a substitution at three positions corresponding to any of positions 111, 152, 155, and 162.
[122] The variant of any of paragraphs 110-119, which comprises a substitution at each position corresponding to positions 111, 152, 155, and 162.
[123] The variant of any of paragraphs 110-119, which comprises one or more substitutions selected from the group consisting of L111V, D152S, M155L, and A162W.
[124] The variant of any of paragraphs 1-123, which further comprises a substitution at positions corresponding to positions 96, 98, 200, 202, and 204 of the mature polypeptide of SEQ ID NO: 30, wherein the variant has cellulolytic enhancing activity.
[125] The variant of paragraph 124, wherein the number of additional substitutions is 1-5, e.g., such as 1, 2, 3, 4, or 5 substitutions.
[126] The variant of paragraph 124 or 125, which comprises a substitution at a position corresponding to position 96.
[127] The variant of paragraph 126, wherein the substitution is Val.
[128] The variant of any of paragraphs 124-127, which comprises a substitution at a position corresponding to position 98.
[129] The variant of paragraph 128 wherein the substitution is Leu.
[130] The variant of any of paragraphs 124-129, which comprises a substitution at a position corresponding to position 200.
[131] The variant of paragraph 130, wherein the substitution is Ile.
[132] The variant of any of paragraphs 124-131, which comprises a substitution at a position corresponding to position 202.
[133] The variant of paragraph 132, wherein the substitution is Leu.
[134] The variant of any of paragraphs 124-133, which comprises a substitution at a position corresponding to position 204.
[135] The variant of paragraph 134, wherein the substitution is Val.
[136] The variant of any of paragraphs 124-135, which comprises a substitution at two positions corresponding to any of positions 96, 98, 200, 202, and 204.
[137] The variant of any of paragraphs 124-135, which comprises a substitution at three positions corresponding to any of positions 96, 98, 200, 202, and 204.
[138] The variant of any of paragraphs 124-135, which comprises a substitution at four positions corresponding to any of positions 96, 98, 200, 202, and 204.
[139] The variant of any of paragraphs 124-135, which comprises a substitution at each position corresponding to positions 96, 98, 200, 202, and 204.
[140] The variant of any of paragraphs 124-135, which comprises one or more substitutions selected from the group consisting of I96V, F98L, F200I, I202L, and I204V.
[141] The variant of any of paragraphs 1-140, which further comprises a substitution at positions corresponding to positions 105, 154, 188, 189, 216, and 229 of the mature polypeptide of SEQ ID NO: 30, wherein the variant has cellulolytic enhancing activity.
[142] The variant of paragraph 141, wherein the number of additional substitutions is 1-6, e.g., 1, 2, 3, 4, 5, or 6 substitutions.
[143] The variant of paragraph 141 or 142, which comprises a substitution at a position corresponding to position 105.
[144] The variant of paragraph 143, wherein the substitution is Pro or Lys.
[145] The variant of any of paragraphs 141-144, which comprises a substitution at a position corresponding to position 154.
[146] The variant of paragraph 145, wherein the substitution is Leu.
[147] The variant of any of paragraphs 141-146, which comprises a substitution at a position corresponding to position 188.
[148] The variant of paragraph 147, wherein the substitution is Ala or Trp.
[149] The variant of any of paragraphs 141-148, which comprises a substitution at a position corresponding to position 189.
[150] The variant of paragraph 149, wherein the substitution is Lys.
[151] The variant of any of paragraphs 141-150, which comprises a substitution at a position corresponding to position 216.
[152] The variant of paragraph 151, wherein the substitution is Leu or Tyr.
[153] The variant of any of paragraphs 141-152, which comprises a substitution at a position corresponding to position 229.
[154] The variant of paragraph 153, wherein the substitution is Trp, His, Ile, or Tyr.
[155] The variant of any of paragraphs 141-154, which comprises one or more substitutions selected from the group consisting of E105P,K; E154I, L; G188F, M, A,W; N189H, K; A216L,Y; and A229W,H,I,Y.
[156] The variant of any of paragraphs 1-155, which has an increased thermostability of at least 1.01-fold, e.g., at least 1.05-fold, at least 1.1-fold, at least 1.2-fold, at least 1.3-fold, at least 1.4-fold, at least 1.5-fold, at least 1.8-fold, at least 2-fold, at least 5-fold, at least 10-fold, at least 15-fold, at least 20-fold, at least 25-fold, at least 30-fold, at least 50-fold, at least 75-fold, or at least 100-fold compared to the parent.
[157] An isolated polynucleotide encoding the variant of any of paragraphs 1-156.
[158] A nucleic acid construct comprising the polynucleotide of paragraph 157.
[159] An expression vector comprising the polynucleotide of paragraph 157.
[160] A recombinant host cell comprising the polynucleotide of paragraph 157.
[161] A method of producing a GH61 polypeptide variant, comprising: cultivating the host cell of paragraph 160 under conditions suitable for expression of the variant.
[162] The method of paragraph 161, further comprising recovering the variant.
[163] A transgenic plant, plant part or plant cell transformed with the polynucleotide of paragraph 157.
[164] A method of producing a variant of any of paragraphs 1-156, comprising: cultivating a transgenic plant or a plant cell comprising a polynucleotide encoding the variant under conditions conducive for production of the variant.
[165] The method of paragraph 164, further comprising recovering the variant.
[166] A method for obtaining a GH61 polypeptide variant, comprising introducing into a parent GH61 polypeptide a substitution at one or more positions corresponding to positions 26, 32, 34, 40, 41, 42, 47, 56, 72, 102, 123, 138, 149, 152, 163, 164, 166, 169, 186, 200, 207, 213, 219, 222, 234, 246, 249, and 250 of the mature polypeptide of SEQ ID NO: 30, wherein the variant has cellulolytic enhancing activity; and recovering the variant.
[167] The method of paragraph 166, further comprising introducing into the parent GH61 polypeptide a substitution at one or more positions corresponding to positions 111, 152, 155, and 162 of the mature polypeptide of SEQ ID NO: 30, wherein the variant has cellulolytic enhancing activity.
[168] The method of paragraph 166 or 167, further comprising introducing into the parent GH61 polypeptide a substitution at one or more positions corresponding to positions 96, 98, 200, 202, and 204 of the mature polypeptide of SEQ ID NO: 30, wherein the variant has cellulolytic enhancing activity.
[169] The method of any of paragraphs 166-168, further comprising introducing into the parent GH61 polypeptide a substitution at one or more positions corresponding to positions 105, 154, 188, 189, 216, and 229 of the mature polypeptide of SEQ ID NO: 30, wherein the variant has cellulolytic enhancing activity.
[170] A process for degrading or converting a cellulosic material, comprising: treating the cellulosic material with an enzyme composition in the presence of the GH61 polypeptide variant having cellulolytic enhancing activity of any of paragraphs 1-156.
[171] The process of paragraph 170, wherein the cellulosic material is pretreated.
[172] The process of paragraph 170 or 171, further comprising recovering the degraded cellulosic material.
[173] The process of any of paragraphs 170-172, wherein the enzyme composition comprises one or more enzymes selected from the group consisting of a cellulase, a polypeptide having cellulolytic enhancing activity, a hemicellulase, an esterase, an expansin, a laccase, a ligninolytic enzyme, a pectinase, a peroxidase, a protease, and a swollenin.
[174] The process of paragraph 173, wherein the cellulase is one or more enzymes selected from the group consisting of an endoglucanase, a cellobiohydrolase, and a beta-glucosidase.
[175] The process of paragraph 173, wherein the hemicellulase is one or more enzymes selected from the group consisting of a xylanase, an acetylxylan esterase, a feruloyl esterase, an arabinofuranosidase, a xylosidase, and a glucuronidase.
[176] The process of any of paragraphs 170-175, wherein the degraded cellulosic material is a sugar.
[177] The process of paragraph 176, wherein the sugar is selected from the group consisting of glucose, xylose, mannose, galactose, and arabinose.
[178] A process for producing a fermentation product, comprising: (a) saccharifying a cellulosic material with an enzyme composition in the presence of the GH61 polypeptide variant having cellulolytic enhancing activity of any of paragraphs 1-156; (b) fermenting the saccharified cellulosic material with one or more fermenting microorganisms to produce the fermentation product; and (c) recovering the fermentation product from the fermentation.
[179] The process of paragraph 178, wherein the cellulosic material is pretreated.
[180] The process of paragraph 178 or 179, wherein the enzyme composition comprises one or more enzymes selected from the group consisting of a cellulase, a polypeptide having cellulolytic enhancing activity, a hemicellulase, an esterase, an expansin, a laccase, a ligninolytic enzyme, a pectinase, a peroxidase, a protease, and a swollenin.
[181] The process of paragraph 180, wherein the cellulase is one or more enzymes selected from the group consisting of an endoglucanase, a cellobiohydrolase, and a beta-glucosidase.
[182] The process of paragraph 180, wherein the hemicellulase is one or more enzymes selected from the group consisting of a xylanase, an acetylxylan esterase, a feruloyl esterase, an arabinofuranosidase, a xylosidase, and a glucuronidase.
[183] The process of any of paragraphs 178-182, wherein steps (a) and (b) are performed simultaneously in a simultaneous saccharification and fermentation.
[184] The process of any of paragraphs 178-183, wherein the fermentation product is an alcohol, an alkane, a cycloalkane, an alkene, an amino acid, a gas, isoprene, a ketone, an organic acid, or polyketide.
[185] A process of fermenting a cellulosic material, comprising: fermenting the cellulosic material with one or more fermenting microorganisms, wherein the cellulosic material is saccharified with an enzyme composition in the presence of the GH61 polypeptide variant having cellulolytic enhancing activity of any of paragraphs 1-156.
[186] The process of paragraph 185, wherein the cellulosic material is pretreated before saccharification.
[187] The process of paragraph 185 or 186, wherein the enzyme composition comprises one or more enzymes selected from the group consisting of a cellulase, a polypeptide having cellulolytic enhancing activity, a hemicellulase, an esterase, an expansin, a laccase, a ligninolytic enzyme, a pectinase, a peroxidase, a protease, and a swollenin.
[188] The process of paragraph 187, wherein the cellulase is one or more enzymes selected from the group consisting of an endoglucanase, a cellobiohydrolase, and a beta-glucosidase.
[189] The process of paragraph 187, wherein the hemicellulase is one or more enzymes selected from the group consisting of a xylanase, an acetylxylan esterase, a feruloyl esterase, an arabinofuranosidase, a xylosidase, and a glucuronidase.
[190] The process of any of paragraphs 185-189, wherein the fermenting of the cellulosic material produces a fermentation product.
[191] The process of paragraph 190, further comprising recovering the fermentation product from the fermentation.
[182] The process of paragraph 190 or 191, wherein the fermentation product is an alcohol, an alkane, a cycloalkane, an alkene, an amino acid, a gas, isoprene, a ketone, an organic acid, or polyketide.
[193] A composition comprising the variant of any of paragraphs 1-156.
[194] A whole broth formulation or cell culture composition, comprising the variant of any of paragraphs 1-156.
[195] A detergent composition, comprising a surfactant and the variant of any of paragraphs 1-156.
[196] The composition of paragraph 195, further comprising one or more enzymes selected from the group consisting of an amylase, arabinase, cutinase, carbohydrase, cellulase, galactanase, laccase, lipase, mannanase, oxidase, pectinase, peroxidase, protease, and xylanase.
[197] The composition of paragraph 195 or 196, which is formulated as a bar, a tablet, a powder, a granule, a paste, or a liquid.
[198] A method for cleaning or washing a hard surface or laundry, the method comprising contacting the hard surface or the laundry with the composition of any of paragraphs 195-197.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-20. (canceled)

21. A variant of a parent GH61 polypeptide having cellulolytic enhancing activity, comprising a substitution at one or more positions corresponding to positions 138, 219, 26, 32, 34, 40, 41, 42, 47, 56, 72, 102, 123, 149, 152, 163, 164, 166, 169, 186, 200, 207, 213, 222, 234, 246, 249, and 250 of the mature polypeptide of SEQ ID NO: 2, 4, 6, 8, 10, 12, 14, 16, 18, 20, 22, 24, 26, 28, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 56, 158, 160, 162, 164, 166, 168, 409, or 411, wherein the substitution at position 138 is with Cys, Glu, Gly, Lys, Leu, or Met, the substitution at position 219 is with Glu, Met, Gln, or Cys, the substitution at position 26 is with Ile, the substitution at position 32 is with Glu or Ser, the substitution at position 34 is with Phe, the substitution at position 40 is with Ala, the substitution at position 41 is with Thr, the substitution at position 42 is with Ile, Glu, or Val, the substitution at position 47 is with Glu, Leu, or Arg, the substitution at position 56 is with Cys, Glu, or Thr, the substitution at position 72 is with Gln or Thr, the substitution at position 102 is with Lys or Pro, the substitution at position 123 is with Arg, the substitution at position 149 is with Ile, the substitution at position 152 is with Ser, the substitution at position 163 is with Glu, Phe, or Val, the substitution at position 164 is with Cys or Leu, the substitution at position 166 is with Leu, the substitution at position 169 is with Arg or Cys, the substitution at position 186 is with Phe, Lys, Thr, or Tyr, the substitution at position 200 is with Ile or Val, the substitution at position 207 is with Pro, the substitution at position 213 is with Glu, the substitution at position 222 is with Arg, the substitution at position 234 is with Gly or Lys, the substitution at position 246 is with Pro, the substitution at position 249 is with Gln, Arg, or Cys, and the substitution at position 250 is with Cys, wherein the variant has cellulolytic enhancing activity, wherein the variant has increased thermostability relative to the parent GH61 polypeptide, and wherein the parent GH61 polypeptide is selected from the group consisting of:
(a) a GH61 polypeptide having at least 90% sequence identity to the mature polypeptide of SEQ ID NO: 14, SEQ ID NO: 34, SEQ ID NO: 60, SEQ ID NO: 62, or SEQ ID NO: 64; and
(b) a GH61 polypeptide encoded by a polynucleotide having at least 90% sequence identity to the mature polypeptide coding sequence of SEQ ID NO: 13, SEQ ID NO: 33, SEQ ID NO: 59, SEQ ID NO: 61 or SEQ ID NO: 63;
wherein the variant has at least 90% but less than 100% sequence identity to the mature polypeptide of SEQ ID NO: 14, SEQ ID NO: 34, SEQ ID NO: 60, SEQ ID NO: 62, or SEQ ID NO: 64.

22. The variant of claim 21, wherein the parent GH61 polypeptide has at least 95% sequence identity to the mature polypeptide of SEQ ID NO: 14, SEQ ID NO: 34, SEQ ID NO: 60, SEQ ID NO: 62, or SEQ ID NO: 64.

23. The variant of claim 21, wherein the parent GH61 polypeptide has at least 99% sequence identity to the mature polypeptide of SEQ ID NO: 14, SEQ ID NO: 34, SEQ ID NO: 60, SEQ ID NO: 62, or SEQ ID NO: 64.

24. The variant of claim 21, wherein the parent GH61 polypeptide comprises the mature polypeptide of SEQ ID NO: 14, SEQ ID NO: 34, SEQ ID NO: 60, SEQ ID NO: 62, or SEQ ID NO: 64.

25. The variant of claim 21, wherein the parent GH61 polypeptide consists of the mature polypeptide of SEQ ID NO: 14, SEQ ID NO: 34, SEQ ID NO: 60, SEQ ID NO: 62, or SEQ ID NO: 64.

26. The variant of claim 21, which comprises one or more substitutions selected from the group consisting of S26I; G32E,S; Y34F; V40A; N41T; Q42I,E,V; S47E,L,R; S56C,E,T; S72Q,T; T102K,P; A123R; Q138C,E,G,K,L,M; V149I; D152S; T163E,F,V; V164C,L; I166L; S169R,C; S186F,K,T,Y; F200I,V; G207P; S213E; S219E,M,Q,C; K222R; S234G,K; A246P; N249Q,R,C, and A250C.

27. The variant of claim 21, which further comprises a substitution at one or more positions corresponding to positions 111, 152, 155, 162, 96, 98, 200, 202, 204, 105, 154, 188, 189, and 216 of the mature polypeptide of SEQ ID NO: 30, wherein the variant has cellulolytic enhancing activity and wherein the substitution at position 111 is with Val, the substitution at position 152 is with Ser, the substitution at position 155 is with Leu, the substitution at position 162 is with Trp, the substitution at position 96 is with Val, the substitution at position 98 is with Leu, the substitution at position 200 is with Ile, the substitution at position 202 is with Leu, the substitution at position 204 is with Val, the substitution at position 105 is with Pro or Lys, the substitution at position 154 is with Leu, the substitution at position 188 is with Ala or Trp, the substitution at position 189 is with Lys, and the substitution at position 152 is with Leu or Tyr.

28. The variant of claim 27, which comprises one or more substitutions selected from the group consisting of L111V, D152S, M155L, A162W, I96V, F98L, F200I, I202L, I204V, E105P,K; E154L; G188A,W; N189K; and A216L,Y.

29. A composition comprising the variant of claim 21.

30. A whole broth formulation or cell culture composition, comprising the variant of claim 21.

31. A detergent composition, comprising a surfactant and the variant of claim 21.

32. An isolated polynucleotide encoding the variant of claim 21.

33. A recombinant host cell comprising the polynucleotide of claim 32.

34. A method of producing a GH61 polypeptide variant, comprising: cultivating the recombinant host cell of claim 33 under conditions suitable for expression of the variant.

35. A transgenic plant, plant part or plant cell transformed with the polynucleotide of claim 32.

36. A method of producing the variant of claim 21, comprising: cultivating a transgenic plant or a plant cell comprising a polynucleotide encoding the variant under conditions conducive for production of the variant.

37. A process for degrading or converting a cellulosic material, comprising: treating the cellulosic material with an enzyme composition comprising the GH61 polypeptide variant having cellulolytic enhancing activity of claim 21.

38. The process of claim 37, wherein the cellulosic material is pretreated.

39. The process of claim 37, wherein the enzyme composition further comprises one or more enzymes selected from the group consisting of a cellulase, a polypeptide having cellulolytic enhancing activity, a hemicellulase, an esterase, an expansin, a laccase, a ligninolytic enzyme, a pectinase, a peroxidase, a protease, and a swollenin.

40. The process of claim 37, further comprising recovering the degraded or converted cellulosic material.

41. The process of claim 40, wherein the degraded or converted cellulosic material is a sugar selected from the group consisting of glucose, xylose, mannose, galactose, and arabinose.

42. A process for producing a fermentation product, comprising: (a) saccharifying a cellulosic material with an enzyme composition comprising the GH61 polypeptide variant having cellulolytic enhancing activity of claim 21; (b) fermenting the saccharified cellulosic material with one or more fermenting microorganisms to produce the fermentation product; and (c) recovering the fermentation product from the fermentation.

43. The process of claim 42, wherein the cellulosic material is pretreated.

44. The process of claim 42, wherein the enzyme composition further comprises one or more enzymes selected from the group consisting of a cellulase, a polypeptide having cellulolytic enhancing activity, a hemicellulase, an esterase, an expansin, a laccase, a ligninolytic enzyme, a pectinase, a peroxidase, a protease, and a swollenin.

45. The process of claim 42, wherein steps (a) and (b) are performed simultaneously in a simultaneous saccharification and fermentation.

46. The process of claim 42, wherein the fermentation product is an alcohol, an alkane, a cycloalkane, an alkene, an amino acid, a gas, isoprene, a ketone, an organic acid, or polyketide.

47. A process of fermenting a cellulosic material, comprising: fermenting the cellulosic material with one or more fermenting microorganisms, wherein the cellulosic material is saccharified with an enzyme composition comprising the GH61 polypeptide variant having cellulolytic enhancing activity of claim 21.

48. The process of claim 47, wherein the cellulosic material is pretreated.

49. The process of claim 47, wherein the enzyme composition further comprises one or more enzymes selected from the group consisting of a cellulase, a polypeptide having cellulolytic enhancing activity, a hemicellulase, an esterase, an expansin, a laccase, a ligninolytic enzyme, a pectinase, a peroxidase, a protease, and a swollenin.

50. The process of claim 47, wherein the fermenting of the cellulosic material produces a fermentation product.

51. The process of claim 50, further comprising recovering the fermentation product from the fermentation.

52. The process of claim 50, wherein the fermentation product is an alcohol, an alkane, a cycloalkane, an alkene, an amino acid, a gas, isoprene, a ketone, an organic acid, or polyketide.
